(19) United States
(12) Patent Application Publication
(10) Pub. No.: US 2022/0265693 A1
(43) Pub. Date: Aug. 25, 2022

(54) USE OF E-SELECTIN ANTAGONISTS TO ENHANCE THE SURVIVAL OF RECONSTITUTED, BONE MARROW-DEPLETED HOSTS

(71) Applicant: GLYCOMIMETICS, INC., Rockville, MD (US)

(72) Inventors: John L. MAGNANI, Gaithersburg, MD (US); William E. FOGLER, Lutherville-Timonium, MD (US)

(73) Assignee: GLYCOMIMETICS, INC., Rockville, MD (US)

(21) Appl. No.: 17/597,910

(22) PCT Filed: Jul. 31, 2020
(86) PCT No.: PCT/US2020/044449

§ 371 Date: Jan. 28, 2022

Related U.S. Application Data
(60) Provisional application No. 62/881,307, filed on Jul. 31, 2019.
(60) Provisional application No. 62/910,738, filed on Oct. 4, 2019.
(60) Provisional application No. 63/032,680, filed on May 31, 2020.

(51) Int. Cl. A61K 31/7056; A61K 31/7028; A61K 35/28; A61K 38/19

(52) U.S. Cl. CPC A61K 31/7056; A61K 38/193; A61K 35/28; A61K 31/7028

(57) ABSTRACT

Hematopoietic stem cell (HSC) transplantation is a promising treatment for patients with various hematological diseases, immunodeficiency, autoimmune disorders, and other genetic disorders. Considerable work continues to strive toward the identification of critical factors involved in the successful engraftment and reconstitution of HSC recipients. The identification of these critical components and the understanding of how they may be therapeutically targeted would result in improved patient survival. E-selectin inhibitors for use in increasing survival of individuals that receive HSC transplantation or for reconstitution of depleted and compromised bone marrow are disclosed.

This application claims priority to U.S. Provisional Patent Application Nos. 62/881,307, filed Jul. 31, 2019; 62/910,738, filed Oct. 4, 2019; and 63/032,680, filed May 31, 2020, the disclosures of all of which are incorporated herein by reference in their entireties.

Hematopoietic stem cell (HSC) transplantation represents a curative modality for the treatment of patients with hematological malignant and non-malignant diseases, immunodeficiency, autoimmune disorders, and other genetic disorders. Considerable work continues to strive toward the identification of critical factors involved in the successful engraftment and reconstitution of HSC recipients. The identification of these critical components and the understanding of how they may be therapeutically targeted would result in improved patient survival.

Selectins are a group of structurally similar cell surface receptors important for mediating leukocyte binding to endothelial cells. These proteins are type I membrane proteins and are composed of an amino terminal lectin domain, an epidermal growth factor (EGF)-like domain, a variable number of complement receptor related repeats, a hydrophobic domain spanning region and a cytoplasmic domain. The binding interactions appear to be mediated by contact of the lectin domain of the selectins and various carbohydrate ligands.

There are three known selectins: E-selectin, P-selectin, and L-selectin. E-selectin is found on the surface of activated endothelial cells and binds to the carbohydrate sialyl-Lewis^{x} (SLe^{x}) which is presented as a glycoprotein or glycolipid on the surface of certain leukocytes (monocytes and neutrophils) and helps these cells adhere to capillary walls in areas where surrounding tissue is infected or damaged. E-selectin also binds to sialyl-Lewis^{a} (SLe^{a}) which is expressed on many tumor cells. P-selectin is expressed on inflamed endothelium and platelets and also recognizes SLe^{x} and SLe^{a} but also contains a second site that interacts with sulfated tyrosine. The expression of E-selectin and P-selectin is generally increased when the tissue adjacent to a capillary is infected or damaged. L-selectin is expressed on leukocytes.

Previous studies have investigated the involvement of E-selectin and its interaction with E-selectin ligands in transplantation of HSC (Winkler et al. 2012; Winkler et al. 2014). These studies first demonstrated a novel function for E-selectin that involved the activation of otherwise dormant HSC with the induction of lineage commitment.

However, previous work has also suggested that an E-selectin antagonist could have either a negative or a positive effect on early and/or late complications in patients with transplantation of HSC. For example, antagonism of E-selectin in an HSC recipient could lead to an inhibition of homing and subsequent lack of engraftment and reconstitution with donor cells. Lethally irradiated recipient mice deficient in both P- and E-selectins (P/E−/−), reconstituted with minimal numbers of wild-type bone marrow cells, poorly survived the procedure compared with wild-type recipients (P. S. Frenette et al., 1998). Excess mortality in P/E−/− mice, after a lethal dose of irradiation, was likely caused by a defect of hematopoietic progenitor cell (HPC) homing, since it was observed that the recruitment of HPC to the BM was reduced in P/E−/− animals. Moreover, homing into the bone marrow (BM) of P/E−/− recipient mice was further compromised when a function-blocking VCAM-1 antibody was administered. However, since these studies used mice deficient in both P- and E-selectin, it is not possible to ascertain the direct impact of E-selectin deficiency on HSC recruitment.

Therefore, a need exists in the field to resolve and clarify the role of selectin inhibition, particularly that of E-selectin, as a beneficial factor in increased overall survival of HSC-reconstituted subjects.

In the following description, certain specific details are set forth in order to provide a thorough understanding of various embodiments. However, one skilled in the art will understand that the disclosed embodiments may be practiced without these details. In other instances, well-known structures have not been shown or described in detail to avoid unnecessarily obscuring descriptions of the embodiments. These and other embodiments will become apparent upon reference to the following detailed description and attached drawings.

### BRIEF DESCRIPTION OF THE DRAWINGS

FIG. 1 is a diagram illustrating the prophetic synthesis of compound 11.

FIG. 2 is a diagram illustrating the prophetic synthesis of compound 14.

FIG. 3 is a diagram illustrating the prophetic synthesis of multimeric compounds 21 and 22.

FIG. 4 is a diagram illustrating the prophetic synthesis of multimeric compounds 36 and 37.

FIG. 5 is a diagram illustrating the prophetic synthesis of multimeric compounds 44, 45, and 46.

FIG. 6 is a diagram illustrating the prophetic synthesis of multimeric compounds 55 and 56.

FIG. 7 is a diagram illustrating the prophetic synthesis of compound 60.

FIG. 8 is a diagram illustrating the prophetic synthesis of compound 65.

FIG. 9 is a diagram illustrating the prophetic synthesis of multimeric compounds 66, 67, and 68.

FIG. 10 is a diagram illustrating the prophetic synthesis of multimeric compounds 72 and 73.

FIG. 11 is a diagram illustrating the prophetic synthesis of multimeric compounds 76, 77, and 78.

FIG. 12 is a diagram illustrating the prophetic synthesis of multimeric compounds 86 and 87.

FIG. 13 is a diagram illustrating the prophetic synthesis of multimeric compound 95.

FIG. 14 is a diagram illustrating the prophetic synthesis of multimeric compound 146.

FIG. 15 is a diagram illustrating a prophetic synthesis of multimeric compound 197.

FIG. 16 is a diagram illustrating a synthesis of compound 205.

FIG. 17 is a diagram illustrating the synthesis of multimeric compound 206.

FIG. 18 is a diagram illustrating the synthesis of compound 214.

FIG. 19 is a diagram illustrating the synthesis of multimeric compounds 218, 219, and 220.

FIG. 20 is a diagram illustrating the synthesis of multimeric compound 224.

FIG. 21 is a diagram illustrating the prophetic synthesis of compound 237.

FIG. 22 is a diagram illustrating the prophetic synthesis of compound 241.

FIG. 23 is a diagram illustrating the prophetic synthesis of compound 245.

FIG. 24 is a diagram illustrating the prophetic synthesis of multimeric compound 257.

FIG. 25 is a diagram illustrating the prophetic synthesis of multimeric compounds 261, 262, and 263.

FIG. 26 is a diagram illustrating the prophetic synthesis of multimeric compounds 274, 275, and 276.

FIG. 27 is a diagram illustrating the prophetic synthesis of compound 291.

FIG. 28 is a diagram illustrating the prophetic synthesis of multimeric compounds 294 and 295.

FIG. 29 is a diagram illustrating the prophetic synthesis of multimeric compounds 305, 306, and 307.

FIG. 30 is a diagram illustrating the synthesis of compound 316.

FIG. 31 is a diagram illustrating the synthesis of compound 318.

FIG. 32 is a diagram illustrating the synthesis of compound 145.

FIG. 33 is a diagram illustrating the synthesis of compound 332.

FIG. 34 is a schematic illustrating an experimental model to determine hematopoietic reconstitution of lethally irradiated C57/BL6 (CD45.2+) mice with CD45.1+ congenic B6.SJL cells.

FIG. 35 is a graph illustrating the effect of compound A on the survival of bone marrow-depleted, reconstituted mice.

FIG. 36 is a chart illustrating flow cytometry data evaluating percentage of donor versus recipient cells in blood and bone marrow among reconstituted mice at day 30 post-ablation.

In order to better understand the disclosure, certain exemplary embodiments are discussed herein. In addition, certain terms are discussed to aid in the understanding.

Disclosed herein are methods of increasing survival of subjects that receive HSC transplantation by treating them with an effective amount of at least one E-selectin inhibitor. Also disclosed herein are methods of increasing engraftment and reconstitution in subjects receiving HSC transplantation with the use of at least one E-selectin inhibitor.

In these embodiments, when subjects suffering from a condition resulting in depletion or compromise of bone marrow receive a transplantation of HSCs to reconstitute the absent marrow, inhibition and/or antagonism of selectins may result in increased survival of the subjects.

According to one embodiment, the HSC quiescence and/or HSC mobilization in the subject may be increased. In some embodiments, the subject is a transplant donor. In some embodiments, the subject is a transplant recipient.

In these embodiments, the subject may be suffering from a hematological disease, which may be malignant or non-malignant. Examples of diseases include, but are not limited to, multiple myeloma, Hodgkin and non-Hodgkin lymphoma, acute myeloid leukemia (AML), acute lymphoblastic leukemia (ALL), myelodysplastic syndrome, chronic myeloid leukemia (CML), chronic lymphocytic leukemia, myelofibrosis, essential thrombocytosis, polycythemia vera, and solid tumors, immunodeficiency, autoimmune disorders, and genetic disorders, aplastic anemia, severe combined immune deficiency syndrome (SCID), thalassemia, sickle cell anemia, chronic granulomatous disease, leukocyte adhesion deficiency, Chediak-Higashi syndrome, Kostman syndrome, Fanconi anemia, Blackfan-Diamond anemia, enzymatic disorders, systemic sclerosis, systemic lupus erythematosus, mucopolysaccharidosis, pyruvate kinase deficiency, and multiple sclerosis.

### Definitions

Unless otherwise defined, all technical and scientific terms used herein have the same meaning as commonly understood by one of ordinary skill in the art to which this disclosure belongs. All references cited herein are incorporated by reference in their entireties. To the extent terms or discussion in references conflict with this disclosure, the latter shall control.

As used herein, the singular forms of a word also include the plural form of the word, unless the context clearly dictates otherwise; as examples, the terms “a,” “an,” and “the” are understood to be singular or plural. By way of example, “an element” means one or more element. The term “or” shall mean “and/or” unless the specific context indicates otherwise.

The term “E-selectin ligand” as used herein, refers to a carbohydrate structure that contains the epitope shared by sialyl Le^{a} and sialyl Le^{x}. Carbohydrates are secondary gene products synthesized by enzymes known as glycosyltransferases which are the primary gene products coded for by DNA. Each glycosyltransferase adds a specific monosaccharide in a specific stereochemical linkage to a specific donor carbohydrate chain.

The terms “E-selectin antagonist” and “E-selectin inhibitor” are used interchangeably herein. E-selectin inhibitors are known in the art. Some E-selectin inhibitors are specific for E-selectin only. Other E-selectin inhibitors have the ability to inhibit not only E-selectin but additionally P-selectin or L-selectin or both P-selectin and L-selectin. In some embodiments, an E-selectin inhibitor inhibits E-selectin, P-selectin, and L-selectin.

In some embodiments, an E-selectin inhibitor is a specific glycomimetic antagonist of E-selectin. Examples of E-selectin inhibitors (specific for E-selectin or otherwise) are disclosed in U.S. Pat. No. 9,109,002, the disclosure of which is expressly incorporated by reference in its entirety.

In some embodiments, the E-selectin antagonists suitable for the disclosed compounds and methods include pan-selectin antagonists.

Non-limiting examples of suitable E-selectin antagonists include small molecules, such as nucleic acids, peptides, polypeptides, peptidomimetics, carbohydrates, glycomimetics, lipids and other organic (carbon containing) or inorganic molecules. Suitably, the selectin antagonist is selected from antigen-binding molecules that are immuno-interactive with a selectin, peptides that bind to the selectin and that block cell-cell adhesion, and carbohydrate or peptide mimetics of selectin ligands. In some embodiments, the E-selectin antagonist reduces the expression of a selectin gene or the level or functional activity of an expression product of that gene. For example, the E-selectin antagonist may antagonize the function of the selectin, including reducing or abrogating the activity of at least one of its ligand-binding sites.

In some embodiments, the E-selectin antagonist inhibits an activity of E-selectin or inhibits the binding of E-selectin to one or more E-selectin ligands (which in turn may inhibit a biological activity of E-selectin).

E-selectin antagonists include the glycomimetic compounds described herein. E-selectin antagonists also include antibodies, polypeptides, peptides, peptidomimetics, and aptamers which bind at or near the binding site on E-selectin to inhibit E-selectin interaction with sialyl Le^{a} (sLe^{a}) or sialyl Le^{x} (sLe^{x}).

Further disclosure regarding E-selectin antagonists suitable for the disclosed methods and compounds may be found in U.S. Pat. No. 9,254,322, issued Feb. 9, 2016, and U.S. Pat. No. 9,486,497, issued Nov. 8, 2016, which are both hereby incorporated by reference in their entireties. In some embodiments, the selectin antagonist is chosen from E-selectin antagonists disclosed in U.S. Pat. No. 9,109,002, issued Aug. 18, 2015, which is hereby incorporated by reference in its entirety. In some embodiments, the E-selectin antagonist is chosen from heterobifunctional antagonists disclosed in U.S. Pat. No. 8,410,066, issued Apr. 2, 2013, and US Publication No. US2017/0305951, published Oct. 26, 2017, which are both hereby incorporated by reference in their entireties. Further disclosure regarding E-selectin antagonists suitable for the disclosed methods and compounds may be found in PCT Publication Nos. WO2018/068010, published Apr. 12, 2018, WO2019/133878, published Jul. 4, 2019, and WO2020/139962, published Jul. 2, 2020, which are hereby incorporated by reference in their entireties.

The term “at least one” refers to one or more, such as one, two, etc. For example, the term “at least one C₁₋₄ alkyl group” refers to one or more C₁₋₄ alkyl groups, such as one C₁₋₄ alkyl group, two C₁₋₄ alkyl groups, etc.

The term “pharmaceutically acceptable salts” includes both acid and base addition salts. Non-limiting examples of pharmaceutically acceptable acid addition salts include chlorides, bromides, sulfates, nitrates, phosphates, sulfonates, methane sulfonates, formates, tartrates, maleates, citrates, benzoates, salicylates, and ascorbates. Non-limiting examples of pharmaceutically acceptable base addition salts include sodium, potassium, lithium, ammonium (substituted and unsubstituted), calcium, magnesium, iron, zinc, copper, manganese, and aluminum salts. Pharmaceutically acceptable salts may, for example, be obtained using standard procedures well known in the field of pharmaceuticals.

The term “prodrug” includes compounds that may be converted, for example, under physiological conditions or by solvolysis, to a biologically active compound described herein. Thus, the term “prodrug” includes metabolic precursors of compounds described herein that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release the active compound(s) as described herein in vivo when such prodrug is administered to a subject. Non-limiting examples of prodrugs include ester and amide derivatives of hydroxy, carboxy, mercapto and amino functional groups in the compounds described herein.

This application contemplates all the isomers of the compounds disclosed herein. “Isomer” as used herein includes optical isomers (such as stereoisomers, e.g., enantiomers and diastereoisomers), geometric isomers (such as Z (zusammen) or E (entgegen) isomers), and tautomers. The present disclosure includes within its scope all the possible geometric isomers, e.g., Z and E isomers (cis and trans isomers), of the compounds as well as all the possible optical isomers, e.g. diastereomers and enantiomers, of the compounds. Furthermore, the present disclosure includes in its scope both the individual isomers and any mixtures thereof, e.g. racemic mixtures. The individual isomers may be obtained using the corresponding isomeric forms of the starting material or they may be separated after the preparation of the end compound according to conventional separation methods. For the separation of optical isomers, e.g., enantiomers, from the mixture thereof conventional resolution methods, e.g. fractional crystallization, may be used.

The present disclosure includes within its scope all possible tautomers. Furthermore, the present disclosure includes in its scope both the individual tautomers and any mixtures thereof. Each compound disclosed herein includes within its scope all possible tautomeric forms. Furthermore, each compound disclosed herein includes within its scope both the individual tautomeric forms and any mixtures thereof. With respect to the methods, uses and compositions of the present application, reference to a compound or compounds is intended to encompass that compound in each of its possible isomeric forms and mixtures thereof. Where a compound of the present application is depicted in one tautomeric form, that depicted structure is intended to encompass all other tautomeric forms.

E-selectin inhibitors, such as compound A, can be useful for increasing survival of individuals that receive HSC transplantation for reconstitution of depleted and compromised bone marrow.

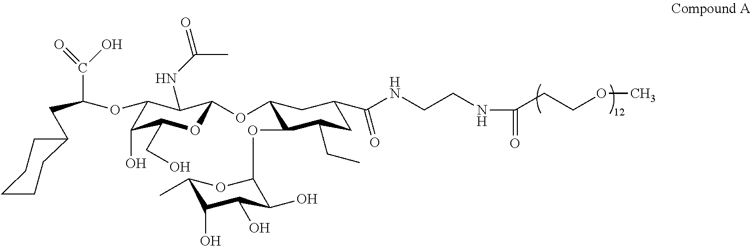

A method of increasing engraftment and reconstitution in a subject receiving HSC transplantation is also comtemplated, wherein the subject in need thereof is administered an effective amount of at least one E-selectin inhibitor, such as compound A.

In some embodiments, the HSC quiescence in the subject is increased. In some embodiments, the HSC mobilization in the subject is increased. In some embodiments, the HSC quiescence and the HSC mobilization in the subject is increased.

In some embodiments, the method further includes inhibiting sinusoidal obstruction syndrome (SOS) in the subject. In some embodiments, the SOS is a hepatic veno-occlusive disease.

In some embodiments, the subject has depleted and/or compromised bone marrow.

In some embodiments, the HSC transplantation is from the subject's peripheral blood. In some embodiments, the HSC transplantation is from the subject's bone marrow.

In some embodiments, the subject is a transplant donor. In some embodiments, the subject is a transplant recipient.

In some embodiments, the subject has received an effective amount of a granulocyte colony-stimulating factor (GCSF).

In some embodiments, the subject has a hematological disease.

In some embodiments the hematological disease is a malignant disease. In some embodiments, the malignant disease is multiple myeloma. In some embodiments, the malignant disease is Hodgkin lymphoma. In some embodiments, the malignant disease is non-Hodgkin lymphoma. In some embodiments, the malignant disease is acute myeloid leukemia (AML). In some embodiments, the malignant disease is acute lymphoblastic leukemia (ALL). In some embodiments, the malignant disease is myelodysplastic syndrome. In some embodiments, the malignant disease is chronic myeloid leukemia (CML). In some embodiments, the malignant disease is chronic lymphocytic leukemia. In some embodiments, the malignant disease is myelofibrosis. In some embodiments, the malignant disease is essential thrombocytosis. In some embodiments, the malignant disease is polycythemia vera. In some embodiments, the malignant disease is a solid tumor.

In some embodiments, the hematological disease is a non-malignant disease. In some embodiments, the non-malignant disease is immunodeficiency. In some embodiments, the non-malignant disease is an autoimmune disorder. In some embodiments, the non-malignant disease is a genetic disorder. In some embodiments, the non-malignant disease is aplastic anemia. In some embodiments, the non-malignant disease is severe combined immune deficiency syndrome (SCID). In some embodiments, the non-malignant disease is thalassemia. In some embodiments, the non-malignant disease is sickle cell anemia. In some embodiments, the non-malignant disease is chronic granulomatous disease. In some embodiments, the non-malignant disease is leukocyte adhesion deficiency. In some embodiments, the non-malignant disease is Chediak-Higashi syndrome. In some embodiments, the non-malignant disease is Kostman syndrome. In some embodiments, the non-malignant disease is Fanconi anemia. In some embodiments, the non-malignant disease is Blackfan-Diamond anemia. In some embodiments, the non-malignant disease is an enzymatic disorder. In some embodiments, the non-malignant disease is systemic sclerosis. In some embodiments, the non-malignant disease is systemic lupus erythematosus. In some embodiments, the non-malignant disease is mucopolysaccharidosis. In some embodiments, the non-malignant disease is pyruvate kinase deficiency. In some embodiments, the non-malignant disease is multiple sclerosis.

In some embodiments, the at least one E-selectin inhibitor is chosen from Compound A:

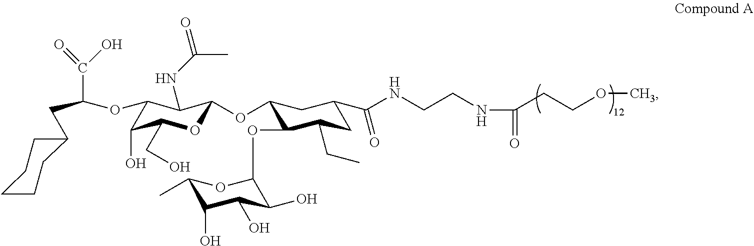

and pharmaceutically acceptable salt thereof.

In some embodiments, the one or more E-selectin inhibitor is administered as a pharmaceutical composition comprising the one or more E-selectin inhibitor, e.g., compound A, in combination with one or more pharmaceutically acceptable excipients.

In some embodiments, the pharmaceutical composition is delivered by subcutaneous delivery. In some embodiments, the pharmaceutical composition is delivered by subcutaneous delivery to the upper arm. In some embodiments, the pharmaceutical composition is delivered by subcutaneous delivery to the abdomen. In some embodiments, the pharmaceutical composition is delivered by subcutaneous delivery to the thigh. In some embodiments, the pharmaceutical composition is delivered by subcutaneous delivery to the upper back. In some pharmaceutical embodiments the composition is delivered by subcutaneous delivery to the buttock. In some embodiments, the pharmaceutical composition is delivered by intravenous infusion.

In various embodiments, the pharmaceutical composition is administered over one or more doses, with one or more intervals between doses. In some embodiments, the pharmaceutical composition is administered over 1, 2, 3, 4, 5, 6, 7, 8, 9, 10, 11, 12, 13, 14, 15, 16, 17, 18, 19, or 20 doses. In some embodiments, the pharmaceutical composition is administered at 6-hour, 12-hour, 18-hour, 24-hour, 48-hour, 72-hour, or 96-hour intervals. In some embodiments, the pharmaceutical composition is administered at one interval, and then administered at a different interval, e.g., 1 dose 24 hours before transplantation, then twice-daily doses throughout transplantation. In some embodiments, the pharmaceutical composition is administered at 1 dose 24 hours before transplantation, then twice-daily doses throughout transplantation up till 48 hours post-transplantation.

The selectin antagonists suitable for the disclosed methods include pan selectin antagonists.

As disclosed herein, any method of inhibiting E-selectin may be used to enhance the survival of reconstituted, bone marrow depleted hosts. Inhibition can be by any means, for example, antibody, small molecule, biologic, inhibitors of gene expression, etc.

Non-limiting examples of suitable selectin antagonists include small molecules, such as nucleic acids, peptides, polypeptides, peptidomimetics, carbohydrates, lipids and other organic (carbon containing) or inorganic molecules. Suitably, the selectin antagonist is selected from antigen-binding molecules that are immuno-interactive with a selectin, peptides that bind to the selectin and that block cell-cell adhesion, and carbohydrate or peptide mimetics of selectin ligands. In some embodiments, the selectin antagonist reduces the expression of a selectin gene or the level or functional activity of an expression product of that gene. For example, the selectin antagonist may antagonize the function of the selectin, including reducing or abrogating the activity of at least one of its ligand-binding sites.

In some embodiments, the E-selectin antagonist is chosen from compounds of Formula Ix:

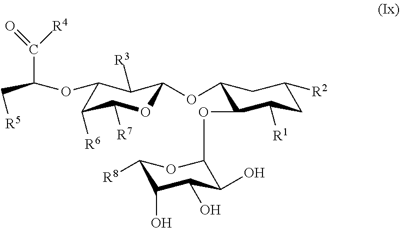

prodrugs of Formula Ix, and pharmaceutically acceptable salts of any of the foregoing, wherein:
R¹ is chosen from C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups;
R² is chosen from H, -M, and -L-M;
R³ is chosen from —OH, —NH₂, —OC(═O)Y¹, —NHC(═O)Y¹, and —NHC(═O)NHY¹ groups, wherein Y¹ is chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₁₈ aryl, and C₁₋₁₃ heteroaryl groups;
R⁴ is chosen from —OH and —NZ¹Z² groups, wherein Z¹ and Z², which may be identical or different, are each independently chosen from H, C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups, wherein Z¹ and Z² may join together to form a ring;
R⁵ is chosen from C₃-C₈ cycloalkyl groups;
R⁶ is chosen from —OH, C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups;
R⁷ is chosen from —CH₂OH, C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups;
R⁸ is chosen from C₁-C₈ alkyl, C₂-C₈ alkenyl, C₂-C₈ alkynyl, C₁-C₈ haloalkyl, C₂-C₈ haloalkenyl, and C₂-C₈ haloalkynyl groups;
L is chosen from linker groups; and
M is a non-glycomimetic moiety chosen from polyethylene glycol, thiazolyl, chromenyl, —C(═O)NH(CH₂)₁₋₄NH₂, C₁₋₈ alkyl, and —C(═O)OY groups, wherein Y is chosen from C₁₋₄ alkyl, C₂₋₄ alkenyl, and C₂₋₄ alkynyl groups.

In some embodiments, the E-selectin antagonist is chosen from compounds of Formula Ix, wherein the non-glycomimetic moiety comprises polyethylene glycol.

In some embodiments, the E-selectin antagonist is chosen from compounds of Formula Ix, wherein the linker is —C(═O)NH(CH₂)₁₋₄NHC(═O)— and the non-glycomimetic moiety comprises polyethylene glycol.

In some embodiments, the E-selectin inhibitor is chosen from the compound of Formula Ix, prodrugs of compounds of Formula Ix and pharmaceutically acceptable salts of any of the foregoing. In some embodiments, the E-selectin inhibitor is the compound of Formula Ix. In some embodiments, the E-selectin inhibitor is chosen from pharmaceutically acceptable salts of the compound of Formula Ix.

In some embodiments, the E-selectin antagonist is chosen from compounds of Formula Ia:

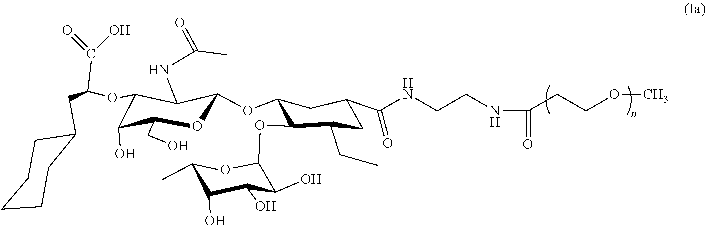

and pharmaceutically acceptable salts thereof, wherein n is chosen from integers ranging from 1 to 100. In some embodiments, n is chosen from 4, 8, 12, 16, 20, 24, and 28. In some embodiments n is 12.

In some embodiments, the E-selectin antagonist is a heterobifunctional antagonist chosen from compounds of Formula II:

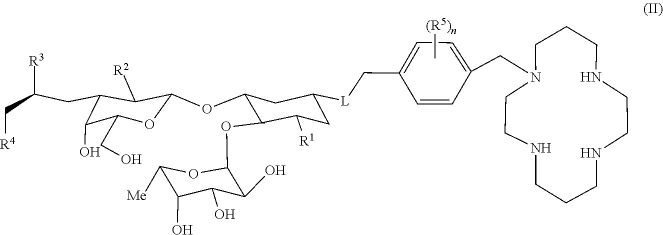

prodrugs of compounds of Formula II, and pharmaceutically acceptable salts of any of the foregoing, wherein:
R¹ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R² is chosen from —OH, —NH₂, —OC(═O)Y¹, —NHC(═O)Y¹, and —NHC(═O)NHY¹ groups, wherein Y¹ is chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₈ aryl, and C₁₋₁₃ heteroaryl groups;
R³ is chosen from —CN, —CH₂CN, and —C(═O)Y² groups, wherein Y² is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Z¹ and Z² may join together to form a ring;
R⁴ is chosen from C₃₋₈ cycloalkyl groups;
R⁵ is independently chosen from H, halo, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
n is chosen from integers ranging from 1 to 4; and
L is chosen from linker groups.

In some embodiments, the E-selectin antagonist is a heterobifunctional antagonist chosen from compounds of Formula IIa:

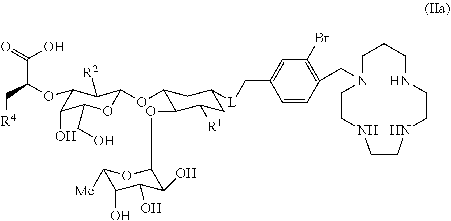

and pharmaceutically acceptable salts thereof.

In some embodiments, the linker groups of Formula Ix and/or Formula II are independently chosen from groups comprising spacer groups, such spacer groups as, for example, —(CH₂)ₚ— and —O(CH₂)ₚ—, wherein p is chosen from integers ranging from 1 to 30. In some embodiments, p is chosen from integers ranging from 1 to 20.

Other non-limiting examples of spacer groups include carbonyl groups and carbonyl-containing groups such as, for example, amide groups. A non-limiting example of a spacer group is

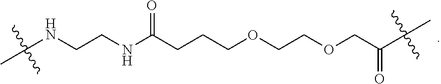 [SMILES: *NCCNC(=O)CCCOCCOCC(*)=O]

In some embodiments, the linker groups of Formula Ix and/or Formula II are independently chosen from

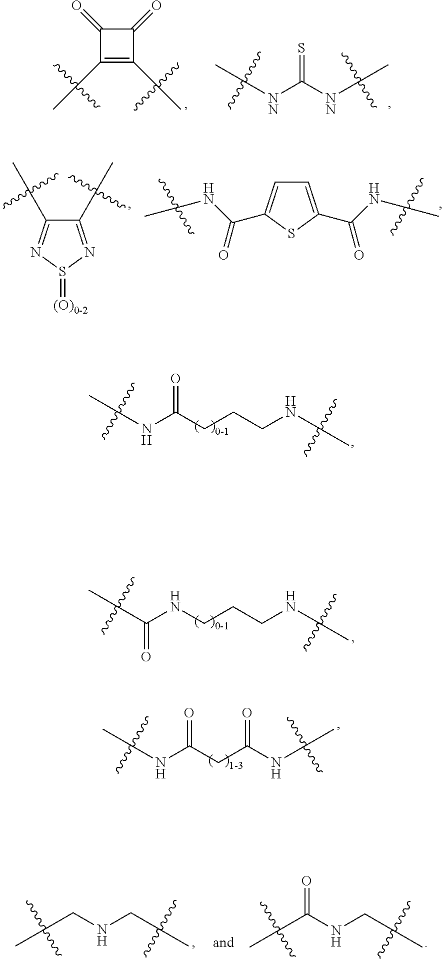

Other linker groups, such as, for example, polyethylene glycols (PEGs) and —C(═O)—NH—(CH₂)ₚ—C(═O)—NH—, wherein p is chosen from integers ranging from 1 to 30, or wherein p is chosen from integers ranging from 1 to 20, will be familiar to those of ordinary skill in the art and/or those in possession of the present disclosure.

In some embodiments, at least one linker group of Formula Ix and/or Formula II is

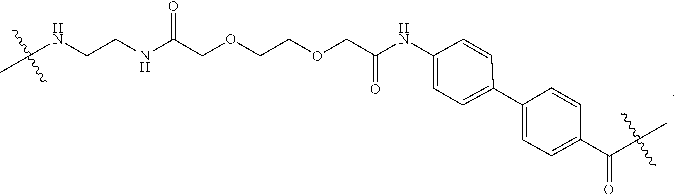 [SMILES: *NCCNC(=O)COCCOCC(=O)Nc1ccc(-c2ccc(C(*)=O)cc2)cc1]

In some embodiments, at least one linker group of Formula Ix and/or Formula II is

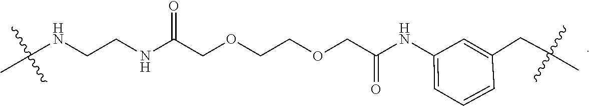 [SMILES: *Cc1cccc(NC(=O)COCCOCC(=O)NCCN*)c1]

In some embodiments, at least one linker group of Formula Ix and/or Formula II is chosen from —C(═O)NH(CH₂)₂NH—, —CH₂NHCH₂—, and —C(═O)NHCH₂—. In some embodiments, at least one linker group is —C(═O)NH(CH₂)₂NH—.

In some embodiments, the E-selectin antagonist is chosen from Compound B:

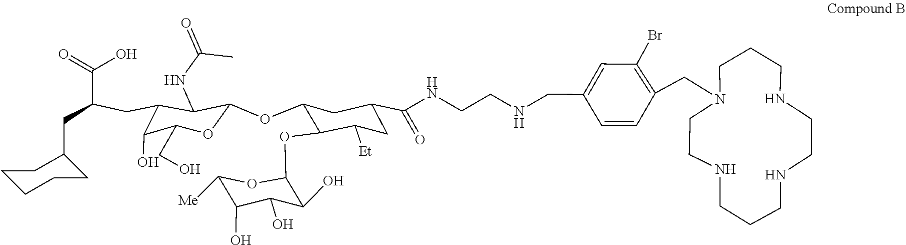 [SMILES: CCC1CC(C(=O)NCCNCc2ccc(CN3CCCN(C)CCNCCCNCC3)c(Br)c2)CC(OC2OC(CO)C(O)C(C[C@@H](CC3CCCCC3)C(=O)O)C2NC(C)=O)C1(C)OC1OC(C)C(O)C(O)C1O]

and pharmaceutically acceptable salts thereof.

In some embodiments, the E-selectin antagonist is chosen from compounds of Formula III:

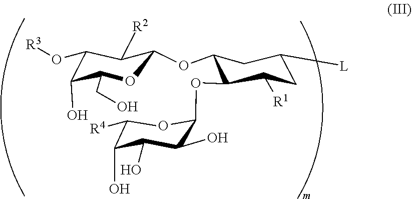

prodrugs of compounds of Formula III, and pharmaceutically acceptable salts of any of the foregoing, wherein:
each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R⁵, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;
each R², which may be identical or different, is independently chosen from halo, —OY¹, —NY¹Y², —OC(═O)Y¹, —NHC(═O)Y¹, and —NHC(═O)NY¹Y² groups, wherein each Y¹ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;
each R³, which may be identical or different, is independently chosen from

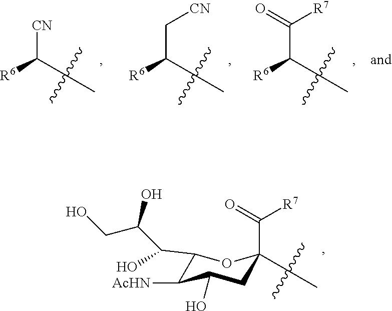

wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;
each R⁴, which may be identical or different, is independently chosen from —CN, C₁₋₄ alkyl, and C₁₋₄ haloalkyl groups;
m is chosen from integers ranging from 2 to 256; and
L is chosen from linker groups.

In some embodiments, the E-selectin antagonist is chosen from compounds of Formula IV:

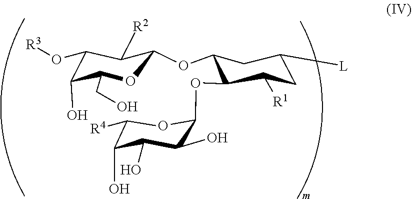

prodrugs of compounds of Formula IV, and pharmaceutically acceptable salts of any of the foregoing, wherein:
each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R⁵, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;
each R², which may be identical or different, is independently chosen from halo, —OY¹, —NY¹Y², —OC(═O)Y¹, —NHC(═O)Y¹, and —NHC(═O)NY¹Y² groups, wherein each Y¹ and each Y², which may be identical or different, are independently chosen from H. C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;
each R, which may be identical or different, is independently chosen from

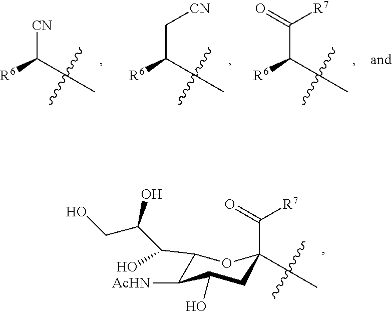

wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;
each R⁴, which may be identical or different, is independently chosen from —CN, C₁₋₄ alkyl, and C₁₋₄ haloalkyl groups;
m is 2; and
L is chosen from

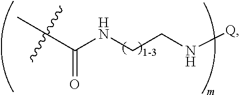

wherein Q is a chosen from

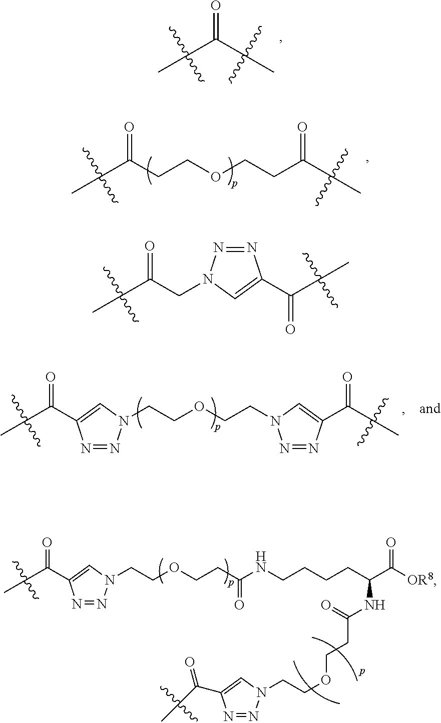

wherein R⁸ is chosen from H, C₁₋₈ alkyl, C₆₋₁₈ aryl, C₇₋₁₉ arylalkyl, and C₁₋₁₃ heteroaryl groups and each p, which may be identical or different, is independently chosen from integers ranging from 0 to 250.

In some embodiments, the E-selectin antagonist of Formula III or Formula IV is chosen from compounds of the following Formula IIIa/IVa (see definitions of L and m for Formula III or IV above):

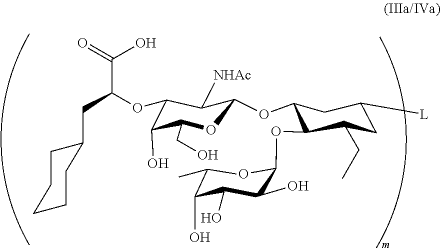

In some embodiments, the E-selectin antagonist of Formula III or Formula IV is chosen from compounds of the following Formula IIIb/IVb (see definitions of L and m for Formula III or IV above):

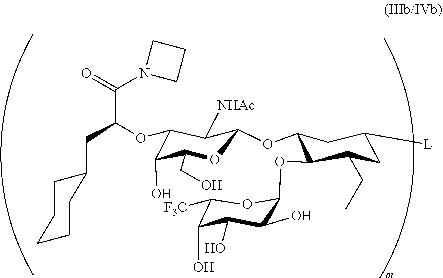

In some embodiments, the E-selectin antagonist is Compound C:

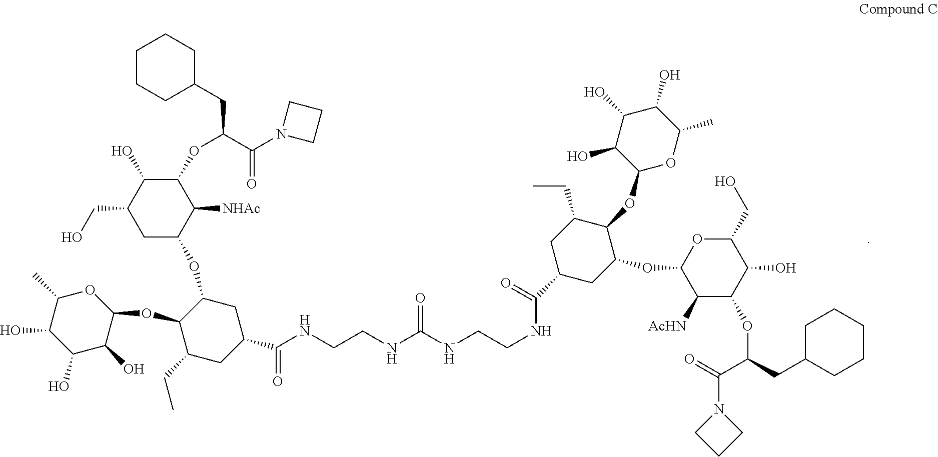 [SMILES: CC[C@H]1C[C@@H](C(=O)NCCNC(=O)NCCNC(=O)[C@@H]2C[C@H](CC)[C@@H](O[C@@H]3O[C@@H](C)[C@@H](O)[C@@H](O)[C@@H]3O)[C@H](O[C@@H]3C[C@H](CO)[C@H](O)[C@H](O[C@@H](CC4CCCCC4)C(=O)N4CCC4)[C@H]3NC(C)=O)C2)C[C@@H](O[C@@H]2O[C@H](CO)[C@H](O)[C@H](O[C@@H](CC3CCCCC3)C(=O)N3CCC3)[C@H]2NC(C)=O)[C@@H]1O[C@@H]1O[C@@H](C)[C@@H](O)[C@@H](O)[C@@H]1O]

In some embodiments, the E-selectin antagonist is a heterobifunctional inhibitor of E-selectin and Galectin-3, chosen from compounds of Formula V:

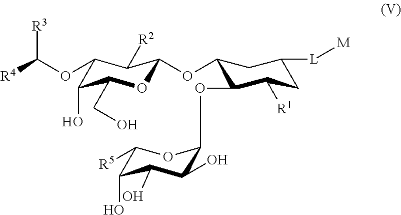

prodrugs of compounds of Formula V, and pharmaceutically acceptable salts of any of the foregoing, wherein:
R¹ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl,

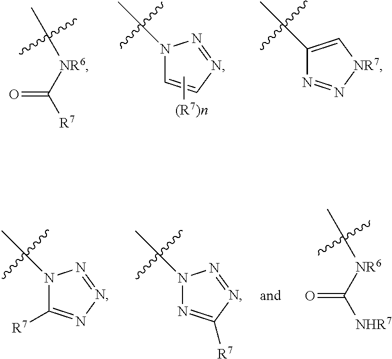

groups, wherein n is chosen from integers ranging from 0 to 2, R⁶ is chosen from H, C₁₋₈ alkyl, C₂₋₈ alkenyl, C₂₋₈ alkynyl, C₄₋₁₆ cycloalkylalkyl, and —C(═O)R⁷ groups, and each R⁷ is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
R² is chosen from —OH, —OY¹, halo, —NH₂, —NY¹Y², —OC(═O)Y¹, —NHC(═O)Y¹, and —NHC(═O)NHY¹ groups, wherein Y¹ and Y², which may be the same or different, are independently chosen from C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl groups, wherein Y¹ and Y² may join together along with the nitrogen atom to which they are attached to form a ring;
R³ is chosen from —CN, —CH₂CN, and —C(═O)Y³ groups, wherein Y³ is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₇₋₁₂ arylalkyl groups, wherein Z¹ and Z² may join together along with the nitrogen atom to which they are attached to form a ring;
R⁴ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, and C₆₋₁₈ aryl groups;
R⁵ is chosen from —CN, C₁₋₈ alkyl, and C₁₋₄ haloalkyl groups;
M is chosen from

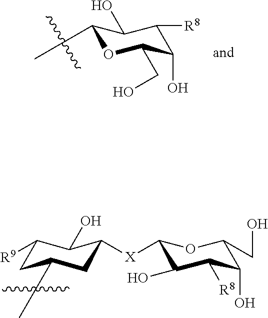

groups, wherein X is chosen from 0 and S, and R⁸ and R⁹, which may be identical or different, are independently chosen from C₆₋₁₈ aryl, C₁₋₁₃ heteroaryl, C₇₋₁₉ arylalkyl, C₇₋₁₉ arylalkoxy, C₂₋₁₄ heteroarylalkyl, C₂₋₁₄ heteroarylalkoxy, and —NHC(═O)Y⁴ groups, wherein Y⁴ is chosen from C₁₋₈ alkyl, C₂₋₁₂ heterocyclyl, C₆₋₁₈ aryl, and C₁₋₁₃ heteroaryl groups; and
L is chosen from linker groups.

In some embodiments, the E-selectin antagonist is chosen from compounds having the following Formulae:

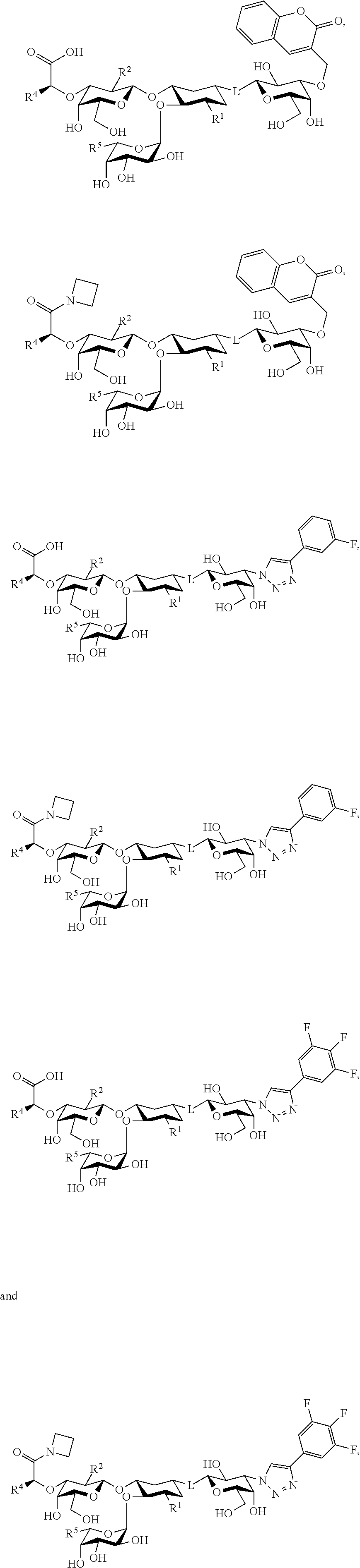

In some embodiments, the E-selectin antagonist is chosen from compounds having the following Formulae:

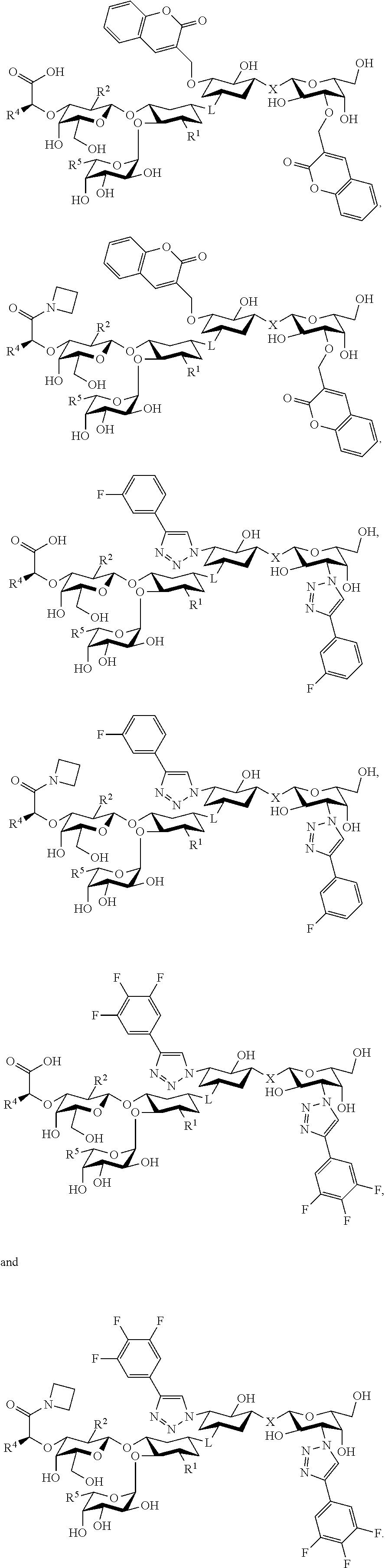

In some embodiments, the E-selectin antagonist is Compound D:

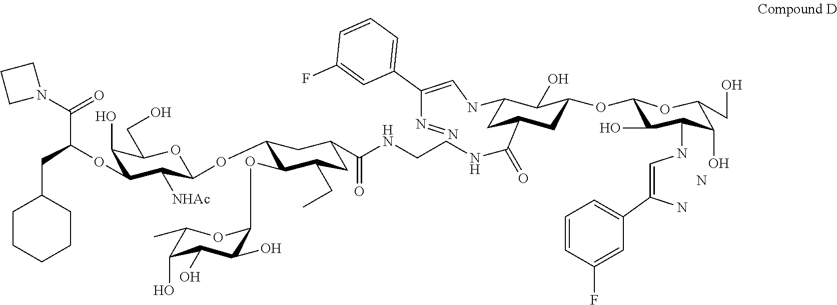

In some embodiments, the E-selectin antagonist is chosen from compounds of Formula VI:

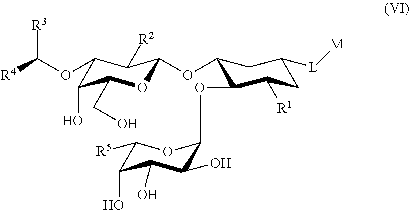

prodrugs of compounds of Formula VI, and pharmaceutically acceptable salts of any of the foregoing, wherein:
R¹ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl,

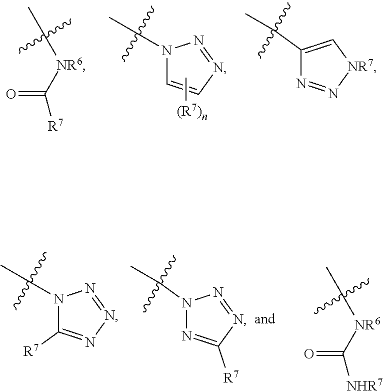

groups, wherein n is chosen from integers ranging from 0 to 2, R⁶ is chosen from H, C₁₋₈ alkyl, C₂₋₈ alkenyl, C₂₋₈ alkynyl, C₄₋₁₆ cycloalkylalkyl, and —C(═O)R groups, and each R is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
R² is chosen from —OH, —OY¹, halo, —NH₂, —NY¹Y². —OC(═O)Y¹, —NHC(═O)Y¹, and —NHC(═O)NHY¹ groups, wherein Y¹ and Y², which may be the same or different, are independently chosen from C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl groups, or Y¹ and Y² join together along with the nitrogen atom to which they are attached to form a ring;
R³ is chosen from —CN, —CH₂CN, and —C(═O)Y³ groups, wherein Y³ is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₇₋₁₂ arylalkyl groups, or Z¹ and Z² join together along with the nitrogen atom to which they are attached to form a ring;
R⁴ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, and C₆₋₁₈ aryl groups;
R⁵ is chosen from —CN, C₁₋₈ alkyl, and C₁₋₄ haloalkyl groups;
M is chosen from

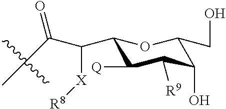

groups,
wherein
X is chosen from —O—, —S—, —C—, and —N(R¹⁰)—, wherein R¹⁰ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups,
Q is chosen from H, halo, and —OZ³ groups, wherein Z³ is chosen from H and C₁₋₈ alkyl groups,
R⁸ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₁₃ heteroaryl, C₇₋₁₉ arylalkyl, and C₂₋₁₄ heteroarylalkyl groups, wherein the C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₁₃ heteroaryl, C₇₋₁₉ arylalkyl, and C₂₋₁₄ heteroarylalkyl groups are optionally substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, —C(═O)NZ⁴Z⁵, and —SO₂Z⁴ groups, wherein Z⁴ and Z⁵, which may be identical or different, are independently chosen from H, C₁₋₈ alkyl, and C₁₋₈ haloalkyl groups, or Z⁴ and Z⁵ join together along with the nitrogen atom to which they are attached to form a ring,
R⁹ is chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from R¹¹, C₁₋₈ alkyl, C₁₋₈ haloalkyl, —C(═O)OZ⁶, and —C(═O)NZ⁶Z groups, wherein R¹¹ is independently chosen from C₆₋₁₈ aryl groups optionally substituted with one or more groups independently chosen from halo, C₁₋₈ alkyl, —OZ⁸, —C(═O)OZ⁸, and —C(═O)NZ⁸Z⁹ groups, wherein Z⁶, Z⁷, Z⁸ and Z⁹, which may be identical or different, are independently chosen from H and C₁₋₈ alkyl groups, or Z⁶ and Z⁷ join together along with the nitrogen atom to which they are attached to form a ring and/or Z⁸ and Z⁹ join together along with the nitrogen atom to which they are attached to form a ring, and
wherein each of Z³, Z⁴, Z⁵, Z⁶, Z⁷, Z⁸, and Z⁹ is optionally substituted with one or more groups independently chosen from halo and —OR¹² groups, wherein R¹² is independently chosen from H and C₁₋₈ alkyl groups; and
L is chosen from linker groups.

In some embodiments of Formula VI, M is chosen from

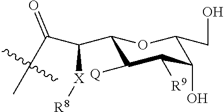

groups.

In some embodiments of Formula VI, M is chosen from

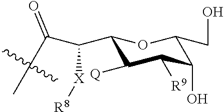

groups.

In some embodiments of Formula VI, linker groups may be chosen from groups comprising spacer groups, such spacer groups as, for example, —(CH₂)ₜ— and —O(CH₂)ₜ—, wherein t is chosen from integers ranging from 1 to 20. Other non-limiting examples of spacer groups include carbonyl groups and carbonyl-containing groups such as, for example, amide groups. A non-limiting example of a spacer group is

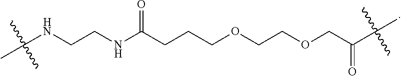

In some embodiments of Formula VI, the linker group is chosen from

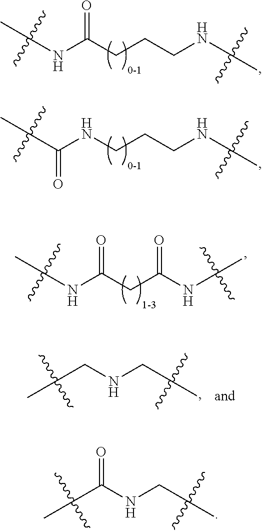

In some embodiments of Formula VI, the linker group is chosen from polyethylene glycols (PEGs), —C(═O)NH(CH₂)O—, —C(═O)NH(CH₂) NHC(═O)—, —C(═O)NHC(═O)(CH₂)NH—, and —C(═O)NH(CH₂)ᵥC(═O)NH— groups, wherein v is chosen from integers ranging from 2 to 20. In some embodiments, v is chosen from integers ranging from 2 to 4. In some embodiments, v is 2. In some embodiments, v is 3. In some embodiments, v is 4.

In some embodiments of Formula VI, the linker group is

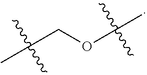

In some embodiments of Formula VI, the linker group is

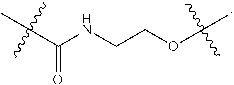

In some embodiments of Formula VI, the linker group is

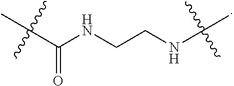

In some embodiments of Formula VI, the linker group is

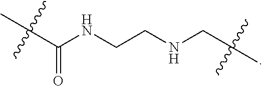

In some embodiments of Formula VI, the linker group is

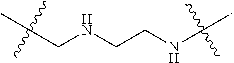

In some embodiments of Formula VI, the linker group is

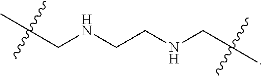

In some embodiments of Formula VI, the linker group is

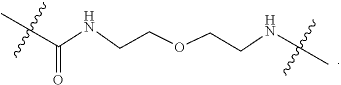 [SMILES: *NCCOCCNC(*)=O]

In some embodiments of Formula VI, the linker group is

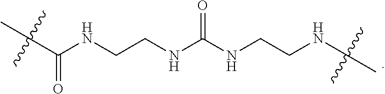 [SMILES: *NCCNC(=O)NCCNC(*)=O]

In some embodiments of Formula VI, the linker group is

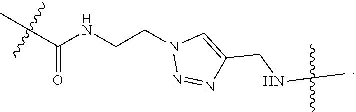 [SMILES: *NCc1cn(CCNC(*)=O)nn1]

In some embodiments, the E-selectin antagonist is a multimeric inhibitor of E-selectin, Galectin-3, and/or CXCR4, chosen from compounds of Formula VII:

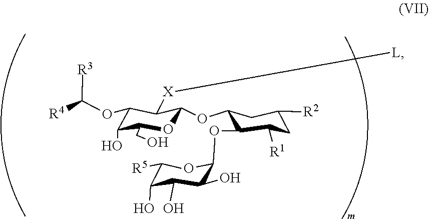

prodrugs of compounds of Formula VII, and pharmaceutically acceptable salts of any of the foregoing, wherein:
each R¹, which may be identical or different, is independently chosen from H, C₁₋₁₂ alkyl, C₂₋₁₂ alkenyl, C₂₋₁₂ alkynyl, Ct-s haloalkyl, C₂₋₈ haloalkenyl, C₂₋₈ haloalkynyl,

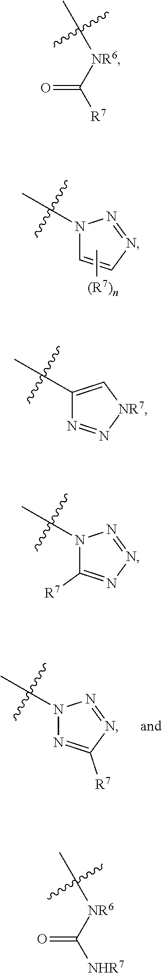

groups, wherein each n, which may be identical or different, is chosen from integers ranging from 0 to 2, each R⁶, which may be identical or different, is independently chosen from H, C₁₋₈ alkyl, C₂₋₈ alkenyl, C₂₋₈ alkynyl, C₄₋₁₆ cycloalkylalkyl, and —C(═O)R⁷ groups, and each R⁷, which may identical or different, is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
each R², which may be identical or different, is independently chosen from H, a non-glycomimetic moiety, and a linker-non-glycomimetic moiety, wherein each non-glycomimetic moiety, which may be identical or different, is independently chosen from galectin-3 inhibitors, CXCR4 chemokine receptor inhibitors, polyethylene glycol, thiazolyl, chromenyl, C₁₋₈ alkyl, R⁸, C₆₋₁₈ aryl-R⁸, C₁₋₁₂ heteroaryl-R⁸,

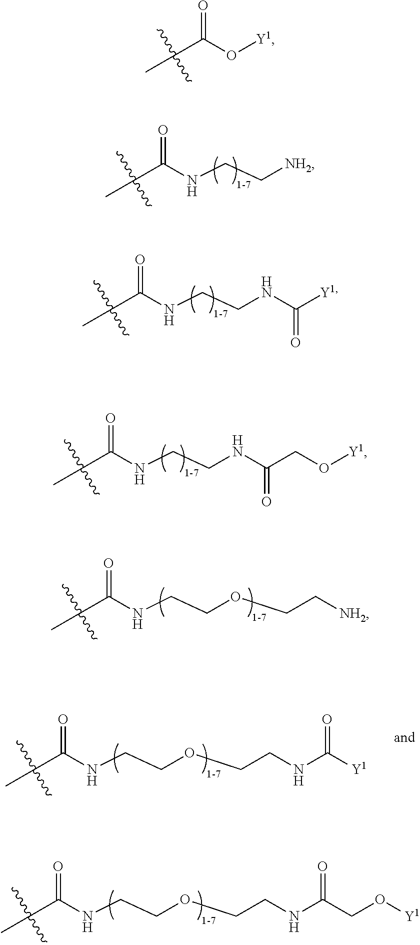

groups,
wherein each Y¹, which may be identical or different, is independently chosen from C₁₋₄ alkyl, C₂₋₄ alkenyl, and C₂₋₄ alkynyl groups and wherein each R⁸, which may be identical or different, is independently chosen from C₁₋₁₂ alkyl groups substituted with at least one substituent chosen from —OH, —OSO₃Q, —OPO₃Q₂, —CO₂Q, and —SO₃Q groups and C₂₋₁₂ alkenyl groups substituted with at least one substituent chosen from —OH, —OSO₃Q, —OPO₃Q₂, —CO₂Q, and —SO₃Q groups, wherein each Q, which may be identical or different, is independently chosen from H and pharmaceutically acceptable cations;
each R³, which may be identical or different, is independently chosen from —CN, —CH₂CN, and —C(═O)Y² groups, wherein each Y², which may be identical or different, is independently chosen from C₁₋₈ alkyl, C₂₋₈ alkenyl, C₂₋₈ alkynyl, —OZ¹, —NHOH, —NHOCH₃, —NHCN, and —NZ¹Z² groups, wherein each Z¹ and Z², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, and C₇₋₁₂ arylalkyl groups, wherein Z¹ and Z² may join together along with the nitrogen atom to which they are attached to form a ring;
each R⁴, which may be identical or different, is independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₄₋₁₆ cycloalkylalkyl, and C₆₋₁₈ aryl groups;
each R⁵, which may be identical or different, is independently chosen from —CN, C₁₋₁₂ alkyl, and C₁₋₁₂ haloalkyl groups;
each X, which may be identical or different, is independently chosen from —O— and —N(R⁹)—, wherein each R⁹, which may be identical or different, is independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
m is chosen from integers ranging from 2 to 256; and
L is independently chosen from linker groups.

In some embodiments of Formula VII, at least one linker group is chosen from groups comprising spacer groups, such spacer groups as, for example, —(CH₂)_{z}— and —O(CH₂)_{z}—, wherein z is chosen from integers ranging from 1 to 250. Other non-limiting examples of spacer groups include carbonyl groups and carbonyl-containing groups such as, for example, amide groups. A non-limiting example of a spacer group is

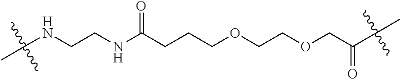 [SMILES: *NCCNC(=O)CCCOCCOCC(*)=O]

In some embodiments of Formula VII, at least one linker group is chosen from

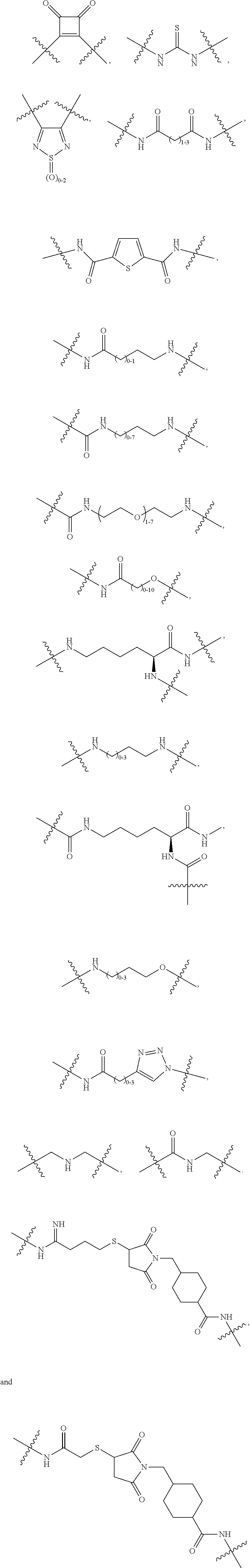

groups.

Other linker groups for certain embodiments of Formula VII, such as, for example, polyethylene glycols (PEGs) and —C(═O)—NH—(CH₂)_{z}—C(═O)—NH—, wherein z is chosen from integers ranging from 1 to 250, will be familiar to those of ordinary skill in the art and/or those in possession of the present disclosure.

In some embodiments of Formula VII, at least one linker group is

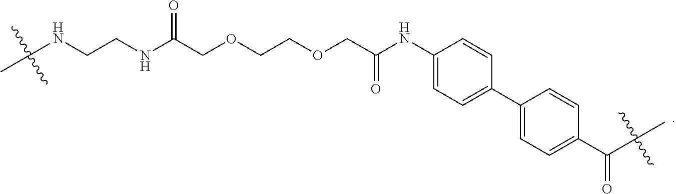 [SMILES: *NCCNC(=O)COCCOCC(=O)Nc1ccc(-c2ccc(C(*)=O)cc2)cc1]

In some embodiments of Formula VII, at least one linker group is

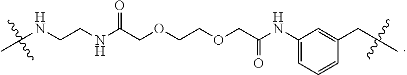 [SMILES: *Cc1cccc(NC(=O)COCCOCC(=O)NCCN*)c1]

In some embodiments of Formula VII, at least one linker group is chosen from —C(═O)NH(CH₂)₂NH—, —CH₂NHCH₂—, and —C(═O)NHCH₂—. In some embodiments of Formula VII, at least one linker group is —C(═O)NH(CH₂)₂NH—.

In some embodiments of Formula VII, L is chosen from dendrimers. In some embodiments of Formula VII, L is chosen from polyamidoamine (“PAMAM”) dendrimers. In some embodiments of Formula VII, L is chosen from PAMAM dendrimers comprising succinamic. In some embodiments of Formula VII, L is PAMAM GO generating a tetramer. In some embodiments of Formula VII, L is PAMAM G1 generating an octamer. In some embodiments of Formula VII, L is PAMAM G2 generating a 16-mer. In some embodiments of Formula VII, L is PAMAM G3 generating a 32-mer. In some embodiments of Formula VII, L is PAMAM G4 generating a 64-mer. In some embodiments, L is PAMAM G5 generating a 128-mer.

In some embodiments of Formula VII, m is 2 and L is chosen from

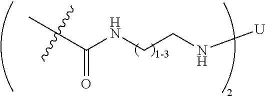

groups,
wherein U is chosen from

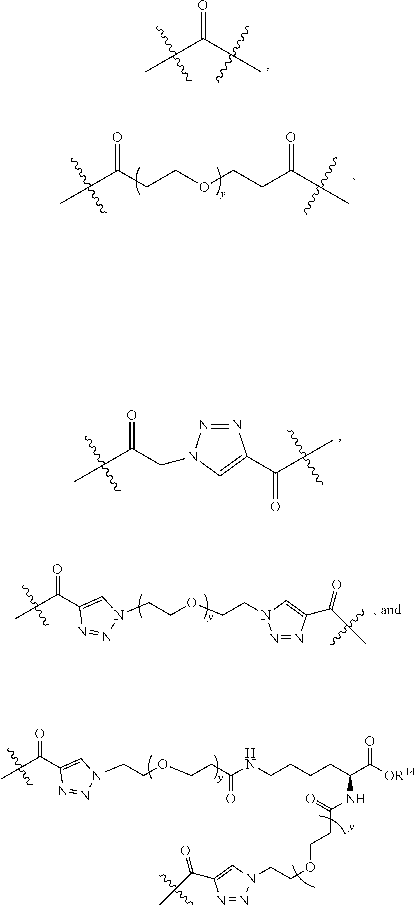

groups,
wherein R¹⁴ is chosen from H, C₁₋₈ alkyl, C₆₋₁₈ aryl, C₇₋₁₉ arylalkyl, and C₁₋₁₃ heteroaryl groups and each y, which may be identical or different, is independently chosen from integers ranging from 0 to 250. In some embodiments of Formula VII, R¹⁴ is chosen from C₁₋₈ alkyl. In some embodiments of Formula VII, R¹⁴ is chosen from C₇₋₁₉ arylalkyl. In some embodiments of Formula VII, R¹⁴ is H. In some embodiments of Formula VII, R¹⁴ is benzyl.

In some embodiments of Formula VII, L is chosen from

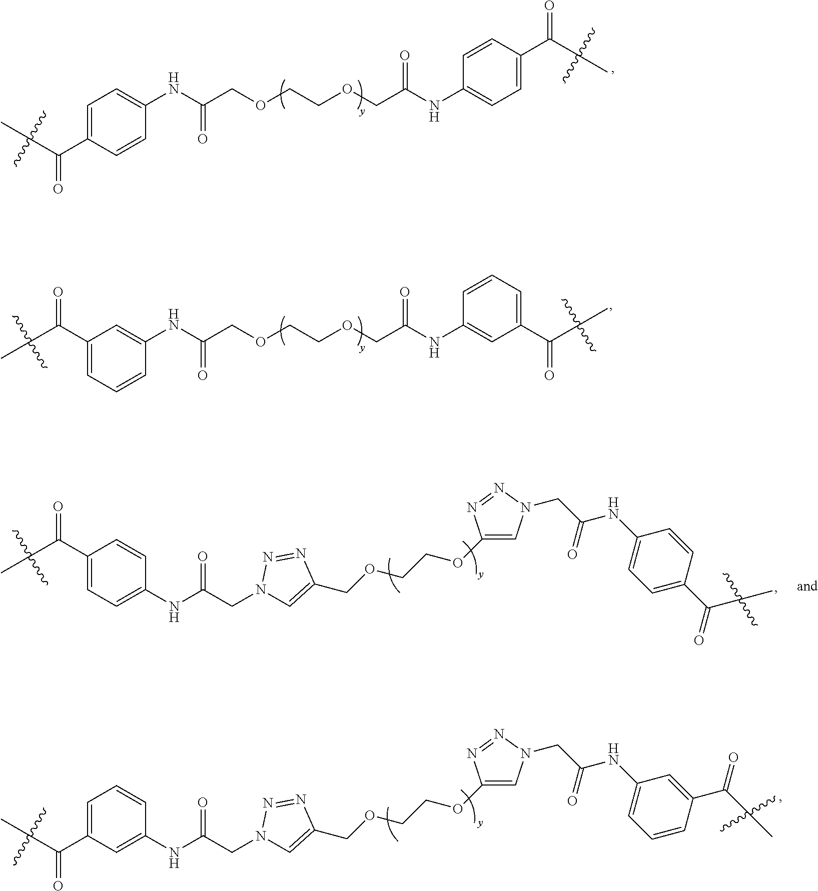

wherein y is chosen from integers ranging from 0 to 250.

In some embodiments of Formula VII, L is chosen from

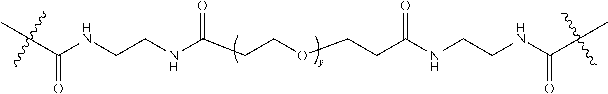

wherein y is chosen from integers ranging from 0 to 250.

In some embodiments of Formula VII, L is

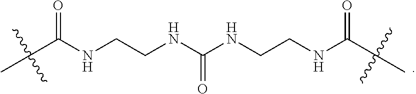 [SMILES: *C(=O)NCCNC(=O)NCCNC(*)=O]

In some embodiments of Formula VII, L is chosen from

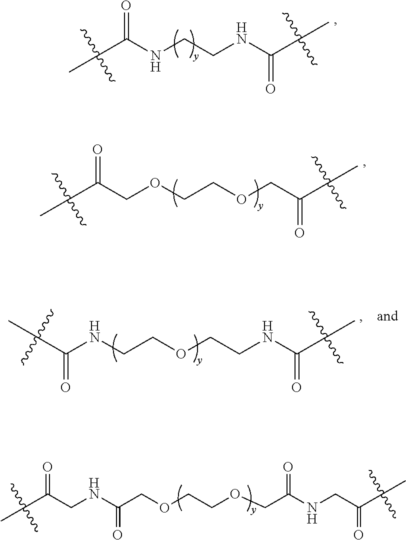

groups,
wherein y is chosen from integers ranging from 0 to 250.

In some embodiments of Formula VII, L is chosen from

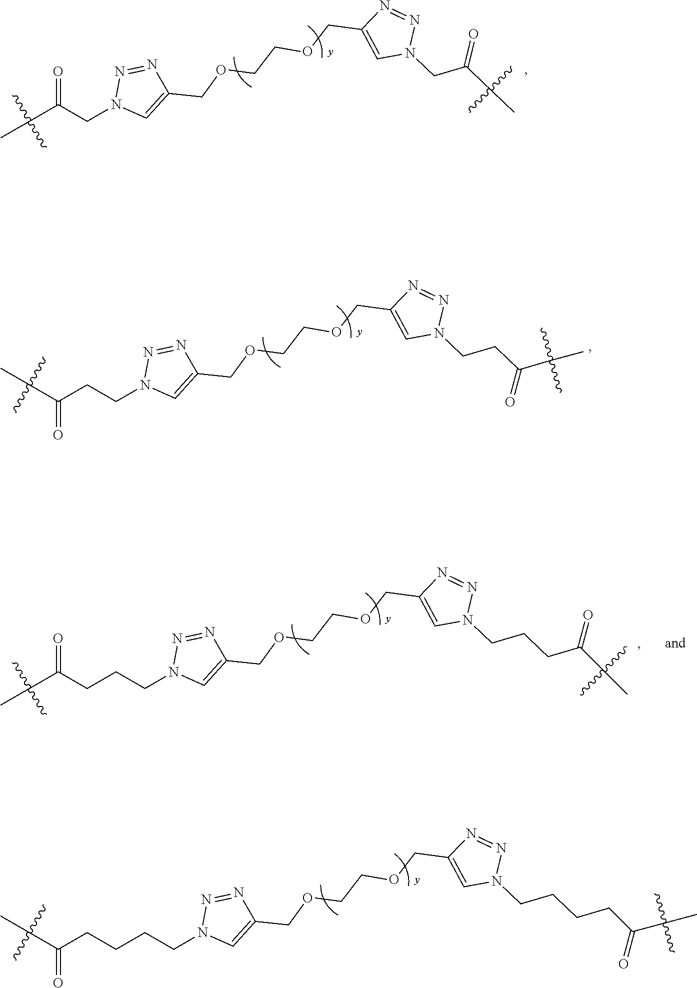

groups,
wherein y is chosen from integers ranging from 0 to 250.

In some embodiments of Formula VII, L is chosen from

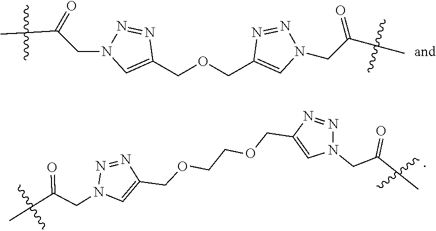 [SMILES: *C(=O)Cn1cc(COCc2cn(CC(*)=O)nn2)nn1 ; *C(=O)Cn1cc(COCCOCc2cn(CC(*)=O)nn2)nn1]

In some embodiments of Formula VII, L is

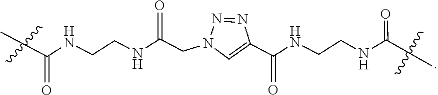 [SMILES: *C(=O)NCCNC(=O)Cn1cc(C(=O)NCCNC(*)=O)nn1]

In some embodiments of Formula VII, L is chosen from

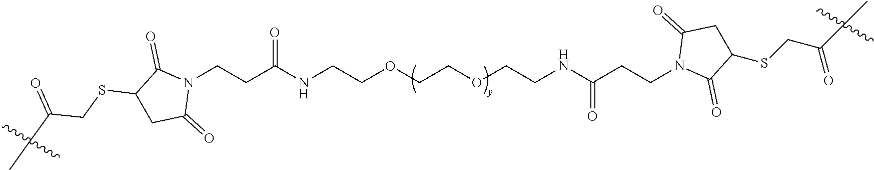

groups,
wherein y is chosen from integers ranging from 0 to 250.

In some embodiments of Formula VII, L is

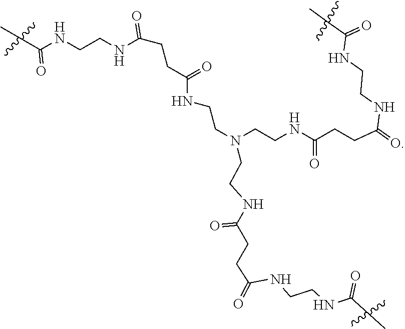 [SMILES: *C(=O)NCCNC(=O)CCC(=O)NCCN(CCNC(=O)CCC(=O)NCCNC(*)=O)CCNC(=O)CCC(=O)NCCNC(*)=O]

In some embodiments of Formula VII, L is

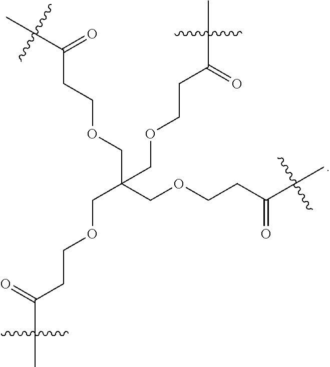 [SMILES: *C(=O)CCOCC(COCCC(*)=O)(COCCC(*)=O)COCCC(*)=O]

In some embodiments of Formula VII, L is

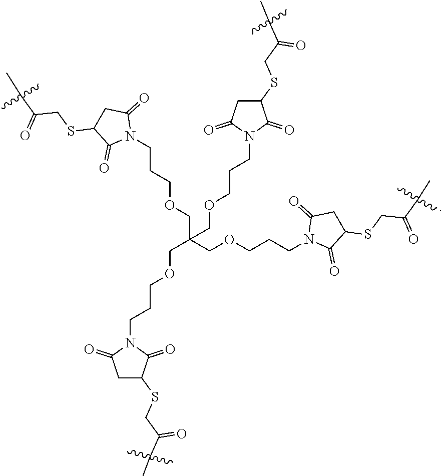 [SMILES: *C(=O)CSC1CC(=O)N(CCCOCC(COCCCN2C(=O)CC(SCC(*)=O)C2=O)(COCCCN2C(=O)CC(SCC(*)=O)C2=O)COCCCN2C(=O)CC(SCC(*)=O)C2=O)C1=O]

In some embodiments of Formula VII, L is chosen from

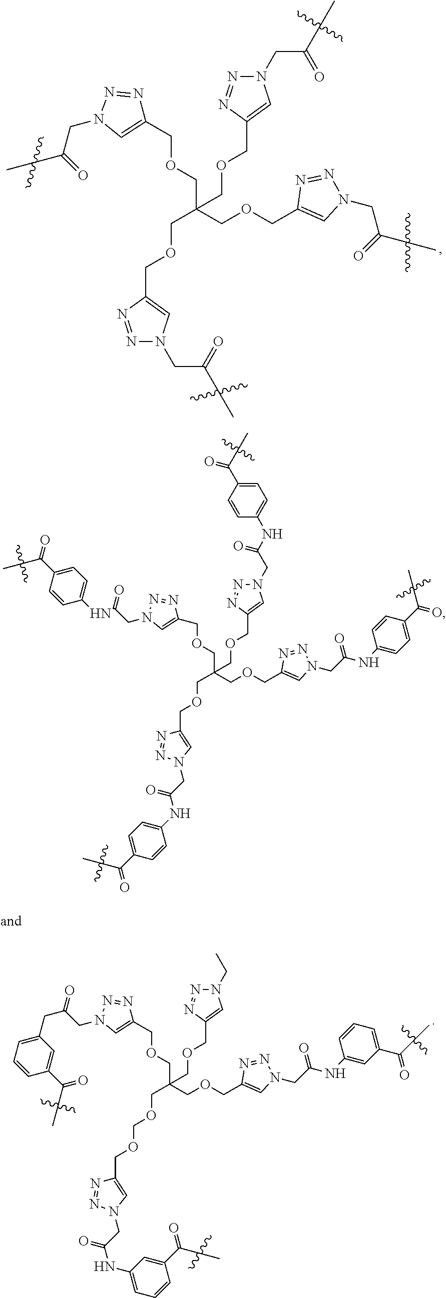 [SMILES: *C(=O)Cn1cc(COCC(COCc2cn(CC(*)=O)nn2)(COCc2cn(CC(*)=O)nn2)COCc2cn(CC(*)=O)nn2)nn1 ; *C(=O)c1ccc(NC(=O)Cn2cc(COCC(COCc3cn(CC(=O)Nc4ccc(C(*)=O)cc4)nn3)(COCc3cn(CC(=O)Nc4ccc(C(*)=O)cc4)nn3)COCc3cn(CC(=O)Nc4ccc(C(*)=O)cc4)nn3)nn2)cc1 ; *C(=O)c1cccc(CC(=O)Cn2cc(COCC(COCOCc3cn(CC(=O)Nc4cccc(C(*)=O)c4)nn3)(COCc3cn(CC)nn3)COCc3cn(CC(=O)Nc4cccc(C(*)=O)c4)nn3)nn2)c1]

In some embodiments of Formula VII, L is

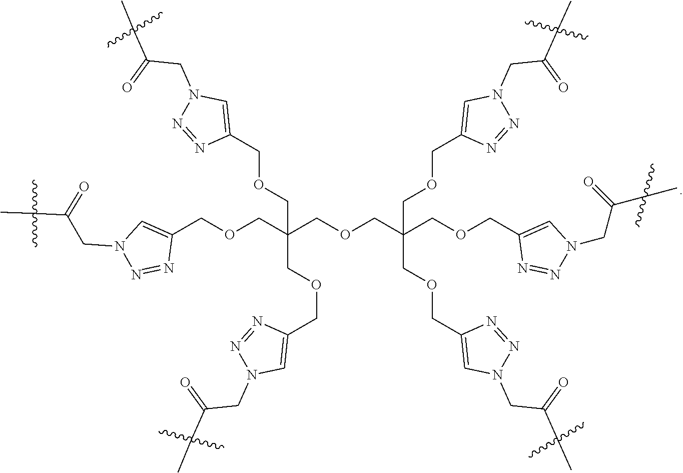 [SMILES: *C(=O)Cn1cc(COCC(COCc2cn(CC(*)=O)nn2)(COCc2cn(CC(*)=O)nn2)COCC(COCc2cn(CC(*)=O)nn2)(COCc2cn(CC(*)=O)nn2)COCc2cn(CC(*)=O)nn2)nn1]

In some embodiments of Formula VII, L is chosen from

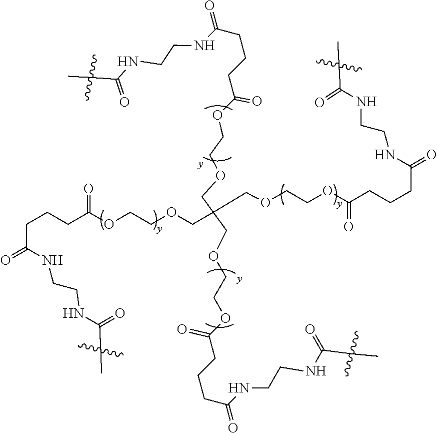

groups,
wherein each y, which may be identical or different, is independently chosen from integers ranging from 0 to 250.

In some embodiments Formula VII, L is chosen from

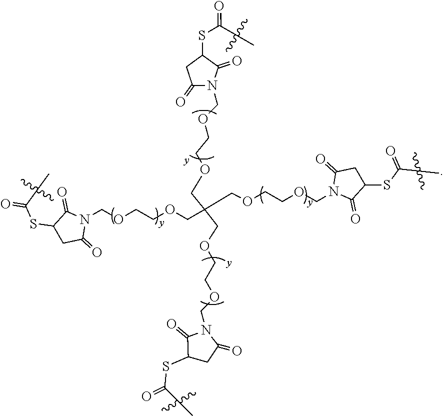

wherein each y, which may be identical or different, is independently chosen from integers ranging from 0 to 250.

In some embodiments of Formula VII, L is chosen from

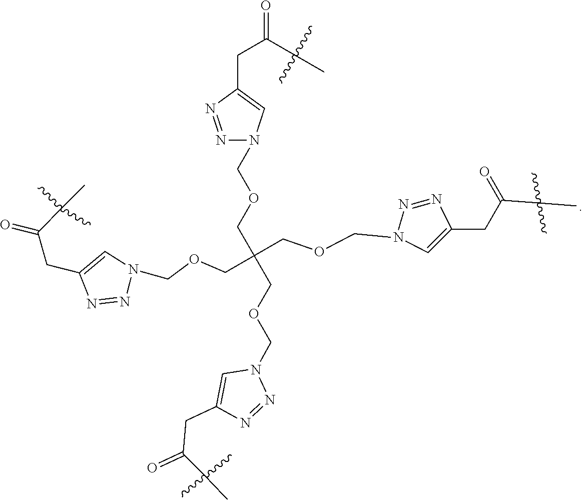 [SMILES: *C(=O)Cc1cn(COCC(COCn2cc(CC(*)=O)nn2)(COCn2cc(CC(*)=O)nn2)COCn2cc(CC(*)=O)nn2)nn1]

In some embodiments, at least one compound is chosen from compounds of Formula VII, wherein each R¹ is identical, each R² is identical, each R³ is identical, each R⁴ is identical, each R⁵ is identical, and each X is identical. In some embodiments, at least one compound is chosen from compounds of Formula VII, wherein said compound is symmetrical.

Provided are pharmaceutical compositions comprising at least one compound chosen from compounds of Formula Ix, Ia, II, IIa, III, IV, IIIa/IVa, IIIb/IVb, V, VI, and VII, and pharmaceutically acceptable salts of any of the foregoing. Also provided are pharmaceutical compositions comprising at least one compound chosen from compound A, compound B, compound C, and compound D, and pharmaceutically acceptable salts of any of the foregoing. In some embodiments, the pharmaceutically acceptable salts is a sodium salt. These compounds and compositions may be used in the methods described herein.

### EXAMPLES

### Example 1

### Prophetic Synthesis of Multimeric Compound 21

Compound 3: A mixture of compound 1 (preparation described in WO 2007/028050) and compound 2 (preparation described in WO 2013/096926) (1.7 eq) is azeotroped 3 times from toluene. The mixture is dissolved in DCM under argon and cooled on an ice bath. To this solution is added boron trifluoride etherate (1.5 eq). The reaction mixture is stirred 12 hours at room temperature. The reaction is quenched by the addition of triethylamine (2 eq). The reaction mixture is transferred to a separatory funnel and washed 1 time with half saturated sodium bicarbonate solution and 1 time with water. The organic phase is dried over sodium sulfate, filtered, and concentrated. The residue is purified by flash chromatography to afford compound 3.

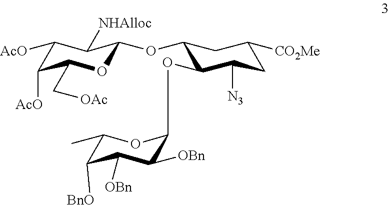

Compound 4: Compound 3 is dissolved in methanol at room temperature. A solution of sodium methoxide in methanol (0.1 eq) is added and the reaction mixture stirred overnight at room temperature. The reaction mixture is quenched by the addition of acetic acid. The reaction mixture is diluted with ethyl acetate, transferred to a separatory funnel and washed 2 times with water. The organic phase is dried over magnesium sulfate, filtered and concentrated. The residue is separated by flash chromatography to afford compound 4.

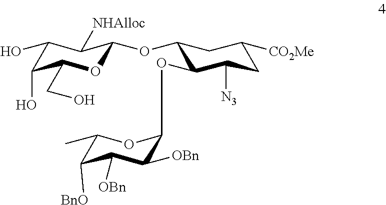

Compound 5: To a solution of compound 4 in dichloromethane cooled on an ice bath is added DABCO (1.5 eq) followed by monomethyoxytrityl chloride (1.2 eq). The reaction mixture is stirred overnight allowing to warm to room temperature. The reaction mixture is transferred to a separatory funnel and washed 2 times with water. The organic phase is concentrated and the residue is purified by flash chromatography to afford compound 5.

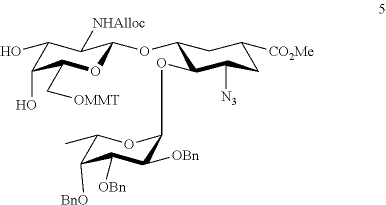

Compound 7: To a solution of compound 5 in methanol is added dibutyltin oxide (1.1 eq). The reaction mixture is refluxed for 3 hours then concentrated. The residue is suspended in DME. To this suspension is added compound 6 (preparation described in Thoma et. al. J. Med. Chem., 1999, 42, 4909) (1.5 eq) followed by cesium fluoride (1.2 eq). The reaction mixture is stirred at room temperature overnight. The reaction mixture is diluted with ethyl acetate, transferred to a separatory funnel, and washed with water. The organic phase is dried over sodium sulfate, filtered and concentrated. The residue is purified by flash chromatography to afford compound 7.

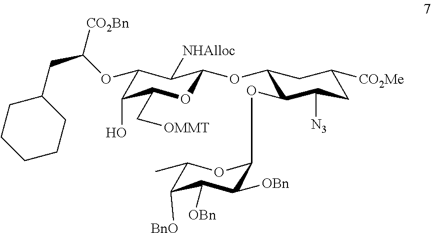

Compound 8: To a degassed solution of compound 7 in anhydrous DCM at 0° C. is added Pd(PPh₃)₄ (0.1 eq), Bu₃SnH (1.1 eq) and N-trifluoroacetyl glycine anhydride (2.0 eq) (preparation described in Chemische Berichte (1955), 88(1), 26). The resulting solution is stirred for 12 hrs allowing the temperature to increase to room temperature. The reaction mixture is diluted with DCM, transferred to a separatory funnel, and washed with water. The organic phase is dried over Na₂SO₄, then filtered and concentrated. The residue is purified by flash chromatography to afford compound 8.

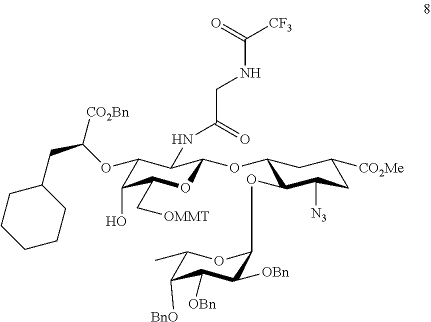

Compound 9: To a stirred solution of compound 8 in DCM/MeOH (25/1) at room temperature is added orotic acid chloride (5 eq) and triphenylphosphine (5 eq). The reaction mixture is stirred 24 hours. The solvent is removed and the residue is separated by column chromatography to afford compound 9.

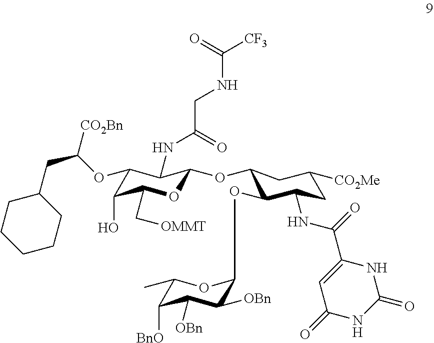

Compound 10: Compound 9 is dissolved in methanol and degassed. To this solution is added Pd(OH)₂/C. The reaction mixture is vigorously stirred under a hydrogen atmosphere for 12 hours. The reaction mixture is filtered through a Celite pad. The filtrate is concentrated under reduced pressure to give compound 10.

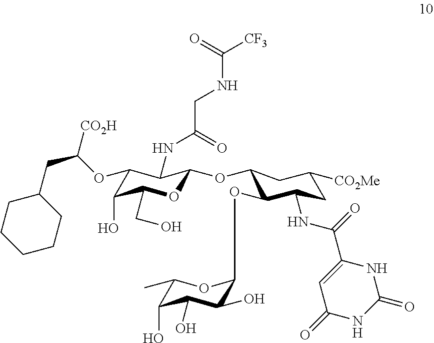

Compound 11: Compound 10 is dissolved in methanol at room temperature. A solution of sodium methoxide in methanol (1.1 eq) is added and the reaction mixture stirred overnight at room temperature. The reaction mixture is quenched by the addition of acetic acid. The reaction mixture is concentrated. The residue is separated by C-18 reverse phase chromatography to afford compound 11.

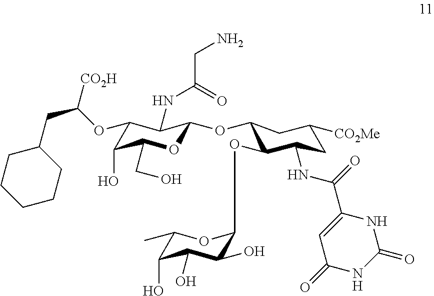

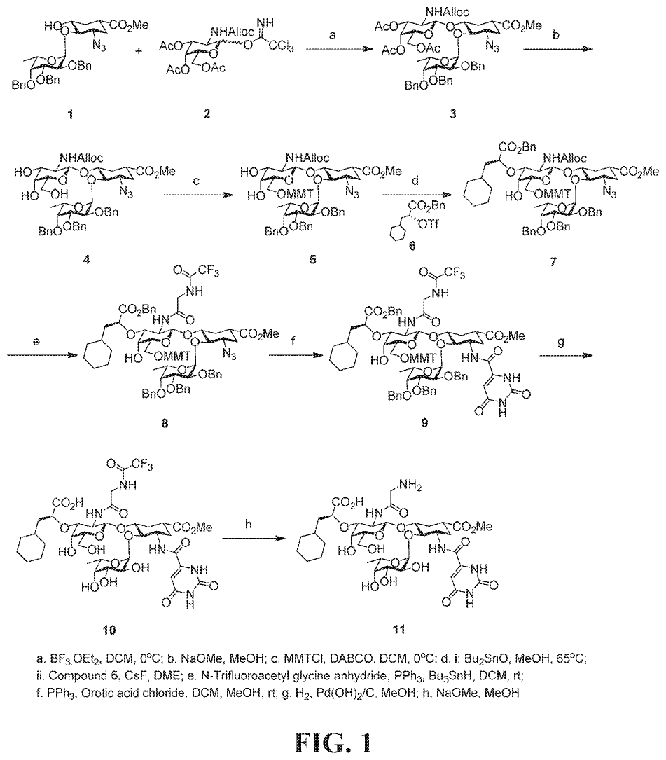

Compound 12: Compound 12 can be prepared in an analogous fashion to FIG. 1 by substituting (acetylthio)acetyl chloride for N-trifluoroacetyl glycine anhydride in step e.

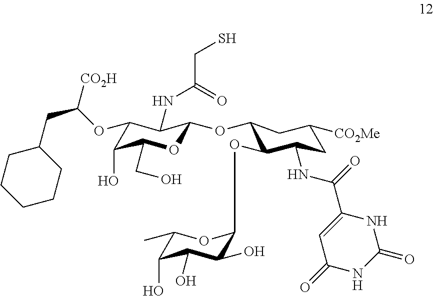

Compound 13: Compound 10 is dissolved in DMF and cooled on an ice bath. Diisopropylethylamine (1.5 eq) is added followed by HATU (1.1 eq). The reaction mixture is stirred 15 minutes on the ice bath then azetidine (2 eq) is added. The ice bath is removed and the reaction mixture is stirred overnight at room temperature. The solvent is removed under reduced pressure and the residue is separated by flash chromatography to afford compound 13.

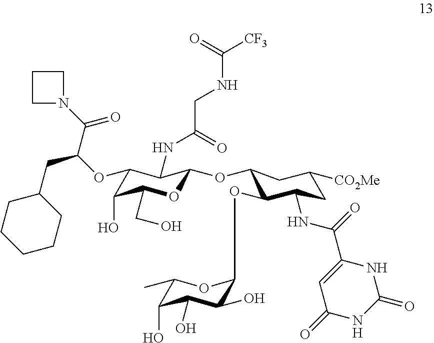

Compound 14: Compound 13 is dissolved in methanol at room temperature. A solution of sodium methoxide in methanol (0.3 eq) is added and the reaction mixture stirred overnight at room temperature. The reaction mixture is quenched by the addition of acetic acid. The reaction mixture is concentrated. The residue is separated by C-18 reverse phase chromatography to afford compound 14.

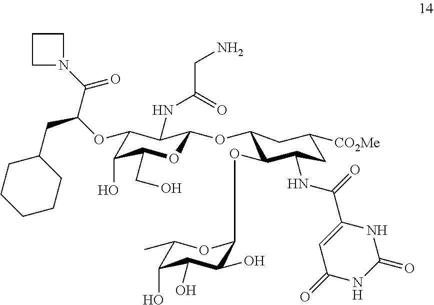

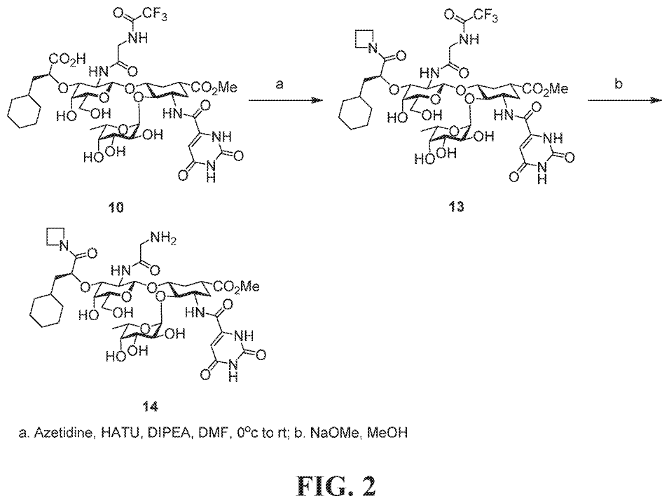

Compound 15: Compound 15 can be prepared in an analogous fashion to FIG. 2 by using methylamine in place of azetidine in step a.

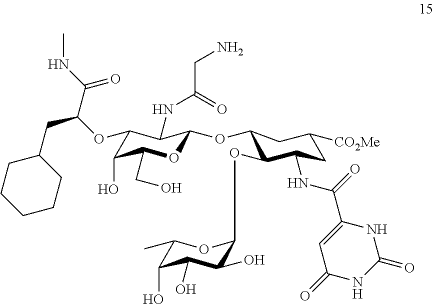

Compound 16: Compound 16 can be prepared in an analogous fashion to FIG. 2 by using dimethylamine in place of azetidine in step a.

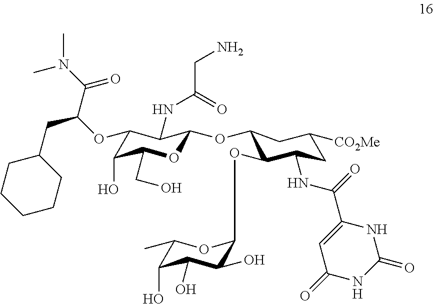

Compound 17: Compound 17 can be prepared in an analogous fashion to FIG. 2 by using 2-methoxyethylamine in place of azetidine in step a.

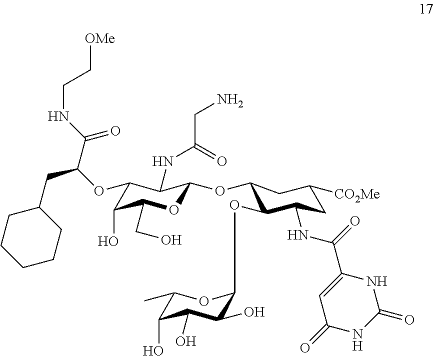

Compound 18: Compound 18 can be prepared in an analogous fashion to FIG. 2 by using piperidine in place of azetidine in step a.

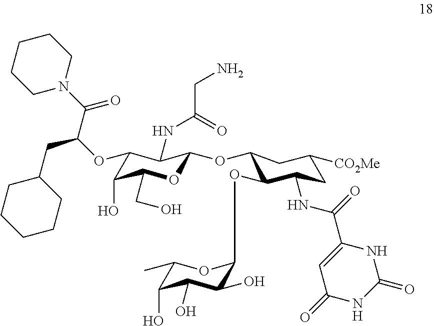

Compound 19: Compound 19 can be prepared in an analogous fashion to FIG. 2 by using morpholine in place of azetidine in step a.

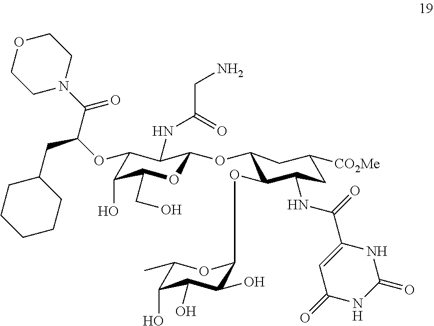

Compound 21: A solution of compound 20 (0.4 eq) in DMSO is added to a solution of compound 11 (1 eq) and DIPEA (10 eq) in anhydrous DMSO at room temperature. The resulting solution is stirred overnight. The solution is dialyzed against distilled water for 3 days with dialysis tube MWCO 1000 while distilled water is changed every 12 hours. The solution in the tube is lyophilized to give compound 21.

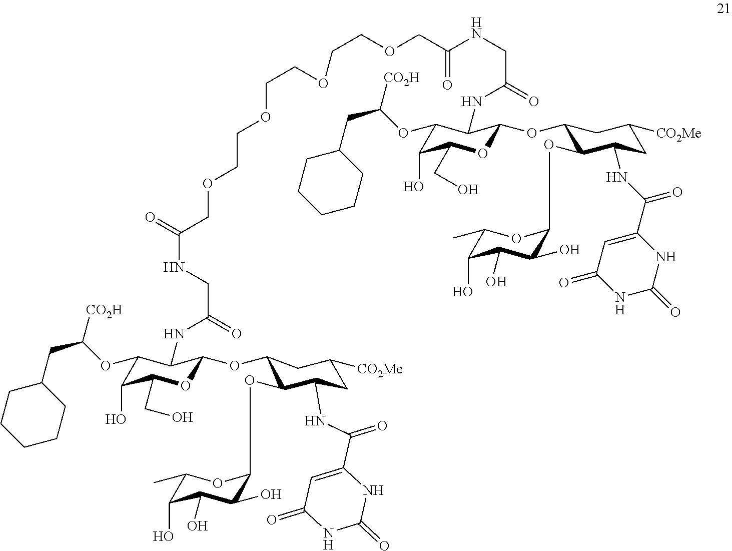

### Example 2

### Prophetic Synthesis of Multimeric Compound 22

Compound 22: A solution of compound 21 in ethylenediamine is stirred overnight at 70° C. The reaction mixture is concentrated under reduced pressure and the residue is purified by reverse phase chromatography to give compound 22.

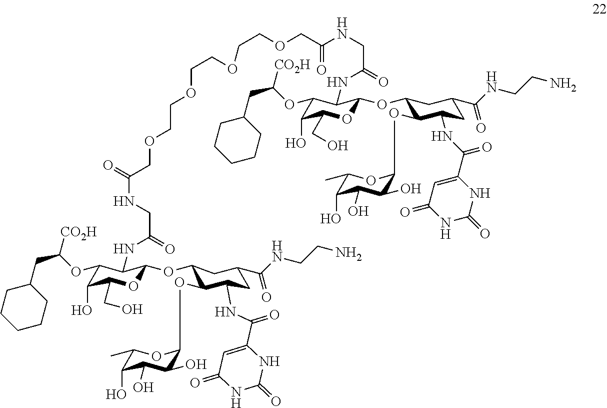

### Example 3

### Prophetic Synthesis of Multimeric Compound 23

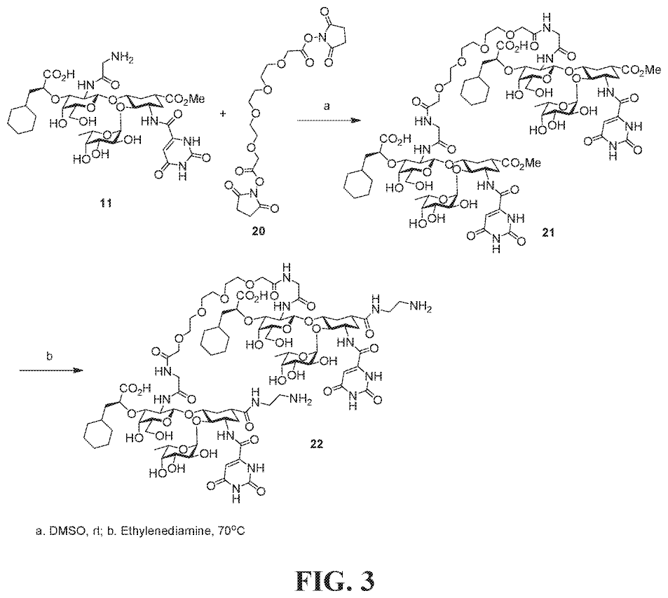

Compound 23: Compound 23 can be prepared in an analogous fashion to FIG. 3 by replacing compound 20 with PEG-li diacetic acid di-NHS ester in step a.

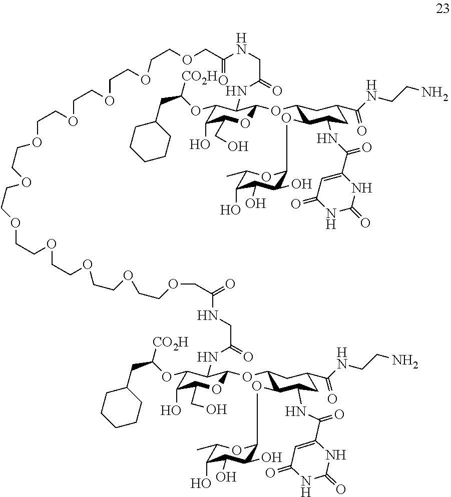

### Example 4

### Prophetic Synthesis of Multimeric Compound 24

Compound 24: Compound 24 can be prepared in an analogous fashion to FIG. 3 by replacing compound 20 with PEG-15 diacetic acid di-NHS ester in step a.

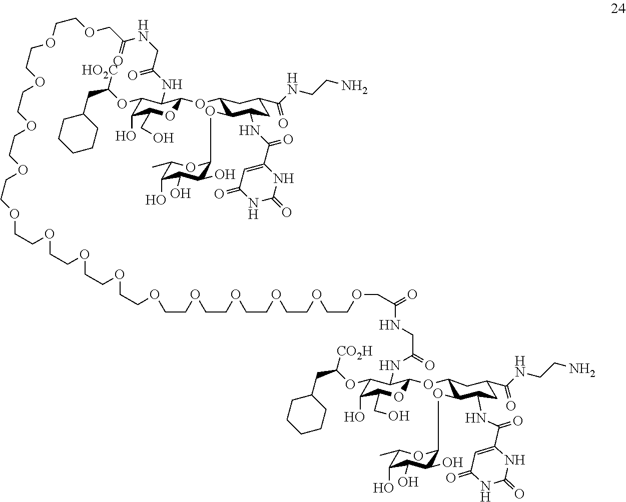

### Example 5

### Prophetic Synthesis of Multimeric Compound 25

Compound 25: Compound 25 can be prepared in an analogous fashion to FIG. 3 by replacing compound 20 with ethylene glycol diacetic acid di-NHS ester in step a.

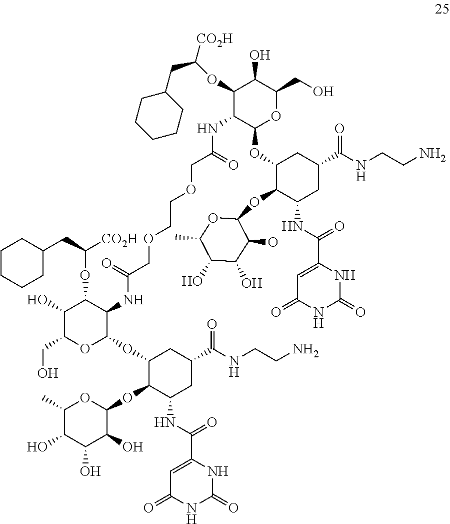 [SMILES: C[C@@H]1O[C@@H](O[C@@H]2[C@@H](NC(=O)c3cc(=O)[nH]c(=O)[nH]3)C[C@@H](C(=O)NCCN)C[C@H]2O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3C[C@H](C(=O)NCCN)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H](O)[C@H](O)[C@@H]1O]

### Example 6

### Prophetic Synthesis of Multimeric Compound 26

Compound 26: Compound 26 can be prepared in an analogous fashion to FIG. 3 by replacing compound 20 with 3,3′-[[2,2-bis[[3-[(2,5-dioxo-1-pyrrolidinyl)oxy]-3-oxopropoxy]methyl]-1,3-propanediyl]bis(oxy)]bis-, 1,1′-bis(2,5-dioxo-1-pyrrolidinyl)-propanoic acid ester in step a.

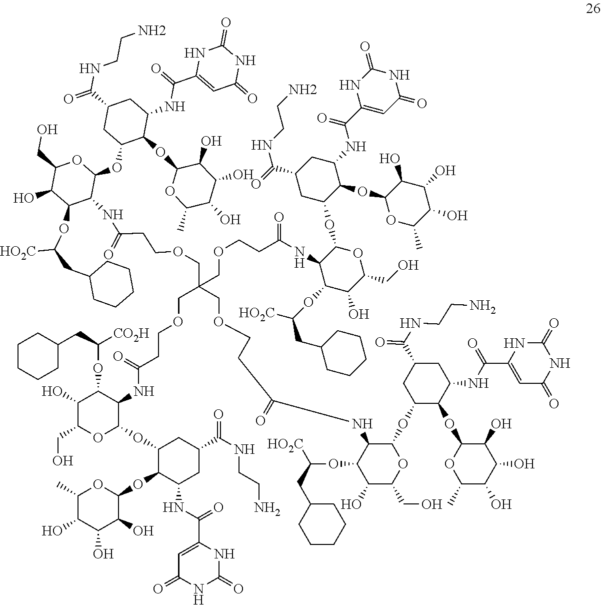

### Example 7

### Prophetic Synthesis of Multimeric Compound 27

Compound 27: Compound 27 can be prepared in an analogous fashion to FIG. 3 by replacing ethylenediamine with 2-aminoethyl ether in step b.

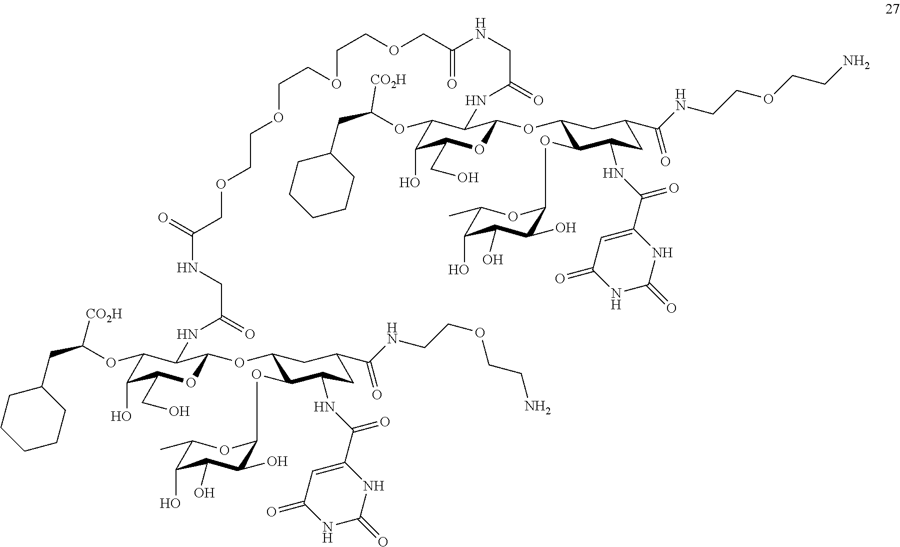

### Example 8

### Prophetic Synthesis of Multimeric Compound 28

Compound 28: Compound 28 can be prepared in an analogous fashion to FIG. 3 by replacing ethylenediamine with 1,5-diaminopentane in step b.

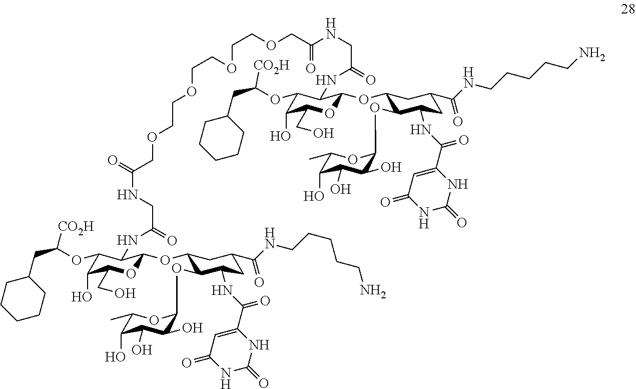

### Example 9

### Prophetic Synthesis of Multimeric Compound 29

Compound 29: Compound 29 can be prepared in an analogous fashion to FIG. 3 by replacing ethylenediamine with 1,2-bis(2-aminoethoxy)ethane in step b.

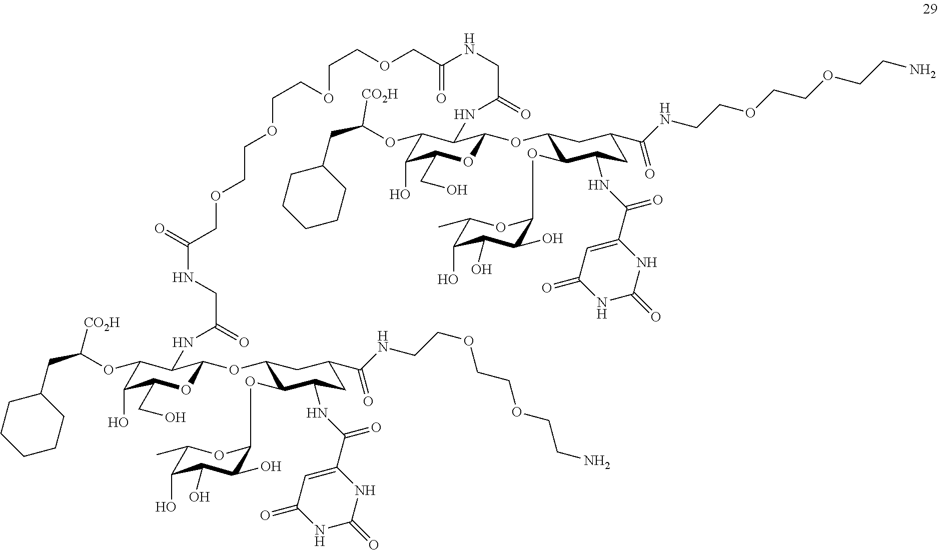

### Example 10

### Prophetic Synthesis of Multimeric Compound 30

Compound 30: Compound 30 can be prepared in an analogous fashion to FIG. 3 by replacing compound 11 with compound 14 and compound 20 with PEG-11 diacetic acid di-NHS ester in step a.

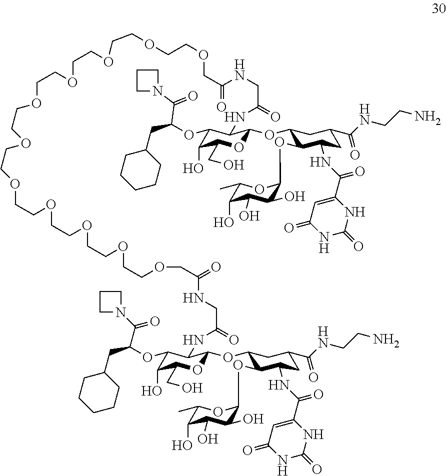

### Example 11

### Prophetic Synthesis of Multimeric Compound 31

Compound 31: Compound 31 can be prepared in an analogous fashion to FIG. 3 by replacing compound 11 with compound 15 in step a.

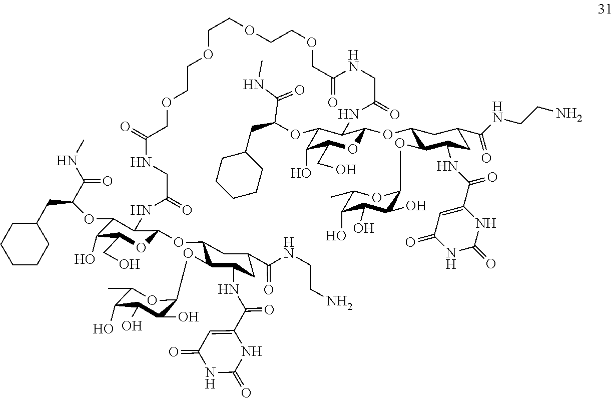

### Example 12

### Prophetic Synthesis of Multimeric Compound 32

Compound 32: Compound 32 can be prepared in an analogous fashion to FIG. 3 by replacing compound 11 with compound 17 and compound 20 with PEG-15 diacetic acid di-NHS ester in step a.

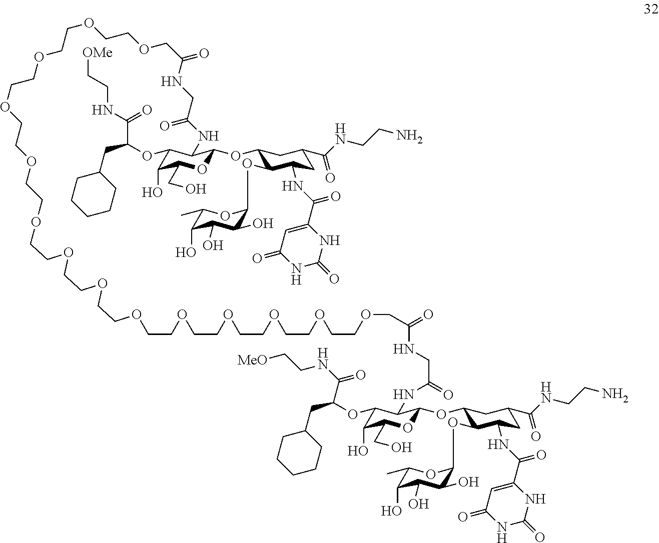

### Example 13

### Prophetic Synthesis of Multimeric Compound 33

Compound 33: Compound 33 can be prepared in an analogous fashion to FIG. 3 by replacing compound 11 with compound 16 and compound 20 with ethylene glycol diacetic acid di-NHS ester in step a.

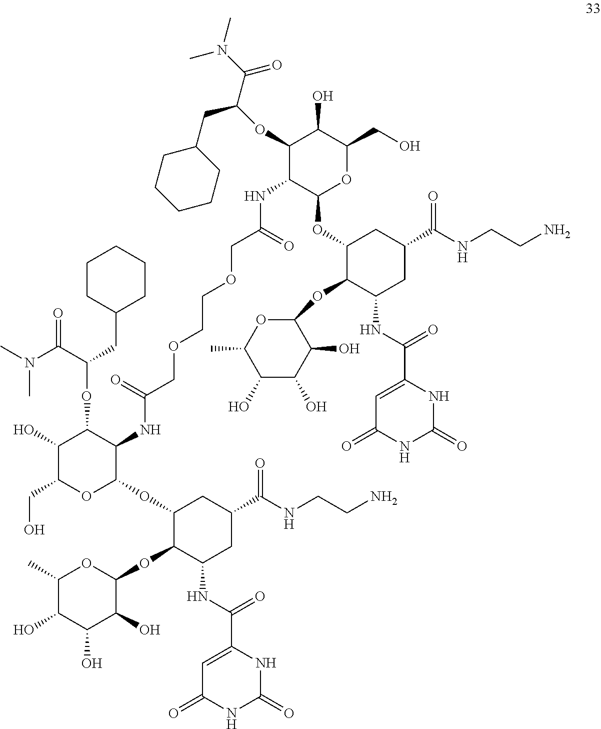 [SMILES: C[C@@H]1O[C@@H](O[C@@H]2[C@@H](NC(=O)c3cc(=O)[nH]c(=O)[nH]3)C[C@@H](C(=O)NCCN)C[C@H]2O[C@@H]2O[C@H](CO)[C@H](O)[C@H](O[C@@H](CC3CCCCC3)C(=O)N(C)C)[C@H]2NC(=O)COCCOCC(=O)N[C@H]2[C@H](O[C@@H]3C[C@H](C(=O)NCCN)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N(C)C)[C@@H](O)[C@H](O)[C@@H]1O]

### Example 14

### Prophetic Synthesis of Multimeric Compound 24

Compound 34: Compound 34 can be prepared in an analogous fashion to FIG. 3 by replacing compound 11 with compound 18 in step a and replacing ethylenediamine with 2-aminoethyl ether in step b.

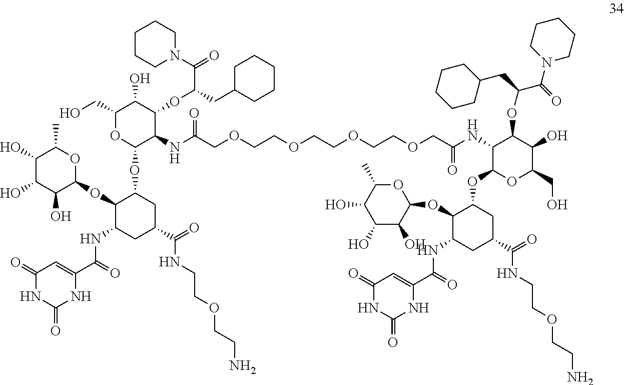 [SMILES: C[C@@H]1O[C@@H](O[C@@H]2[C@@H](NC(=O)c3cc(=O)[nH]c(=O)[nH]3)C[C@@H](C(=O)NCCOCCN)C[C@H]2O[C@@H]2O[C@H](CO)[C@H](O)[C@H](O[C@@H](CC3CCCCC3)C(=O)N3CCCCC3)[C@H]2NC(=O)COCCOCCOCCOCC(=O)N[C@H]2[C@H](O[C@@H]3C[C@H](C(=O)NCCOCCN)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCCCC2)[C@@H](O)[C@H](O)[C@@H]1O]

### Example 15

### Prophetic Synthesis of Multimeric Compound 36

Compound 36: To a solution of compound 12 in MeOH at room temperature is added compound 35 followed by cesium acetate (2.5 eq). The reaction mixture is stirred at room temperature until completion. The solvent is removed under reduced pressure. The product is purified by reverse phase chromatography to give compound 36.

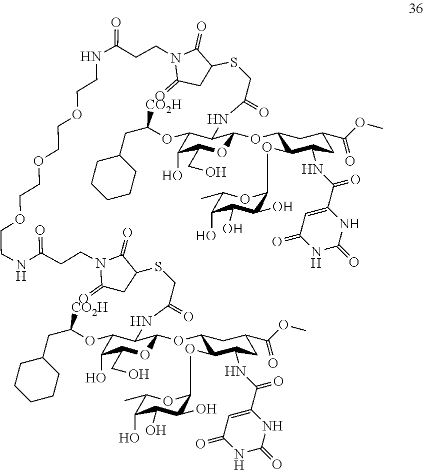

### Example 16

### Prophetic Synthesis of Multimeric Compound 37

Compound 37: Compound 36 is dissolved in ethylenediamine and the reaction mixture is stirred overnight at 70° C. The reaction mixture is concentrated under reduced pressure and the residue is purified by reverse phase chromatography to give compound 37.

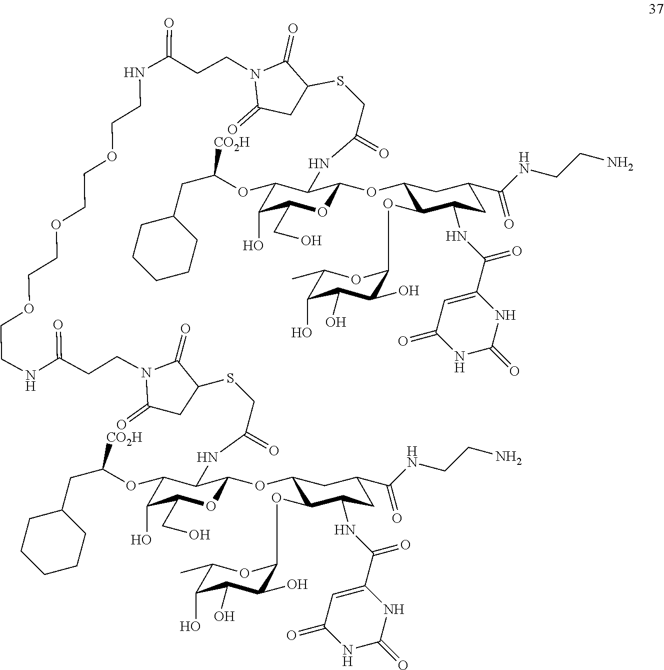

### Example 17

### Prophetic Synthesis of Multimeric Compound 38

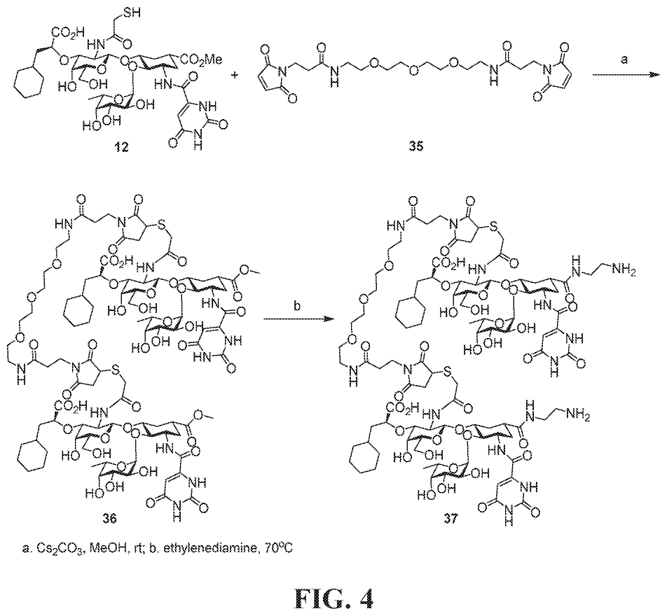

Compound 38: Compound 38 can be prepared in an analogous fashion to FIG. 4 by substituting PEG-6-bis maleimidoylpropionamide for compound 35 in step a.

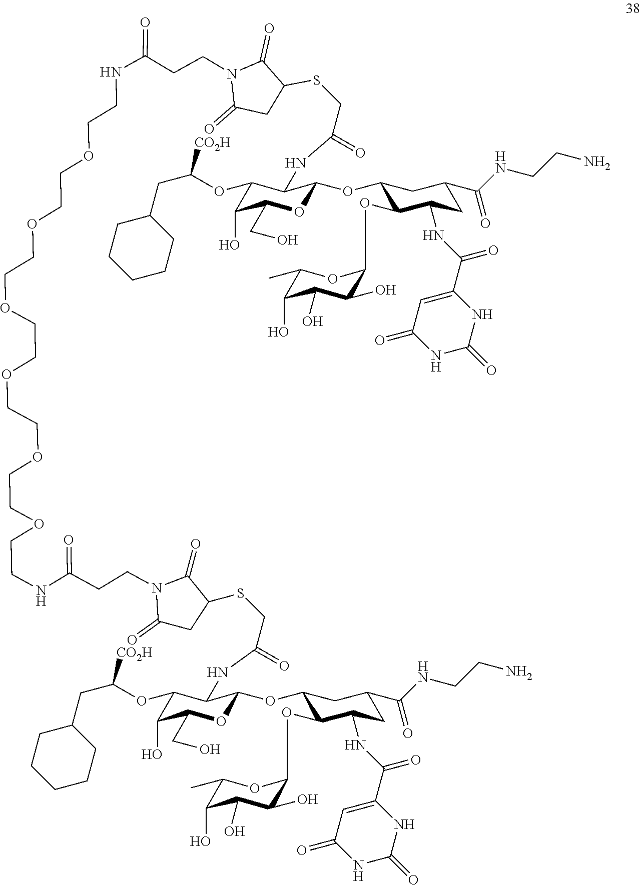

### Example 18

### Prophetic Synthesis of Multimeric Compound 39

Compound 39: Compound 39 can be prepared in an analogous fashion to FIG. 4 by substituting compound 35 for, 1,1′-[[2,2-bis[[3-(2,5-dihydro-2,5-dioxo-1H-pyrrol-1-yl) propoxy]methyl]-1,3-propanediyl]bis(oxy-3,1-propanediyl)]bis-1H-pyrrole-2,5-dione in step a.

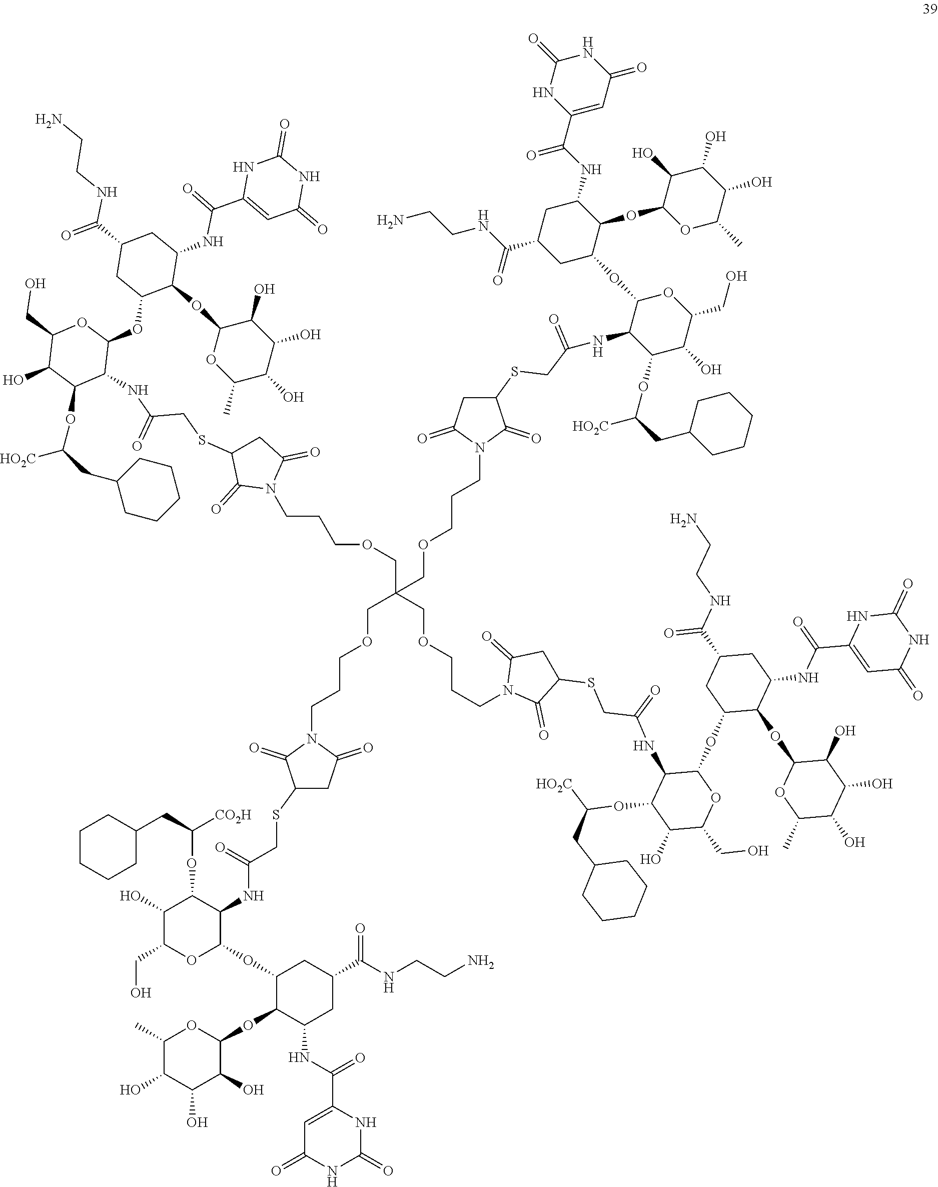

### Example 19

### Prophetic Synthesis of Multimeric Compound 40

Compound 40: Compound 40 can be prepared in an analogous fashion to FIG. 4 by substituting propylenediamine for ethylenediamine in step b.

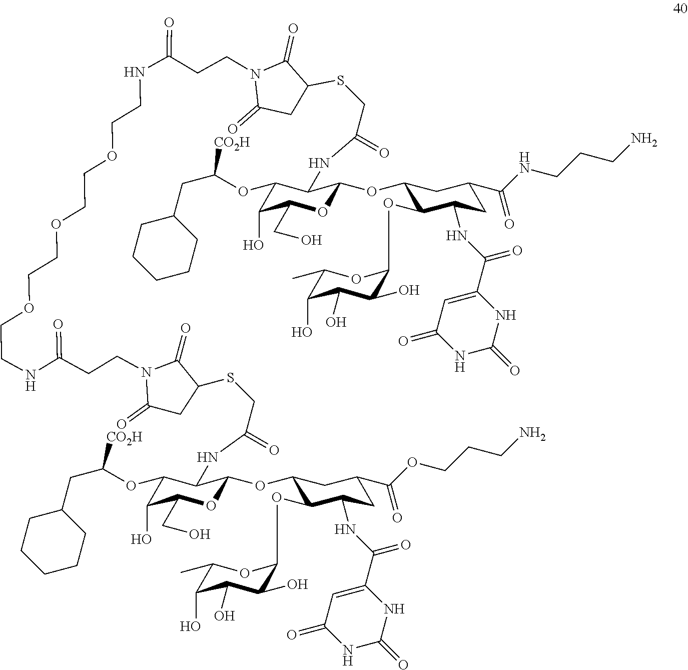

### Example 20

### Prophetic Synthesis of Multimeric Compound 44

Compound 41: To a stirred solution of compound 7 in DCM/MeOH (25/1) at room temperature is added orotic acid chloride (5 eq) and triphenylphosphine (5 eq). The reaction mixture is stirred 24 hours. The solvent is removed and the residue is separated by column chromatography to afford compound 41.

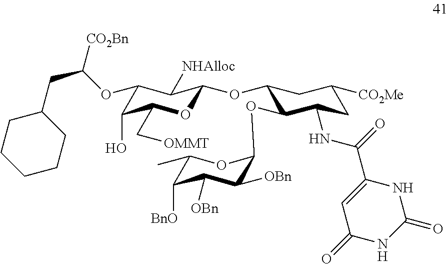

Compound 42: To a degassed solution of compound 41 in anhydrous DCM at 0° C. is added Pd(PPh₃)₄ (0.1 eq), Bu₃SnH (1.1 eq) and azidoacetic anhydride (2.0 eq). The ice bath is removed and the solution is stirred for 12 hrs under a N₂ atmosphere at room temperature. The reaction mixture is diluted with DCM, washed with water, dried over Na₂SO₄, then concentrated. The crude product is purified by column chromatography to give compound 42.

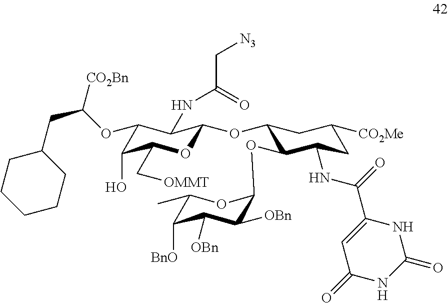

Compound 44: A solution of bispropagyl PEG-5 (compound 43) and compound 42 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at 70° C. The solution is cooled to room temperature and concentrated under reduced pressure. The crude product is purified by chromatography to give compound 44.

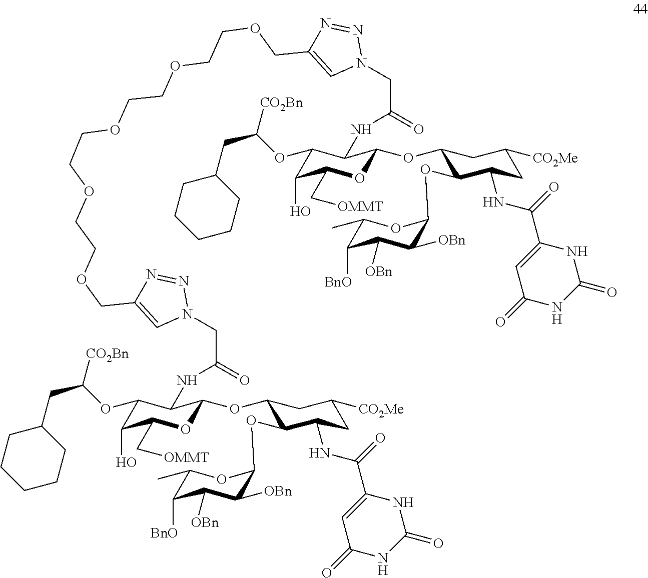

### Example 21

### Prophetic Synthesis of Multimeric Compound 45

Compound 45: Compound 44 is dissolved in MeOH/i-PrOH (2/1) and hydrogenated in the presence of Pd(OH)₂ (20 wt %) at 1 atm of H₂ gas pressure for 24 hrs at room temperature. The solution is filtered through a Celite pad. The filtrate is concentrated to give compound 45.

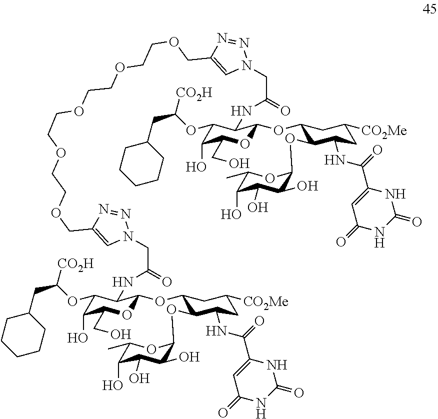

### Example 22

### Prophetic Synthesis of Multimeric Compound 46

Compound 46: Compound 45 is dissolved in ethylenediamine and stirred for 12 hrs at 70° C. The reaction mixture is concentrated under reduced pressure. The crude product is purified by C-18 column chromatography followed by lyophilization to give a compound 46.

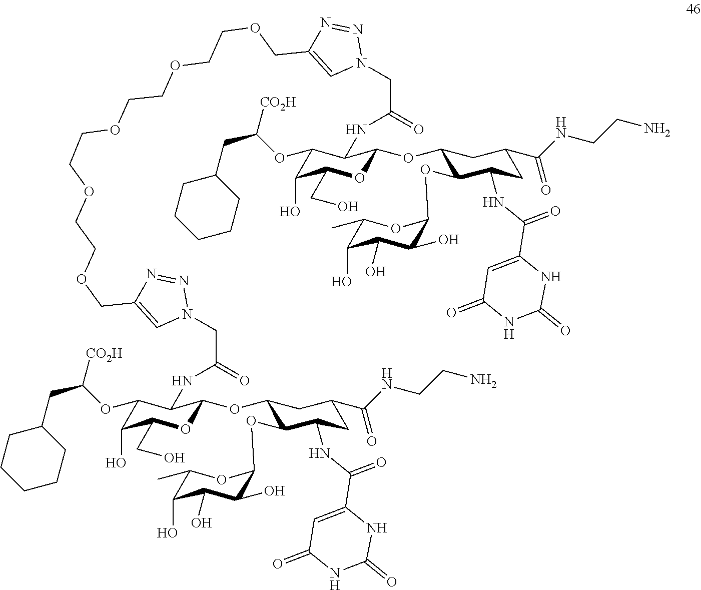

### Example 23

### Prophetic Synthesis of Multimeric Compound 47

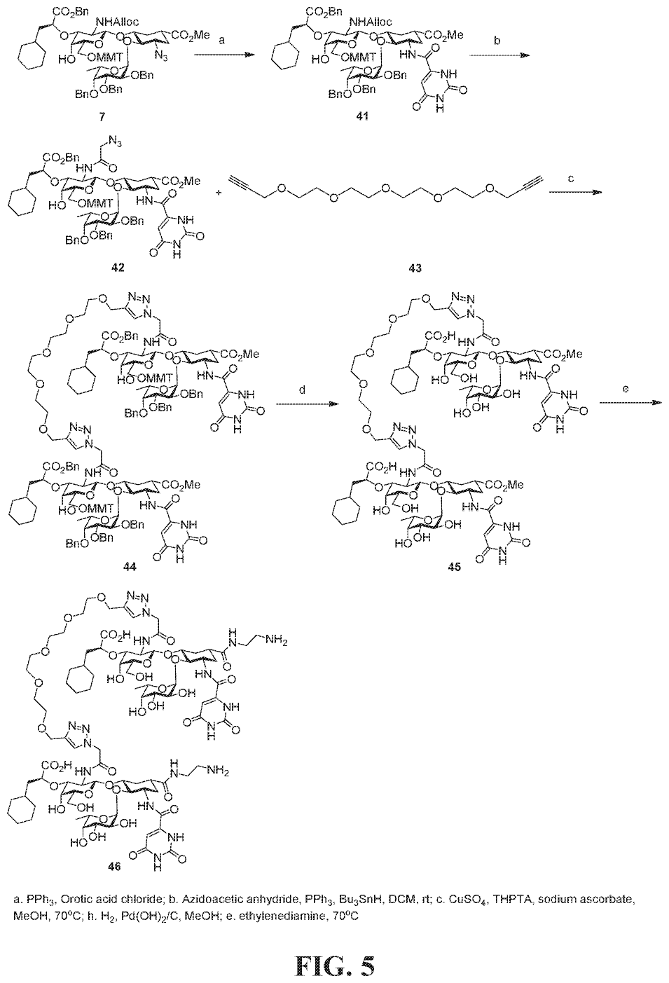

Compound 47: Compound 47 can be prepared in an analogous fashion to FIG. 5 using 3-azidopropanoic anhydride (Yang, C. et. al. JACS, (2013) 135(21), 7791-7794) in place of azidoacetic anhydride in step b.

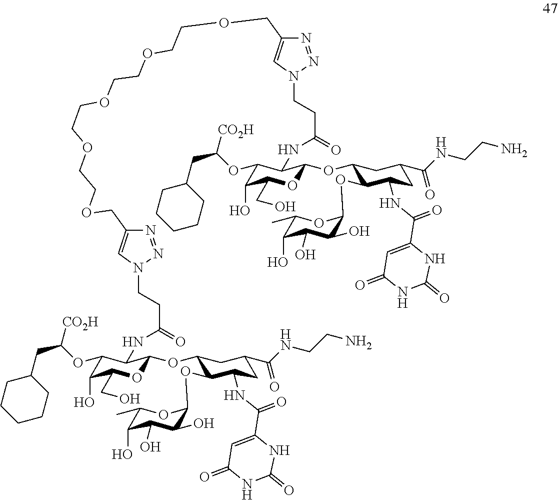

### Example 24

### Prophetic Synthesis of Multimeric Compound 48

Compound 48: Compound 48 can be prepared in an analogous fashion to FIG. 5 using 4-azidobutanoic anhydride (Yang, C. et. al. JACS, (2013) 135(21), 7791-7794) in place of azidoacetic anhydride in step b.

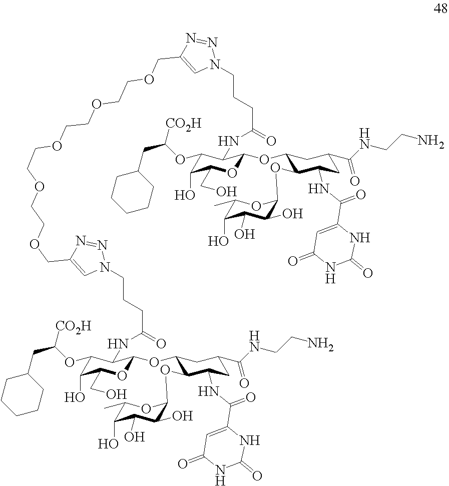

### Example 25

### Prophetic Synthesis of Multimeric Compound 49

Compound 49: Compound 49 can be prepared in an analogous fashion to FIG. 5 using 4-azidobutanoic anhydride (Yang, C. et. al. JACS, (2013) 135(21), 7791-7794) in place of azidoacetic anhydride in step b and using 1,2-bis(2-propynyloxy) ethane in place of compound 43 in step c.

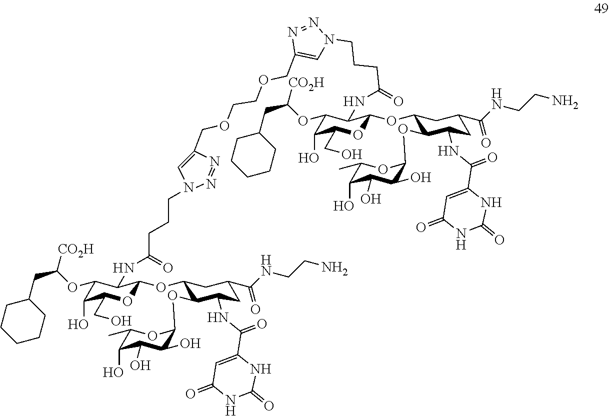

### Example 26

### Prophetic Synthesis of Multimeric Compound 50

Compound 50: Compound 50 can be prepared in an analogous fashion to FIG. 5 using 4,7,10,13,16,19,22,25,28,31-decaoxatetratriaconta-1,33-diyne in place of compound 43 in step c.

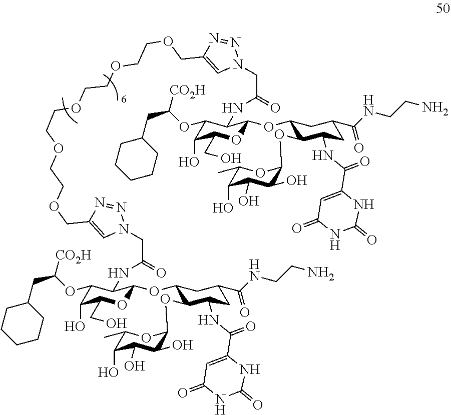

### Example 27

### Prophetic Synthesis of Multimeric Compound 51

Compound 51: Compound 51 can be prepared in an analogous fashion to FIG. 5 using 3,3′-[[2,2-bis[(2-propyn-1-yloxy)methyl]-1,3-propanediyl]bis(oxy)]bis-1-propyne in place of compound 43 in step c.

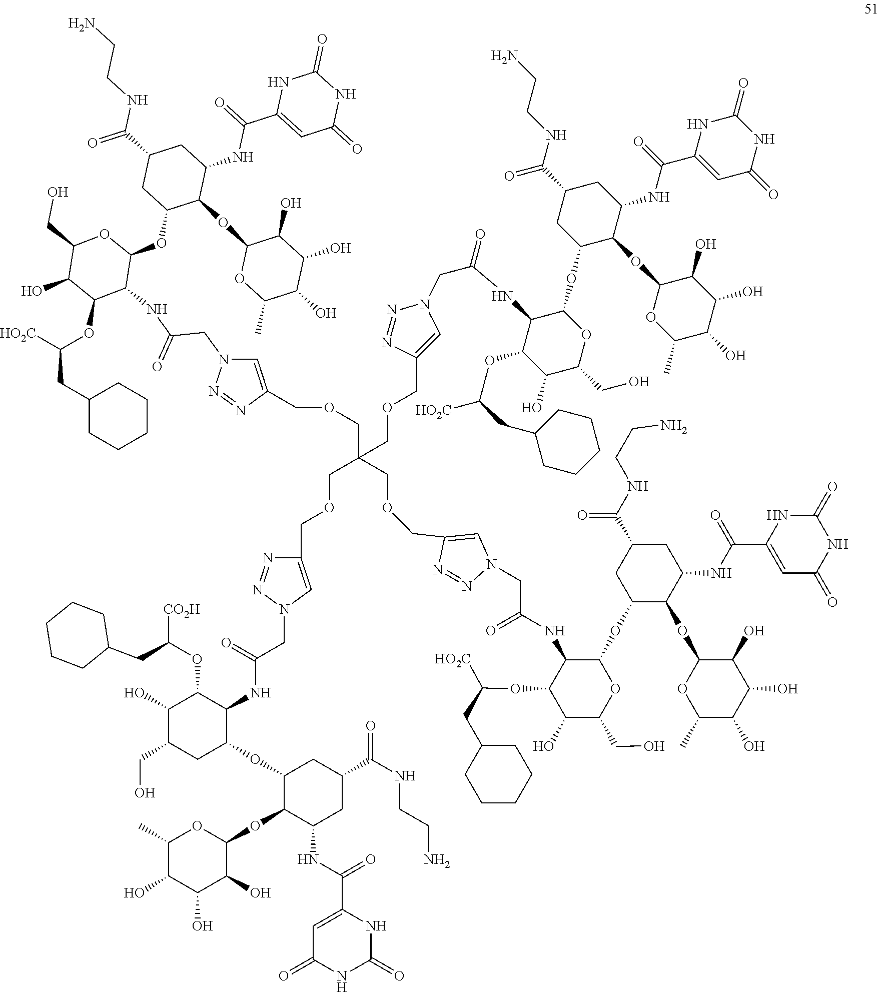

### Example 28

### Prophetic Synthesis of Multimeric Compound 52

Compound 52: Compound 52 can be prepared in an analogous fashion to FIG. 5 using 3,3′-[oxybis[[2,2-bis[(2-propyn-1-yloxy)methyl]-3,1-propanediyl]oxy]]bis-1-propyne in place of compound 43 in step c.

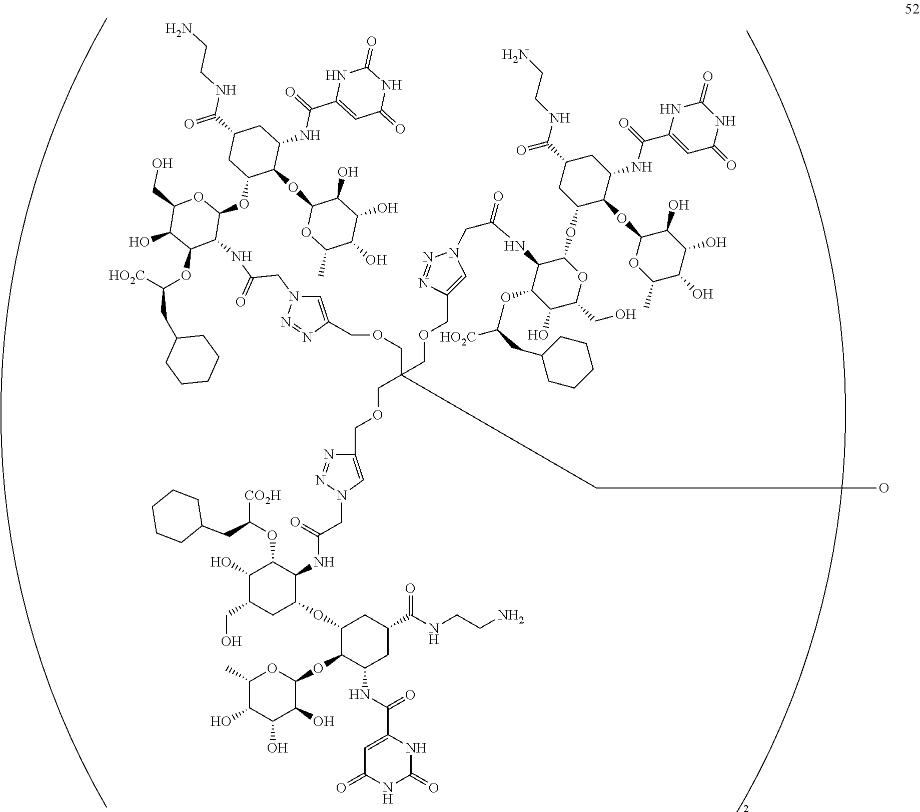

### Example 29

### Prophetic Synthesis of Multimeric Compound 53

Compound 53: Compound 53 can be prepared in an analogous fashion to FIG. 5 using butylenediamine in place of ethylenediamine in step e.

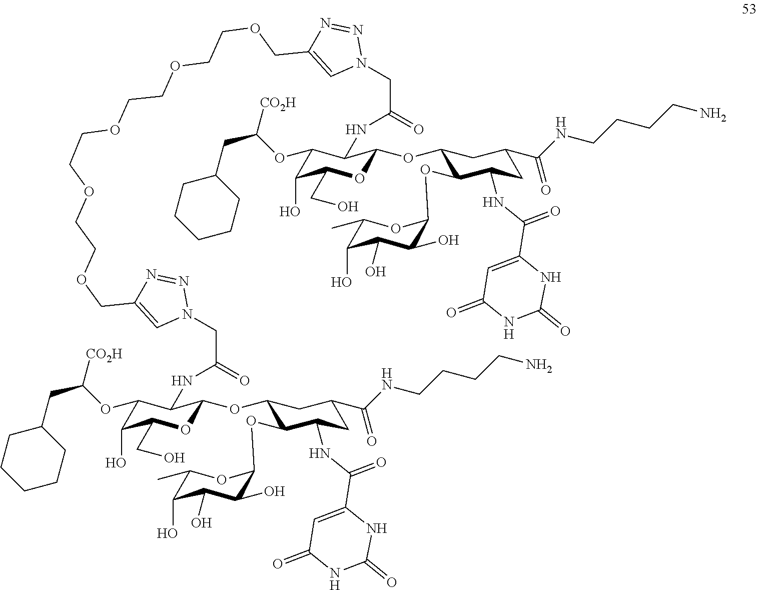

### Example 30

### Prophetic Synthesis of Multimeric Compound 54

Compound 54: Compound 54 can be prepared in an analogous fashion to FIG. 5 using 4-azidobutanoic anhydride (Yang, C. et. al. JACS, (2013) 135(21), 7791-7794) in place of azidoacetic anhydride in step b and using 1,2-bis(2-propynyloxy) ethane in place of compound 43 in step c and using 2-aminoethyl ether in step e.

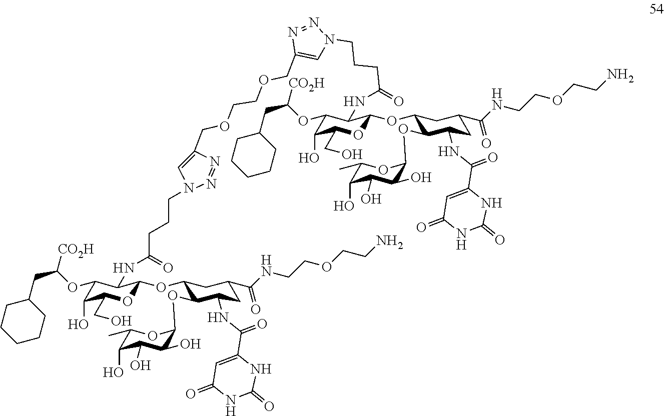

### Example 31

### Prophetic Synthesis of Multimeric Compound 55

Compound 55: Compound 54 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by flash chromatography to afford compound 55.

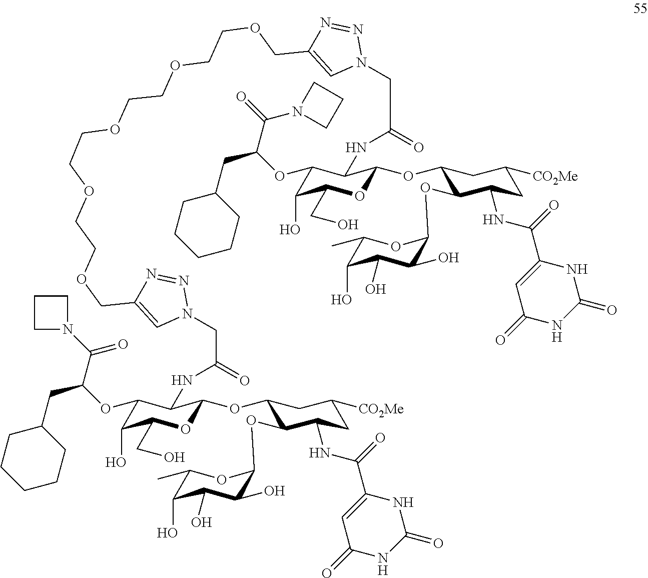

### Example 32

### Prophetic Synthesis of Multimeric Compound 56

Compound 56: Compound 55 is dissolved in ethylenediamine and stirred for 12 hrs at 70° C. The reaction mixture is concentrated under reduced pressure. The crude product is purified by C-18 column chromatography followed by lyophilization to give a compound 56.

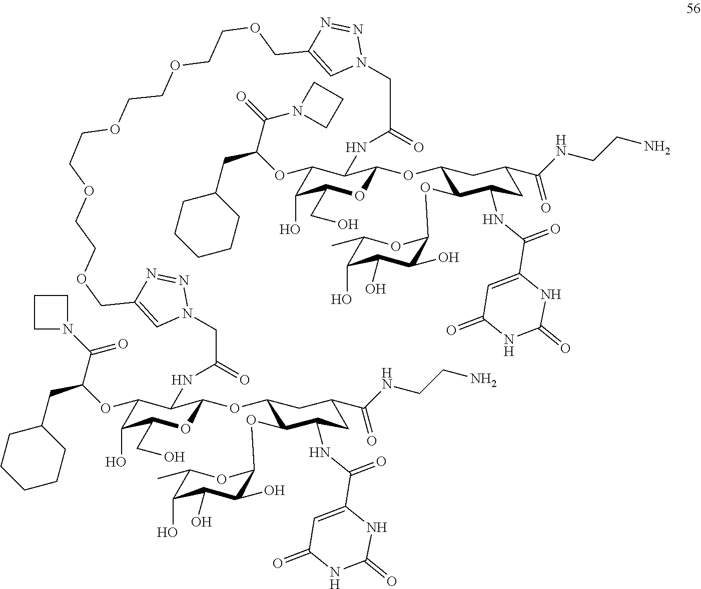

### Example 33

### Prophetic Synthesis of Multimeric Compound 57

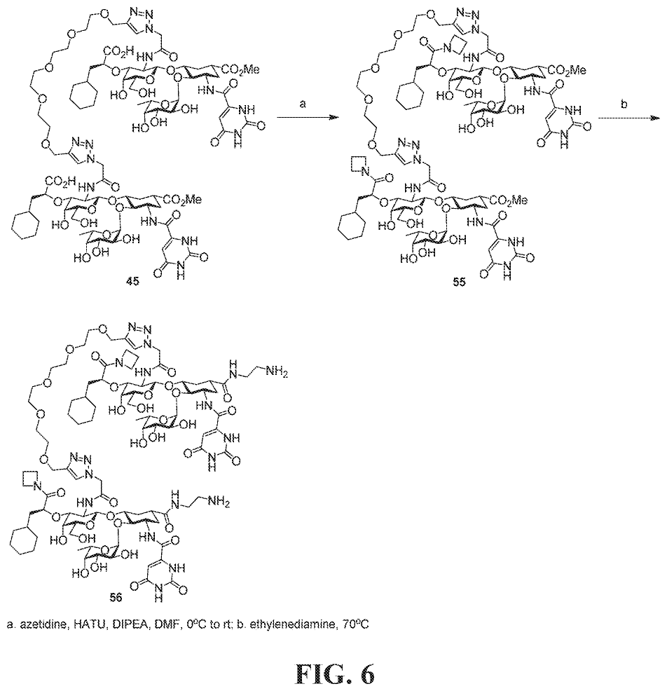

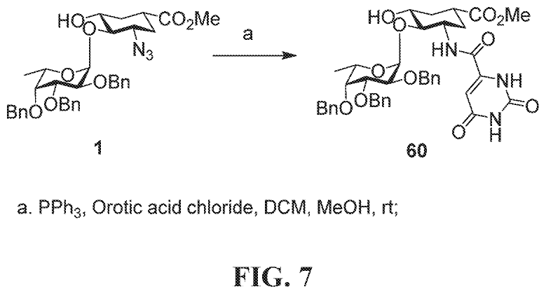

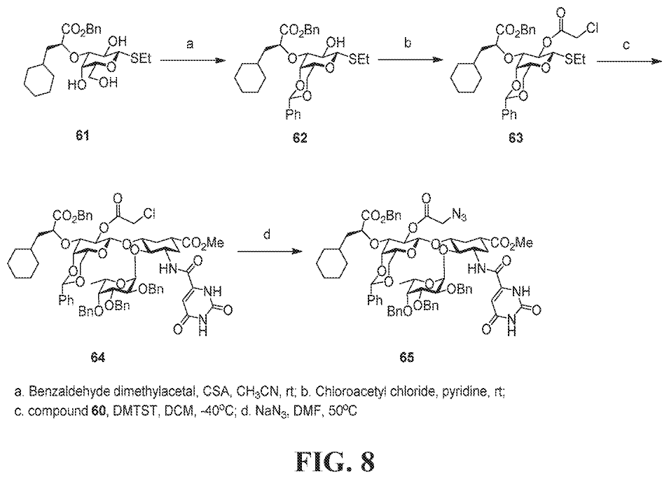

Compound 57: Compound 57 can be prepared in an analogous fashion to FIG. 6 using ethylamine in place of azetidine in step a.

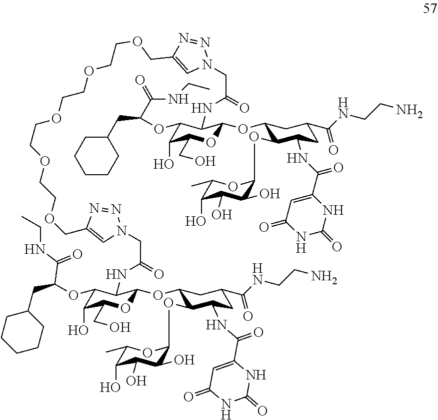

### Example 34

### Prophetic Synthesis of Multimeric Compound 58

Compound 58: Compound 58 can be prepared in an analogous fashion to FIG. 6 using dimethylamine in place of azetidine in step a.

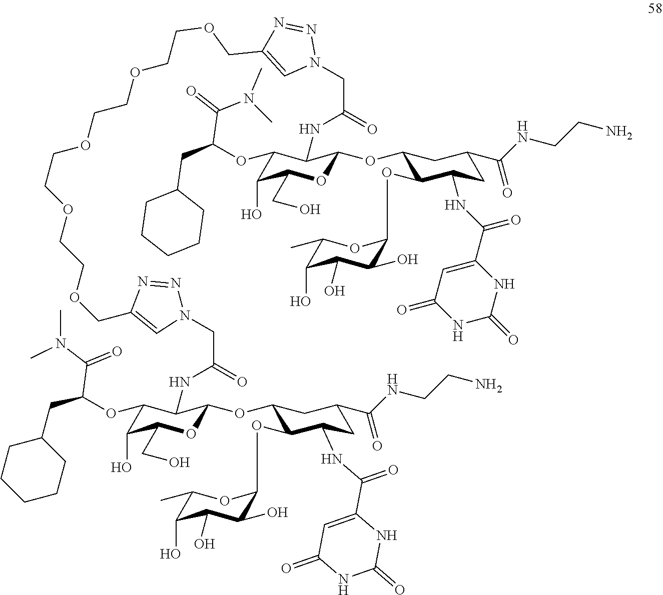

### Example 35

### Prophetic Synthesis of Multimeric Compound 59

Compound 59: Compound 59 can be prepared in an analogous fashion to FIG. 6 using 1,2-bis(2-aminoethoxy)ethane in place of ethylenediamine in step b.

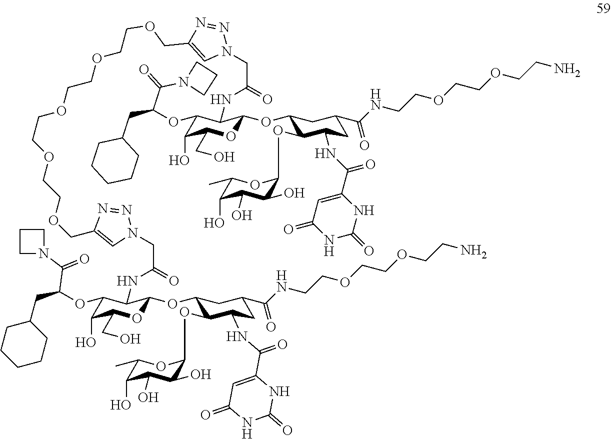

### Example 36

### Prophetic Synthesis of Multimeric Compound 66

Compound 60: To a stirred solution of compound 1 in DCM/MeOH (25/1) at room temperature is added orotic acid chloride (5 eq) and triphenylphosphine (5 eq). The reaction mixture is stirred 24 hours. The solvent is removed and the residue is separated by column chromatography to afford compound 60.

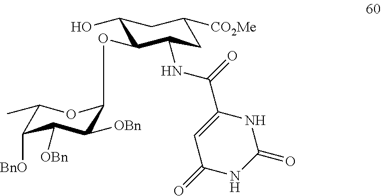

Compound 62: Compound 61 is dissolved in acetonitrile at room temperature. Benzaldehyde dimethylacetal (1.1 eq) is added followed by camphorsulfonic acid (0.2 eq). The reaction mixture is stirred until completion. Triethylamine is added. The solvent is removed and the residue separated by flash chromatography to afford compound 62.

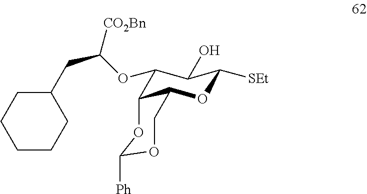

Compound 63: Compound 62 is dissolved in pyridine at room temperature. Dimethylaminopyridine (0.01 eq) is added followed by chloroacetyl chloride (2 eq). The reaction mixture is stirred until completion. The solvent is removed under educed pressure. The residue is dissolved in ethyl acetate, transferred to a separatory funnel and washed two times with 0.1N HCl and two times with water. The organic phase is dried over sodium sulfate, filtered, and concentrated. The residue is separated by column chromatograph to afford compound 63.

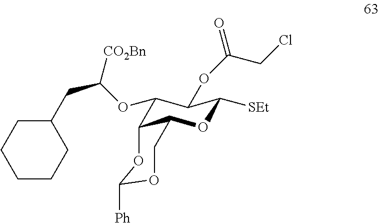

Compound 64: Activated powdered 4 Å molecular sieves are added to a solution of compound 60 and compound 63 (2 eq) in dry DCM under argon. The mixture is stirred for 2 hours at room temperature. Solid DMTST (1.5 eq) is added in 4 portions over 1.5 hours. The reaction mixture is stirred overnight at room temperature. The reaction mixture is filtered through Celite, transferred to a separatory funnel and washed two times with half saturated sodium bicarbonate and two times with water. The organic phase is dried over sodium sulfate, filtered and concentrated. The residue is separated by flash chromatography to afford compound 64.

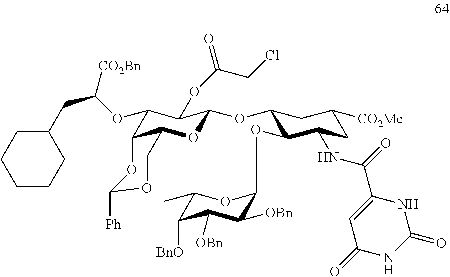

Compound 65: Compound 64 is dissolved in DMF. Sodium azide (1.5 eq) is added and the reaction mixture is stirred at 50° C. until completion. The reaction mixture is cooled to room temperature, diluted with ethyl acetate and transferred to a separatory funnel. The organic phase is washed 4 times with water then dried over sodium sulfate and concentrated. The residue is separated by column chromatography to afford compound 65.

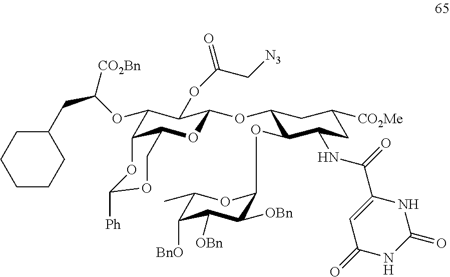

Compound 66: A solution of bispropagyl PEG-5 (compound 43) and compound 65 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at 50° C. The solution is concentrated under reduced pressure. The crude product is purified by chromatography to give a compound 66.

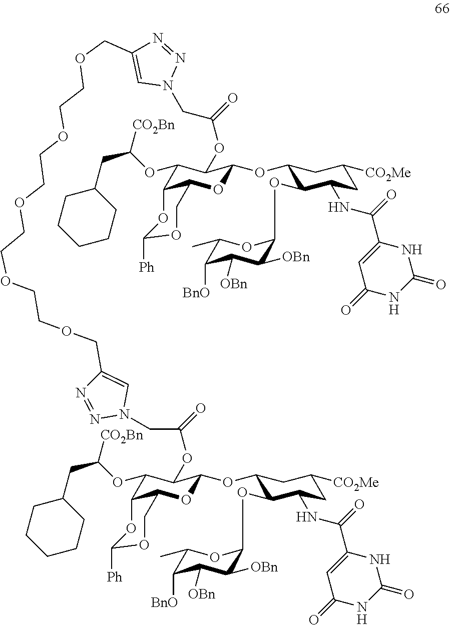

### Example 37

### Prophetic Synthesis of Multimeric Compound 67

Compound 67: To a solution of compound 66 in dioxane/water (4/1) is added Pd(OH)₂/C. The reaction mixture is stirred vigorously overnight under a hydrogen atmosphere. The reaction mixture is filtered through Celite and concentrated. The residue is purified by C-19 reverse phase column chromatography to afford compound 67.

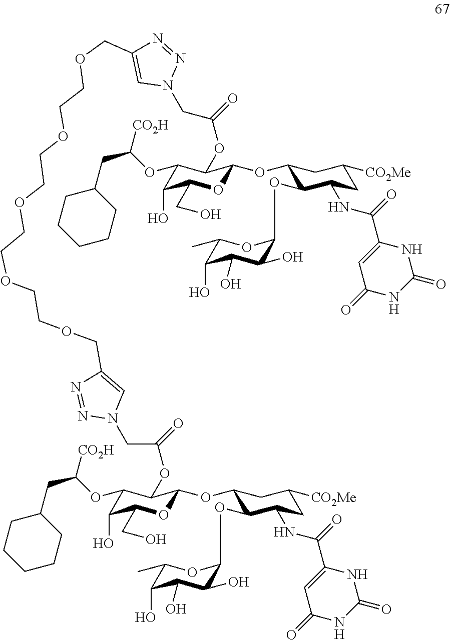

### Example 38

### Prophetic Synthesis of Multimeric Compound 68

Compound 68: Compound 67 is dissolved in ethylenediamine and stirred for 12 hrs at 70° C. The reaction mixture is concentrated under reduced pressure. The crude product is purified by C-18 column chromatography followed by lyophilization to afford compound 68.

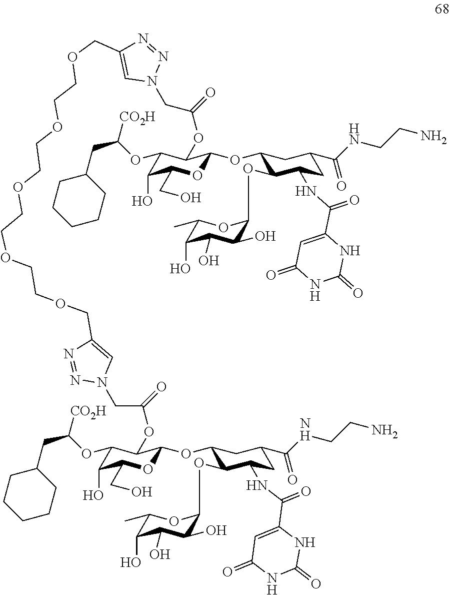

### Example 39

### Prophetic Synthesis of Multimeric Compound 69

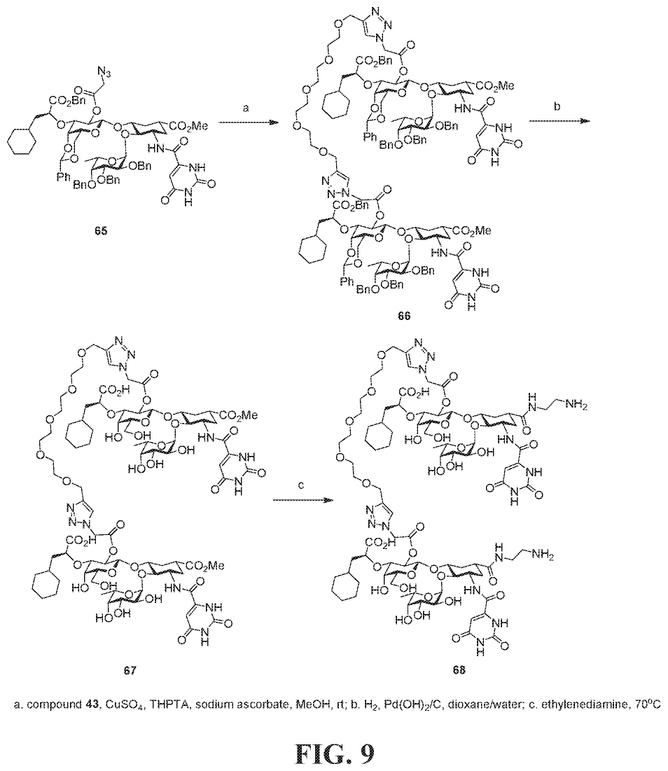

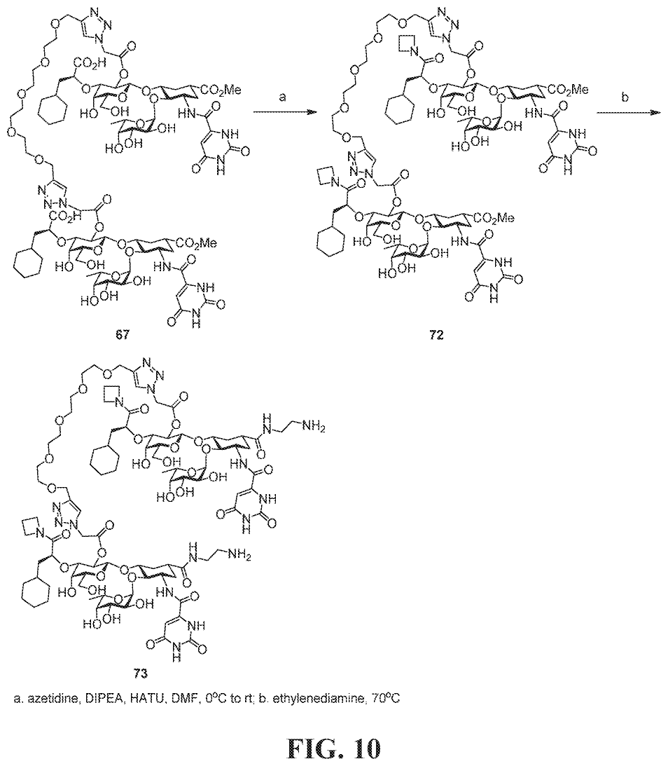

Compound 69: Compound 69 can be prepared in an analogous fashion to FIG. 9 by replacing compound 43 with PEG-8 bis propargyl ether in step a.

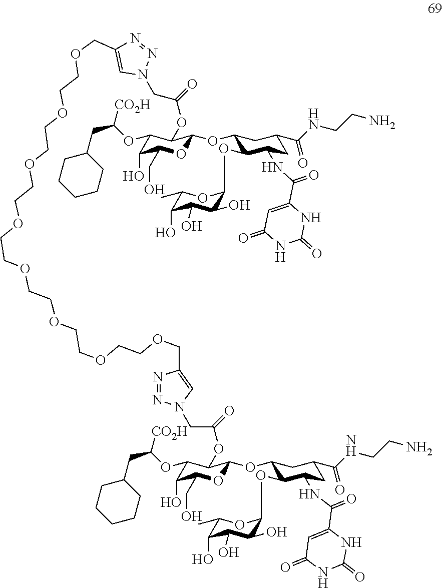

### Example 40

### Prophetic Synthesis of Multimeric Compound 70

Compound 70: Compound 70 can be prepared in an analogous fashion to FIG. 9 by replacing compound 43 with ethylene glycol bis propargyl ether in step a.

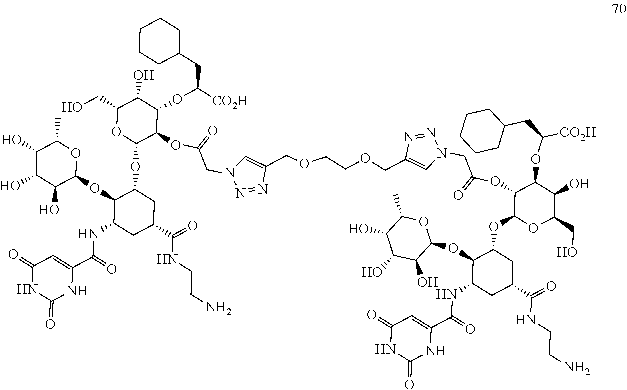 [SMILES: C[C@@H]1O[C@@H](O[C@@H]2[C@@H](NC(=O)c3cc(=O)[nH]c(=O)[nH]3)C[C@@H](C(=O)NCCN)C[C@H]2O[C@@H]2O[C@H](CO)[C@H](O)[C@H](O[C@@H](CC3CCCCC3)C(=O)O)[C@H]2OC(=O)Cn2cc(COCCOCc3cn(CC(=O)O[C@H]4[C@H](O[C@@H]5C[C@H](C(=O)NCCN)C[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 41

### Prophetic Synthesis of Multimeric Compound 71

Compound 71: Compound 71 can be prepared in an analogous fashion to FIG. 9 using 3,3′-[[2,2-bis[(2-propyn-1-yloxy)methyl]-1,3-propanediyl]bis(oxy)]bis-1-propyne in place of compound 43 in step a.

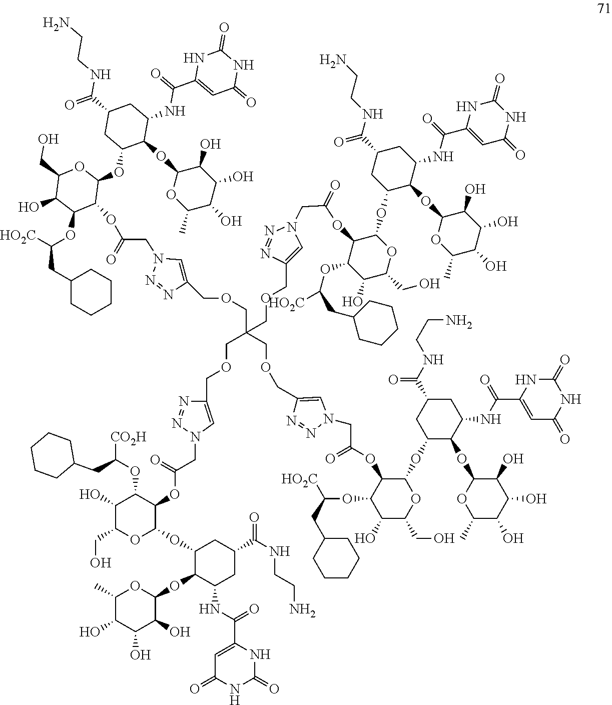

### Example 42

### Prophetic Synthesis of Multimeric Compound 72

Compound 72: Compound 67 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by flash chromatography to afford compound 72.

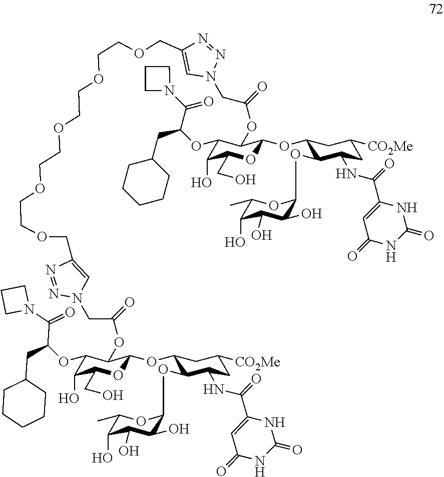

### Example 43

### Prophetic Synthesis of Multimeric Compound 73

Compound 73: Compound 72 is dissolved in ethylenediamine and stirred for 12 hrs at 70° C. The reaction mixture is concentrated under reduced pressure. The crude product is purified by C-18 column chromatography followed by lyophilization to afford compound 73.

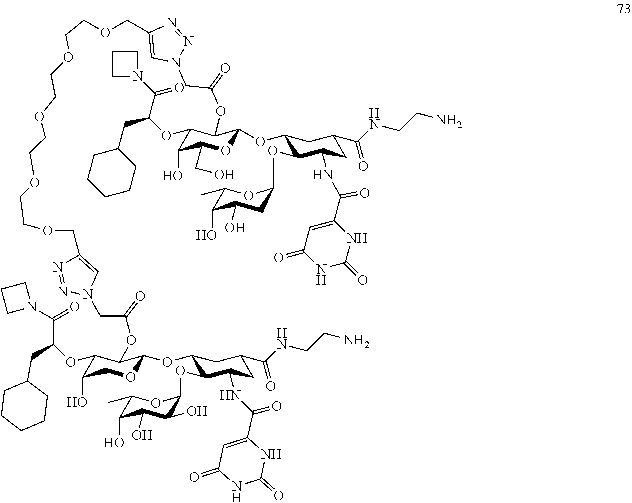

### Example 44

### Synthesis of Multimeric Compound 76

Compound 75: To a degassed solution of compound 74 (synthesis described in WO 2013/096926) (0.5 g, 0.36 mmole) in anhydrous DCM (10 mL) at 0° C. was added Pd(PPh₃)₄ (42 mg, 36.3 μmole, 0.1 eq), Bu₃SnH (110 μL, 0.4 μmole, 1.1 eq) and azidoacetic anhydride (0.14 g, 0.73 mmole, 2.0 eq). The resulting solution was stirred for 12 hrs under N₂ atmosphere while temperature was gradually increased to room temperature. After the reaction was completed, the solution was diluted with DCM (20 mL), washed with distilled water, dried over Na₂SO₄, then concentrated. The crude product was purified by combi-flash (EtOAc/Hex, Hex only—3/2, v/v) to give compound 75 (0.33 g, 67%). MS: Calculated (C₈₁H₉₅N₄O₁₆, 1376.6), ES-Positive (1400.4, M+Na)).

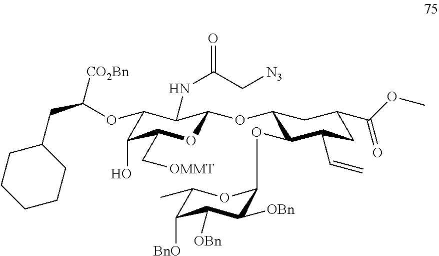

Compound 76: A solution of bispropargyl PEG-5 (compound 43, 27 mg, 0.1 mmole) and compound 75 (0.33 g, 0.24 mmole, 2.4 eq) in a mixed solution (MeOH/1,4 dioxane, 2/1, v/v, 12 mL) was degassed at room temperature. A solution of CuSO₄/THPTA in distilled water (0.04 M) (0.5 mL, 20 μmole, 0.2 eq) and sodium ascorbate (4.0 mg, 20 μmole, 0.2 eq) were added successively and the resulting solution was stirred 12 hrs at 70° C. The solution was cooled to room temperature and concentrated under reduced pressure. The crude product was purified by combi-flash (EtOAc/MeOH, EtOAc only—4/1, v/v) to give a compound 76 as a white foam (0.23 g, 70%).

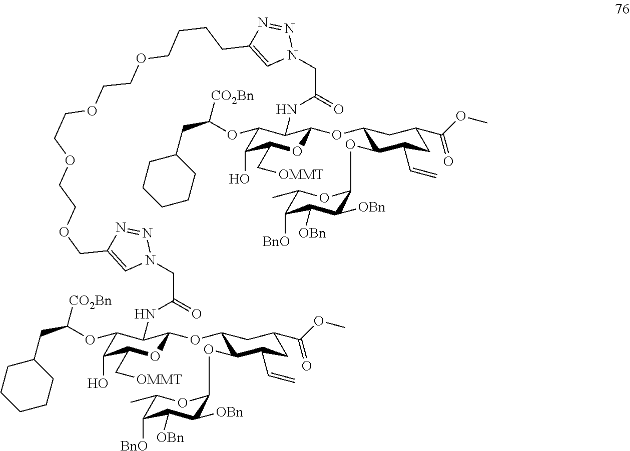

### Example 45

### Synthesis of Multimeric Compound 77

Compound 77: A solution of compound 76 (0.23 g, 0.76 μmole) in solution of MeOH/i-PrOH (2/1, v/v, 12 mL) was hydrogenated in the presence of Pd(OH)₂ (0.2 g) and 1 atm of H₂ gas pressure for 24 hrs at room temperature. The solution was filtered through a Celite pad and the cake was washed with MeOH. The combined filtrate was concentrated under reduced pressure. The crude product was washed with hexane and dried under high vacuum to give compound 77 as a white solid (0.14 g, quantitative). MS: Calculated (C₈₀H₁₃₀N₈O₃₅, 1762.8), ES-positive (1785.4, M+Na), ES-Negative (1761.5, M−1, 879.8).

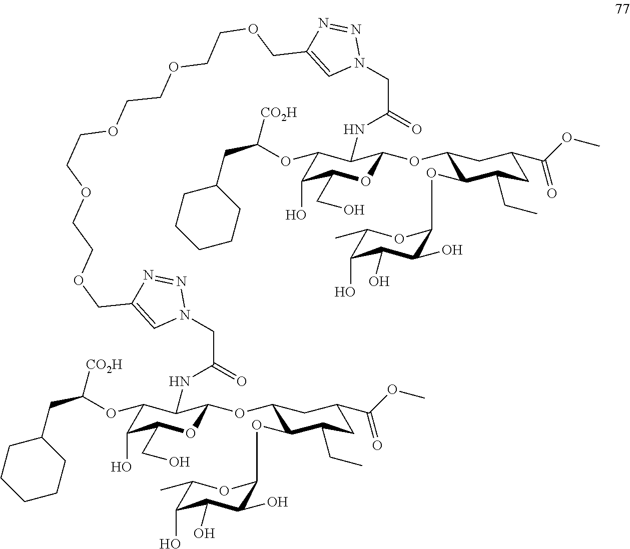

### Example 46

### Prophetic Synthesis of Multimeric Compound 78

Compound 78: Compound 77 (60 mg, 34.0 μmole) was dissolved in ethylenediamine (3 mL) and the homogeneous solution was stirred for 12 hrs at 70° C. The reaction mixture was concentrated under reduced pressure and the residue was dialyzed against distilled water with MWCO 500 dialysis tube. The crude product was further purified by C-18 column chromatography with water/MeOH (9/1-1/9, v/v) followed by lyophilization to give a compound 78 as a white solid (39 mg, 63%).

1H NMR (400 MHz, Deuterium Oxide) δ 8.00 (s, 2H), 5.26-5.14 (two d, J=16.0 Hz, 4H), 4.52 (d, J=4.0 Hz, 2H), 4.84 (dd, J=8.0 Hz, J=4.0 Hz, 2H), 4.66 (s, 4H), 4.54 (broad d, J=12 Hz, 2H), 3.97 (broad t, 2H), 3.91-3.78 (m, 6H), 3.77-3.58 (m, 28H), 3.57-3.46 (m, 4H), 3.42 (t, J=8.0 Hz, 6H), 3.24 (t, J=12.0 Hz, 2H), 3.02 (t, J=6.0 Hz, 4H), 2.67 (s, 2H), 2.32 (broad t, J=12 Hz, 2H), 2.22-2.06 (m, 2H), 1.96-1.74 (m, 4H), 1.73-1.39 (m, 18H), 1.38-1.21 (m, 6H), 1.20-0.99 (m, J=8.0 Hz, 14H), 0.98-0.73 (m, J=8.0 Hz, 10H).

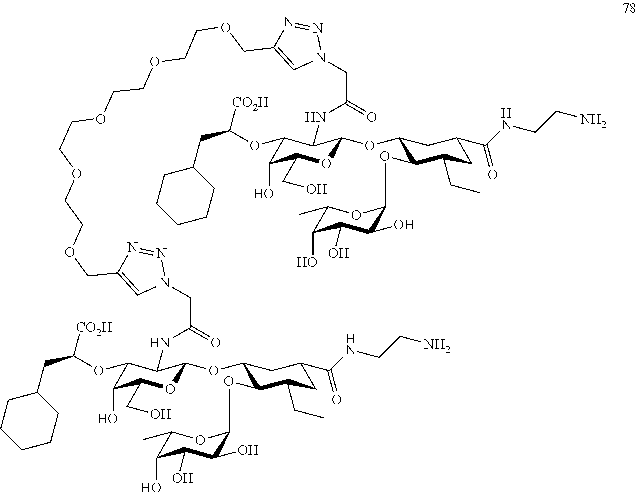

### Example 47

### Prophetic Synthesis of Multimeric Compound 79

Compound 79: Compound 79 can be prepared in an analogous fashion to FIG. 11 using 3-azidopropanoic anhydride (Yang, C. et. al. JACS, (2013) 135(21), 7791-7794) in place of azidoacetic anhydride in step a.

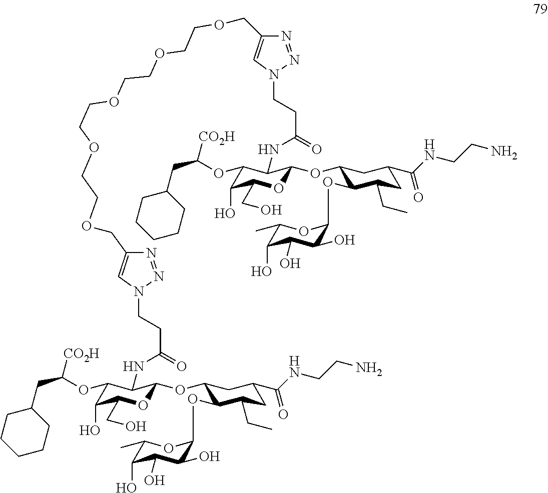

### Example 48

### Prophetic Synthesis of Multimeric Compound 80

Compound 80: Compound 80 can be prepared in an analogous fashion to FIG. 11 using 4-azidobutanoic anhydride (Yang, C. et. al. JACS, (2013) 135(21), 7791-7794) in place of azidoacetic anhydride in step a.

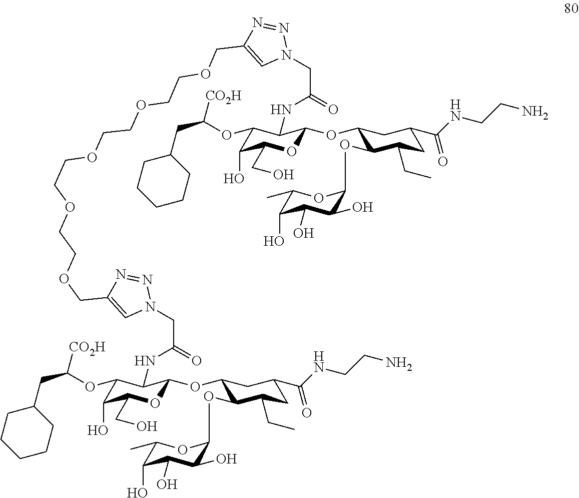

### Example 49

### Prophetic Synthesis of Multimeric Compound 81

Compound 81: Compound 81 can be prepared in an analogous fashion to FIG. 11 using 4-azidobutanoic anhydride (Yang. C. et. al. JACS, (2013) 135(21), 7791-7794) in place of azidoacetic anhydride in step a and using 1,2-bi(2-propynyloxy) ethane in place of compound 43 in step b.

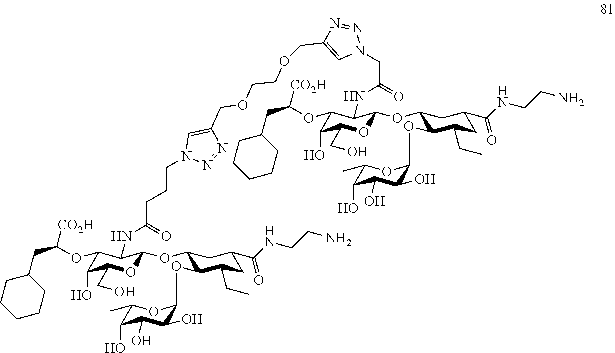

### Example 50

### Prophetic Synthesis of Multimeric Compound 82

Compound 82: Compound 82 can be prepared in an analogous fashion to FIG. 11 using 4,7,10,13,16,19,22,25,28,31-decaoxatetratriaconta-1,33-diyne in place of compound 43 in step b.

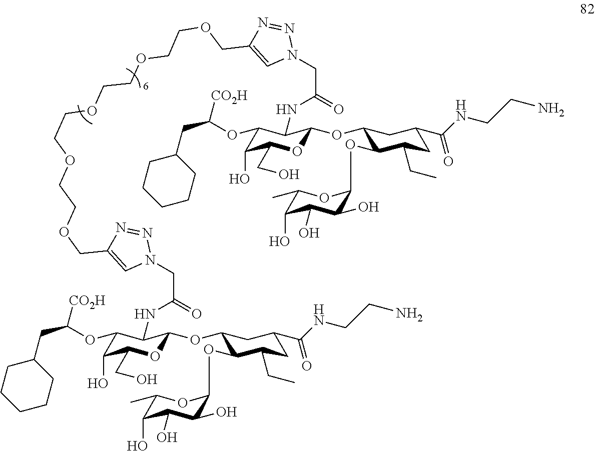

### Example 51

### Prophetic Synthesis of Multimeric Compound 83

Compound 83: Compound 83 can be prepared in an analogous fashion to FIG. 11 using 2-aminoethylether in place of ethylenediamine in step d.

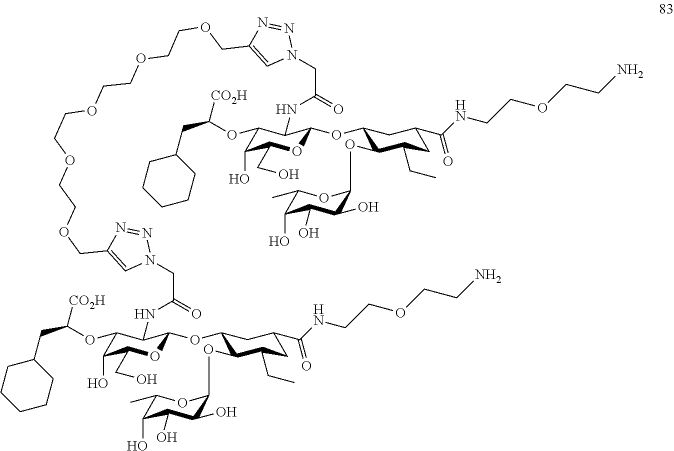

### Example 52

### Prophetic Synthesis of Multimeric Compound 84

Compound 84: Compound 84 can be prepared in an analogous fashion to FIG. 11 using 1,2-bi(2-propynyloxy) ethane in place of compound 43 in step b.

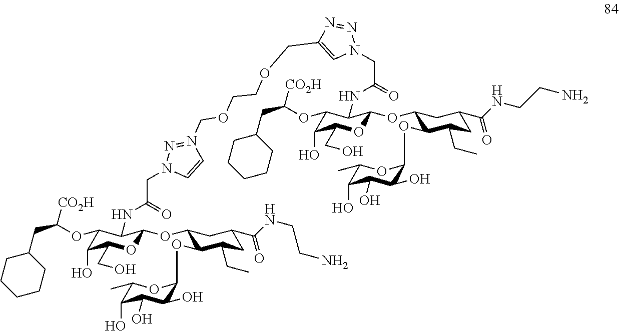

### Example 53

### Prophetic Synthesis of Multimeric Compound 85

Compound 85: Compound 85 can be prepared in an analogous fashion to FIG. 11 using PEG-8 dipropargyl ether in place of compound 43 in step b and 1,5-diaminopentane in place of ethylenediamine in step d.

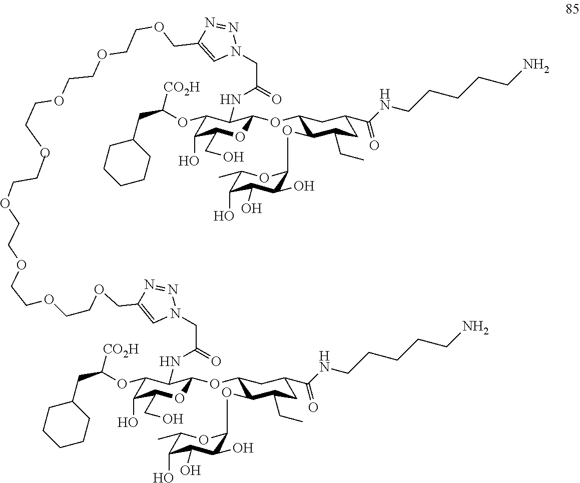

### Example 54

### Prophetic Synthesis of Multimeric Compound 86

Compound 86: Compound 77 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by flash chromatography to afford compound 86.

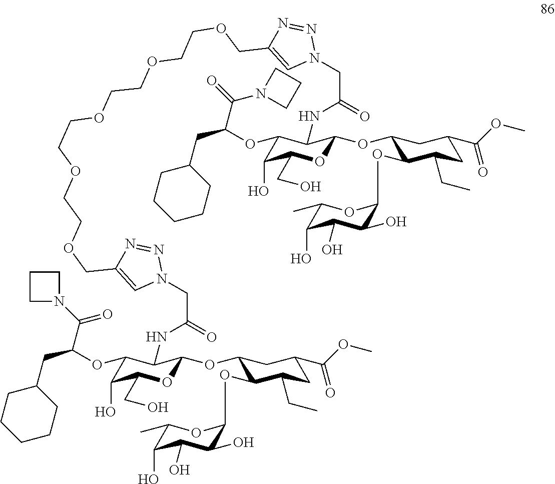

### Example 55

### Prophetic Synthesis of Multimeric Compound 87

Compound 87: Compound 86 is dissolved in ethylenediamine stirred for 12 hrs at 70° C. The reaction mixture was concentrated under reduced pressure. The residue was purified by C-18 column chromatography followed by lyophilization to give a compound 87.

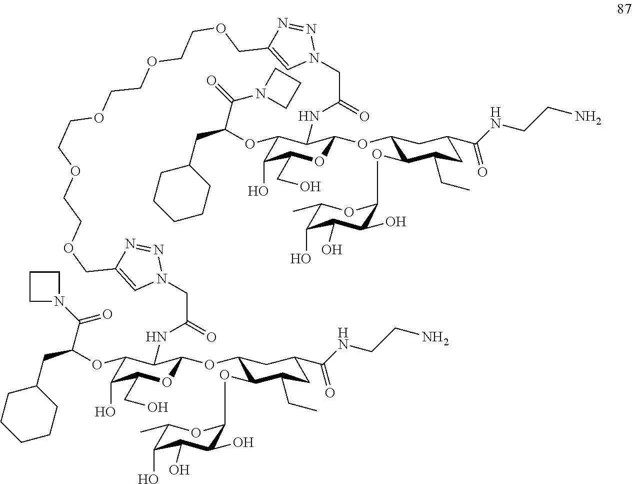

### Example 56

### Prophetic Synthesis of Multimeric Compound 88

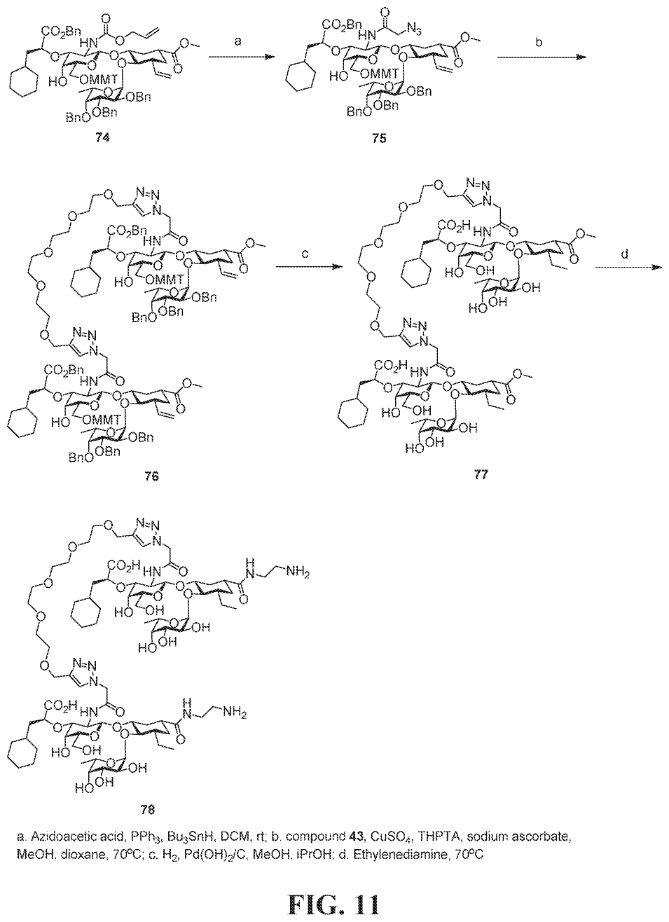

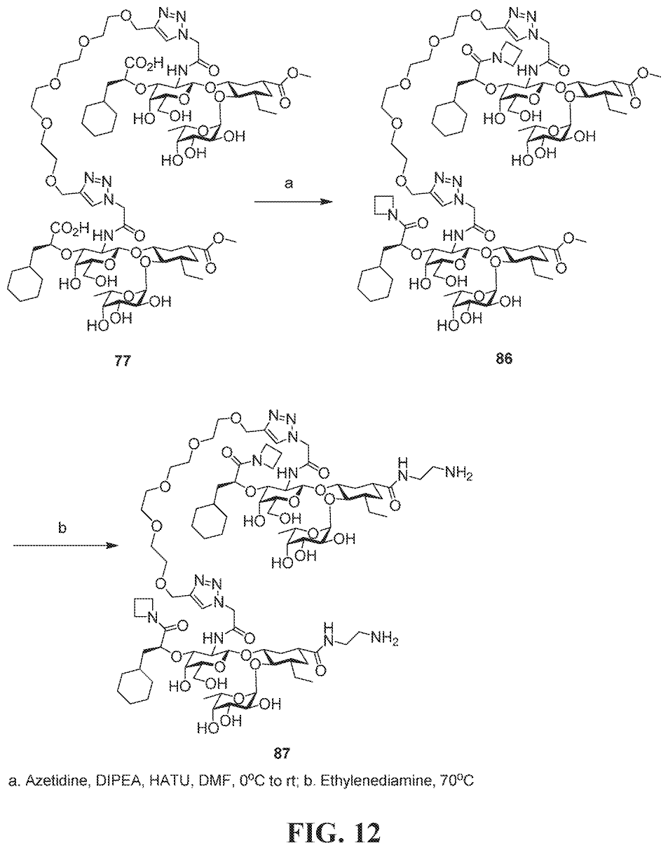

Compound 88: Compound 88 can be prepared in an analogous fashion to FIG. 12 using 2-aminoethylether in place of ethylenediamine in step b.

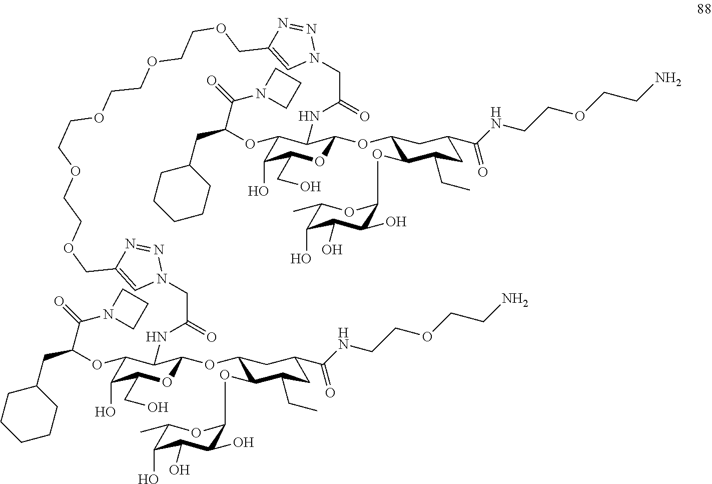

### Example 57

### Prophetic Synthesis of Multimeric Compound 89

Compound 89: Compound 89 can be prepared in an analogous fashion to FIG. 12 using dimethylamine in place of azetidine in step a and 2-aminoethylether in place of ethylenediamine in step b.

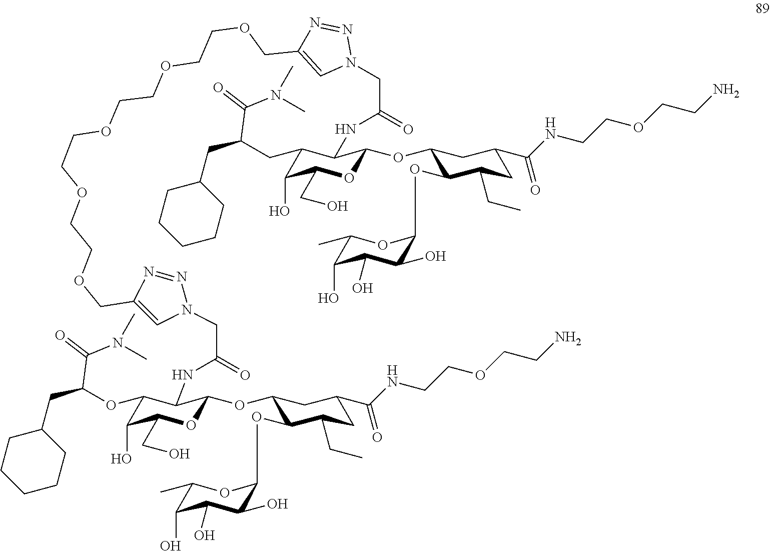

### Example 58

### Prophetic Synthesis of Multimeric Compound 90

Compound 90: Compound 90 can be prepared in an analogous fashion to FIG. 12 using piperidine in place of azetidine in step a.

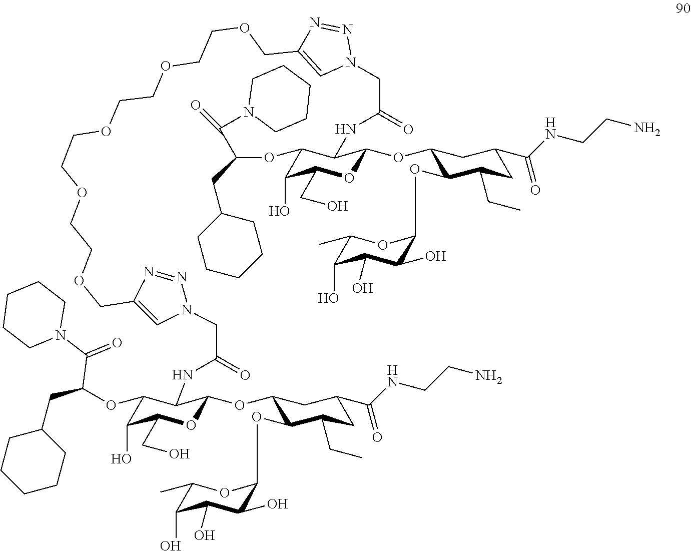

### Example 59

### Prophetic Synthesis of Multimeric Compound 91

Compound 91: Compound 91 can be prepared in an analogous fashion to FIGS. 11 and 12 using in PEG-9 bis-propargyl ether in place of compound 43 in step b of Scheme 11.

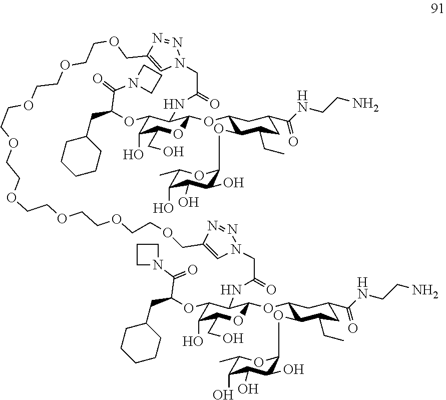

### Example 60

### Prophetic Synthesis of Multimeric Compound 92

Compound 92: Compound 92 can be prepared in an analogous fashion to FIGS. 11 and 12 using 1,2-bi(2-propynyloxy) ethane in place of compound 43 in step b in Scheme 11.

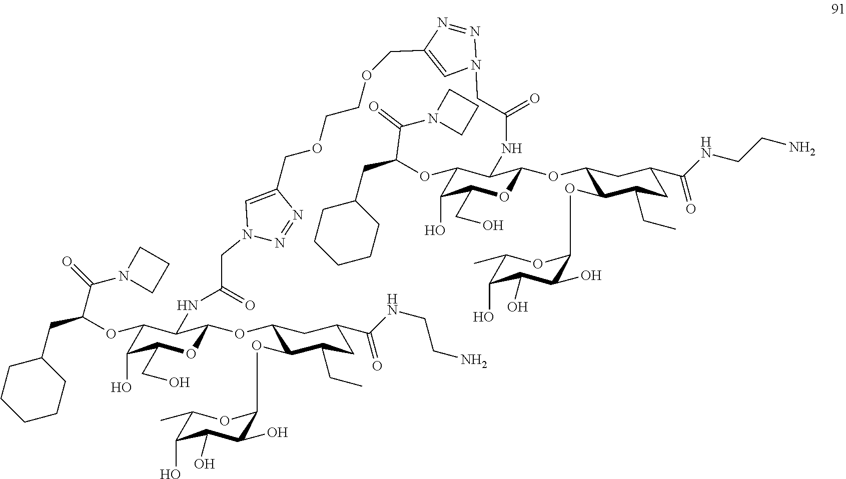

### Example 61

### Prophetic Synthesis of Multimeric Compound 93

Compound 93: Compound 93 can be prepared in an analogous fashion to FIGS. 11 and 12 using 1,2-bi(2-propynyloxy) ethane in place of compound 43 in step b in Scheme 11 and using 2-aminoethyl ether in place of ethylenediamine in step b of Scheme 12.

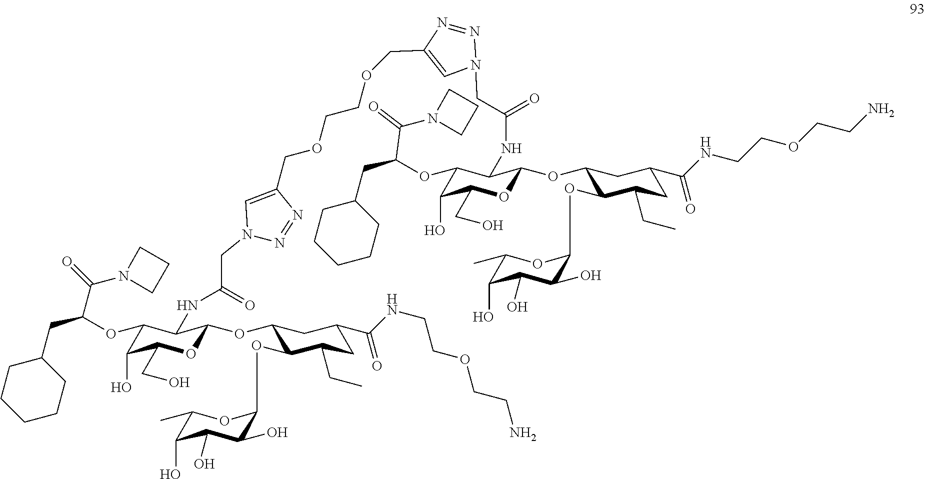

### Example 62

### Synthesis of Multimeric Compound 95

Compound 95: Compound 22 and compound 94 (5 eq)(preparation described in WO/2016089872) is co-evaporated 3 times from methanol and stored under vacuum for 1 hour. The mixture is dissolved in methanol under an argon atmosphere and stirred for 1 hour at room temperature. Sodium triacetoxy borohydride (15 eq) is added and the reaction mixture is stirred overnight at room temperature. The solvent is removed and the residue is separated by C-18 reverse phase chromatography.

The purified material is dissolved in methanol at room temperature. The pH is adjusted to 12 with 1N NaOH. The reaction mixture is stirred at room temperature until completion. The pH is adjusted to 9. The solvent is removed under vacuum and the residue is separated by C-18 reverse phase chromatography to afford compound 95.

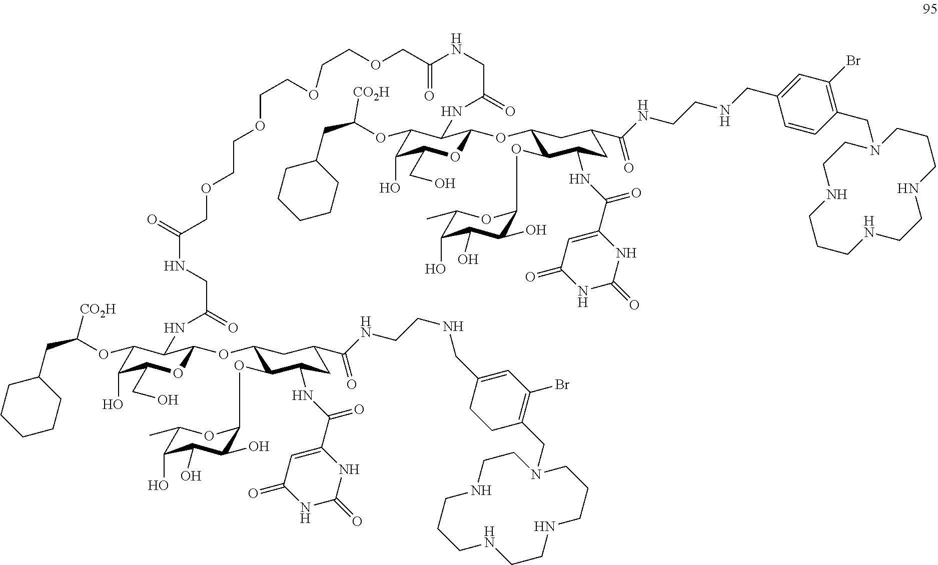

### Example 63

### Prophetic Synthesis of Multimeric Compound 96

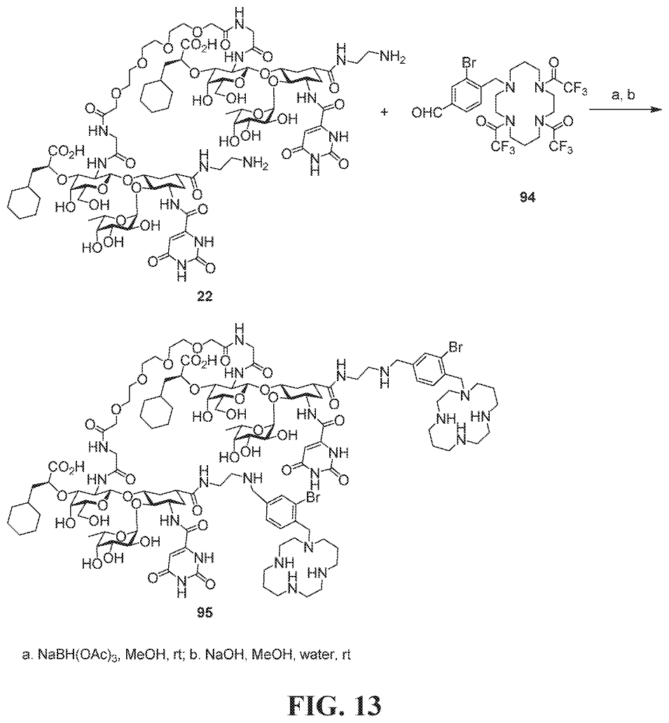

Compound 96: Compound 96 can be prepared in an analogous fashion to FIG. 13 by replacing compound 22 with compound 23 in step a.

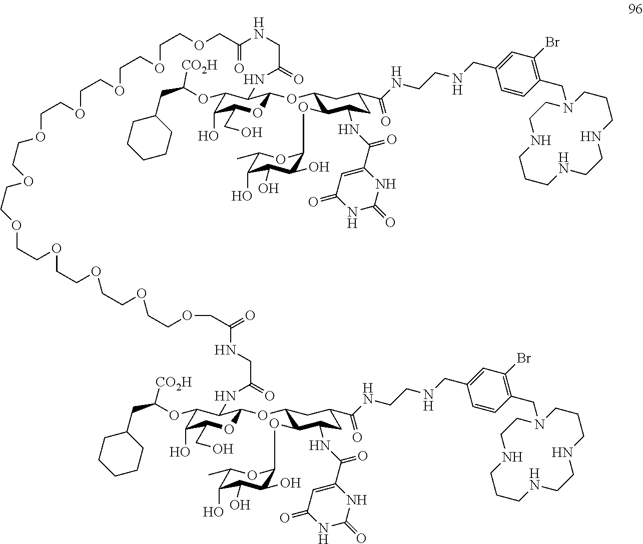

### Example 64

### Prophetic Synthesis of Multimeric Compound 97

Compound 97: Compound 97 can be prepared in an analogous fashion to FIG. 13 by replacing compound 22 with compound 24 in step a.

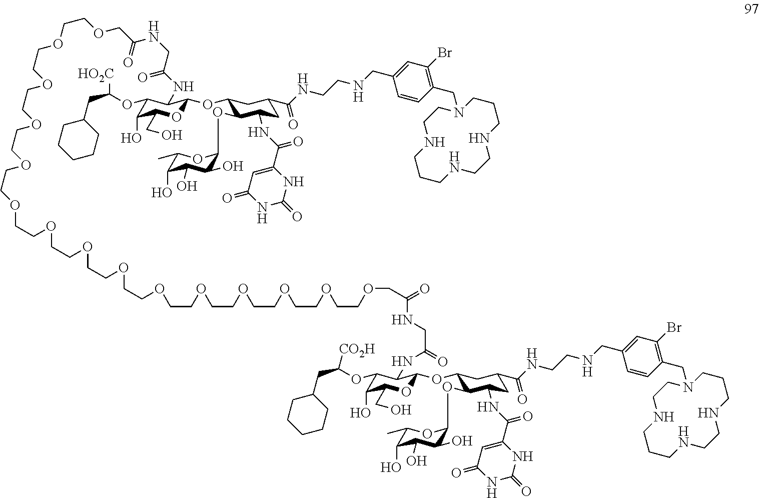

### Example 65

### Prophetic Synthesis of Multimeric Compound 98

Compound 98: Compound 98 can be prepared in an analogous fashion to FIG. 13 by replacing compound 22 with compound 25 in step a.

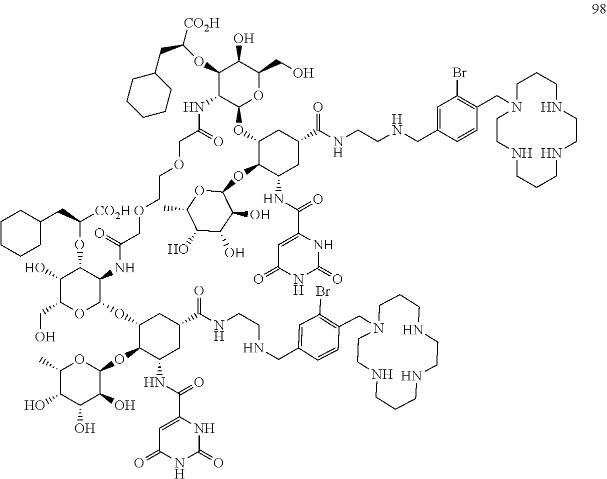 [SMILES: C[C@@H]1O[C@@H](O[C@@H]2[C@@H](NC(=O)c3cc(=O)[nH]c(=O)[nH]3)C[C@@H](C(=O)NCCNCc3ccc(CN4CCCNCCNCCCNCC4)c(Br)c3)C[C@H]2O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3C[C@H](C(=O)NCCNCc4ccc(CN5CCCNCCNCCCNCC5)c(Br)c4)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H](O)[C@H](O)[C@@H]1O]

### Example 66

### Prophetic Synthesis of Multimeric Compound 99

Compound 99: Compound 99 can be prepared in an analogous fashion to FIG. 13 by replacing compound 22 with compound 26 in step a.

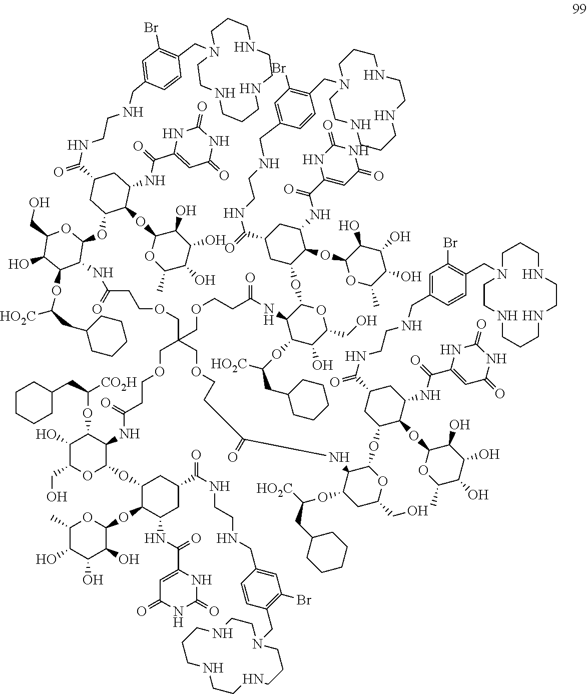

### Example 67

### Prophetic Synthesis of Multimeric Compound 100

Compound 100: Compound 100 can be prepared in an analogous fashion to FIG. 13 by replacing compound 22 with compound 27 in step a.

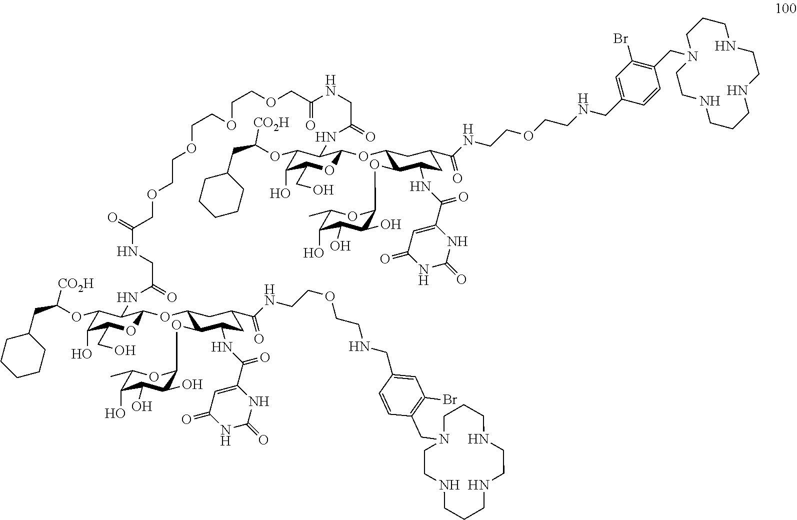

### Example 68

### Prophetic Synthesis of Multimeric Compound 101

Compound 101: Compound 101 con be prepared in an analogous fashion to FIG. 13 by replacing compound 22 with compound 28 in step a.

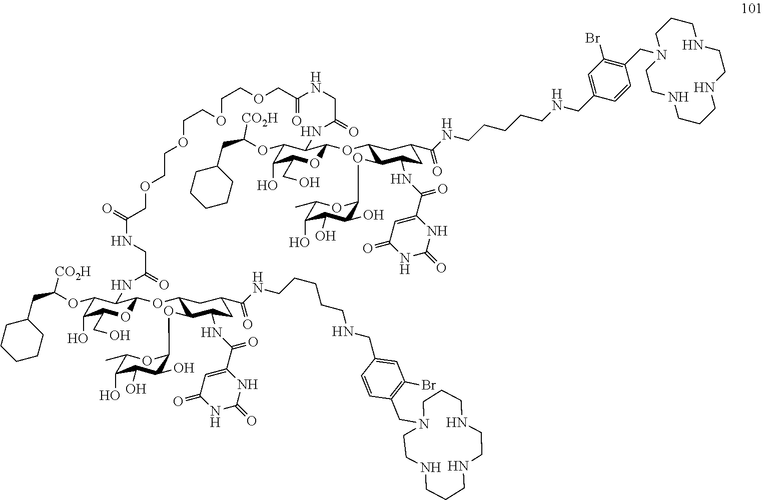

### Example 69

### Prophetic Synthesis of Multimeric Compound 102

Compound 102: Compound 102 can be prepared in an analogous fashion to FIG. 13 by replacing compound 22 with compound 29 in step a.

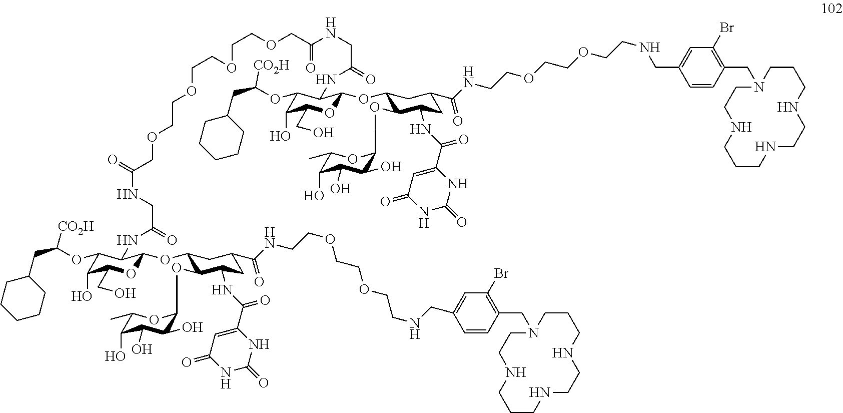

### Example 70

### Prophetic Synthesis of Multimeric Compound 103

Compound 103: Compound 103 can be prepared in an analogous fashion to FIG. 13 by replacing compound 22 with compound 30 in step a.

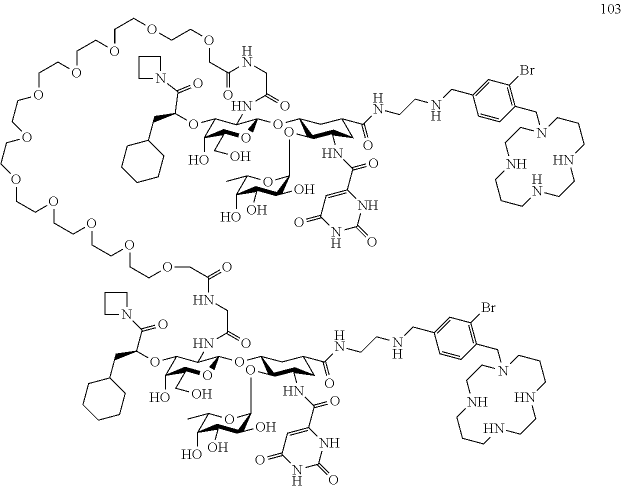

### Example 71

### Prophetic Synthesis of Multimeric Compound 104

Compound 104: Compound 104 can be prepared in an analogous fashion to FIG. 13 by replacing compound 22 with compound 31 in step a.

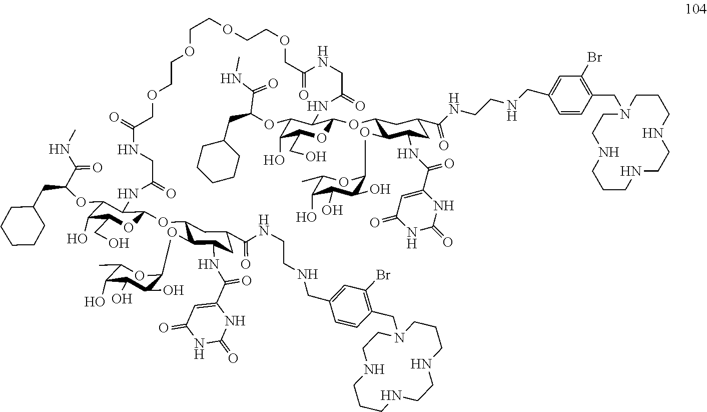

### Example 72

### Prophetic Synthesis of Multimeric Compound 105

Compound 105: Compound 105 can be prepared in an analogous fashion to FIG. 13 by replacing compound 22 with compound 32 in step a.

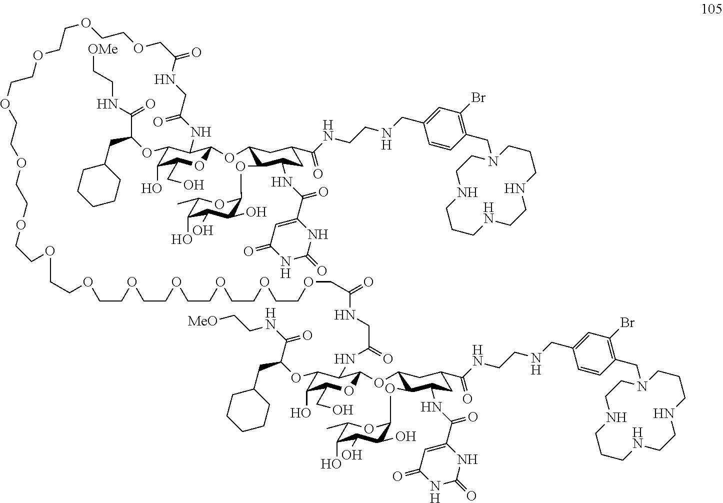

### Example 73

### Prophetic Synthesis of Multimeric Compound 106

Compound 106: Compound 106 can be prepared in an analogous fashion to FIG. 13 by replacing compound 22 with compound 33 in step a.

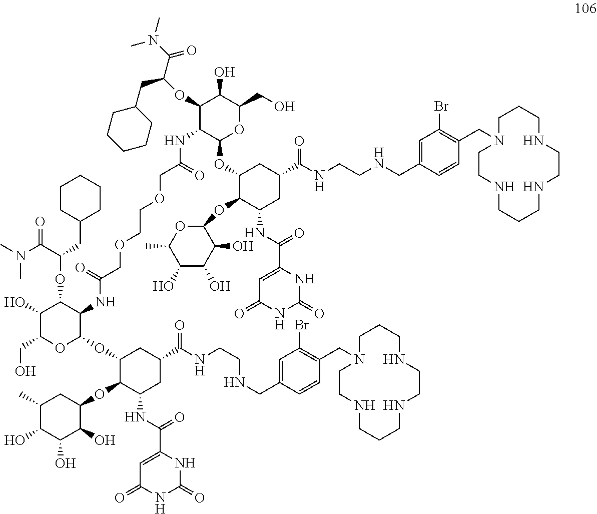 [SMILES: C[C@@H]1C[C@@H](O[C@@H]2[C@@H](NC(=O)c3cc(=O)[nH]c(=O)[nH]3)C[C@@H](C(=O)NCCNCc3ccc(CN4CCCNCCNCCCNCC4)c(Br)c3)C[C@H]2O[C@@H]2O[C@H](CO)[C@H](O)[C@H](O[C@@H](CC3CCCCC3)C(=O)N(C)C)[C@H]2NC(=O)COCCOCC(=O)N[C@H]2[C@H](O[C@@H]3C[C@H](C(=O)NCCNCc4ccc(CN5CCCNCCNCCCNCC5)c(Br)c4)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N(C)C)[C@@H](O)[C@H](O)[C@@H]1O]

### Example 74

### Prophetic Synthesis of Multimeric Compound 107

Compound 107: Compound 107 can be prepared in an analogous fashion to FIG. 13 by replacing compound 22 with compound 34 in step a.

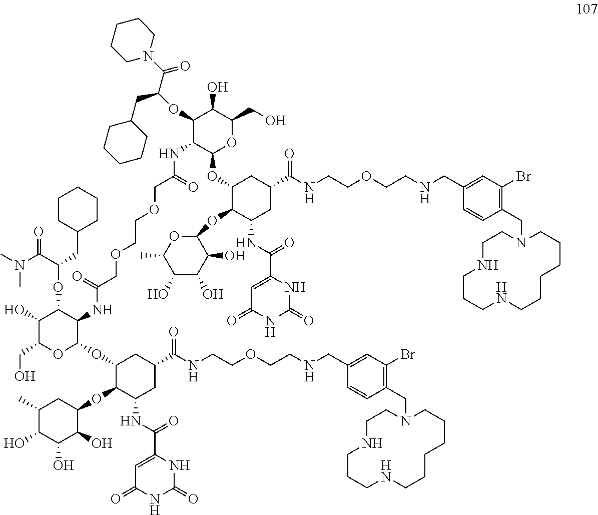 [SMILES: C[C@@H]1C[C@@H](O[C@@H]2[C@@H](NC(=O)c3cc(=O)[nH]c(=O)[nH]3)C[C@@H](C(=O)NCCOCCNCc3ccc(CN4CCCCCCNCCCNCC4)c(Br)c3)C[C@H]2O[C@@H]2O[C@H](CO)[C@H](O)[C@H](O[C@@H](CC3CCCCC3)C(=O)N(C)C)[C@H]2NC(=O)COCCOCC(=O)N[C@H]2[C@H](O[C@@H]3C[C@H](C(=O)NCCOCCNCc4ccc(CN5CCCCCCNCCCNCC5)c(Br)c4)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCCCC2)[C@@H](O)[C@H](O)[C@@H]1O]

### Example 75

### Prophetic Synthesis of Multimeric Compound 108

Compound 108: Compound 108 can be prepared in an analogous fashion to FIG. 13 by replacing compound 22 with compound 37 in step a.

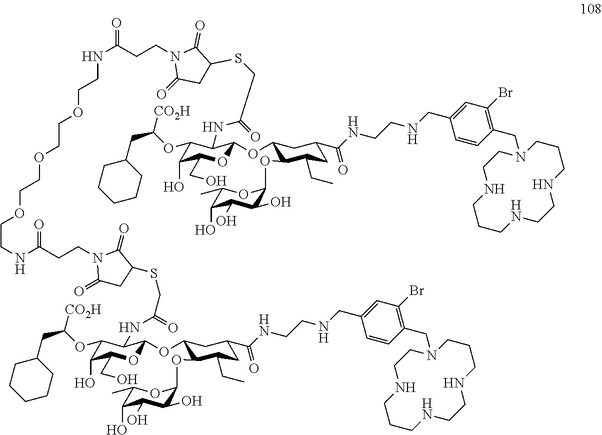

### Example 76

### Prophetic Synthesis of Multimeric Compound 109

Compound 109: Compound 109 can be prepared in an analogous fashion to FIG. 13 by replacing compound 22 with compound 38 in step a.

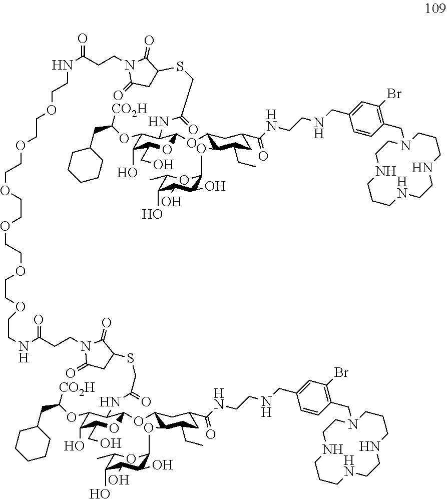

### Example 77

### Prophetic Synthesis of Multimeric Compound 110

Compound 110: Compound 110 can be prepared in an analogous fashion to FIG. 13 by replacing compound 22 with compound 39 in step a.

### Prophetic Synthesis of Multimeric Compound 111

Compound 111: Compound 111 can be prepared in an analogous fashion to FIG. 13 by replacing compound 22 with compound 40 in step a.

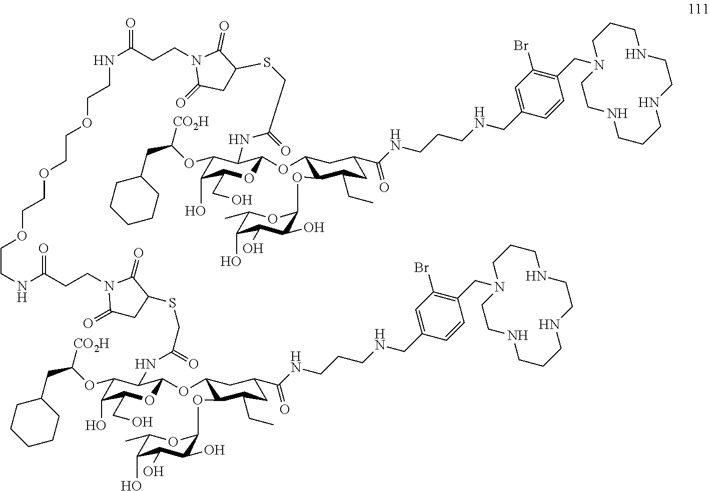

### Example 78

### Prophetic Synthesis of Multimeric Compound 112

Compound 112: Compound 112 can be prepared in an analogous fashion to FIG. 13 by replacing compound 22 with compound 46 in step a.

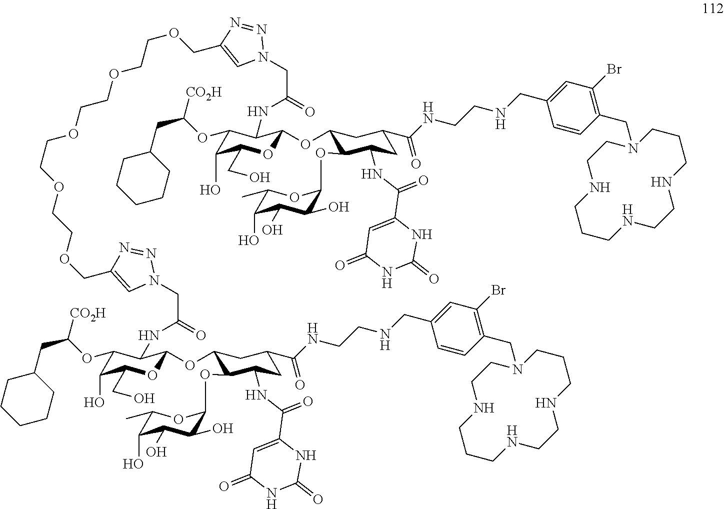

### Example 79

### Prophetic Synthesis of Multimeric Compound 113

Compound 113: Compound 113 can be prepared in an analogous fashion to FIG. 13 by replacing compound 22 with compound 47 in step a.

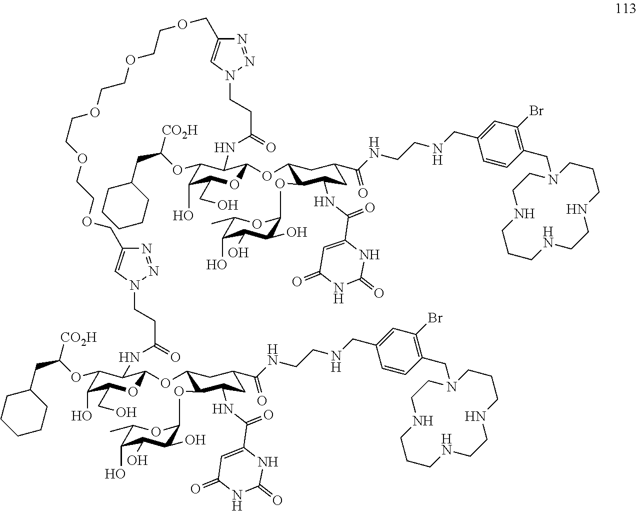

### Example 80

### Prophetic Synthesis of Multimeric Compound 114

Compound 114: Compound 114 can be prepared in an analogous fashion to FIG. 13 by replacing compound 22 with compound 48 in step a.

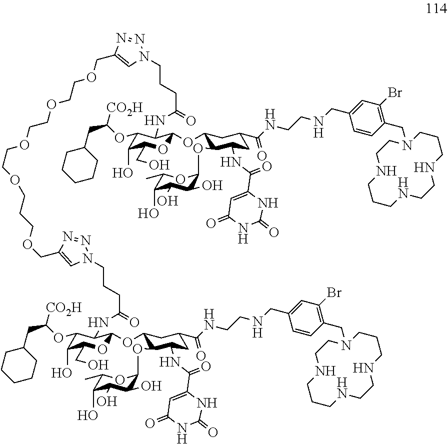

### Example 81

### Prophetic Synthesis of Multimeric Compound 115

Compound 115: Compound 115 can be prepared in an analogous fashion to FIG. 13 by replacing compound 22 with compound 49 in step a.

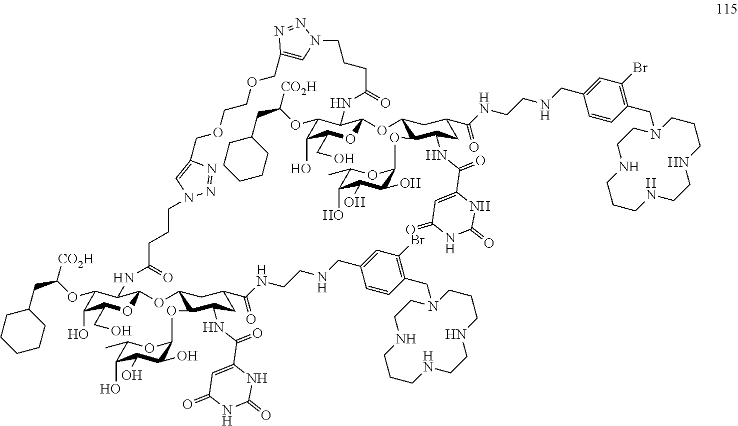

### Example 82

### Prophetic Synthesis of Multimeric Compound 116

Compound 116: Compound 116 can be prepared in an analogous fashion to FIG. 13 by replacing compound 22 with compound 50 in step a.

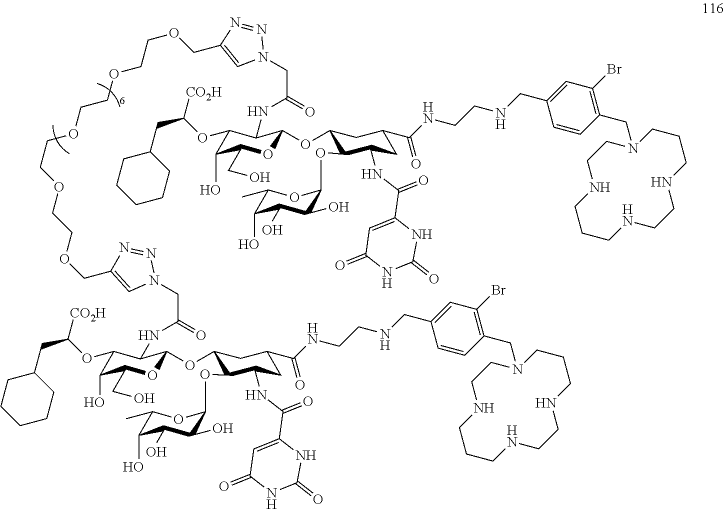

### Example 83

### Prophetic Synthesis of Multimeric Compound 117

Compound 117: Compound 117 can be prepared in an analogous fashion to FIG. 13 by replacing compound 22 with compound 51 in step a.

### Example 84

### Prophetic Synthesis of Multimeric Compound 118

Compound 118: Compound 118 can be prepared in an analogous fashion to FIG. 13 by replacing compound 22 with compound 52 in step a.

### Example 85

### Prophetic Synthesis of Multimeric Compound 119

Compound 119: Compound 119 can be prepared in an analogous fashion to FIG. 13 by replacing compound 22 with compound 53 in step a.

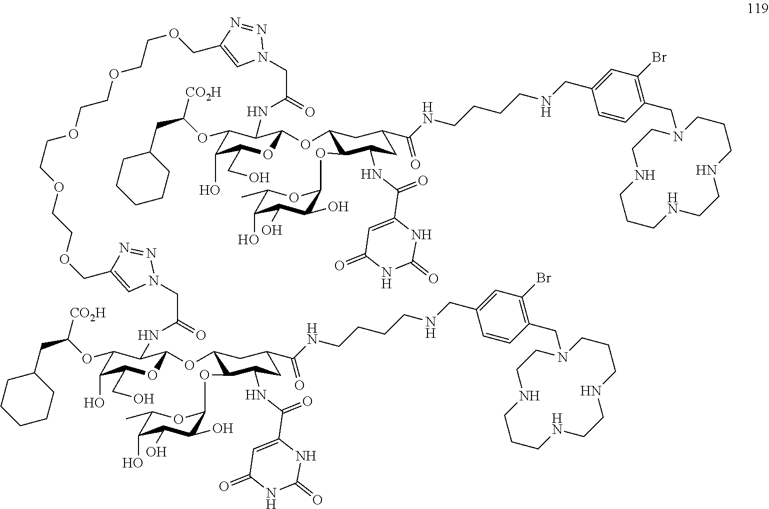

### Example 86

### Prophetic Synthesis of Multimeric Compound 120

Compound 120: Compound 120 can be prepared in an analogous fashion to FIG. 13 by replacing compound 22 with compound 54 in step a.

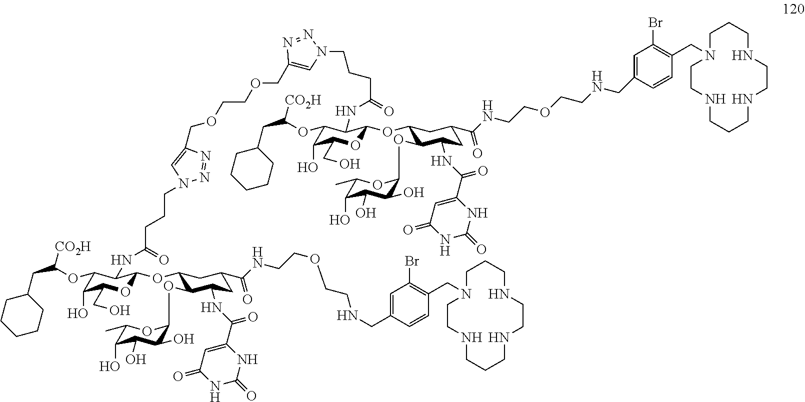

### Example 87

### Prophetic Synthesis of Multimeric Compound 121

Compound 121: Compound 121 can be prepared in an analogous fashion to FIG. 13 by replacing compound 22 with compound 56 in step a.

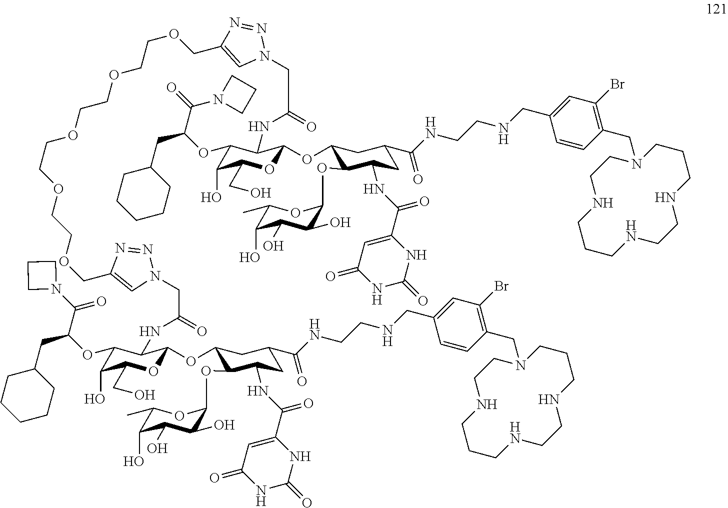

### Example 88

### Prophetic Synthesis of Multimeric Compound 122

Compound 122: Compound 122 can be prepared in an analogous fashion to FIG. 13 by replacing compound 22 with compound 57 in step a.

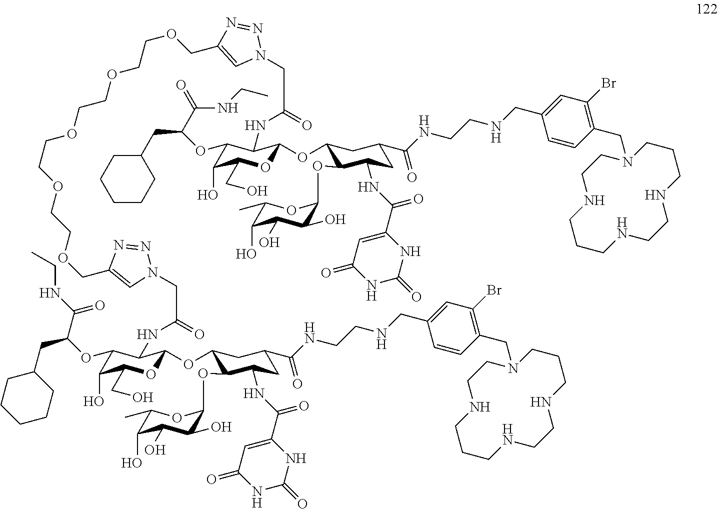

### Example 89

### Prophetic Synthesis of Multimeric Compound 123

Compound 123: Compound 123 can be prepared in an analogous fashion to FIG. 13 by replacing compound 22 with compound 58 in step a.

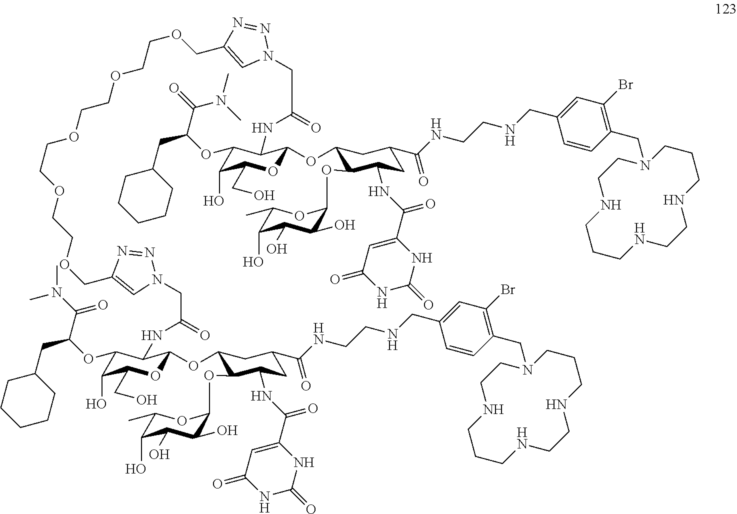

### Example 90

### Prophetic Synthesis of Multimeric Compound 124

Compound 124: Compound 124 can be prepared in an analogous fashion to FIG. 13 by replacing compound 22 with compound 59 in step a.

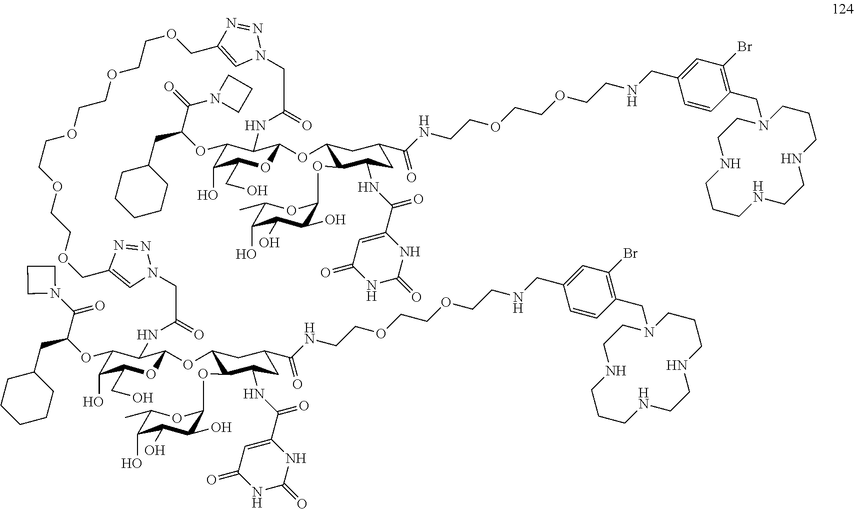

### Example 91

### Prophetic Synthesis of Multimeric Compound 125

Compound 125: Compound 125 con be prepared in an analogous fashion to FIG. 13 by replacing compound 22 with compound 68 in step a.

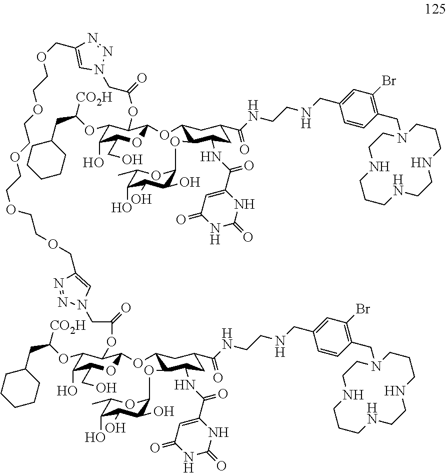

### Example 92

### Prophetic Synthesis of Multimeric Compound 126

Compound 126: Compound 126 can be prepared in an analogous fashion to FIG. 13 by replacing compound 22 with compound 69 in step a.

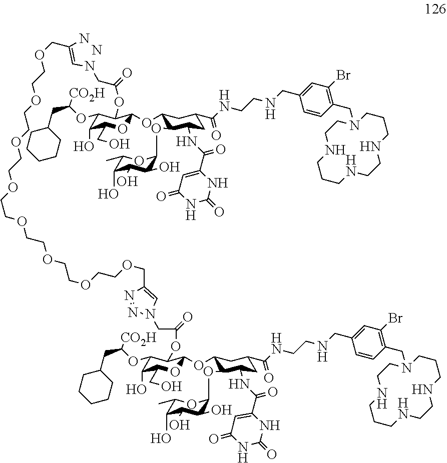

### Example 93

### Prophetic Synthesis of Multimeric Compound 127

Compound 127: Compound 127 can be prepared in an analogous fashion to FIG. 13 by replacing compound 22 with compound 70 in step a.

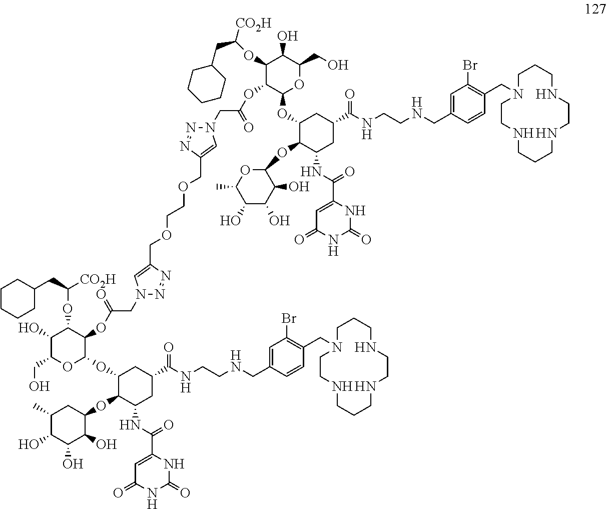 [SMILES: C[C@@H]1C[C@@H](O[C@@H]2[C@@H](NC(=O)c3cc(=O)[nH]c(=O)[nH]3)C[C@@H](C(=O)NCCNCc3ccc(CN4CCCNCCNCCCNCC4)c(Br)c3)C[C@H]2O[C@@H]2O[C@H](CO)[C@H](O)[C@H](O[C@@H](CC3CCCCC3)C(=O)O)[C@H]2OC(=O)Cn2cc(COCCOCc3cn(CC(=O)O[C@H]4[C@H](O[C@@H]5C[C@H](C(=O)NCCNCc6ccc(CN7CCCNCCNCCCNCC7)c(Br)c6)C[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 94

### Prophetic Synthesis of Multimeric Compound 128

Compound 128: Compound 128 can be prepared in an analogous fashion to FIG. 13 by replacing compound 22 with compound 71 in step a.

### Example 95

### Prophetic Synthesis of Multimeric Compound 129

Compound 129: Compound 129 can be prepared in an analogous fashion to FIG. 13 by replacing compound 22 with compound 73 in step a.

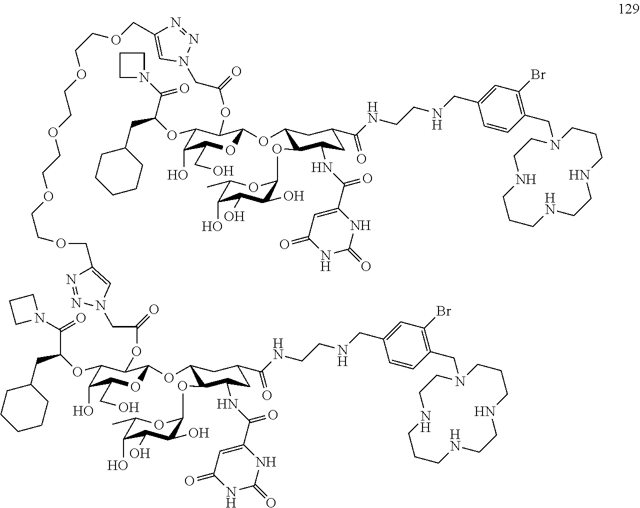

### Example 96

### Prophetic Synthesis of Multimeric Compound 130

Compound 130: Compound 130 can be prepared in an analogous fashion to FIG. 13 by replacing compound 22 with compound 78 in step a.

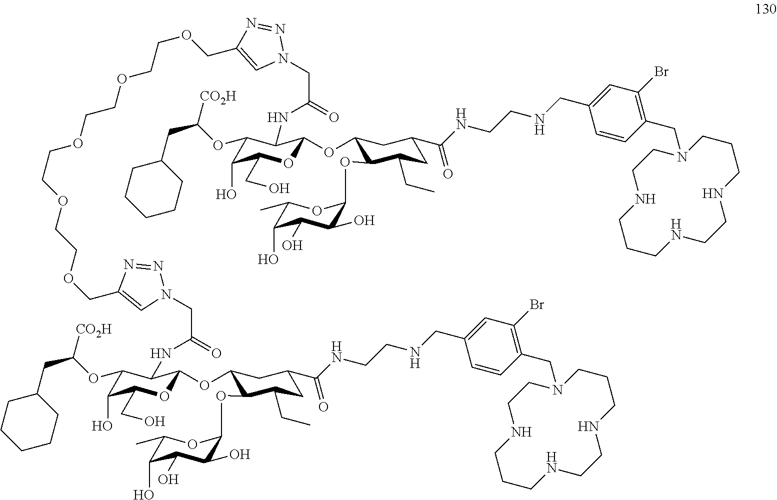

### Example 97

### Prophetic Synthesis of Multimeric Compound 131

Compound 131: Compound 131 can be prepared in an analogous fashion to FIG. 13 by replacing compound 22 with compound 79 in step a.

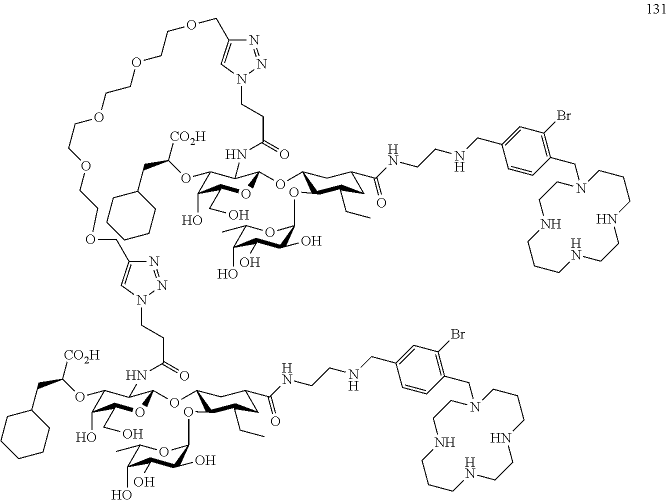

### Example 98

### Prophetic Synthesis of Multimeric Compound 132

Compound 132: Compound 132 can be prepared in an analogous fashion to FIG. 13 by replacing compound 22 with compound 80 in step a.

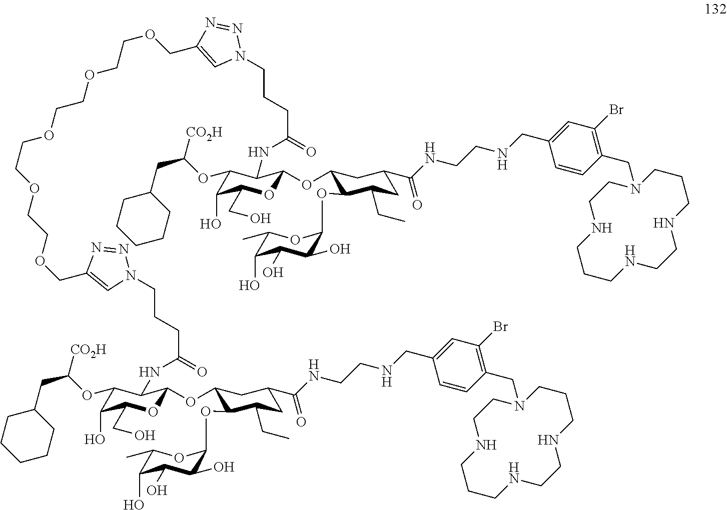

### Example 99

### Prophetic Synthesis of Multimeric Compound 133

Compound 133: Compound 133 can be prepared in an analogous fashion to FIG. 13 by replacing compound 22 with compound 81 in step a.

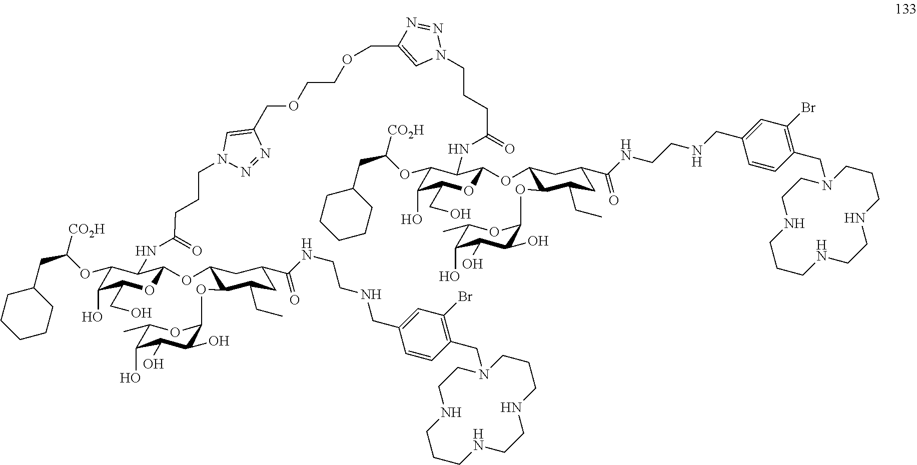

### Example 100

### Prophetic Synthesis of Multimeric Compound 134

Compound 134: Compound 134 can be prepared in an analogous fashion to FIG. 13 by replacing compound 22 with compound 82 in step a.

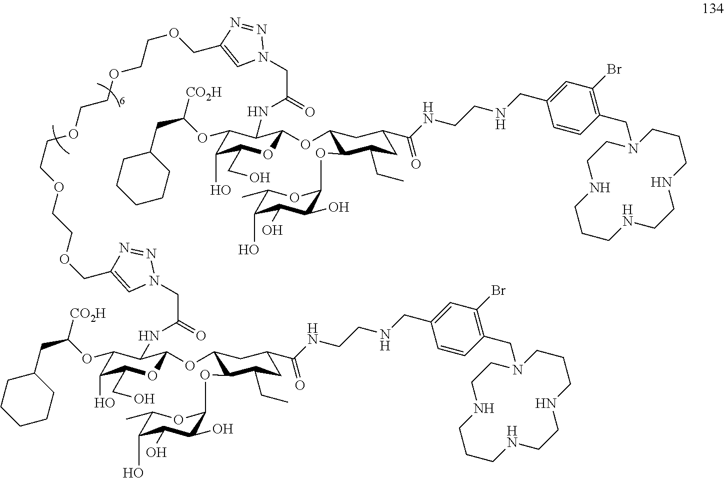

### Example 101

### Prophetic Synthesis of Multimeric Compound 135

Compound 135: Compound 135 can be prepared in an analogous fashion to FIG. 13 by replacing compound 22 with compound 83 in step a.

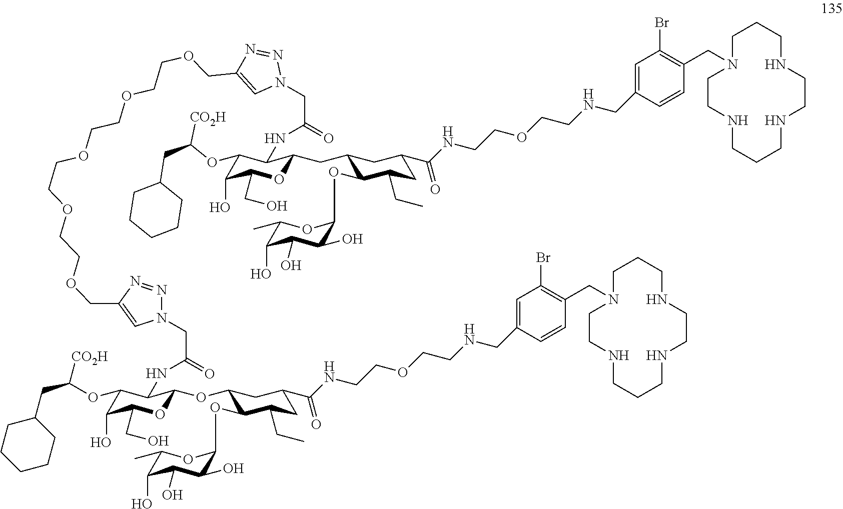

### Example 102

### Prophetic Synthesis of Multimeric Compound 136

Compound 136: Compound 136 can be prepared in an analogous fashion to FIG. 13 by replacing compound 22 with compound 84 in step a.

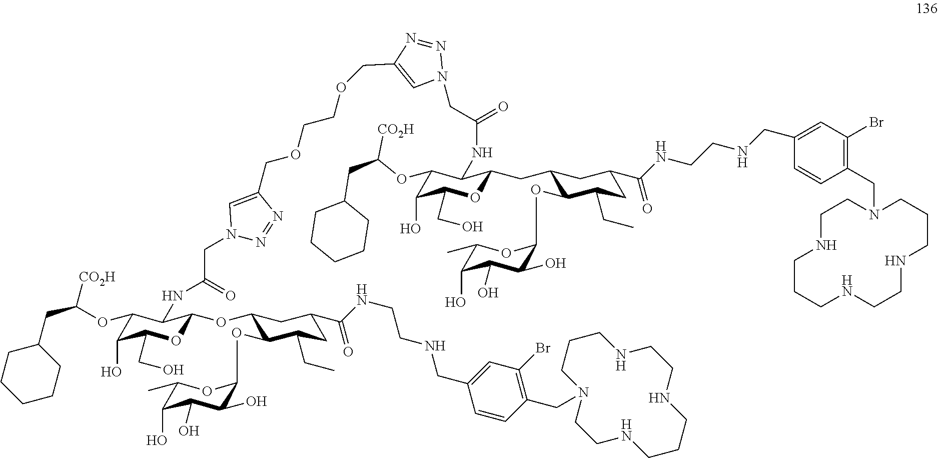

### Example 103

### Prophetic Synthesis of Multimeric Compound 137

Compound 137: Compound 137 can be prepared in an analogous fashion to FIG. 13 by replacing compound 22 with compound 85 in step a.

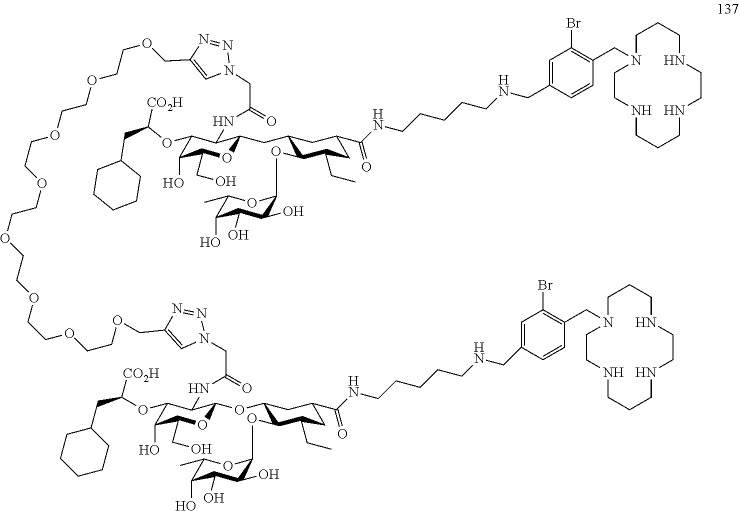

### Example 104

### Prophetic Synthesis of Multimeric Compound 138

Compound 138: Compound 138 can be prepared in an analogous fashion to FIG. 13 by replacing compound 22 with compound 87 in step a.

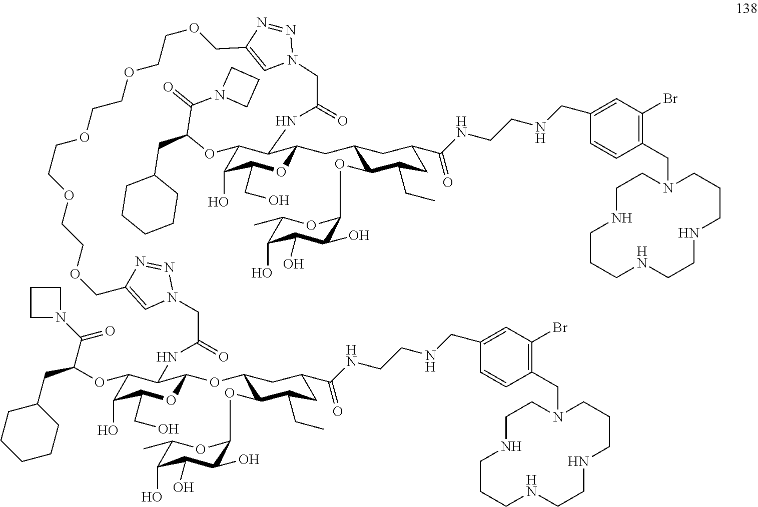

### Example 105

### Prophetic Synthesis of Multimeric Compound 139

Compound 139: Compound 139 can be prepared in an analogous fashion to FIG. 13 by replacing compound 22 with compound 88 in step a.

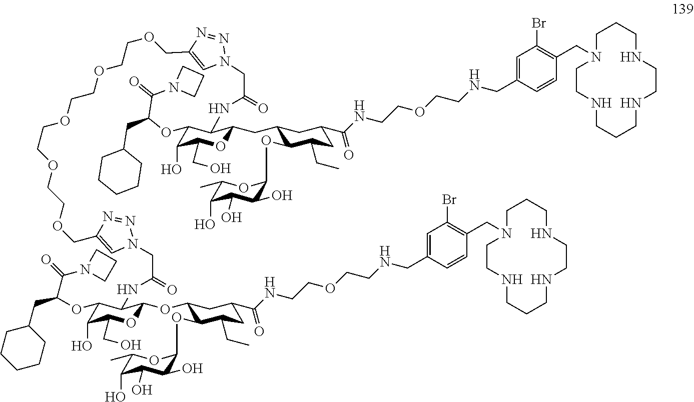

### Example 106

### Prophetic Synthesis of Multimeric Compound 140

Compound 140: Compound 140 can be prepared in an analogous fashion to FIG. 13 by replacing compound 22 with compound 89 in step a.

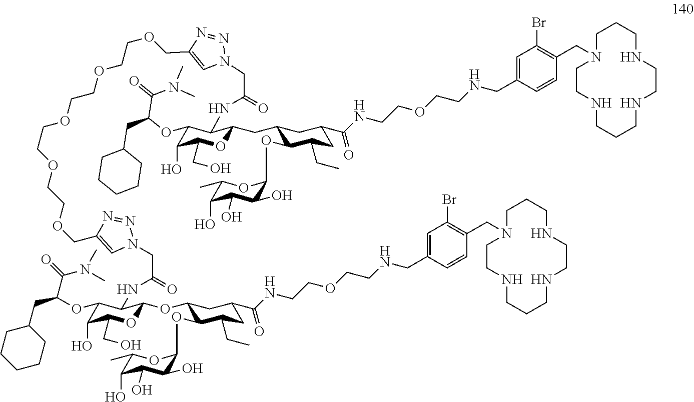

### Example 107

### Prophetic Synthesis of Multimeric Compound 141

Compound 141: Compound 141 can be prepared in an analogous fashion to FIG. 13 by replacing compound 22 with compound 90 in step a.

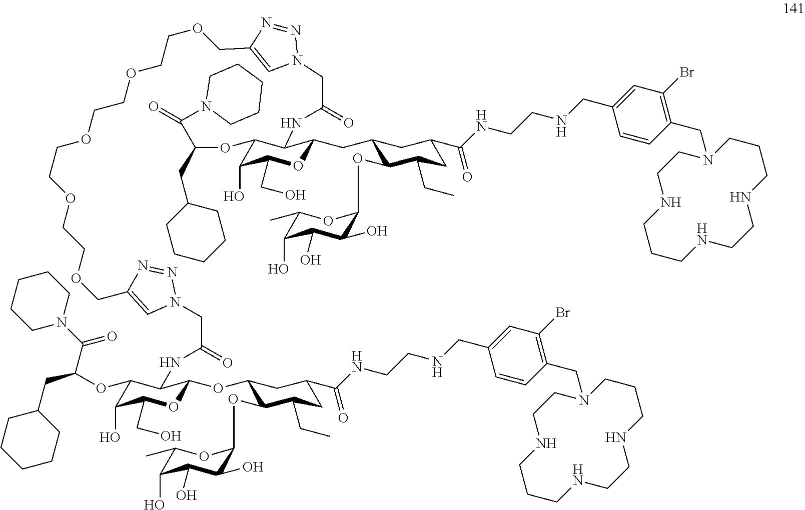

### Example 108

### Prophetic Synthesis of Multimeric Compound 142

Compound 142: Compound 142 can be prepared in an analogous fashion to FIG. 13 by replacing compound 22 with compound 91 in step a.

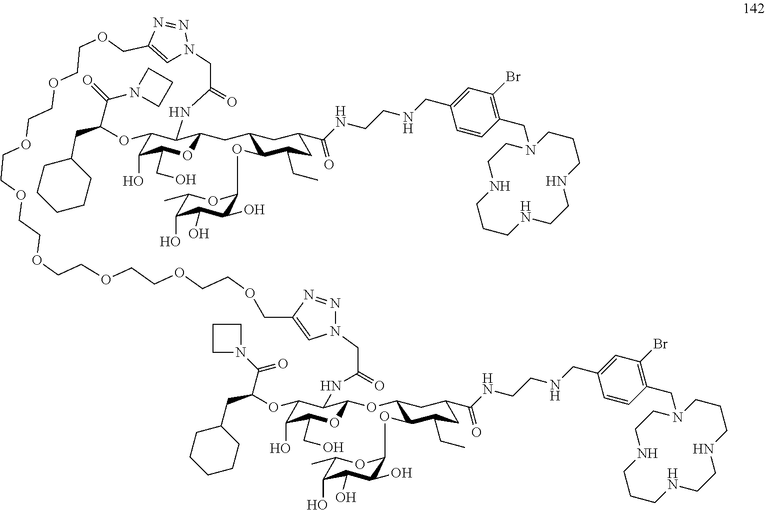

### Example 109

### Prophetic Synthesis of Multimeric Compound 143

Compound 143: Compound 143 can be prepared in an analogous fashion to FIG. 13 by replacing compound 22 with compound 92 in step a.

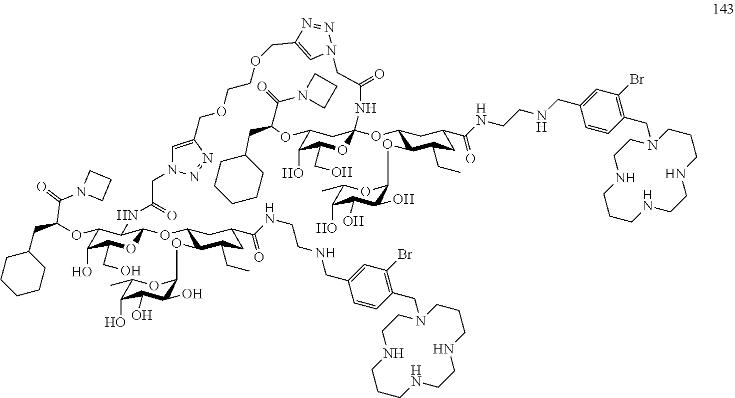

### Example 110

### Prophetic Synthesis of Multimeric Compound 144

Compound 144: Compound 144 can be prepared in an analogous fashion to FIG. 13 by replacing compound 22 with compound 93 in a step a.

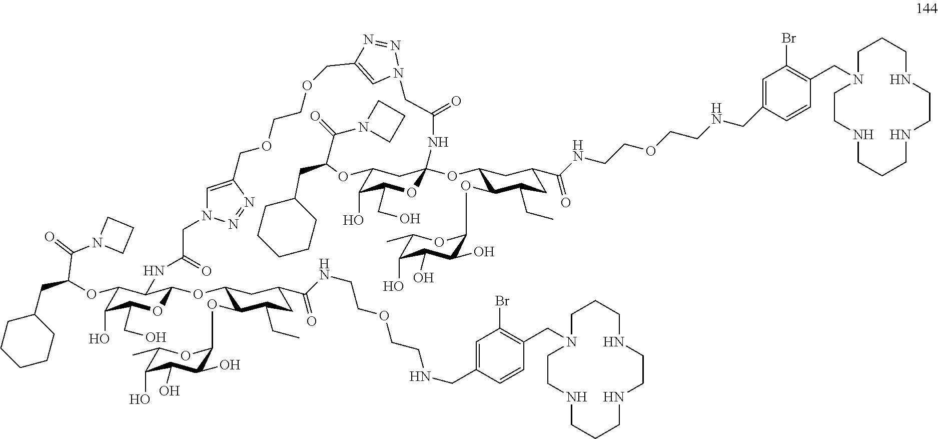

### Example 111

### Prophetic Synthesis of Multimeric Compound 146

Compound 315: To a solution of compound 314 (1 gm, 3.89 mmol) (preparation described in WO 2007/028050) and benzyl trichloroacetaimidate (1.1 ml, 5.83 mmol) in anhydrous dichloromethane (10 ml) was added trimethylsilyl trifluoromethanesulfonate (70 uL, 0.4 mmol). The mixture was stirred at ambient temperature for 12 h. After this period the reaction was diluted with dichloromethane, washed with saturated NaHCO₃, dried over MgSO₄ and concentrated. The residue was purified by column chromatography to give compound 315 (0.8 gm, 60%).

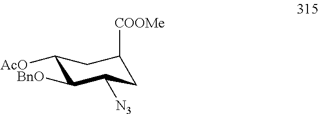

Compound 316: To a solution of compound 315 (800 mg, 2.3 mmol) in anhydrous methanol (1 ml) and anhydrous methyl acetate (5 ml) was added 0.5 M sodium methoxide solution in methanol (9.2 ml). The mixture was stirred at 40° C. for 4 h. The reaction was quenched with acetic acid and concentrated. The residue was purified by column chromatography to afford compound 316 as mixture of epimers at the methyl ester with 75% equatorial and 25% axial epimer (242 mg, 35%).

¹H NMR (400 MHz, Chloroform-d) δ 7.48-7.32 (m, 6H), 4.97 (d, J=11.1 Hz, 1H), 4.72 (dd, J=11.1, 5.7 Hz, 1H), 3.77-3.65 (m, 6H), 3.22-3.15 (m, 1H), 2.92-2.82 (m, 1H), 2.39 (dddd, J=15.7, 10.6, 5.1, 2.7 Hz, 2H), 1.60 (dtd, J=13.9, 11.2, 5.4 Hz, 3H). MS: Calculated for C₁₅H₁₉N₃O₄=305.3, Found ES-positive m/z=306.1 (M+N³⁰).

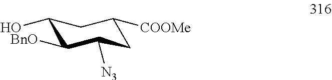

Compound 318: A solution of compound 317 (5 gm, 11.8 mmol) (preparation described in WO 2009/139719) in anhydrous methanol (20 ml) was treated with 0.5M solution of sodium methoxide in methanol (5 ml) for 3 h. Solvent was removed in vacuo and the residue was co-evaporated with toluene (20 ml) three times. The residue was dissolved in pyridine (20 ml) followed by addition of benzoyl chloride (4.1 ml, 35.4 mmol) over 10 minutes. The reaction mixture was stirred at ambient temperature under an atmosphere of argon for 22 h. The reaction mixture was concentrated to dryness, dissolved in dichloromethane, washed with cold 1N hydrochloric acid and cold water, dried over MgSO₄, filtered, and concentrated. The residue was purified by column chromatography to give compound 318. MS: Calculated for C₃₃H₂₇N₃O₇S=609.2, Found ES-positive m/z=610.2 (M+Na⁺).

Compound 319: A mixture of compound 318 (2.4 gm, 3.93 mmol), diphenyl sulfoxide (1.5 gm, 7.3 mmol) and 2,6-di-tert-butyl pyridine (1.8 gm, 7.8 mmol) was dissolved in anhydrous dichloromethane (10 ml) at room temperature. The reaction mixture was cooled to −60° C. Triflic anhydride (0.62 ml, 3.67 mmol) was added dropwise and the mixture was stirred for 15 minutes at the same temperature. A solution of compound 316 (0.8 gm, 2.6 mmol) in anhydrous dichloromethane (10 ml) was added dropwise to the reaction mixture. The mixture was allowed to warm to 0° C. over 2 h. The reaction mixture was diluted with dichloromethane, transferred to a separatory funnel and washed with saturated sodium bicarbonate solution followed by brine. The organic phase was dried over MgSO₄, filtered, and concentrated. The residue was separated by column chromatography to afford compound 319 as a white solid (1.2 gm, 57%). MS: Calculated for C₄₂H₄₀N₆O₁₁=804.3, Found ES-positive m/z=805.3 (M+Na⁺).

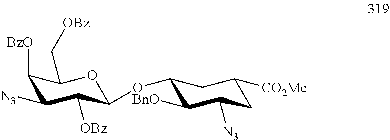

Compound 320: To a solution of compound 319 (1.2 gm 2.067 mmol) and 2-fluorophenyl acetylene (1.2 ml, 10.3 mmol) in methanol (30 ml) was added a stock solution of copper sulfate and tris(3-hydroxypropyltriazolylmethyl) amine in water (2.58 ml). The reaction was initiated by addition of an aqueous solution of sodium ascorbate (0.9 gm, 4.5 mmol) and the mixture was stirred at ambient temperature for 16 hours. The mixture was co-evaporated with dry silica gel and purified by column chromatography to afford compound 320 as a white solid (1.2 gm, 77%).

Stock solution of Copper Sulfate/THETA—(100 mg of copper sulfate pentahydrate and 200 mg of tris(3-hydroxypropyltriazolylmethyl)amine were dissolved in 10 ml of water).

¹H NMR (400 MHz, Chloroform-d) δ 8.07-8.00 (m, 2H), 7.96 (ddd, J=9.8, 8.2, 1.3 Hz, 4H), 7.79 (d, J=5.4 Hz, 2H), 7.65-7.53 (m, 5H), 7.43 (ddt, J=22.4, 10.7, 5.0 Hz, 7H), 7.25-7.01 (m, 9H), 6.92 (td, J=7.6, 7.1, 2.2 Hz, 1H), 6.13-6.02 (m, 2H), 5.58 (dd, J=11.6, 3.2 Hz, 1H), 5.15 (d, J=7.5 Hz, 1H), 4.98 (d, J=10.3 Hz, 1H), 4.68 (dd, J=11.2, 5.7 Hz, 1H), 4.52 (dq, J=22.1, 6.6, 5.6 Hz, 2H), 4.35 (dd, J=11.1, 7.6 Hz, 1H), 4.28-4.18 (m, 1H), 4.11 (d, J=10.3 Hz, 1H), 3.87 (t, J=9.1 Hz, 1H), 3.71 (s, 3H), 2.95 (s, 1H), 2.62-2.43 (m, 3H), 1.55 (dt, J=12.7, 6.1 Hz, 1H). MS: Calculated for C₅₈H₅₀N₆O₁₁=1044.4, Found ES-positive m/z=1045.5 (M+Na⁺).

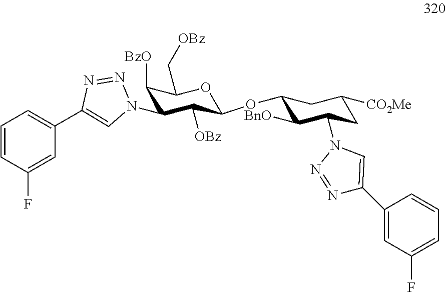

Compound 145: To a solution of compound 320 (1.2 gm, 1.1 mmol) in iso-propanol (40 ml) was added Na-metal (80 mg, 3.4 mmol) at ambient temperature and the mixture was stirred for 12 hours at 50° C. 10%/o aqueous sodium hydroxide (2 ml) was added to the reaction mixture and stirring continued for another 6 hours at 50° C. The reaction mixture was cooled to room temperature and neutralized with 50% aqueous hydrochloric acid. To the mixture was added 10% Pd(OH)₂ on carbon (0.6 gm) and the reaction mixture was stirred under an atmosphere of hydrogen for 12 hours. The reaction mixture was filtered through a Celite pad and concentrated. The residue was separated by HPLC to give compound 145 as a white solid (0.5 gm, 70%). HPLC Conditions—Waters preparative HPLC system was used with ELSD & PDA detectors. Kinetex XB-C18, 100 A, 5 uM, 250×21.2 mm column (from Phenomenex) was used with 0.2% formic acid in water as solvent A and acetonitrile as solvent B at a flow rate of 20 mL/min.

¹H NMR (400 MHz, DMSO-d₆) δ 8.77 (s, 1H), 8.68 (s, 1H), 7.77-7.60 (m, 5H), 7.49 (tdd, J=8.3, 6.1, 2.6 Hz, 3H), 7.15 (tt, J=8.6, 3.2 Hz, 3H), 4.83 (dd, J=10.9, 3.1 Hz, 1H), 4.63 (d, J=7.5 Hz, 1H), 4.53-4.41 (m, 1H), 4.10 (dd, J=10.9, 7.5 Hz, 1H), 3.92 (d, J=3.2 Hz, 1H), 3.74 (h, J=6.0, 5.6 Hz, 3H), 3.65-3.24 (m, 5H), 2.37 (d, J=13.4 Hz, 1H), 2.24-2.04 (m, 2H), 1.93 (q, J=12.5 Hz, 1H), 1.46 (t, J=12.1 Hz, 1H). MS: Calculated for C₂₉H₃₀F₂N₆O₈=628.2, Found ES-positive m/z=629.2 (M+Na⁺).

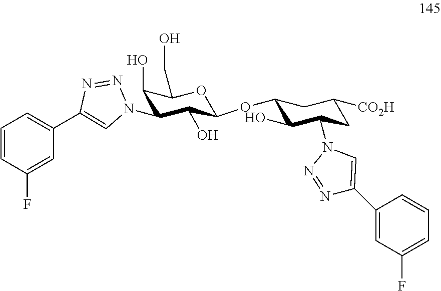

Compound 146: To a solution of compound 145 (3 eq) in anhydrous DMF was added HATU (3.3 eq) and DIPEA (5 eq). The mixture was stirred at ambient temperature for 15 minutes followed by addition of compound 22 (1 eq). The mixture was stirred at ambient temperature for 12 h. The solvent was removed in vacuo and the residue was purified by HPLC to afford compound 146.

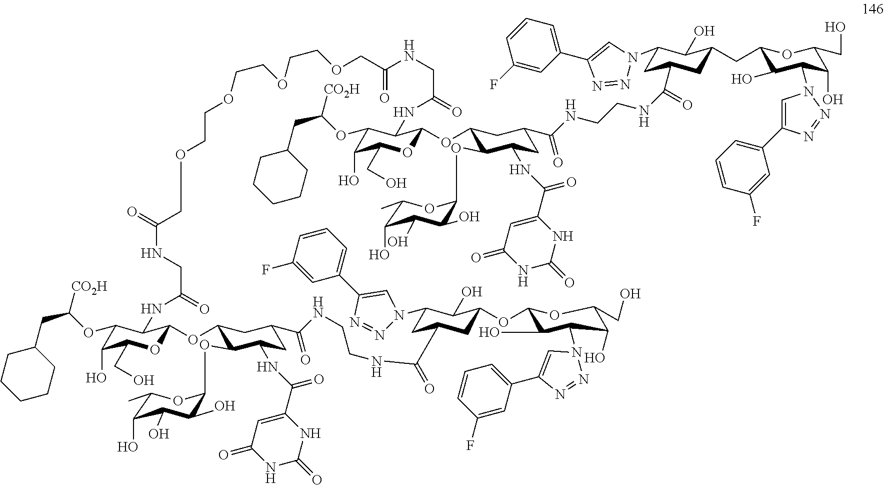

### Example 112

### Prophetic Synthesis of Multimeric Compound 147

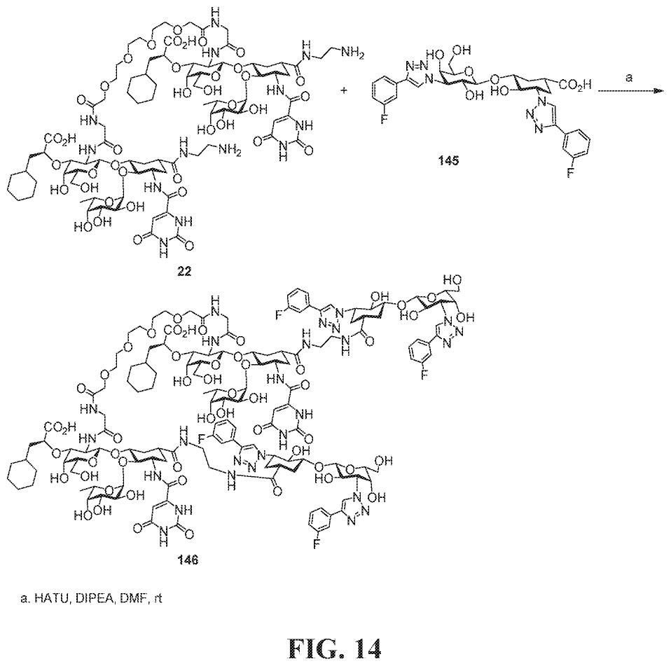

Compound 147: Compound 147 can be prepared in an analogous fashion to FIG. 14 by replacing compound 22 with compound 23.

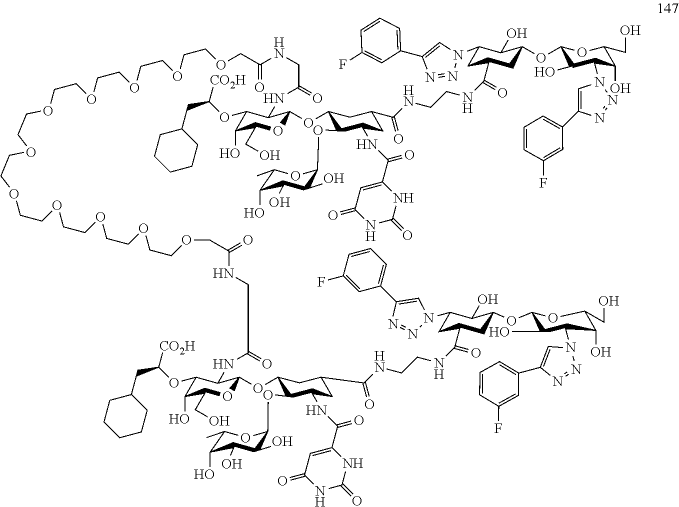

### Example 113

### Prophetic Synthesis of Multimeric Compound 148

Compound 148: Compound 148 can be prepared in an analogous fashion to FIG. 14 by replacing compound 22 with compound 24.

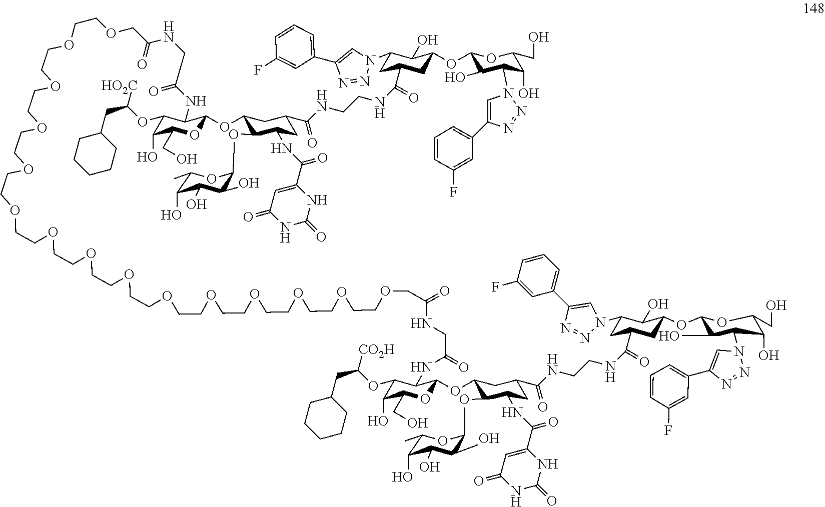

### Example 114

### Prophetic Synthesis of Multimeric Compound 149

Compound 149: Compound 149 can be prepared in an analogous fashion to FIG. 14 by replacing compound 22 with compound 25.

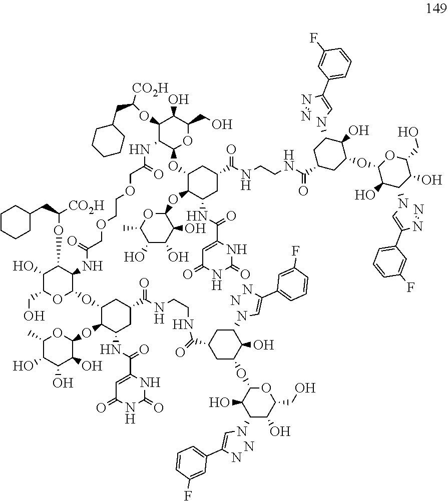

### Example 115

### Prophetic Synthesis of Multimeric Compound 150

Compound 150: Compound 150 can be prepared in an analogous fashion to FIG. 14 by replacing compound 22 with compound 26.

### Example 116

### Prophetic Synthesis of Multimeric Compound 151

Compound 151: Compound 151 can be prepared in an analogous fashion to FIG. 14 by replacing compound 22 with compound 27.

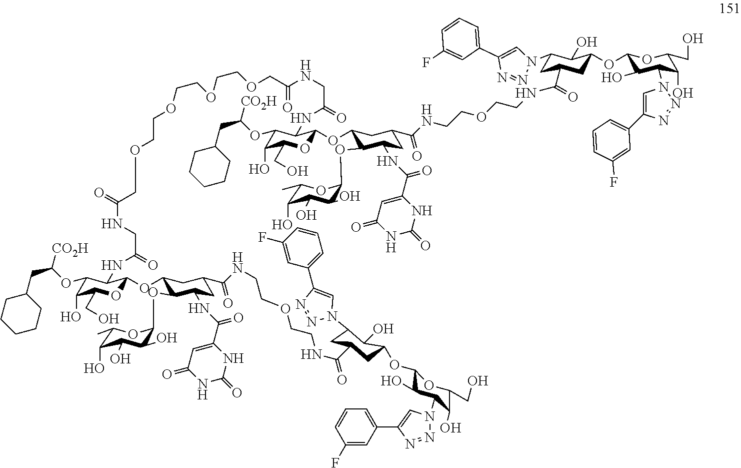

### Example 117

### Prophetic Synthesis of Multimeric Compound 152

Compound 152: Compound 152 can be prepared in an analogous fashion to FIG. 14 by replacing compound 22 with compound 28.

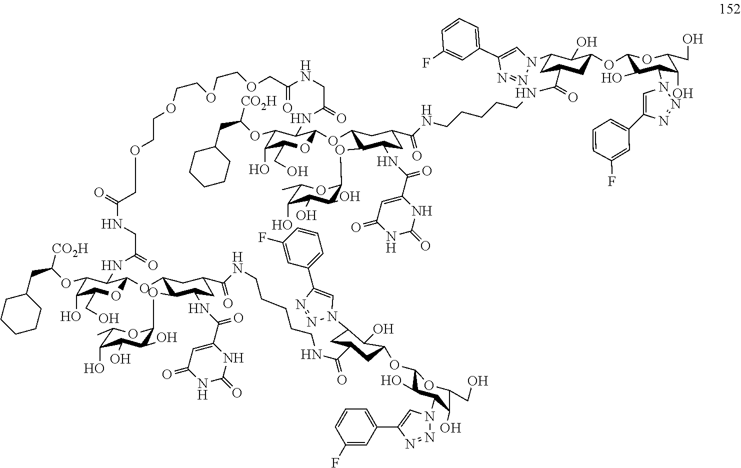

### Example 118

### Prophetic Synthesis of Multimeric Compound 153

Compound 153: Compound 153 can be prepared in an analogous fashion to FIG. 14 by replacing compound 22 with compound 29.

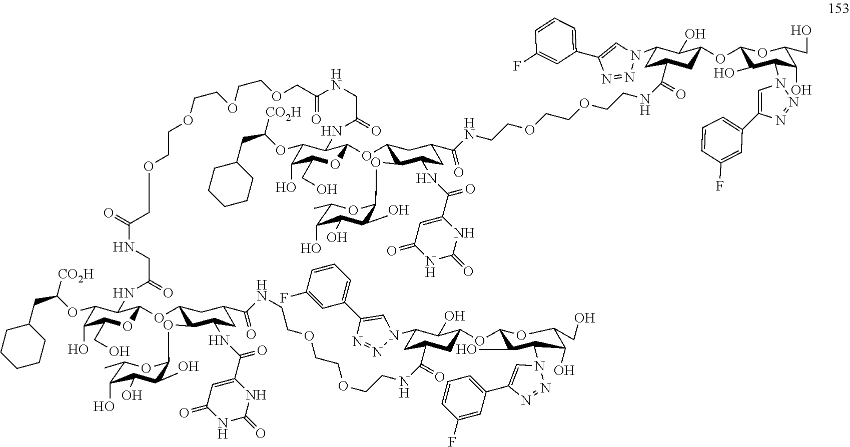

### Example 119

### Prophetic Synthesis of Multimeric Compound 154

Compound 154: Compound 154 can be prepared in an analogous fashion to FIG. 14 by replacing compound 22 with compound 30.

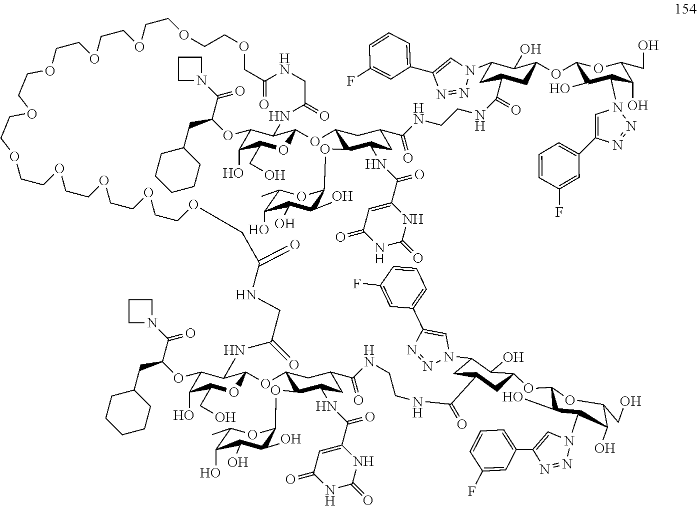

### Example 120

### Prophetic Synthesis of Multimeric Compound 155

Compound 155: Compound 155 can be prepared in an analogous fashion to FIG. 14 by replacing compound 22 with compound 31.

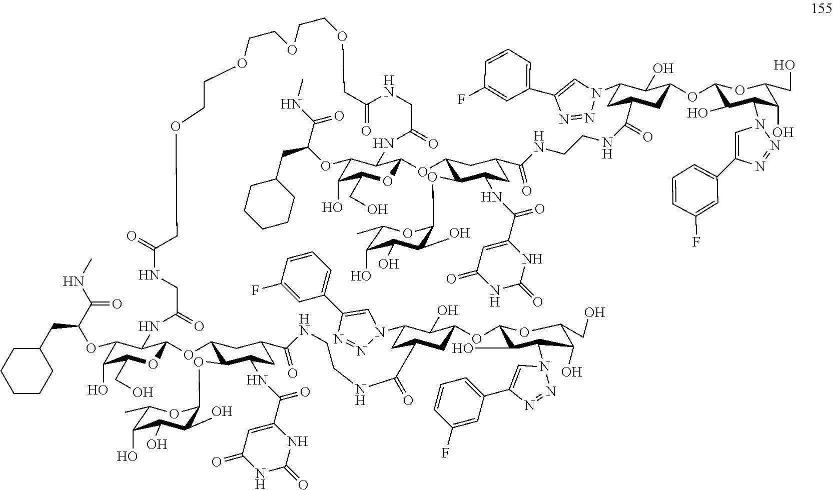

### Example 121

### Prophetic Synthesis of Multimeric Compound 156

Compound 156: Compound 156 can be prepared in an analogous fashion to FIG. 14 by replacing compound 22 with compound 32.

### Example 122

### Prophetic Synthesis of Multimeric Compound 157

Compound 157: Compound 157 can be prepared in an analogous fashion to FIG. 14 by replacing compound 22 with compound 33.

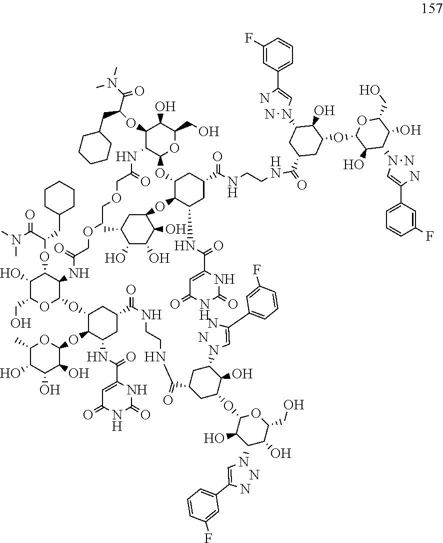

### Example 123

### Prophetic Synthesis of Multimeric Compound 158

Compound 158: Compound 158 can be prepared in an analogous fashion to FIG. 14 by replacing compound 22 with compound 34.

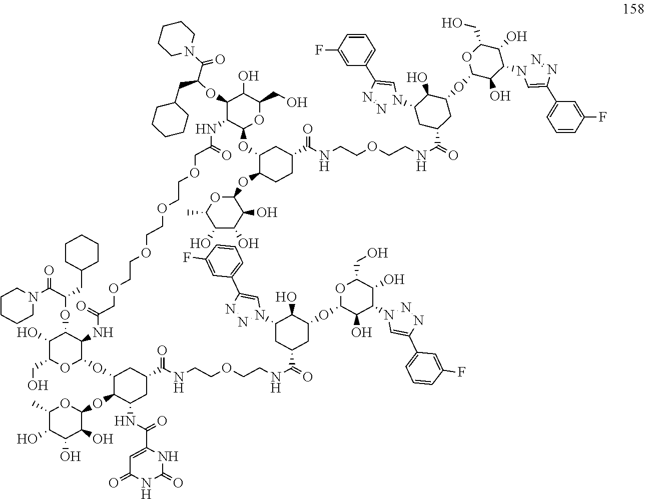

### Example 124

### Prophetic Synthesis of Multimeric Compound 159

Compound 159: Compound 159 can be prepared in an analogous fashion to FIG. 14 by replacing compound 22 with compound 37.

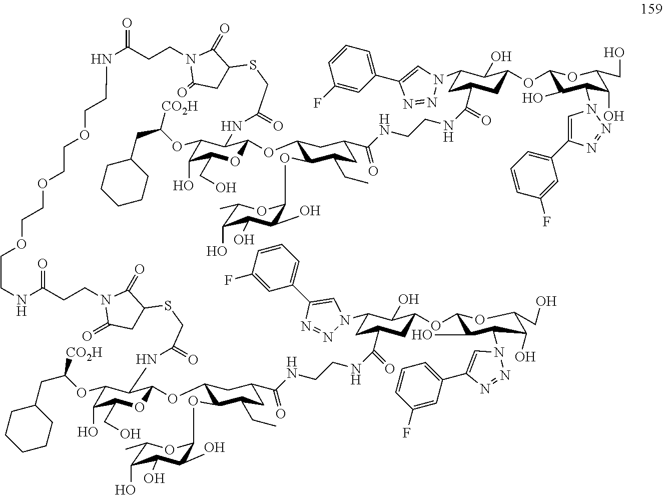

### Example 125

### Prophetic Synthesis of Multimeric Compound 160

Compound 160: Compound 160 can be prepared in an analogous fashion to FIG. 14 by replacing compound 22 with compound 38.

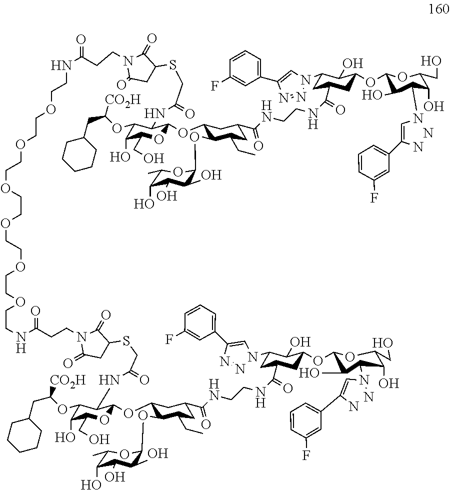

### Example 126

### Prophetic Synthesis of Multimeric Compound 161

Compound 161: Compound 161 can be prepared in an analogous fashion to FIG. 14 by replacing compound 22 with compound 39.

### Example 127

### Prophetic Synthesis of Multimeric Compound 162

Compound 162: Compound 162 can be prepared in an analogous fashion to FIG. 14 by replacing compound 22 with compound 40.

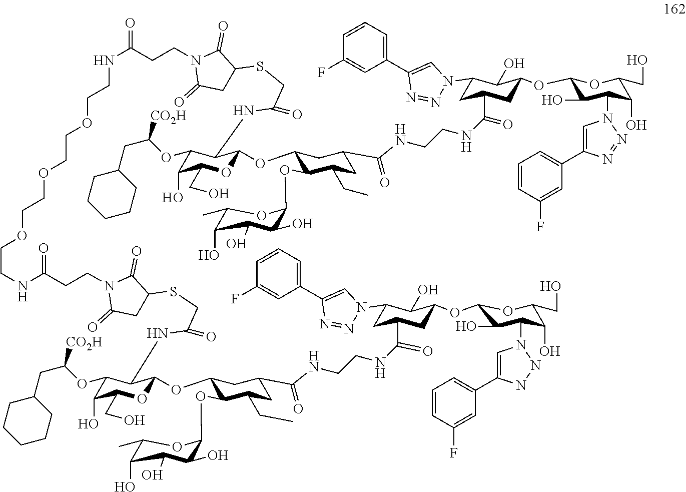

### Example 128

### Prophetic Synthesis of Multimeric Compound 163

Compound 163: Compound 163 can be prepared in an analogous fashion to FIG. 14 by replacing compound 22 with compound 46.

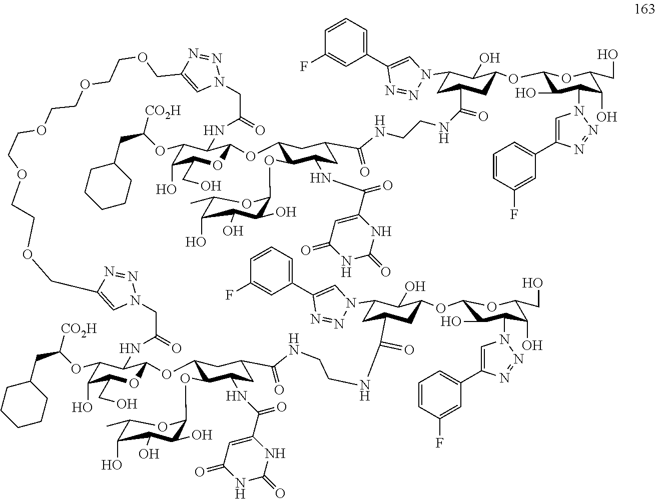

### Example 129

### Prophetic Synthesis of Multimeric Compound 164

Compound 164: Compound 164 can be prepared in an analogous fashion to FIG. 14 by replacing compound 22 with compound 47.

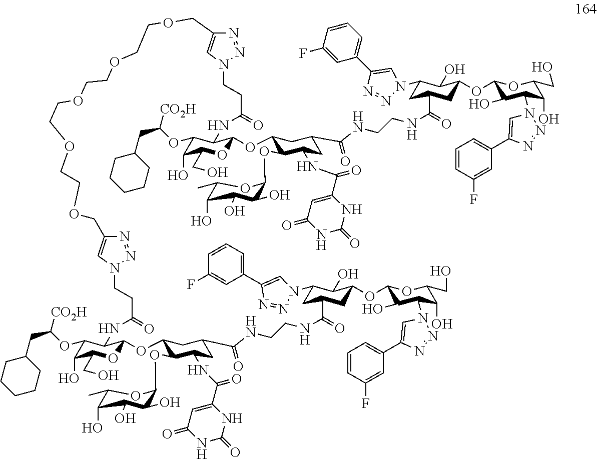

### Example 130

### Prophetic Synthesis of Multimeric Compound 165

Compound 165: Compound 165 can be prepared in an analogous fashion to FIG. 13 by replacing compound 22 with compound 48.

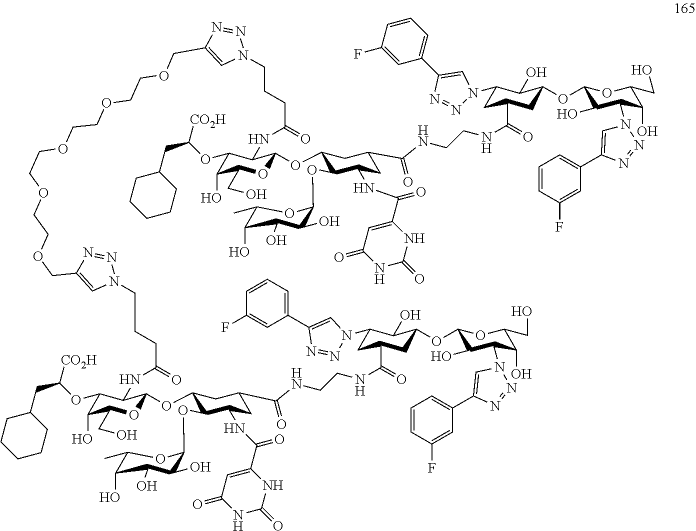

### Example 131

### Prophetic Synthesis of Multimeric Compound 166

Compound 166: Compound 166 can be prepared in an analogous fashion to FIG. 14 by replacing compound 22 with compound 49.

### Example 132

### Prophetic Synthesis of Multimeric Compound 167

Compound 167: Compound 167 can be prepared in an analogous fashion to FIG. 14 by replacing compound 22 with compound 50.

### Example 133

### Prophetic Synthesis of Multimeric Compound 168

Compound 168: Compound 168 can be prepared in an analogous fashion to FIG. 14 by replacing compound 22 with compound 51.

### Example 134

### Prophetic Synthesis of Multimeric Compound 169

Compound 169: Compound 169 can be prepared in an analogous fashion to FIG. 14 by replacing compound 22 with compound 52.

### Example 135

### Prophetic Synthesis of Multimeric Compound 170

Compound 170: Compound 170 can be prepared in an analogous fashion to FIG. 14 by replacing compound 22 with compound 53.

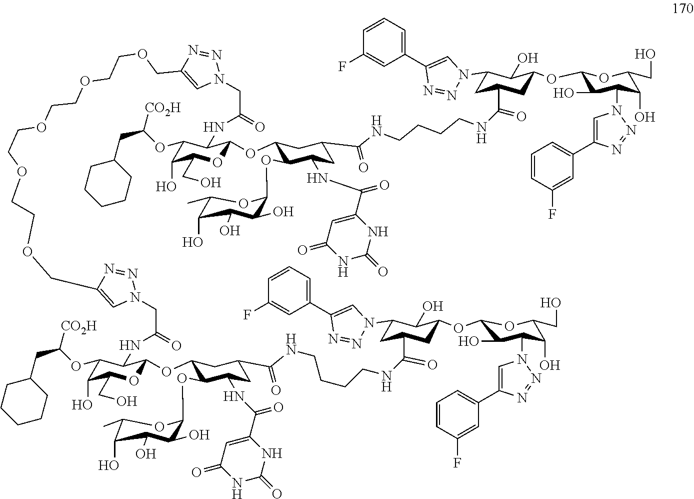

### Example 137

### Prophetic Synthesis of Multimeric Compound 172

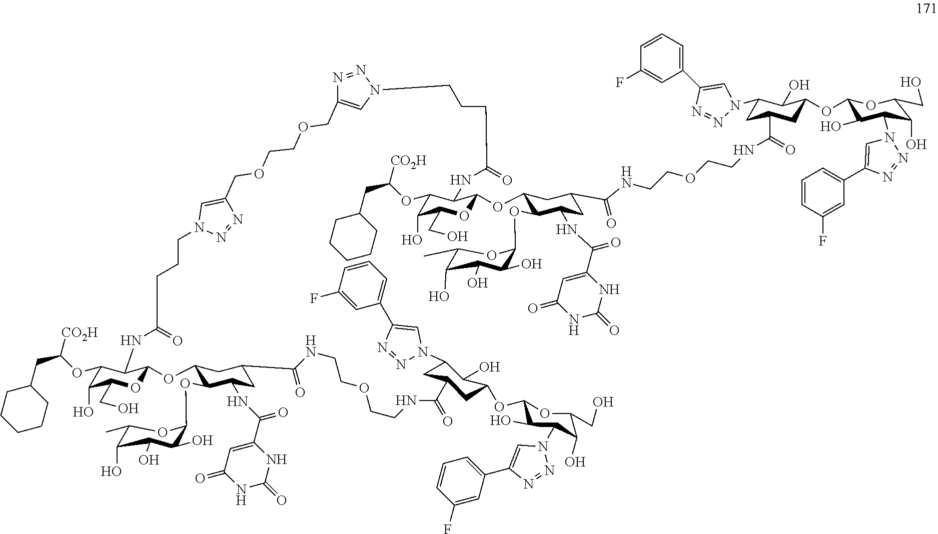

### Example 137

### Prophetic Synthesis of Multimeric Compound 172

Compound 172: Compound 172 can be prepared in an analogous fashion to FIG. 14 by replacing compound 22 with compound 56.

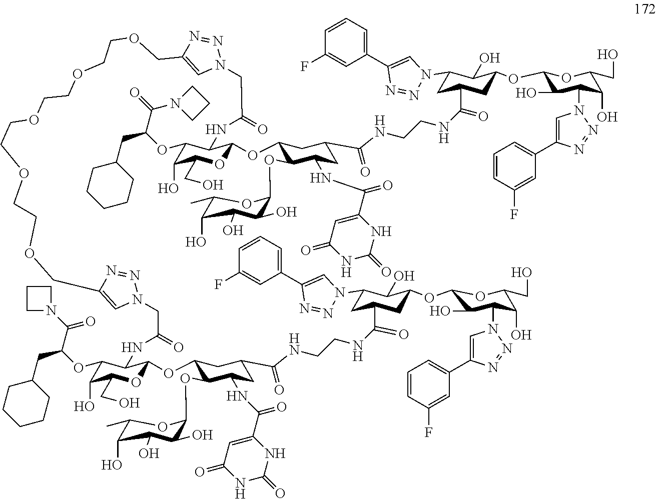

### Example 138

### Prophetic Synthesis of Multimeric Compound 173

Compound 173: Compound 173 can be prepared in an analogous fashion to FIG. 14 by replacing compound 22 with compound 57.

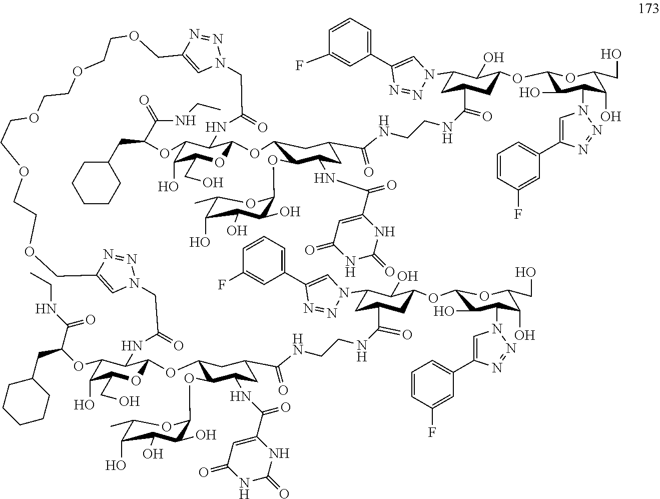

### Example 139

### Prophetic Synthesis of Multimeric Compound 174

Compound 174: Compound 174 can be prepared in an analogous fashion to FIG. 14 by replacing compound 22 with compound 58.

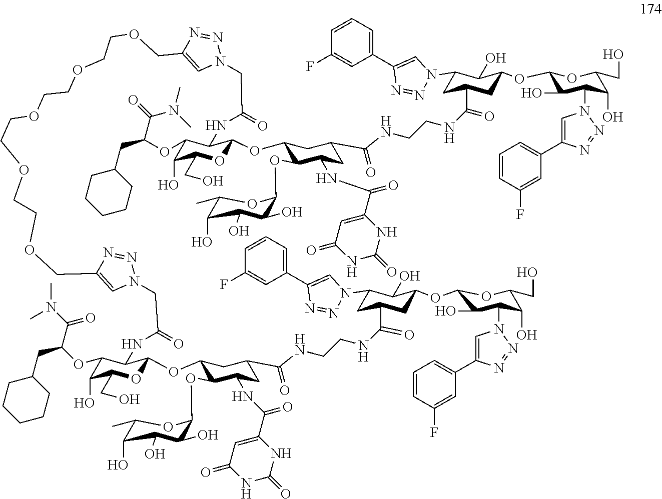

### Example 140

### Prophetic Synthesis of Multimeric Compound 175

Compound 175: Compound 175 can be prepared in an analogous fashion to FIG. 14 by replacing compound 22 with compound 59.

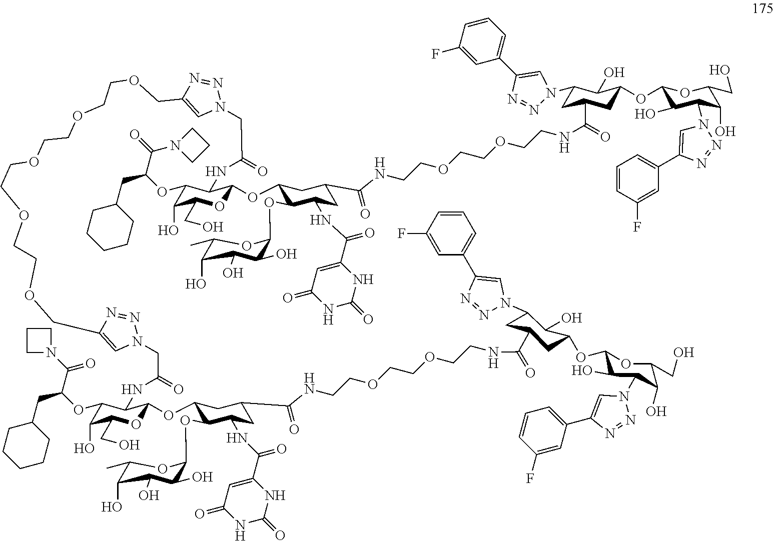

### Example 141

### Prophetic Synthesis of Multimeric Compound 176

Compound 176: Compound 176 can be prepared in an analogous fashion to FIG. 14 by replacing compound 22 with compound 68.

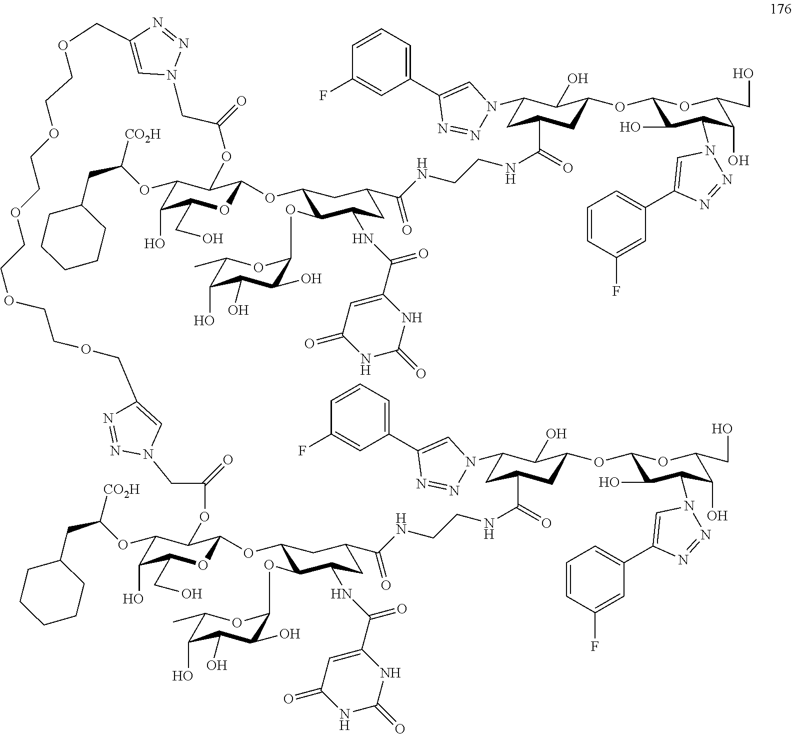

### Example 142

### Prophetic Synthesis of Multimeric Compound 177

Compound 177: Compound 177 can be prepared in an analogous fashion to FIG. 14 by replacing compound 22 with compound 69.

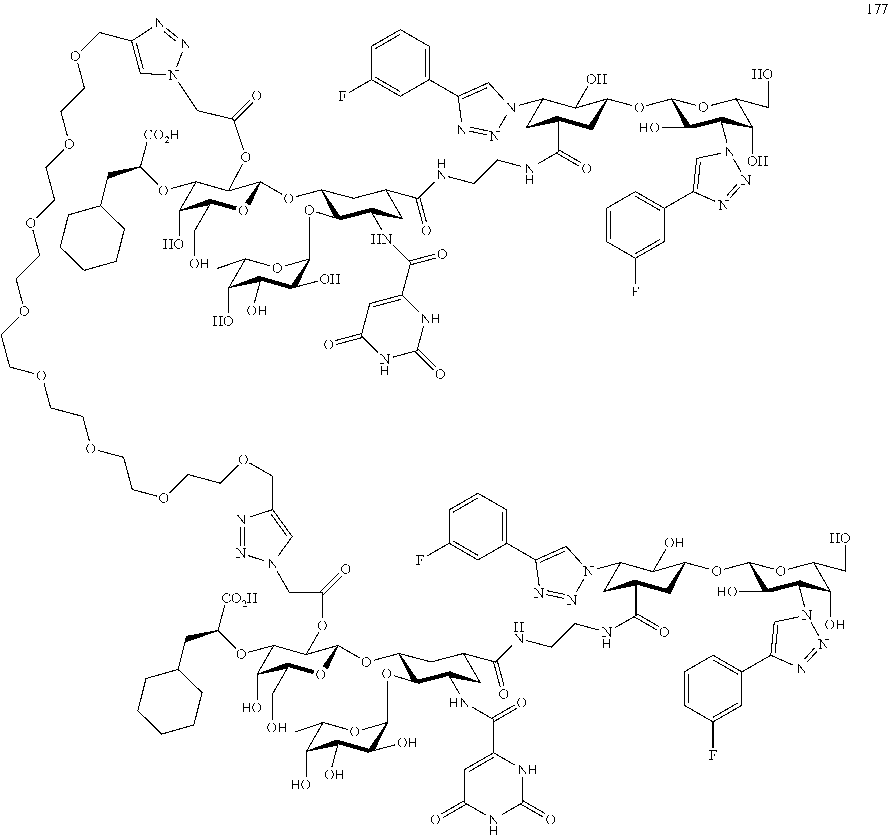

### Example 143

### Prophetic Synthesis of Multimeric Compound 178

Compound 178: Compound 178 can be prepared in an analogous fashion to FIG. 14 by replacing compound 22 with compound 70.

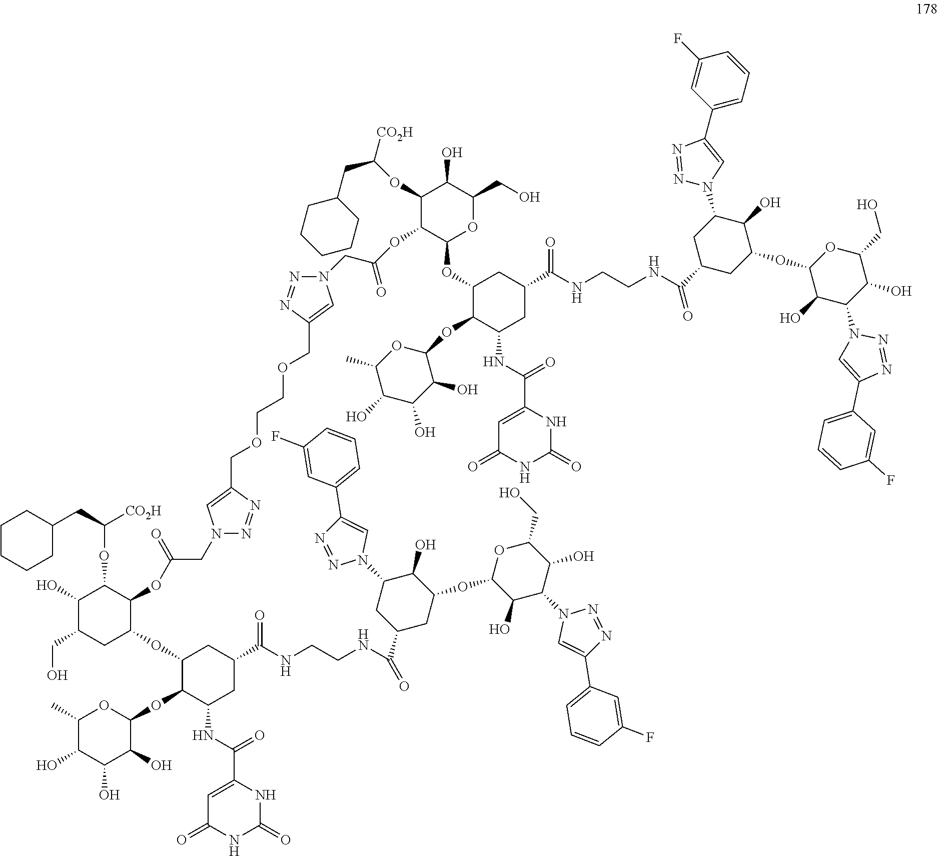

### Example 144

### Prophetic Synthesis of Multimeric Compound 179

Compound 179: Compound 179 can be prepared in an analogous fashion to FIG. 14 by replacing compound 22 with compound 71.

### Example 145

### Prophetic Synthesis of Multimeric Compound 180

Compound 180: Compound 180 can be prepared in an analogous fashion to FIG. 14 by replacing compound 22 with compound 73.

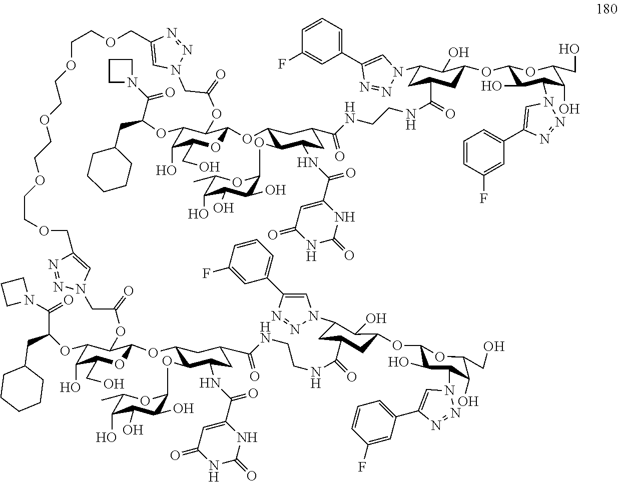

### Example 146

### Prophetic Synthesis of Multimeric Compound 181

Compound 181: Compound 181 can be prepared in an analogous fashion to FIG. 14 by replacing compound 22 with compound 78.

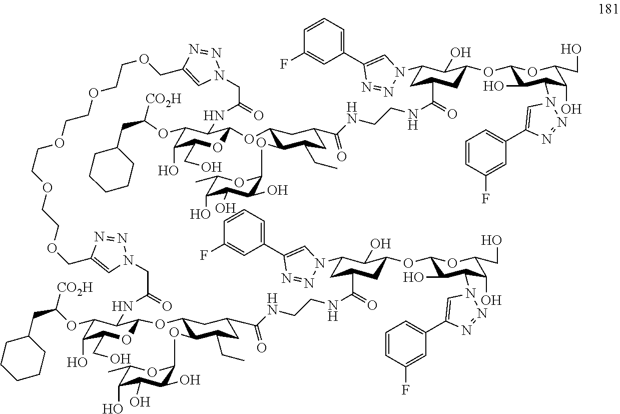

### Example 147

### Prophetic Synthesis of Multimeric Compound 182

Compound 182: Compound 182 can be prepared in an analogous fashion to FIG. 14 by replacing compound 22 with compound 79.

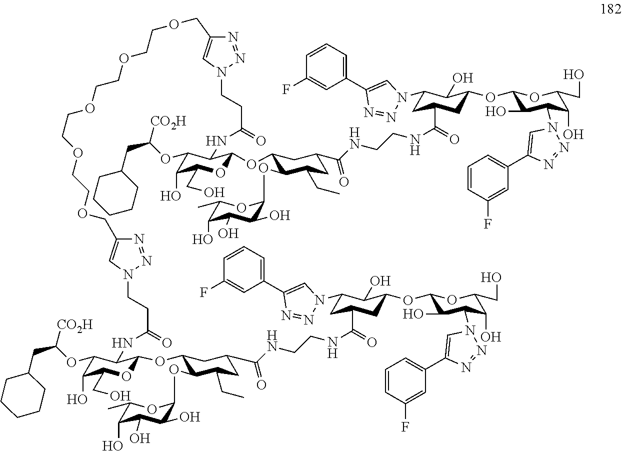

### Example 148

### Prophetic Synthesis of Multimeric Compound 183

Compound 183: Compound 183 can be prepared in an analogous fashion to FIG. 14 by replacing compound 22 with compound 80.

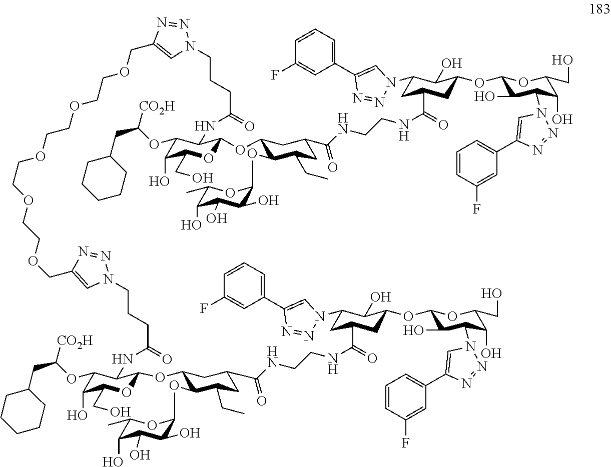

### Example 149

### Prophetic Synthesis of Multimeric Compound 184

Compound 184: Compound 184 can be prepared in an analogous fashion to FIG. 14 by replacing compound 22 with compound 81.

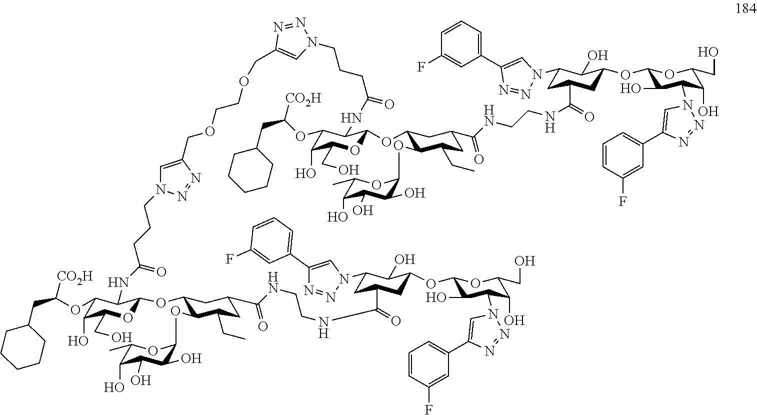

### Example 150

### Prophetic Synthesis of Multimeric Compound 185

Compound 185: Compound 185 can be prepared in an analogous fashion to FIG. 14 by replacing compound 22 with compound 82

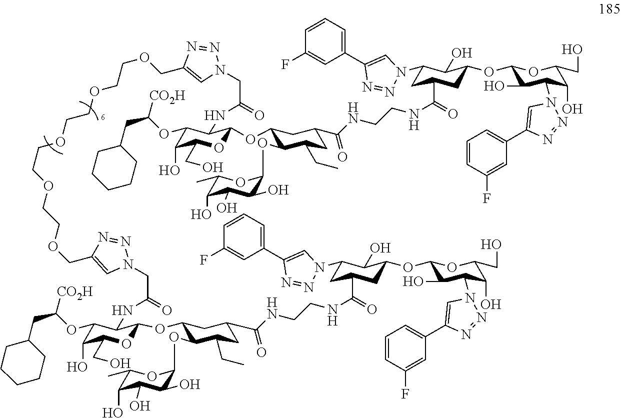

### Example 151

### Prophetic Synthesis of Multimeric Compound 186

Compound 186: Compound 186 can be prepared in an analogous fashion to FIG. 14 by replacing compound 22 with compound 83.

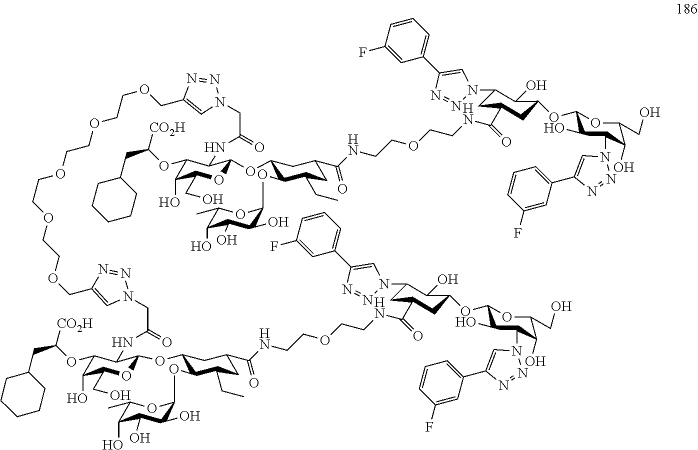

### Example 152

### Prophetic Synthesis of Multimeric Compound 187

Compound 187: Compound 187 can be prepared in an analogous fashion to FIG. 14 by replacing compound 22 with compound 84.

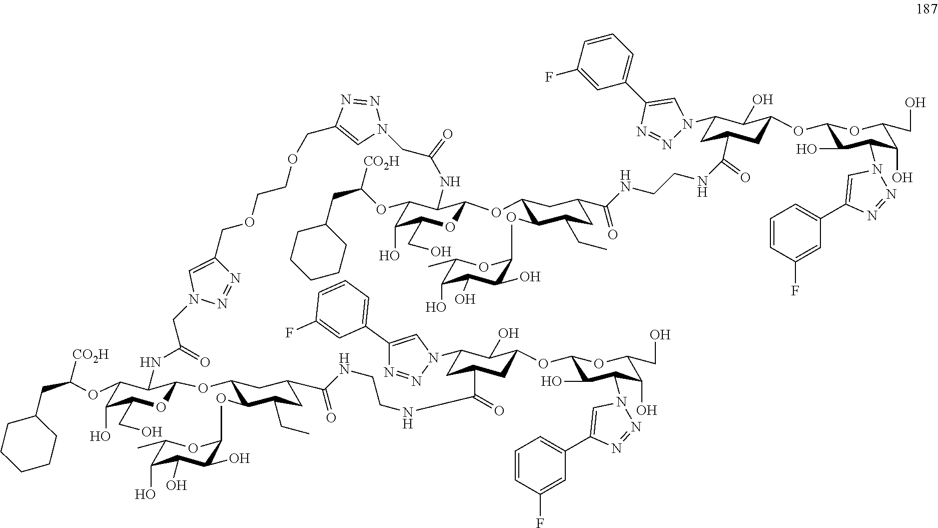

### Example 153

### Prophetic Synthesis of Multimeric Compound 188

Compound 188: Compound 188 can be prepared in an analogous fashion to FIG. 14 by replacing compound 22 with compound 85.

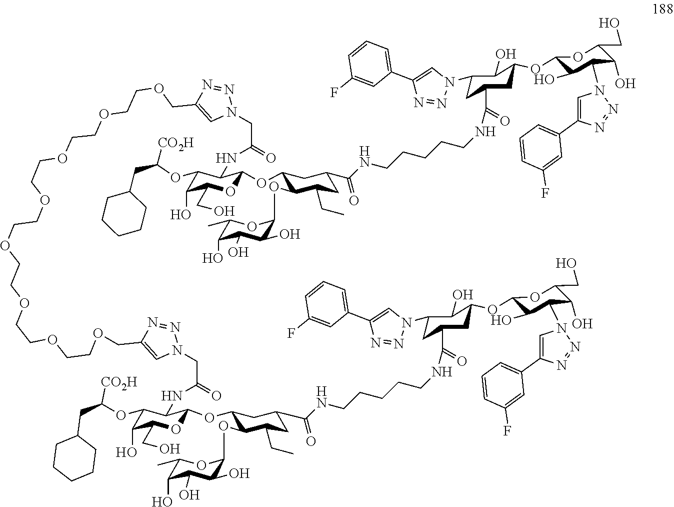

### Example 154

### Prophetic Synthesis of Multimeric Compound 189

Compound 189: Compound 189 can be prepared in an analogous fashion to FIG. 14 by replacing compound 22 with compound 87.

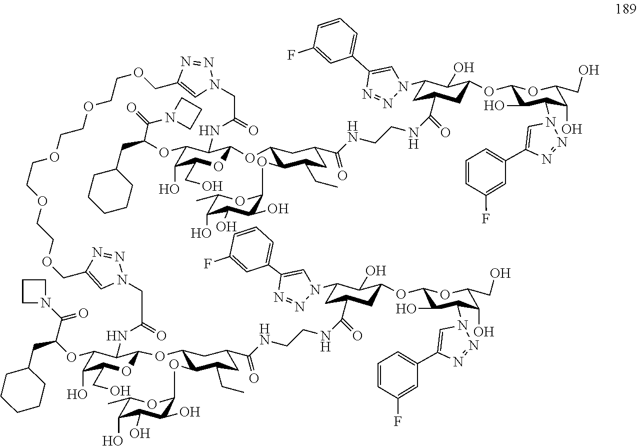

### Example 155

### Prophetic Synthesis of Multimeric Compound 190

Compound 190: Compound 190 can be prepared in an analogous fashion to FIG. 14 by replacing compound 22 with compound 88.

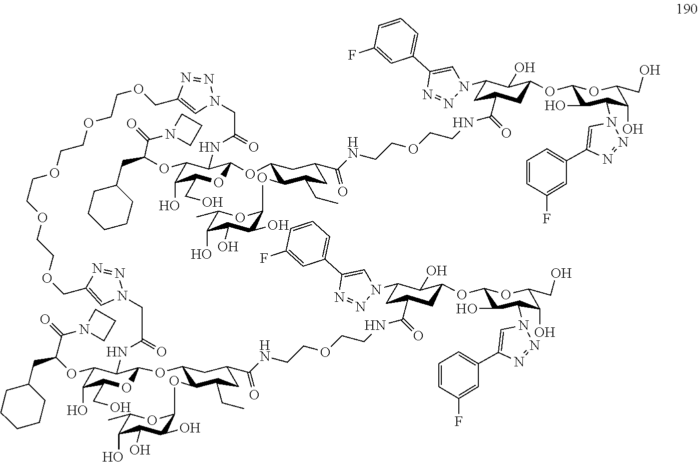

### Example 156

### Prophetic Synthesis of Multimeric Compound 191

Compound 191: Compound 191 can be prepared in an analogous fashion to FIG. 14 by replacing compound 22 with compound 89.

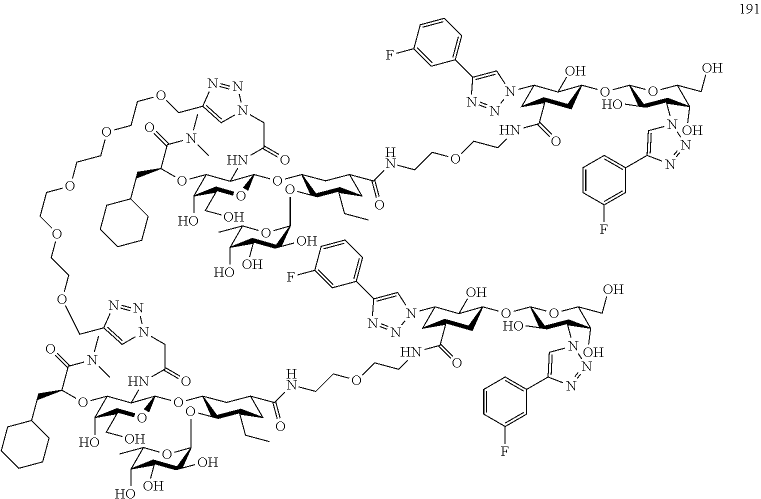

### Example 157

### Prophetic Synthesis of Multimeric Compound 192

Compound 192: Compound 192 can be prepared in an analogous fashion to FIG. 14 by replacing compound 22 with compound 90.

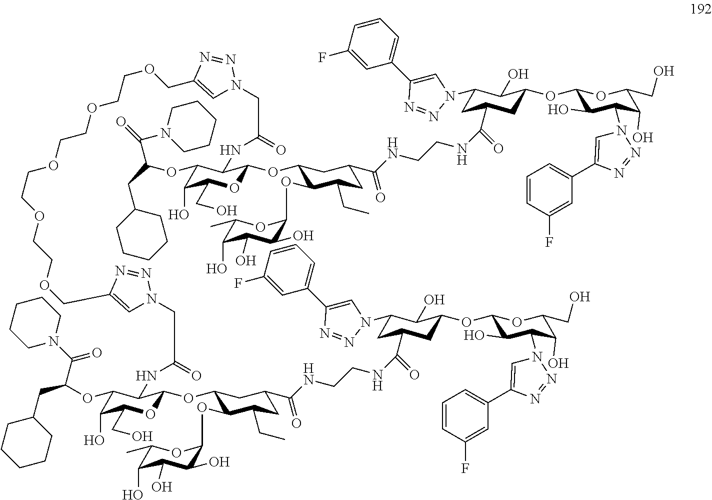

### Example 158

### Prophetic Synthesis of Multimeric Compound 193

Compound 193: Compound 193 can be prepared in an analogous fashion to FIG. 14 by replacing compound 22 with compound 91.

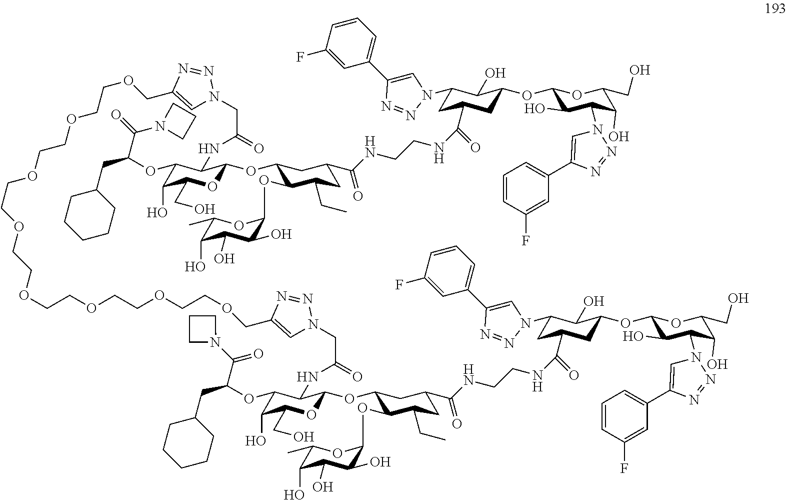

### Example 159

### Prophetic Synthesis of Multimeric Compound 194

Compound 194: Compound 194 can be prepared in an analogous fashion to FIG. 14 by replacing compound 22 with compound 92.

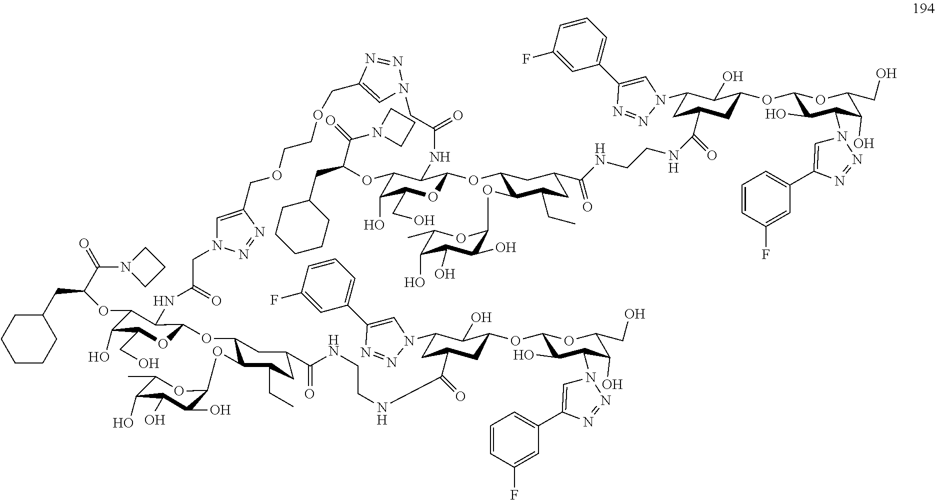

### Example 160

### Prophetic Synthesis of Multimeric Compound 195

Compound 195: Compound 195 can be prepared in an analogous fashion to FIG. 14 by replacing compound 22 with compound 93.

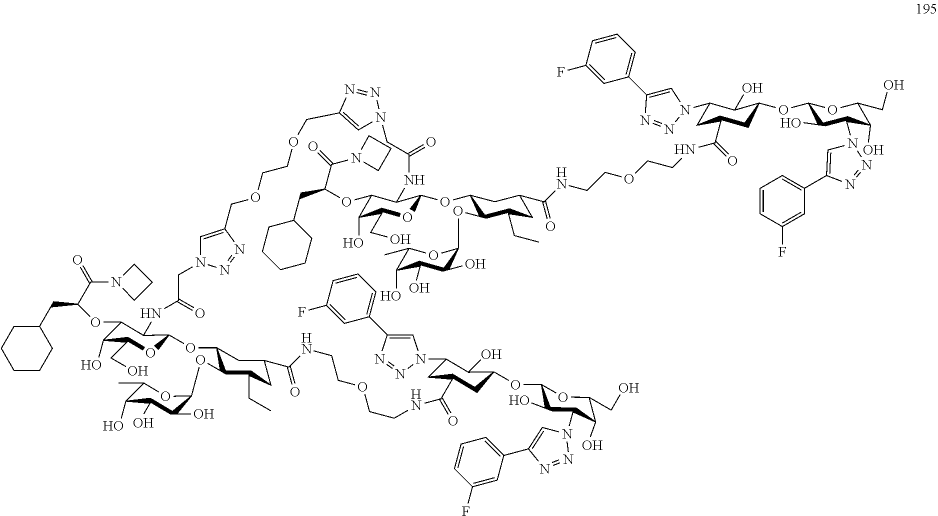

### Example 161

### Prophetic Synthesis of Multimeric Compound 197

Compound 197: To a solution of compound 22 (1 eq) in anhydrous DMSO was acetic acid NHS ester (compound 196)(5 eq). The mixture was stirred at ambient temperature for 12 hours. The solvent was removed in vacuo and the residue was purified by HPLC to afford compound 197.

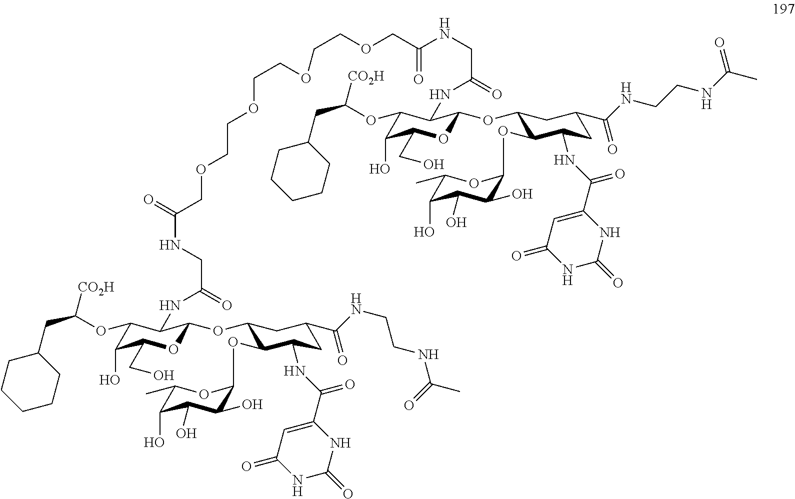

### Example 162

### Prophetic Synthesis of Multimeric Compound 198

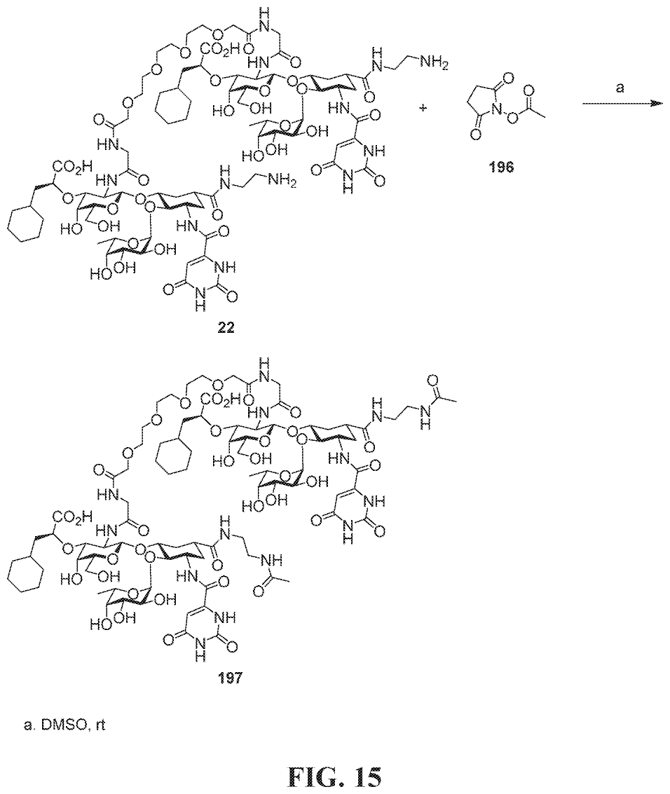

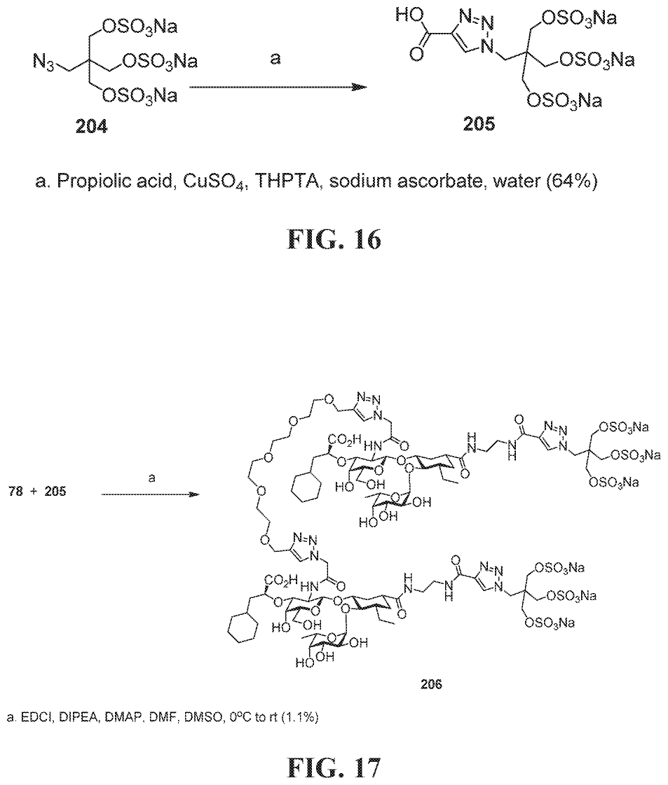

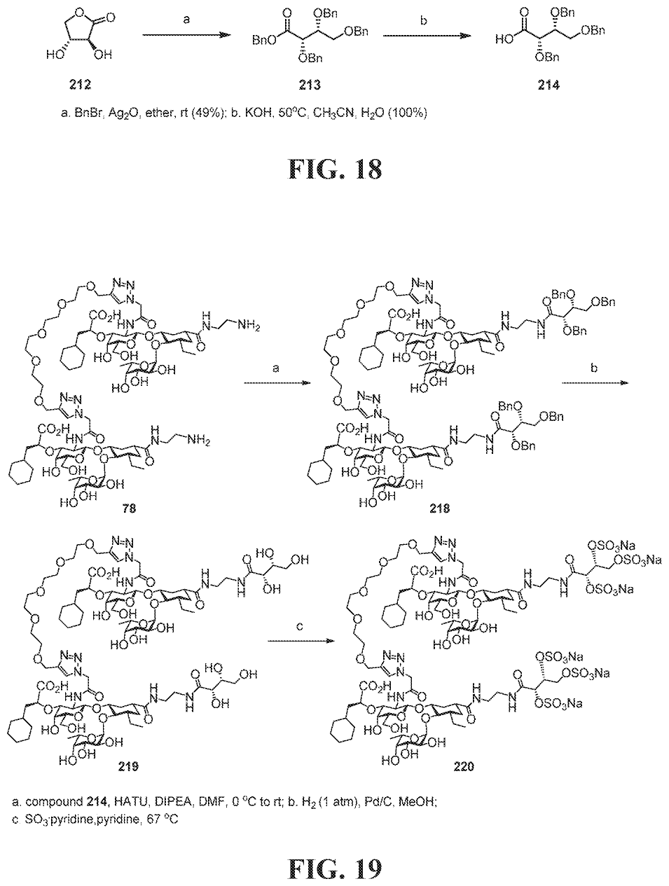

Compound 198: Compound 198 can be prepared in an analogous fashion to FIG. 15 by replacing compound 196 with NHS-methoxyacetate.

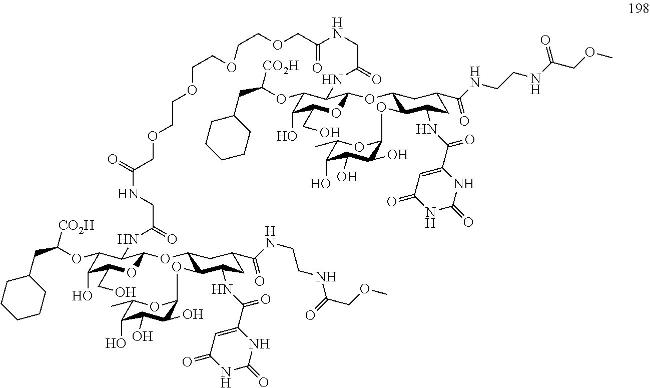

### Example 163

### Prophetic Synthesis of Multimeric Compound 199

Compound 199: Compound 199 can be prepared in an analogous fashion to FIG. 15 by replacing compound 196 with PEG-12 propionic acid NHS ester.

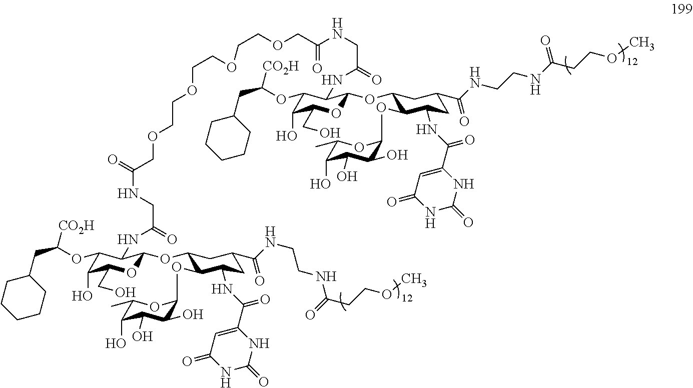

### Example 164

### Prophetic Synthesis of Multimeric Compound 200

Compound 200: Compound 200 can be prepared in an analogous fashion to FIG. 15 by replacing compound 22 with compound 78.

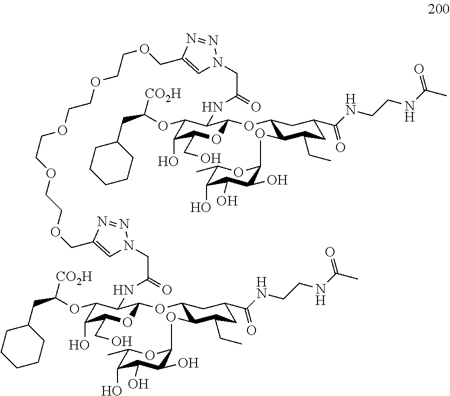

### Example 165

### Prophetic Synthesis of Multimeric Compound 201

Compound 201: Compound 201 can be prepared in an analogous fashion to FIG. 15 by replacing compound 22 with compound 78 and replacing compound 196 with NHS-methoxyacetate.

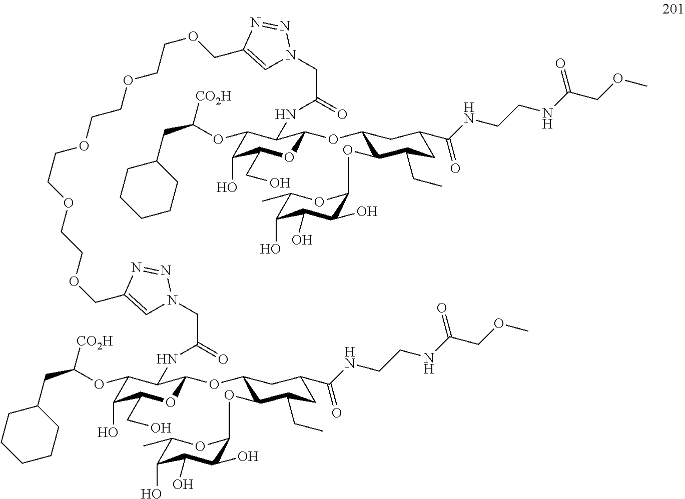

### Example 166

### Prophetic Synthesis of Multimeric Compound 202

Compound 202: Compound 202 can be prepared in an analogous fashion to FIG. 15 by replacing compound 22 with compound 78 and replacing compound 196 with PEG-12 propionic acid NHS ester.

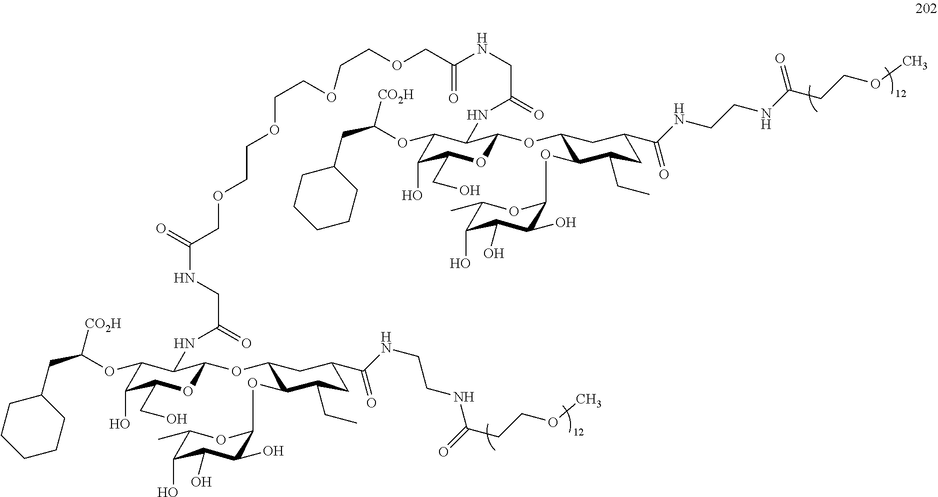

### Prophetic Synthesis of Multimeric Compound 203

Compound 203: Compound 203 can be prepared in an analogous fashion to FIG. 15 by replacing compound 22 with compound 78.

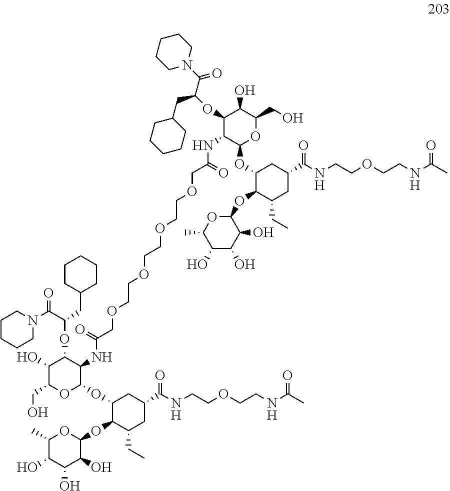 [SMILES: CC[C@H]1C[C@@H](C(=O)NCCOCCNC(C)=O)C[C@@H](O[C@@H]2O[C@H](CO)[C@H](O)[C@H](O[C@@H](CC3CCCCC3)C(=O)N3CCCCC3)[C@H]2NC(=O)COCCOCCOCCOCC(=O)N[C@H]2[C@H](O[C@@H]3C[C@H](C(=O)NCCOCCNC(C)=O)C[C@H](CC)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCCCC2)[C@@H]1O[C@@H]1O[C@@H](C)[C@@H](O)[C@@H](O)[C@@H]1O]

### Example 167

### Prophetic Synthesis of Multimeric Compound 206

Compound 205: A solution of compound 204 (synthesis described in Mead, G. et. al., Bioconj. Chem., 2015, 25, 1444-1452) (0.25 g, 0.53 mmole) and propiolic acid (0.33 mL, 5.30 mmole, 10 eq) in distilled water (1.5 mL) was degassed. A solution of CuSO₄/THPTA in distilled water (0.04 M) (1.3 mL, 53 μmole, 0.1 eq) and sodium ascorbate (21 mg, 0.11 mmole, 0.2 eq) were added successively and the resulting solution was stirred 3 hrs at room temperature. The reaction mixture was concentrated under reduced pressure and partially purified by C-18 column chromatography (water/MeOH, water only—5/5, v/v). The resulting material was further purified by C-18 column chromatography eluting with water to afford compound 205 (0.16 g, 0.34 mmole, 64%). MS: (Calculated for C₈H₁₀₃N₃Na₃O₁₄S₃, 537.34), ES-Negative (513.5, M-Na-1).

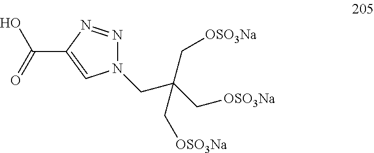

Compound 206: To a solution of compound 205 (7.5 mg, 14 μmole), DIPEA (2.4 μL, 14 μmole) and a catalytic amount of DMAP in DMF/DMSO (3/1, v/v, 0.15 mL) at 0° C. was added EDCI (1.6 mg, 8.22 μmole). The solution was stirred for 20 min. This solution was slowly added to a solution of compound 78 (5.0 mg, 2.7 μmole) in DMF/DMSO (3/1, v/v, 0.2 mL) cooled at 0° C. The resulting solution was stirred 12 hrs allowing the reaction temperature to increase to room temperature. The reaction mixture was purified directly by HPLC. The product portions were collected, concentrated under reduced pressure, then lyophilized to give compound 206 as a white solid (0.4 mg, 1.15 μmole, 1.1%). MS: Calculated (C₉₈H₁₅₄N₁₈Na₆O₅₉S₆, 2856.7), ES-Negative (907.7, M/3; 881.0, M−1SO₃/3; 854.1 M−2SO₃/3; 685.8 M+1Na/4; 680.5 M/4); Fraction of RT=10.65 min, 1399.4, M+7Na−1SO₃/2; 959.3 M+7Na/3; M+7Na−1SO₃/3; 724.8, M+8Na/4; 549.M+1Na/5; 460.9 M+2Na/6; 401.M+4Na/7).

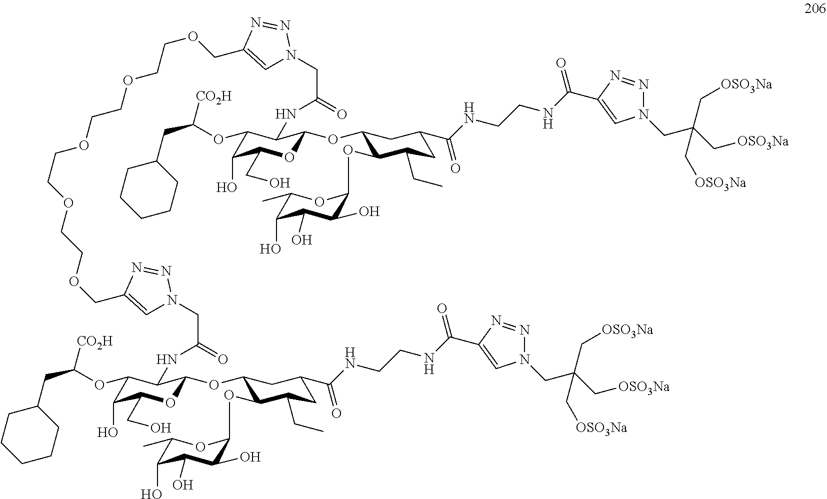

### Example 168

### Prophetic Synthesis of Multimeric Compound 207

Compound 207: Compound 207 can be prepared in an analogous fashion to FIG. 17 by replacing compound 78 with compound 22.

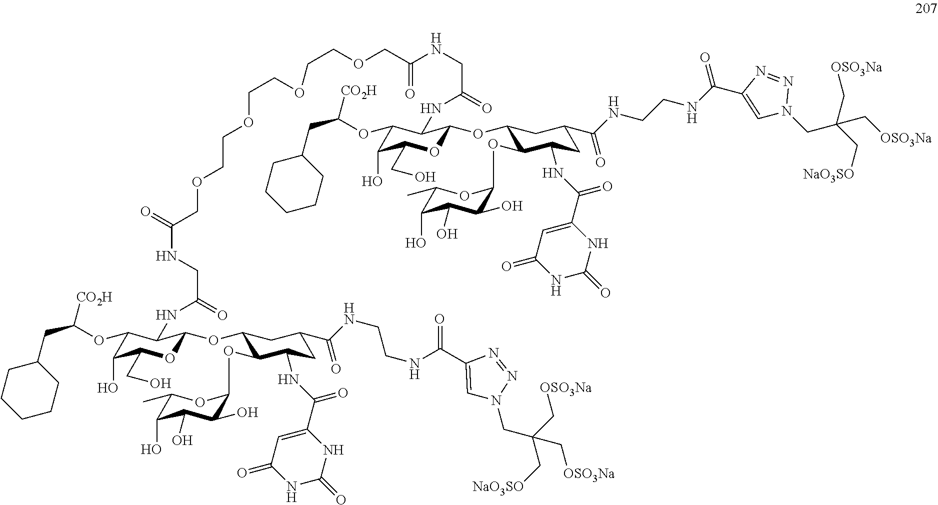

### Example 169

### Prophetic Synthesis of Multimeric Compound 208

Compound 208: Compound 208 can be prepared in an analogous fashion to FIG. 17 using compound 83 in place of compound 78.

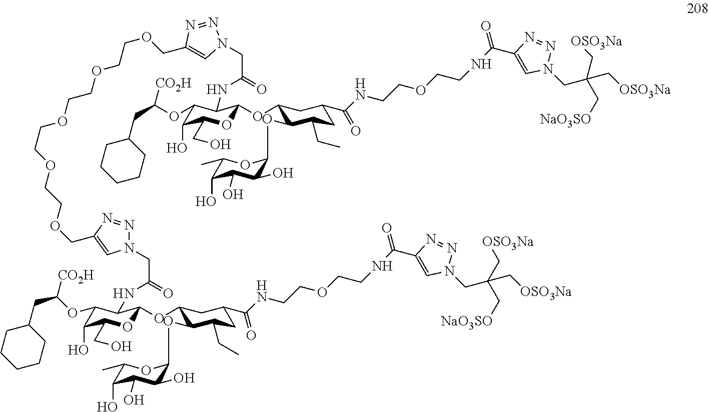

### Example 170

### Prophetic Synthesis of Multimeric Compound 209

Compound 209: Compound 209 can be prepared in an analogous fashion to FIG. 17 using compound 87 in place of compound 78.

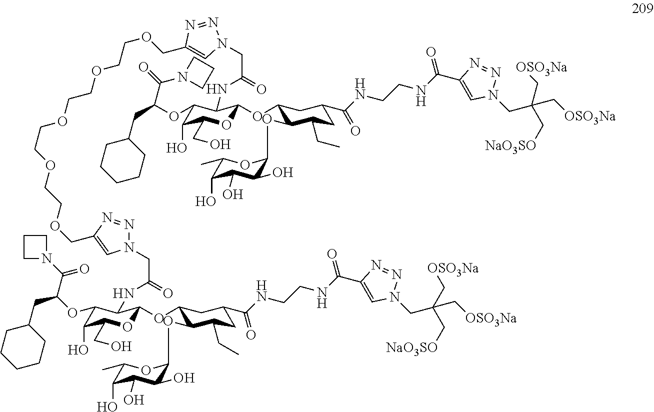

### Example 171

### Prophetic Synthesis of Multimeric Compound 210

Compound 210: Compound 210 can be prepared in an analogous fashion to FIG. 17 using compound 93 in place of compound 78.

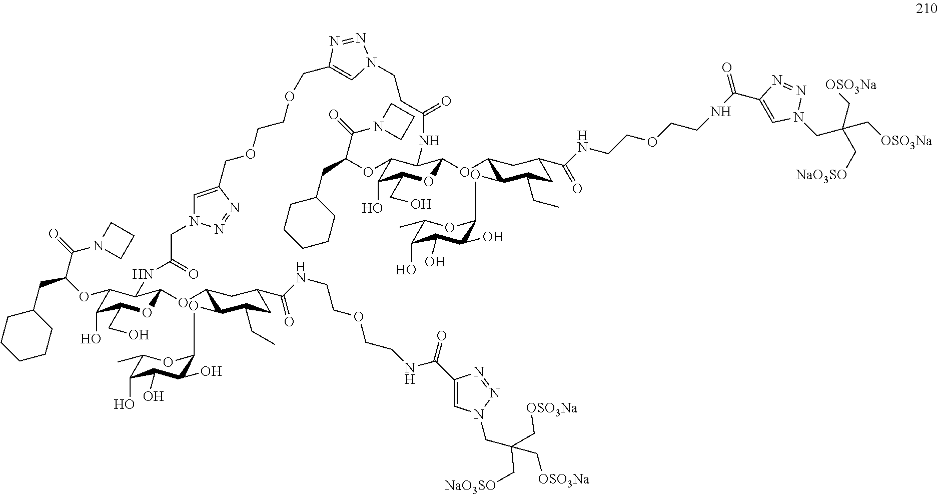

### Example 172

### Prophetic Synthesis of Multimeric Compound 211

Compound 211: Compound 211 can be prepared in an analogous fashion to FIG. 17 using compound 37 in place of compound 78.

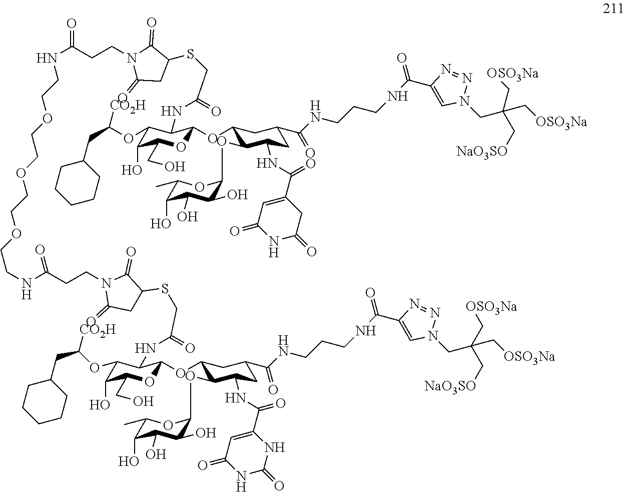

### Example 173

### Synthesis of Multimeric Compound 218

Compound 213: Prepared according to Bioorg. Med. Chem. Lett. 1995, 5, 2321-2324 starting with D-threonolactone.

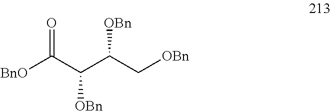 [SMILES: O=C(OCc1ccccc1)[C@@H](OCc1ccccc1)[C@@H](COCc1ccccc1)OCc1ccccc1]

Compound 214: Compound 213 (500 mg, 1 mmol) was dissolved in 9 mL acetonitrile. Potassium hydroxide (1 mL of a 2M solution) was added and the reaction mixture was stirred at 50° C. for 12 hours. The reaction mixture was partitioned between dichloromethane and water. The phases were separated and the aqueous phase was extracted 3 times with dichloromethane. The aqueous phase was acidified with 1N HCl until pH˜1 and extracted 3 times with dichloromethane. The combined dichloromethane extracts from after acidification of the aqueous phase were concentrated in vacuo to give compound 214 as a yellow oil (406 mg). LCMS (C-18; 5-95 H₂O/MeCN): UV (peak at 4.973 min), positive mode: m/z=407 [M+H]⁺; negative mode: m/z=405 [M−H].C₂₅H₂₆O₅ (406).

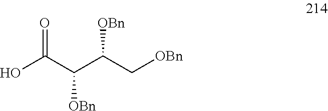 [SMILES: O=C(O)[C@@H](OCc1ccccc1)[C@@H](COCc1ccccc1)OCc1ccccc1]

Compound 215: Prepared in an analogous fashion to compound 214 using L-erythronolactone as the starting material. LCMS (C-18; 5-95 H₂O/MeCN): ELSD (5.08 min), UV (peak at 4.958 min), positive mode: m/z=407 [M+H]⁺; negative mode: m/z=405 [M−H]⁻ C₂₅H₂₆O₅ (406).

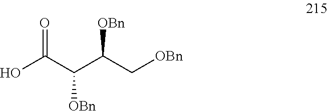 [SMILES: O=C(O)[C@@H](OCc1ccccc1)[C@H](COCc1ccccc1)OCc1ccccc1]

Compound 216: Prepared in an analogous fashion to compound 214 using L-threonolactone as the starting material. LCMS (C-18; 5-95 H₂O/MeCN): ELSD (5.08 min), UV (peak at 4.958 min), positive mode: m/z=407 [M+H]⁺; negative mode: m/z=405 [M−H]⁻ C₂₅H₂₆O₅ (406).

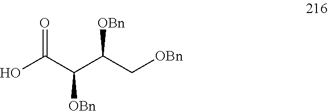 [SMILES: O=C(O)[C@H](OCc1ccccc1)[C@H](COCc1ccccc1)OCc1ccccc1]

Compound 217: Prepared in an analogous fashion to compound 214 using D-erythronolactone as the starting material. LCMS (C-18; 5-95 H₂O/MeCN): ELSD (5.08 min), UV (peak at 4.958 min), positive mode: m/z=407 [M+H]⁺; negative mode: m/z=405 [M−H]⁻ C₂₅H₂₆O₅ (406).

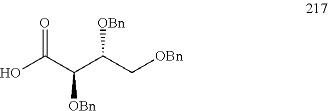 [SMILES: O=C(O)[C@H](OCc1ccccc1)[C@@H](COCc1ccccc1)OCc1ccccc1]

Compound 218: To a solution of compound 214 (3 eq) in anhydrous DMF was added HATU (3.3 eq) and DIPEA (5 eq). The mixture was stirred at ambient temperature for 15 minutes followed by addition of compound 78 (1 eq). The mixture was stirred at ambient temperature for 12 h. The solvent was removed in vacuo and the residue was purified by HPLC to afford compound 218.

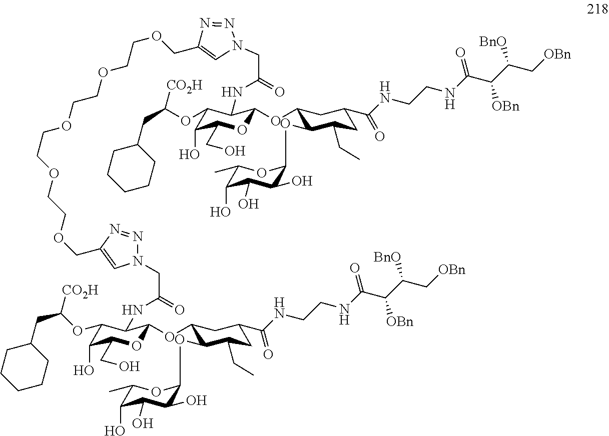

### Example 174

### Prophetic Synthesis of Multimeric Compound 219

Compound 219: Compound 218 is dissolved in methanol and degassed. To this solution is added Pd(OH)₂/C. The reaction mixture is vigorously stirred under a hydrogen atmosphere for 12 hours. The reaction mixture is filtered through a Celite pad. The filtrate is concentrated under reduced pressure to give compound 219.

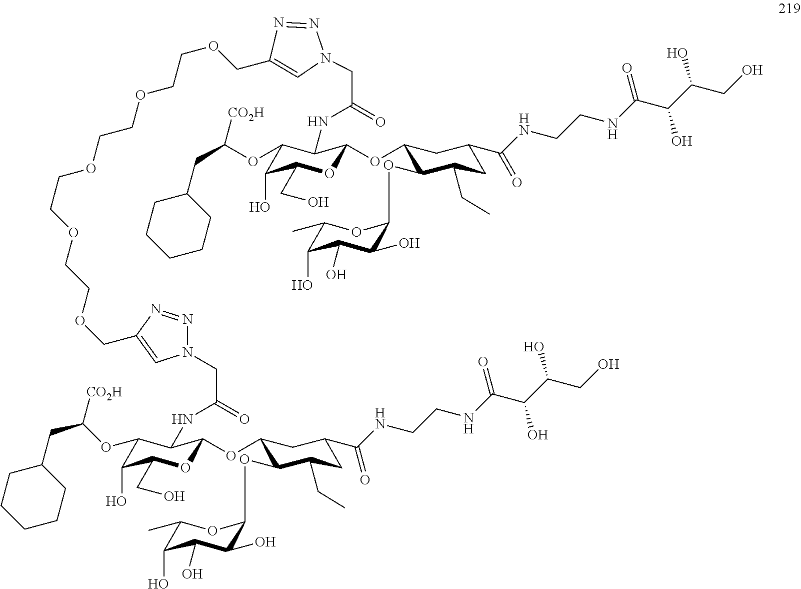

### Example 175

### Synthesis of Multimeric Compound 220

Compound 220: A solution of the sulfur trioxide pyridine complex (100 eq) and compound 219 (1 eq) in pyridine was stirred at 67° C. for 1 h. The reaction mixture was concentrated under vacuum. The resulting solid was dissolved in water and cooled to 0° C. A 1N solution of NaOH was then added slowly until pH-10 and the latter was freeze dried. The resulting residue was purified by Gel Permeation (water as eluent). The collected fractions were lyophilised to give compound 220.

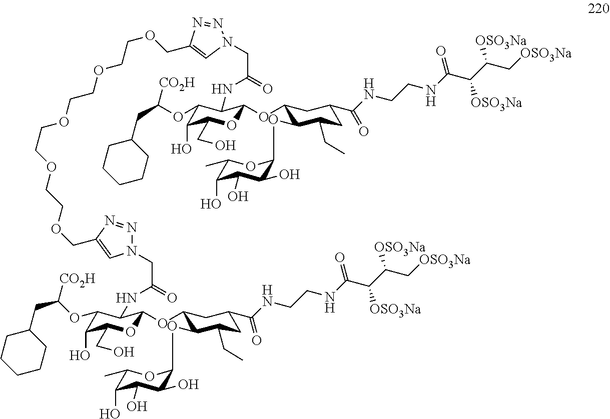

### Example 176

### Prophetic Synthesis of Multimeric Compound 221

Compound 221: Compound 221 can be prepared in an analogous fashion to FIG. 19 by replacing compound 214 with compound 215.

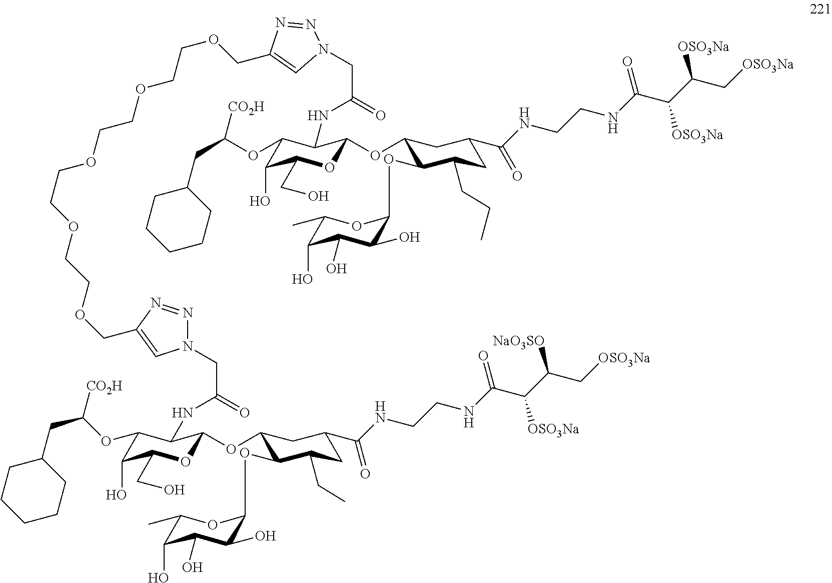

### Example 177

### Prophetic Synthesis of Multimeric Compound 222

Compound 222: Compound 222 can be prepared in an analogous fashion to FIG. 19 by replacing compound 214 with compound 216.

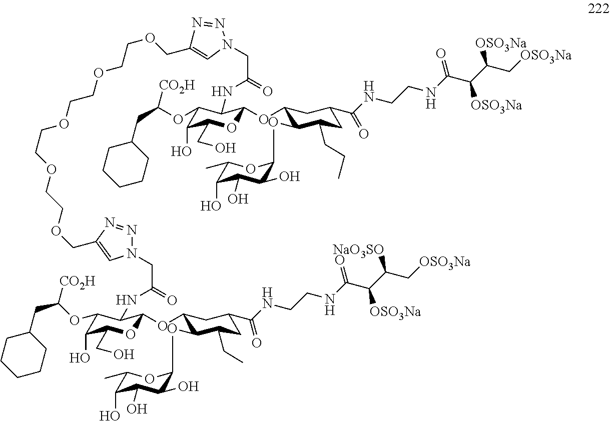

### Example 178

### Prophetic Synthesis of Multimeric Compound 223

Compound 223: Compound 223 can be prepared in an analogous fashion to FIG. 19 by replacing compound 214 with compound 217.

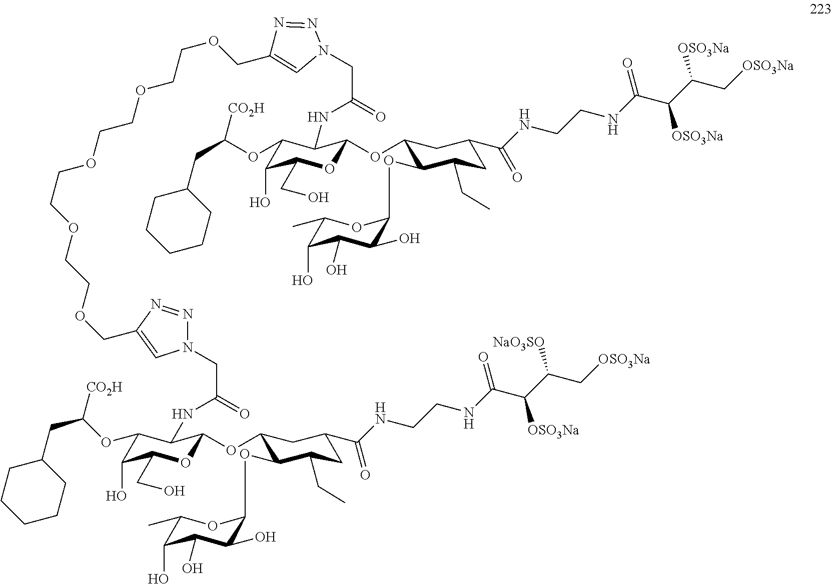

### Example 179

### Synthesis of Multimeric Compound 224

Compound 224: To a solution of compound 78 in anhydrous DMSO was added a drop of DIPEA and the solution was stirred at room temperature until a homogeneous solution was obtained. A solution of succinic anhydride (2.2 eq) in anhydrous DMSO was added and the resulting solution was stirred at room temperature overnight. The solution was lyophilized to dryness and the crude product was purified by HPLC to give compound 224.

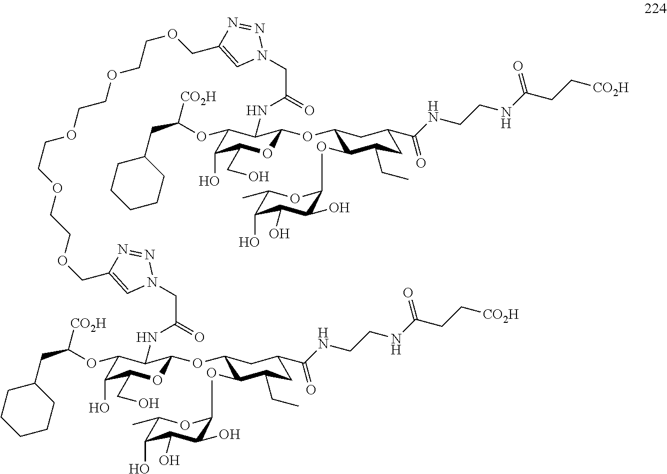

### Example 180

### Prophetic Synthesis of Multimeric Compound 225

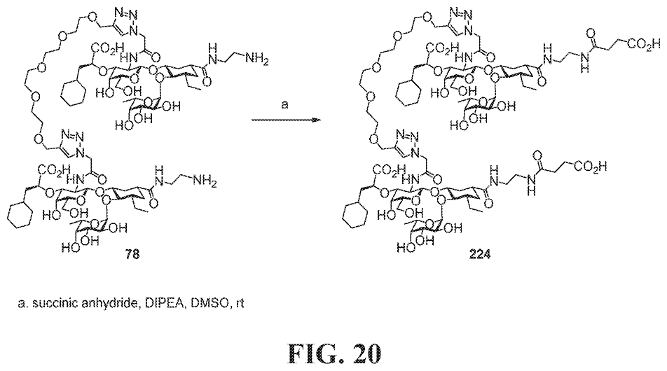

Compound 225: Compound 225 can be prepared in an analogous fashion to FIG. 20 substituting glutaric anhydride for succinic anhydride.

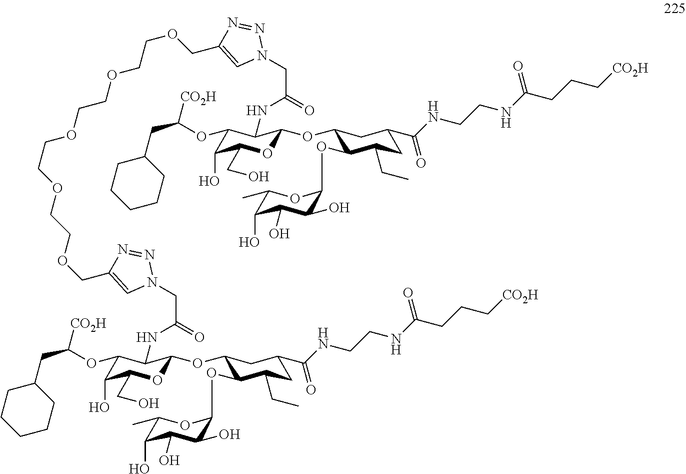

### Example 181

### Prophetic Synthesis of Multimeric Compound 226

Compound 226: Compound 226 can be prepared in an analogous fashion to FIG. 20 substituting compound 87 for compound 78.

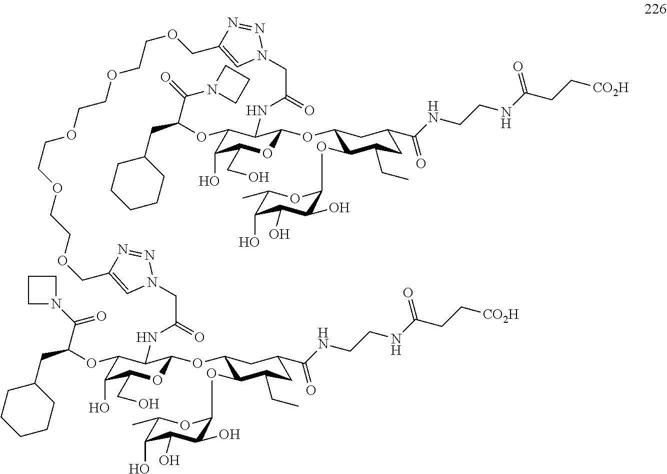

### Example 182

### Prophetic Synthesis of Multimeric Compound 227

Compound 227: Compound 227 can be prepared in an analogous fashion to FIG. 20 substituting phthalic anhydride for succinic anhydride.

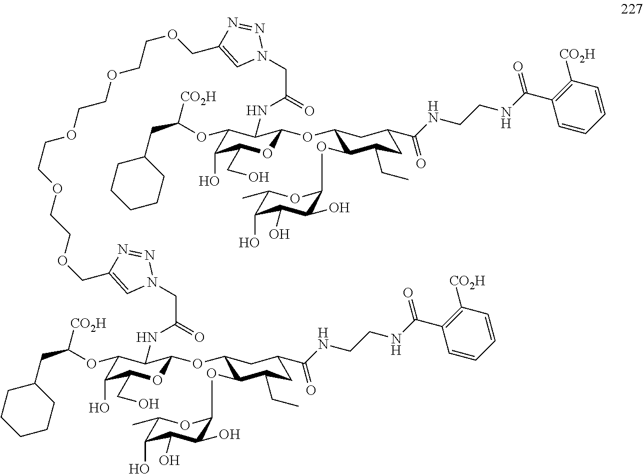

### Example 183

### Prophetic Synthesis of Multimeric Compound 228

Compound 228: Compound 228 can be prepared in an analogous fashion to FIG. 20 using compound 83 in place of compound 78.

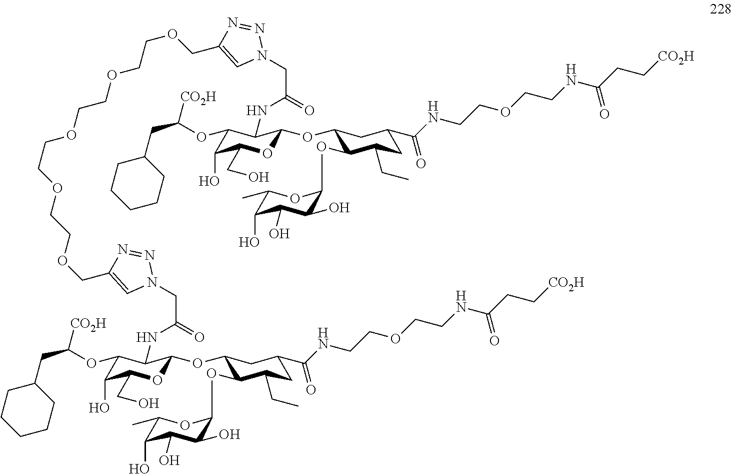

### Example 184

### Prophetic Synthesis of Multimeric Compound 229

Compound 229: Compound 229 can be prepared in an analogous fashion to FIG. 20 using compound 87 in place of compound 78.

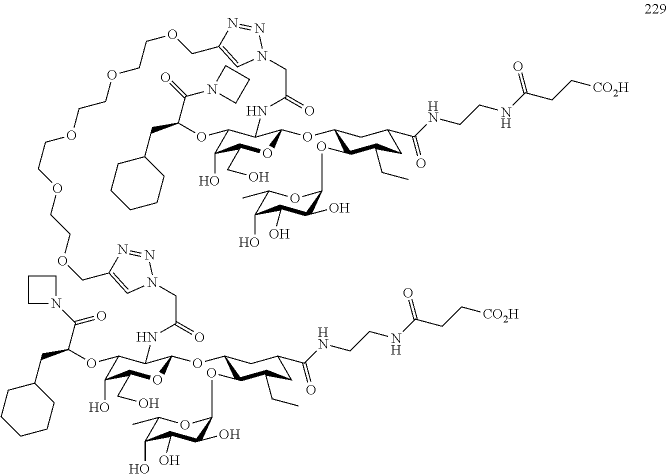

### Example 185

### Prophetic Synthesis of Multimeric Compound 245

Compound 231: A mixture of compound 230 (preparation described in Schwizer, et. al., Chem. Eur. J., 2012, 18, 1342) and compound 2 (preparation described in WO 2013/096926) (1.7 eq) is azeotroped 3 times from toluene. The mixture is dissolved in DCM under argon and cooled on an ice bath. To this solution is added boron trifluoride etherate (1.5 eq). The reaction mixture is stirred 12 hours at room temperature. The reaction is quenched by the addition of triethylamine (2 eq). The reaction mixture is transferred to a separatory funnel and washed 1 time with half saturated sodium bicarbonate solution and 1 time with water. The organic phase is dried over sodium sulfate, filtered, and concentrated. The residue is purified by flash chromatography to afford compound 231.

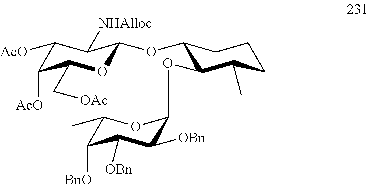

Compound 232: Compound 231 is dissolved in methanol at room temperature. A solution of sodium methoxide in methanol (0.1 eq) is added and the reaction mixture stirred overnight at room temperature. The reaction mixture is quenched by the addition of acetic acid. The reaction mixture is diluted with ethyl acetate, transferred to a separatory funnel and washed 2 times with water. The organic phase is dried over magnesium sulfate, filtered and concentrated. The residue is separated by flash chromatography to afford compound 232.

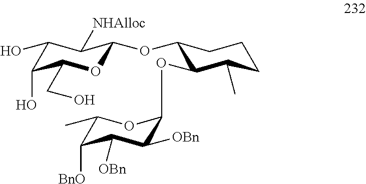

Compound 233: To a solution of compound 232 in dichloromethane cooled on an ice bath is added DABCO (1.5 eq) followed by monomethyoxytrityl chloride (1.2 eq). The reaction mixture is stirred overnight allowing to warm to room temperature. The reaction mixture is concentrated and the residue is purified by flash chromatography to afford compound 233.

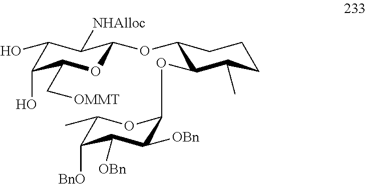

Compound 234: To a solution of compound 233 in methanol is added dibutyltin oxide (1.1 eq). The reaction mixture is refluxed for 3 hours then concentrated. The residue is suspended in DME. To this suspension is added compound 6 (preparation described in Thoma et. al. J. Med. Chem., 1999, 42, 4909) (1.5 eq) followed by cesium fluoride (1.2 eq). The reaction mixture is stirred at room temperature overnight. The reaction mixture is diluted with ethyl acetate, transferred to a separatory funnel, and washed with water. The organic phase is dried over sodium sulfate, filtered and concentrated. The residue is purified by flash chromatography to afford compound 234.

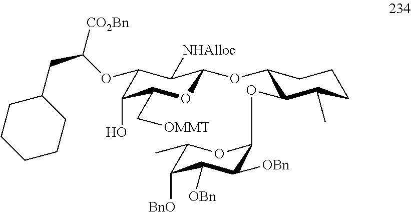

Compound 235: To a degassed solution of compound 234 in anhydrous DCM at 0° C. is added Pd(PPh₃)₄ (0.1 eq), Bu₃SnH (1.1 eq) and N-trifluoroacetyl glycine anhydride (2.0 eq) (preparation described in Chemische Berichte (1955), 88(1), 26). The resulting solution is stirred for 12 hrs allowing the temperature to increase to room temperature. The reaction mixture is diluted with DCM, transferred to a separatory funnel, and washed with water. The organic phase is dried over Na₂SO₄, then filtered and concentrated. The residue is purified by flash chromatography to afford compound 235.

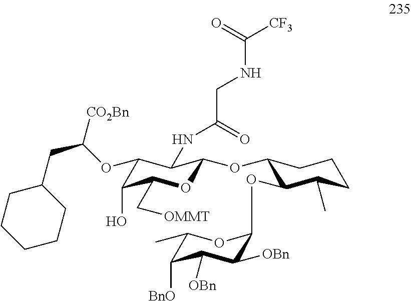

Compound 236: Compound 235 is dissolved in methanol and degassed. To this solution is added Pd(OH)₂/C. The reaction mixture is vigorously stirred under a hydrogen atmosphere for 12 hours. The reaction mixture is filtered through a Celite pad. The filtrate is concentrated under reduced pressure to give compound 236.

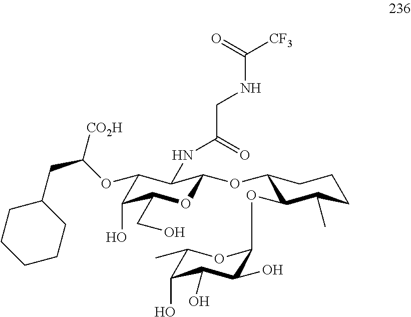

Compound 237: Compound 236 is dissolved in methanol at room temperature. A solution of sodium methoxide in methanol (1.1 eq) is added and the reaction mixture stirred overnight at room temperature. The reaction mixture is quenched by the addition of acetic acid. The reaction mixture is concentrated. The residue is separated by C-18 reverse phase chromatography to afford compound 237.

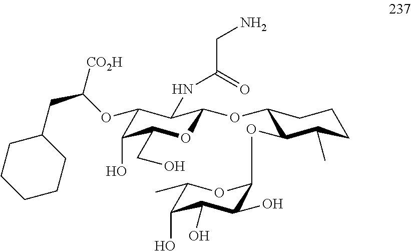

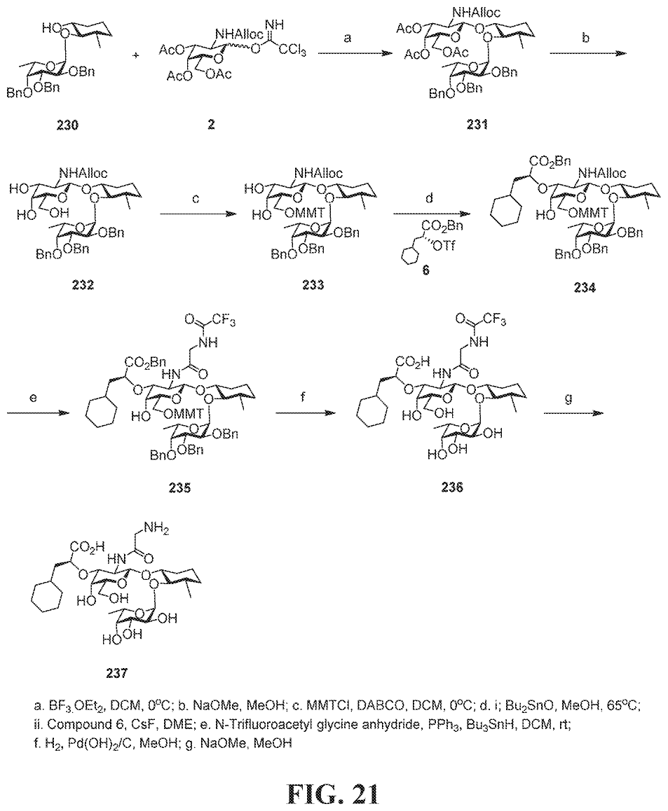

Compound 238: Compound 238 can be prepared in an analogous fashion to FIG. 21 by substituting (acetylthio)acetyl chloride for N-trifluoroacetyl glycine anhydride in step e.

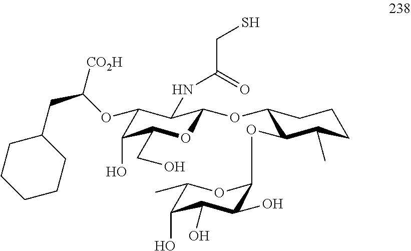

Compound 239: Compound 239 can be prepared in an analogous fashion to FIG. 21 by substituting the vinylcyclohexyl analog of compound 230 (preparation described in Schwizer, et. al., Chem. Eur. J., 2012, 18, 1342) for compound 230 in step a.

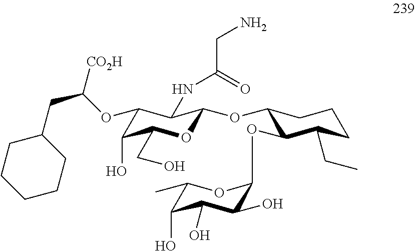

Compound 240: Compound 236 is dissolved in DMF and cooled on an ice bath. Diisopropylethylamine (1.5 eq) is added followed by HATU (1.1 eq). The reaction mixture is stirred 15 minutes on the ice bath then azetidine (2 eq) is added. The ice bath is removed and the reaction mixture is stirred overnight at room temperature. The solvent is removed under reduced pressure and the residue is separated by flash chromatography to afford compound 240.

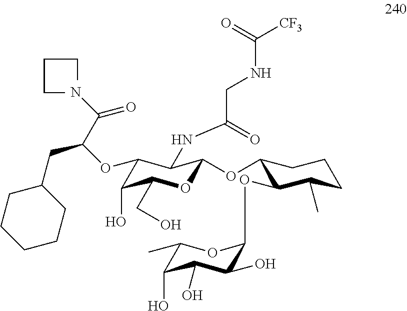

Compound 241: Compound 240 is dissolved in methanol at room temperature. A solution of sodium methoxide in methanol (0.3 eq) is added and the reaction mixture stirred overnight at room temperature. The reaction mixture is quenched by the addition of acetic acid. The reaction mixture is concentrated. The residue is separated by C-18 reverse phase chromatography to afford compound 241.

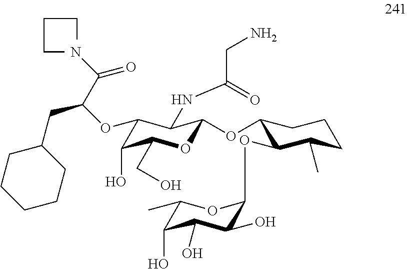

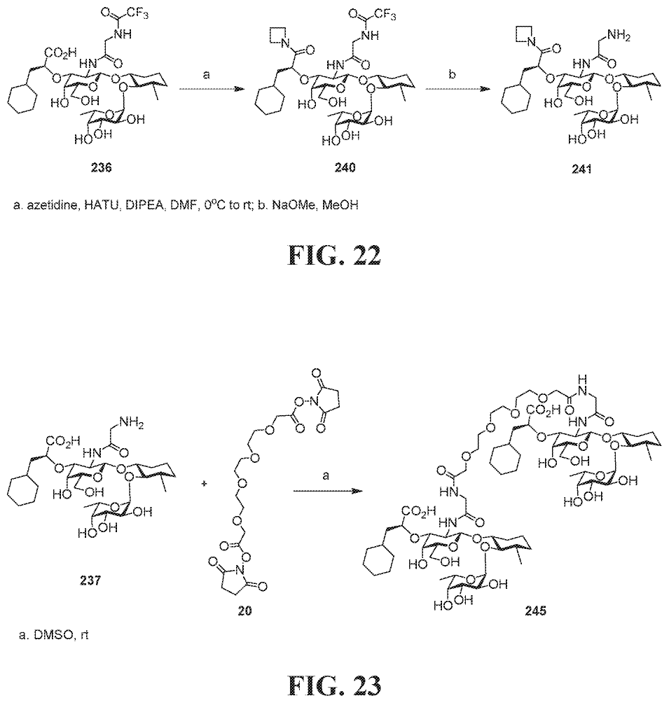

Compound 242: Compound 242 can be prepared in an analogous fashion to FIG. 22 by using methylamine in place of azetidine in step a.

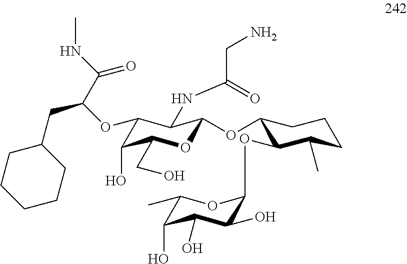

Compound 243: Compound 243 can be prepared in an analogous fashion to FIG. 22 by using dimethylamine in place of azetidine in step a.

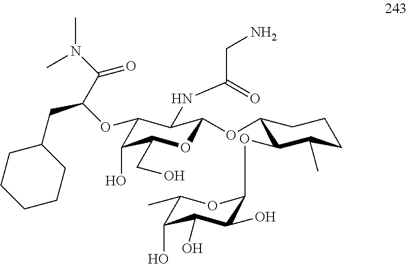

Compound 244: Compound 244 can be prepared in an analogous fashion to FIG. 22 by using the ethylcyclohexyl analog of compound 236 in place of compound 236 in step a.

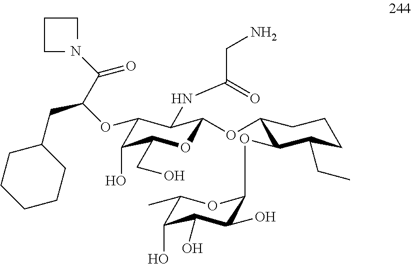

Compound 245: A solution of compound 20 (0.4 eq) in DMSO is added to a solution of compound 237 (1 eq) and DIPEA (10 eq) in anhydrous DMSO at room temperature. The resulting solution is stirred overnight. The reaction mixture is separated by reverse phase chromatography and the product lyophilized to give compound 245.

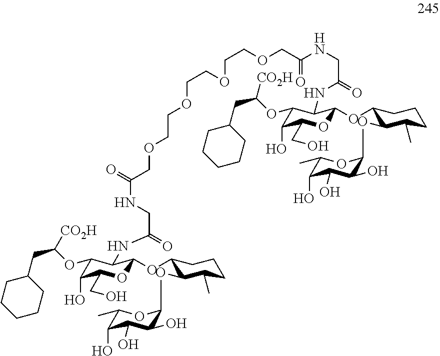

### Example 186

### Prophetic Synthesis of Multimeric Compound 246

Compound 246: Compound 246 can be prepared in an analogous fashion to FIG. 23 by replacing compound 20 with PEG-11 diacetic acid di-NHS ester.

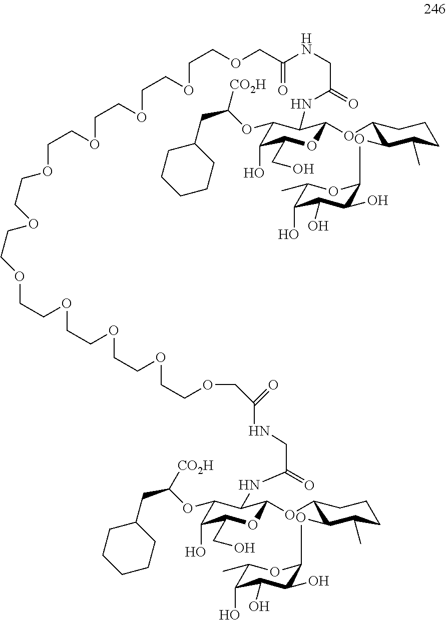

### Example 187

### Prophetic Synthesis of Multimeric Compound 247

Compound 247: Compound 247 can be prepared in an analogous fashion to FIG. 23 by replacing compound 20 with PEG-15 diacetic acid di-NHS ester.

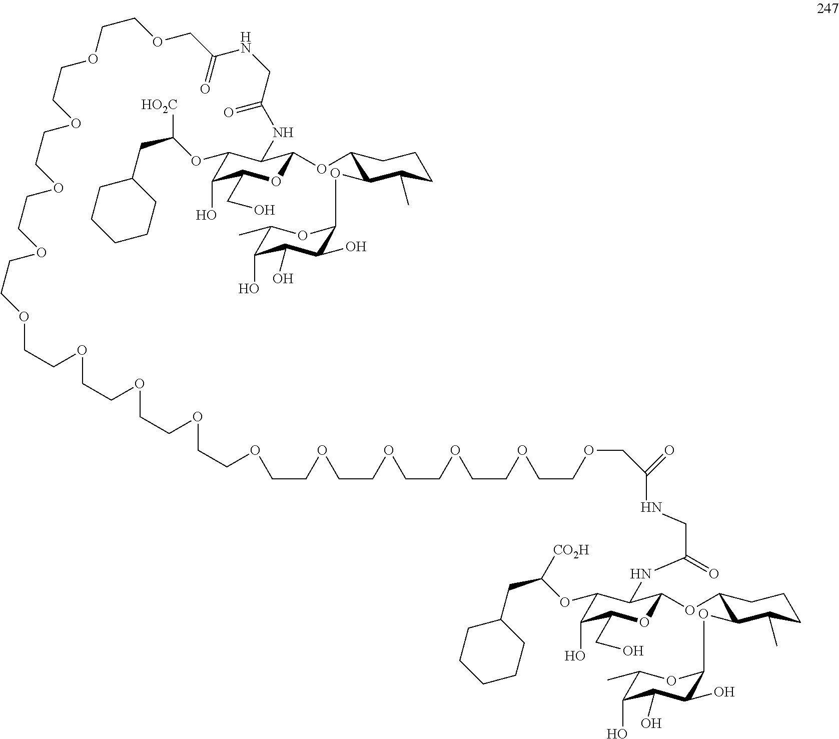

### Example 188

### Prophetic Synthesis of Multimeric Compound 248

Compound 248: Compound 248 can be prepared in an analogous fashion to FIG. 23 by replacing compound 20 with ethylene glycol diacetic acid di-NHS ester.

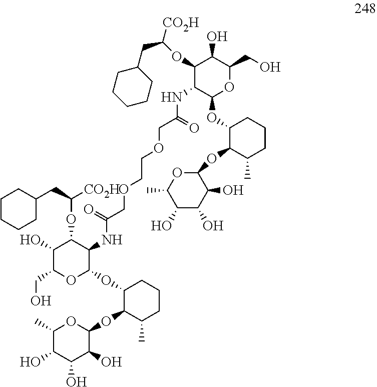 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3CCC[C@H](C)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H](O)[C@H](O)[C@@H]1O]

### Example 189

### Prophetic Synthesis of Multimeric Compound 249

Compound 249: Compound 249 can be prepared in an analogous fashion to FIG. 23 by replacing compound 20 with 3,3′-[[2,2-bis[[3-[(2,5-dioxo-1-pyrrolidinyl)oxy]-3-oxopropoxy]methyl]-1,3-propanediyl]bis(oxy)]bis-, 1,1′-bis(2,5-dioxo-1-pyrrolidinyl)-propanoic acid ester.

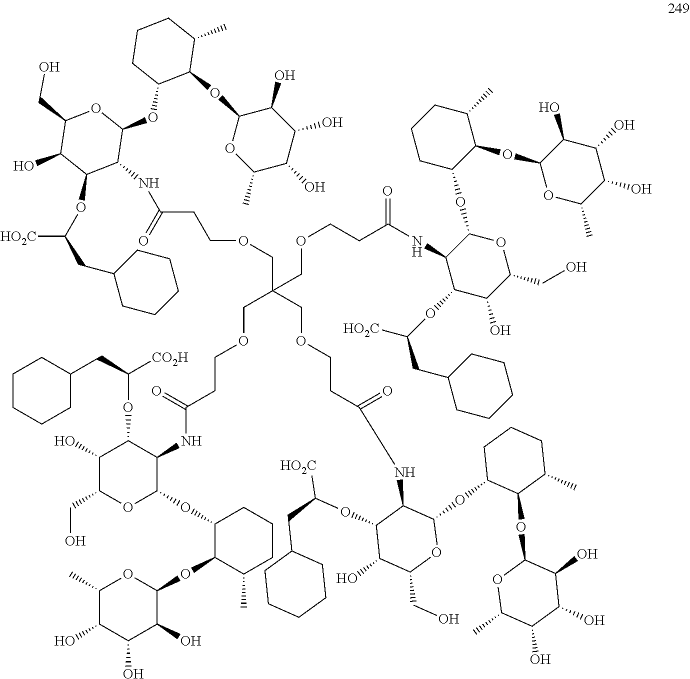

### Example 190

### Prophetic Synthesis of Multimeric Compound 250

Compound 250: Compound 250 can be prepared in an analogous fashion to FIG. 23 by replacing compound 237 with compound 239.

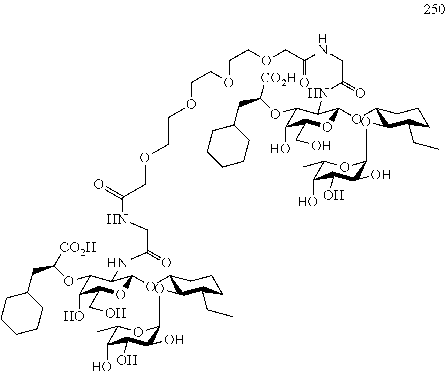

### Example 191

### Prophetic Synthesis of Multimeric Compound 251

Compound 251: Compound 251 can be prepared in an analogous fashion to FIG. 23 by replacing compound 237 with compound 241 and compound 20 with PEG-11 diacetic acid di-NHS ester.

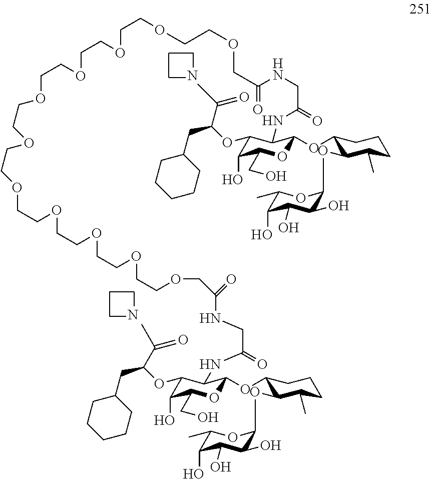

### Example 192

### Prophetic Synthesis of Multimeric Compound 252

Compound 252: Compound 252 can be prepared in an analogous fashion to FIG. 23 by replacing compound 237 with compound 242.

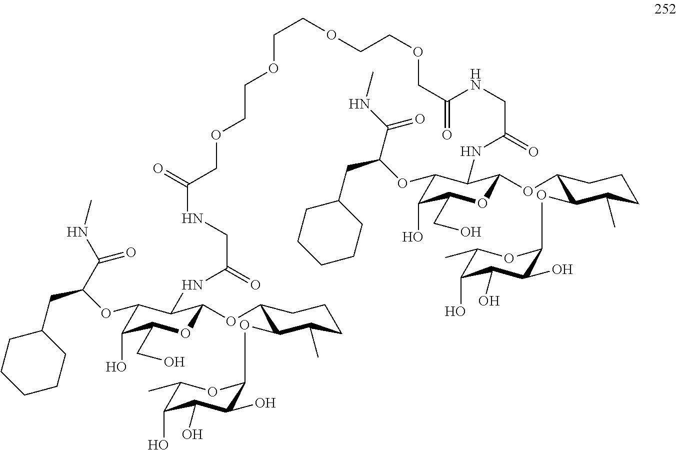

### Example 193

### Prophetic Synthesis of Multimeric Compound 253

Compound 253: Compound 253 can be prepared in an analogous fashion to FIG. 23 by replacing compound 237 with compound 243 and compound 20 with ethylene glycol diacetic acid di-NHS ester.

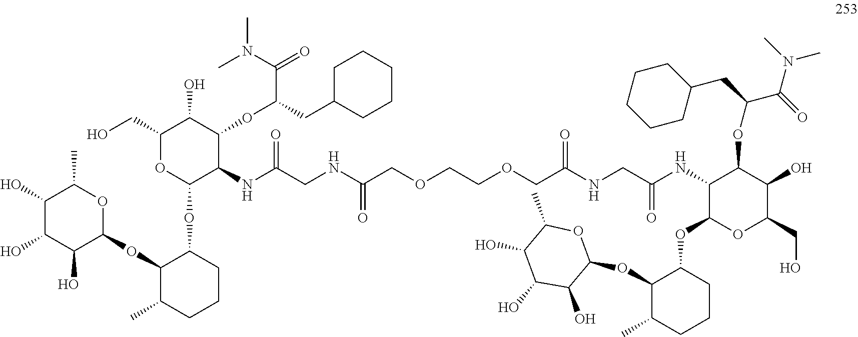 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)N(C)C)[C@H]2NC(=O)CNC(=O)COCCOCC(=O)NCC(=O)N[C@H]2[C@H](O[C@@H]3CCC[C@H](C)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N(C)C)[C@@H](O)[C@H](O)[C@@H]1O]

### Example 194

### Prophetic Synthesis of Multimeric Compound 254

Compound 254: Compound 254 can be prepared in an analogous fashion to FIG. 23 by replacing compound 237 with compound 244 and compound 20 with PEG-11 diacetic acid di-NHS ester.

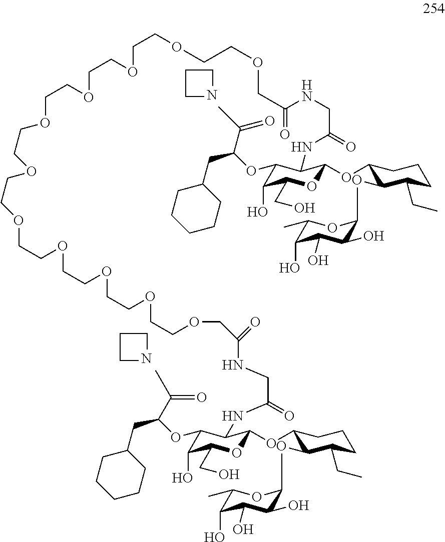

### Example 195

### Prophetic Synthesis of Multimeric Compound 255

Compound 255: Compound 255 can be prepared in an analogous fashion to FIG. 23 by replacing compound 237 with compound 241 and compound 20 with 1,1′-[oxybis[(1-oxo-2,1-ethanediyl)oxy]]bis-2,5-pyrrolidinedione.

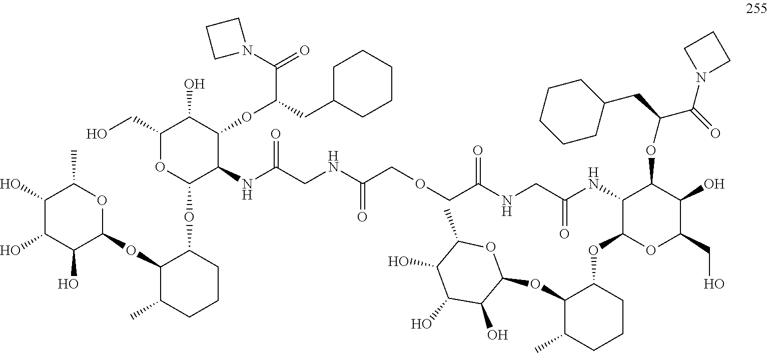 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)N3CCC3)[C@H]2NC(=O)CNC(=O)COCC(=O)NCC(=O)N[C@H]2[C@H](O[C@@H]3CCC[C@H](C)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCC2)[C@@H](O)[C@H](O)[C@@H]1O]

### Example 196

### Prophetic Synthesis of Multimeric Compound 256

Compound 256: Compound 256 can be prepared in an analogous fashion to FIG. 23 by replacing compound 237 with compound 244 and compound 20 with 1,1′-[oxybis[(1-oxo-2,1-ethanediyl)oxy]]bis-2,5-pyrrolidinedione.

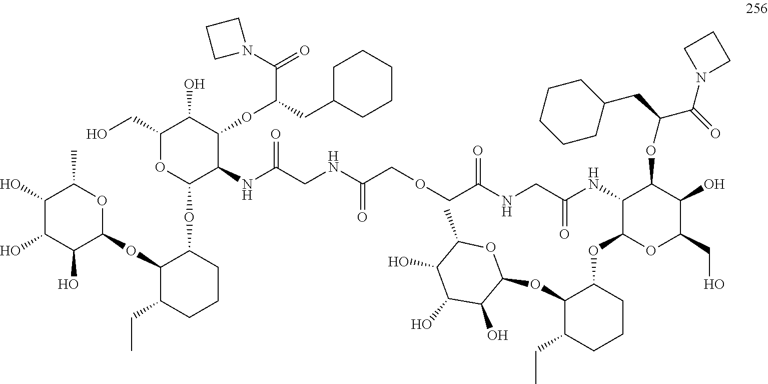 [SMILES: CC[C@H]1CCC[C@@H](O[C@@H]2O[C@H](CO)[C@H](O)[C@H](O[C@@H](CC3CCCCC3)C(=O)N3CCC3)[C@H]2NC(=O)CNC(=O)COCC(=O)NCC(=O)N[C@H]2[C@H](O[C@@H]3CCC[C@H](CC)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCC2)[C@@H]1O[C@@H]1O[C@@H](C)[C@@H](O)[C@@H](O)[C@@H]1O]

### Example 197

### Prophetic Synthesis of Multimeric Compound 257

Compound 257: To a solution of compound 238 in MeOH at room temperature is added compound 35 followed by cesium acetate (2.5 eq). The reaction mixture is stirred at room temperature until completion. The solvent is removed under reduced pressure. The product is purified by reverse phase chromatography to give compound 257.

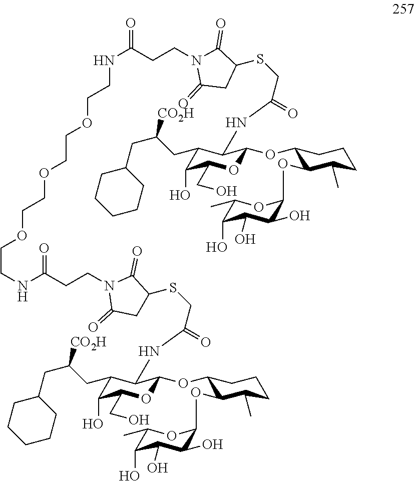

### Example 198

### Prophetic Synthesis of Multimeric Compound 258

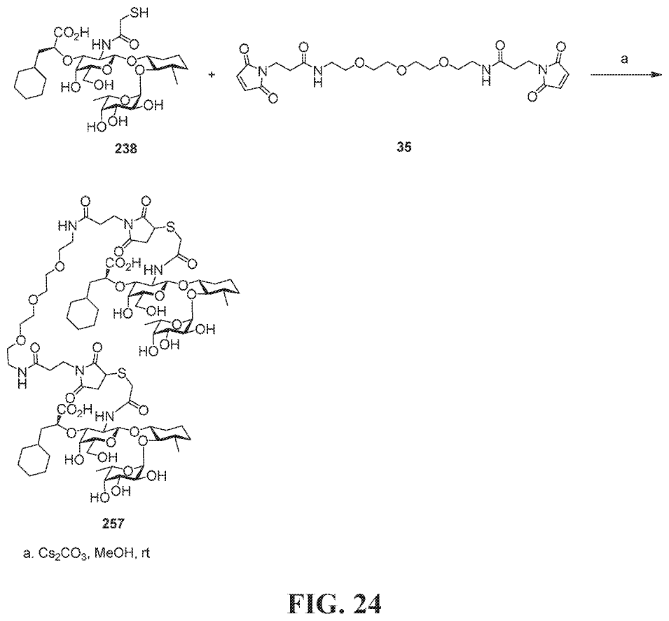

Compound 258: Compound 258 can be prepared in an analogous fashion to FIG. 24 by substituting PEG-6-bis maleimidoylpropionamide for compound 35.

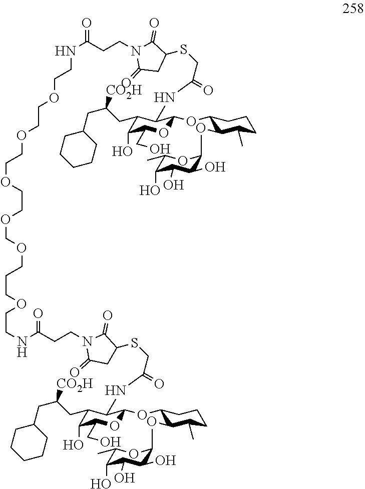

### Example 199

### Prophetic Synthesis of Multimeric Compound 259

Compound 259: Compound 259 can be prepared in an analogous fashion to FIG. 24 by substituting compound 35 for, 1,1′-[[2,2-bis[[3-(2,5-dihydro-2,5-dioxo-1H-pyrrol-1-yl)propoxy]methyl]-1,3-propanediyl]bis(oxy-3,1-propanediyl)]bis-1H-pyrrole-2,5-dione.

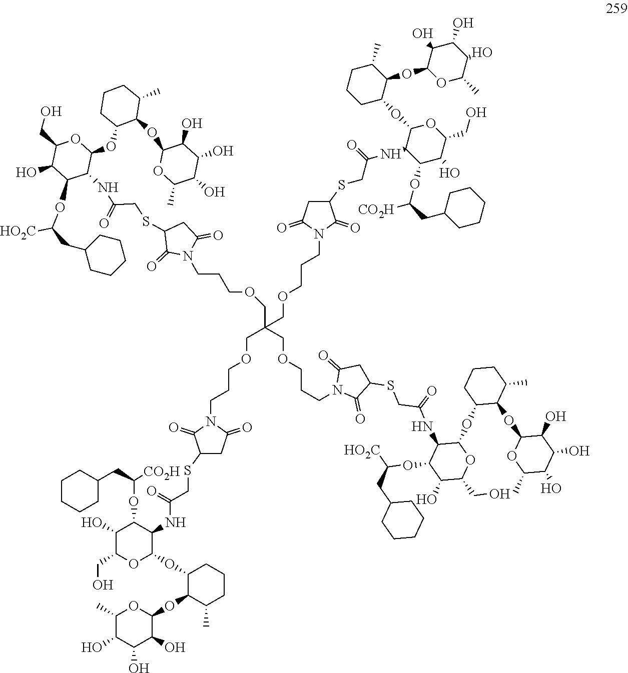

### Example 200

### Prophetic Synthesis of Multimeric Compound 261

Compound 260: To a degassed solution of compound 234 in anhydrous DCM at 0° C. is added Pd(PPh₃)₄ (0.1 eq), Bu₃SnH (1.1 eq) and azidoacetic anhydride (2.0 eq). The ice bath is removed and the solution is stirred for 12 hrs under a N₂ atmosphere at room temperature. The reaction mixture is diluted with DCM, washed with water, dried over Na₂SO₄, then concentrated. The crude product is purified by column chromatography to give compound 260.

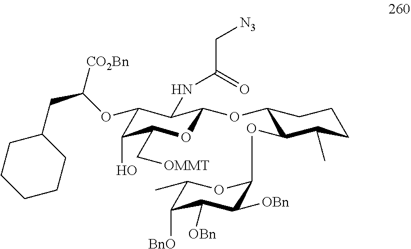

Compound 261: A solution of bis-propagyl PEG-5 (compound 43) and compound 260 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at 70° C. The solution is cooled to room temperature and concentrated under reduced pressure. The crude product is purified by chromatography to give compound 261.

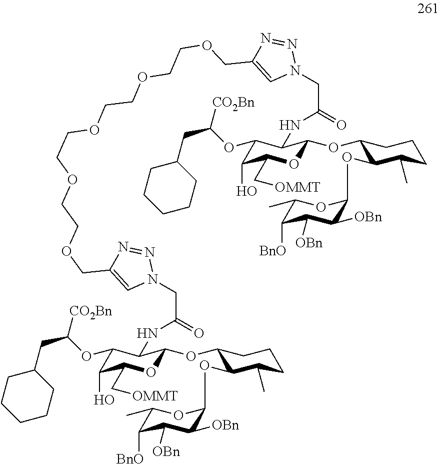

### Example 201

### Prophetic Synthesis of Multimeric Compound 262

Compound 262: Compound 261 is dissolved in MeOH and hydrogenated in the presence of Pd(OH)₂ (20 wt %) at 1 atm of H₂ gas pressure for 24 hrs at room temperature. The solution is filtered through a Celite pad. The filtrate is concentrated to give compound 262.

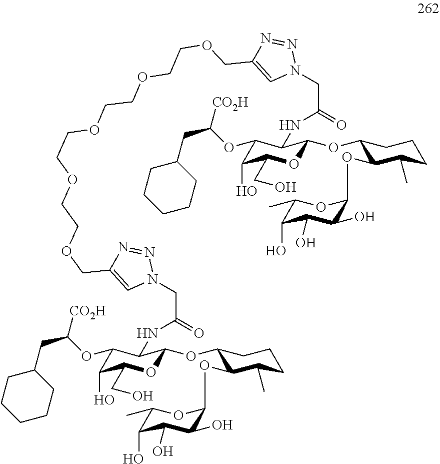

### Example 202

### Prophetic Synthesis of Multimeric Compound 263

Compound 263: Compound 262 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by reverse phase chromatography to afford compound 263.6

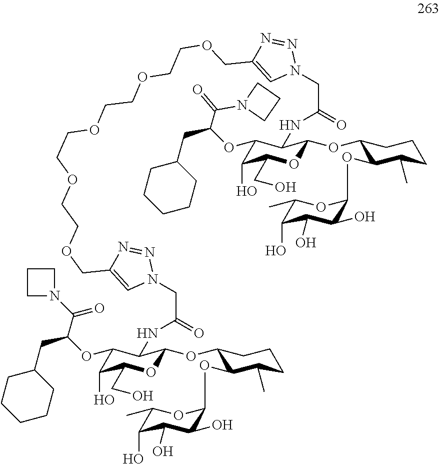

### Example 203

### Prophetic Synthesis of Multimeric Compound 264

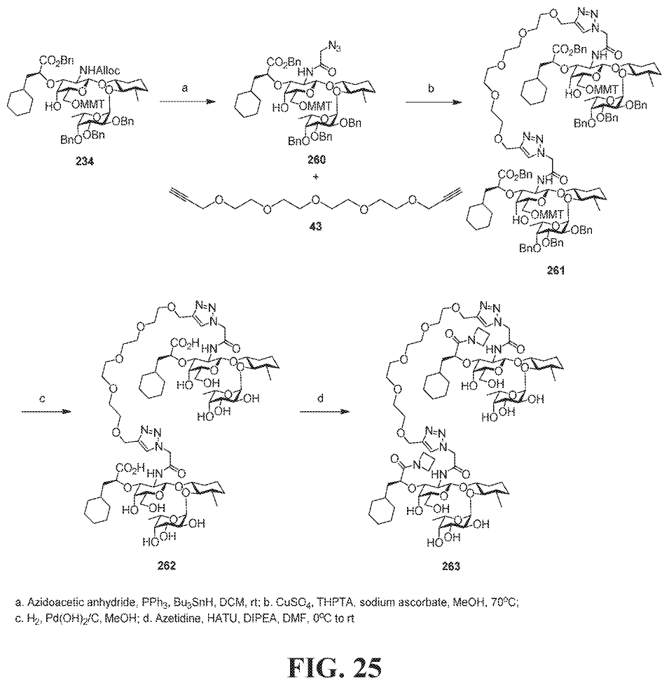

Compound 264: Compound 264 can be prepared in an analogous fashion to FIG. 25 using 4,7,10,13,16,19,22,25,28,31-decaoxatetratriaconta-1,33-diyne in place of compound 43 in step b.

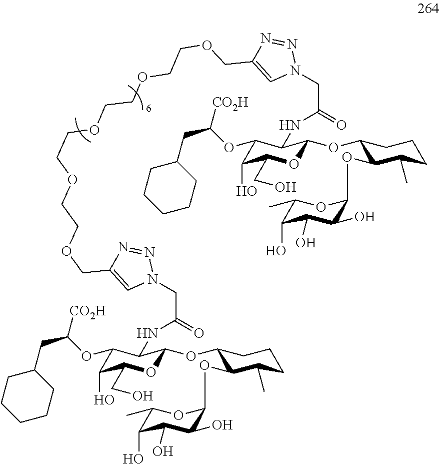

### Example 204

### Prophetic Synthesis of Multimeric Compound 265

Compound 265: Compound 265 can be prepared in an analogous fashion to FIG. 25 using 3,3′-[[2,2-bis[(2-propyn-1-yloxy)methyl]-1,3-propanediyl]bis(oxy)]bis-1-propyne in place of compound 43 in step b.

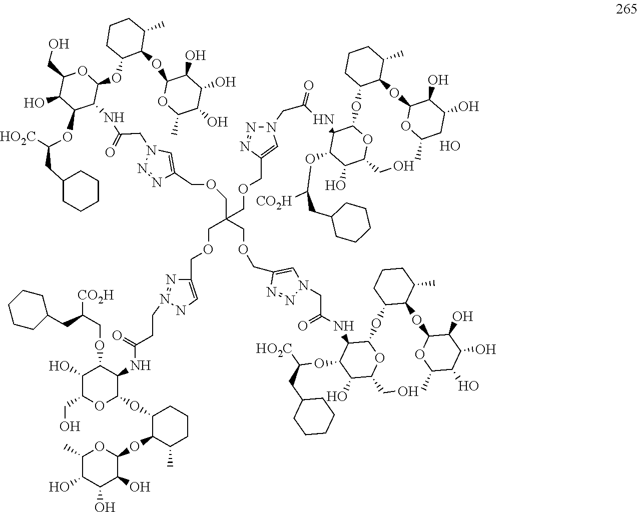

### Example 205

### Prophetic Synthesis of Multimeric Compound 266

Compound 266: Compound 266 can be prepared in an analogous fashion to FIG. 25 using 3,3′-[oxybis[[2,2-bis[(2-propyn-1-yloxy)methyl]-3,1-propanediyl]oxy]]bis-1-propyne in place of compound 43 in step b.

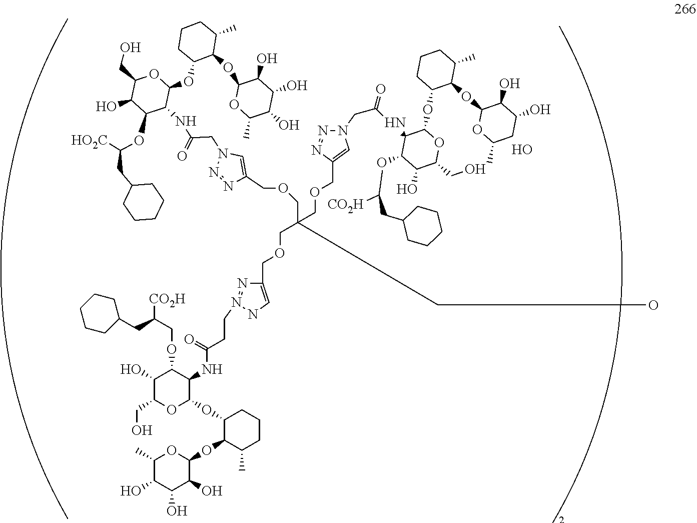

### Example 206

### Prophetic Synthesis of Multimeric Compound 267

Compound 267: Compound 267 can be prepared in an analogous fashion to FIG. 25 using ethylamine in place of azetidine in step d.

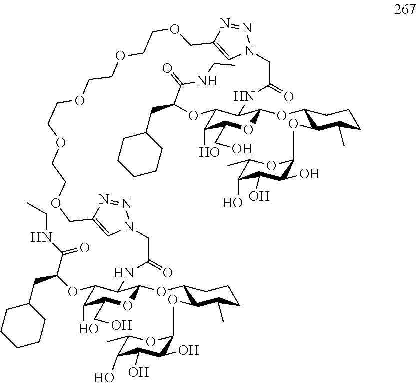

### Example 207

### Prophetic Synthesis of Multimeric Compound 268

Compound 268: Compound 268 can be prepared in an analogous fashion to FIG. 25 using dimethylamine in place of azetidine in step d.

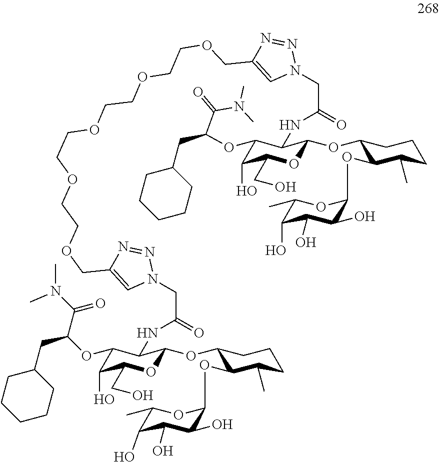

### Example 208

### Prophetic Synthesis of Multimeric Compound 269

Compound 269: Compound 269 can be prepared in an analogous fashion to FIG. 25 using the analog of compound 234 prepared from vinylcyclohexane in place of compound 234 in step a.

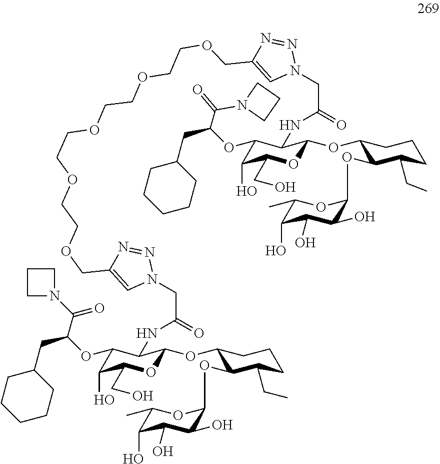

### Example 209

### Prophetic Synthesis of Multimeric Compound 270

Compound 270: Compound 270 can be prepared in an analogous fashion to FIG. 25 using propargyl ether in place of compound 43 in step b.

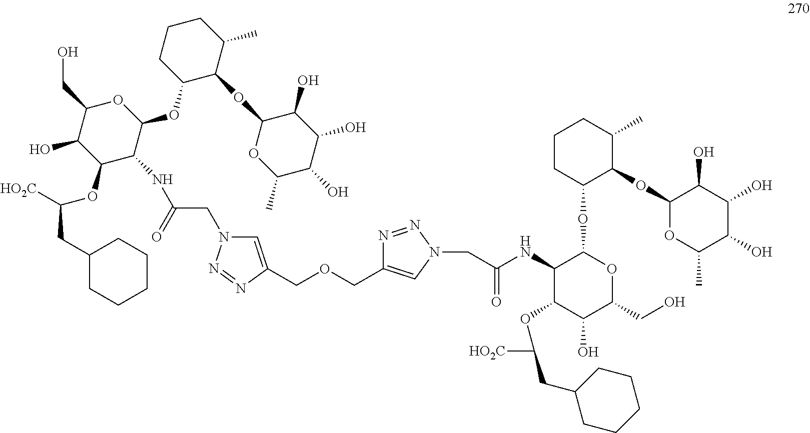 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)O)[C@H]2NC(=O)Cn2cc(COCc3cn(CC(=O)N[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 210

### Prophetic Synthesis of Multimeric Compound 271

Compound 271: Compound 271 can be prepared in an analogous fashion to FIG. 25 using propargyl ether in place of compound 43 in step b.

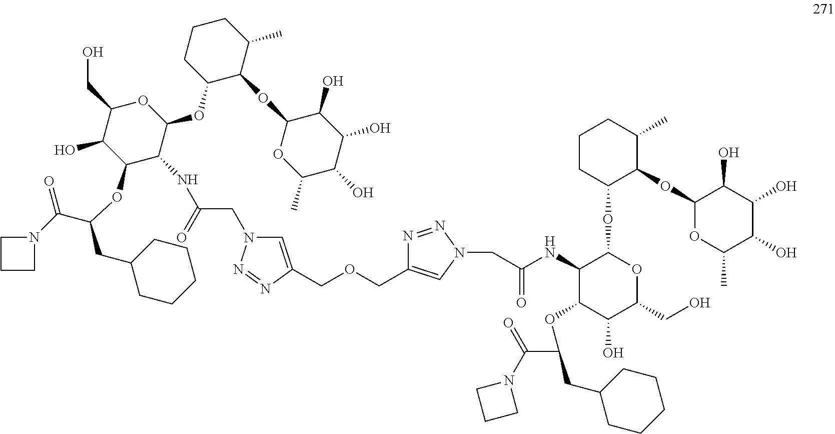 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)N3CCC3)[C@H]2NC(=O)Cn2cc(COCc3cn(CC(=O)N[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)N4CCC4)nn3)nn2)[C@@H](O)[C@H](O)[C@@H]1O]

### Example 211

### Prophetic Synthesis of Multimeric Compound 274

Compound 272: Activated powdered 4 Å molecular sieves are added to a solution of compound 230 and compound 63 (2 eq) in dry DCM under argon. The mixture is stirred for 2 hours at room temperature. Solid DMTST (1.5 eq) is added in 4 portions over 1.5 hours. The reaction mixture is stirred overnight at room temperature. The reaction mixture is filtered through Celite, transferred to a separatory funnel and washed two times with half saturated sodium bicarbonate and two times with water. The organic phase is dried over sodium sulfate, filtered and concentrated. The residue is separated by flash chromatography to afford compound 272.

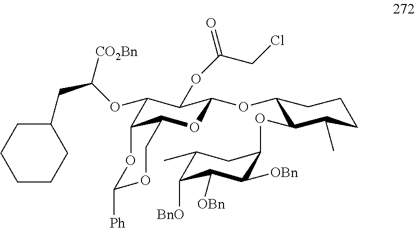

Compound 273: Compound 272 is dissolved in DMF. Sodium azide (1.5 eq) is added and the reaction mixture is stirred at 50° C. until completion. The reaction mixture is cooled to room temperature, diluted with ethyl acetate and transferred to a separatory funnel. The organic phase is washed 4 times with water then dried over sodium sulfate and concentrated. The residue is separated by column chromatography to afford compound 273.

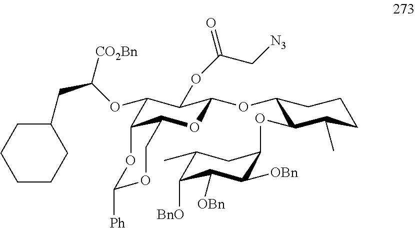

Compound 274: A solution of bispropagyl PEG-5 (compound 43) and compound 273 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at 50° C. The solution is concentrated under reduced pressure. The crude product is purified by chromatography to give a compound 274.

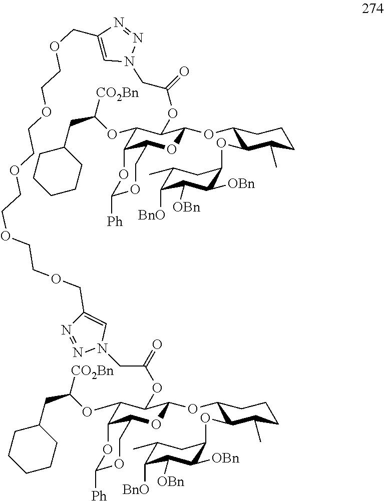

### Example 212

### Prophetic Synthesis of Multimeric Compound 275

Compound 275: To a solution of compound 274 in dioxane/water (4/1) is added Pd(OH)₂/C. The reaction mixture is stirred vigorously overnight under a hydrogen atmosphere. The reaction mixture is filtered through Celite and concentrated. The residue is purified by C-18 reverse phase column chromatography to afford compound 275.

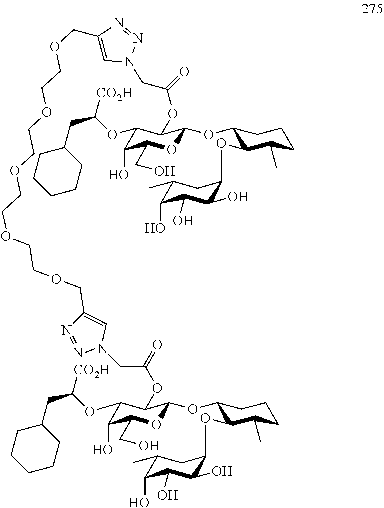

### Example 213

### Prophetic Synthesis of Multimeric Compound 276

Compound 276: Compound 275 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by reverse phase chromatography to afford compound 276.

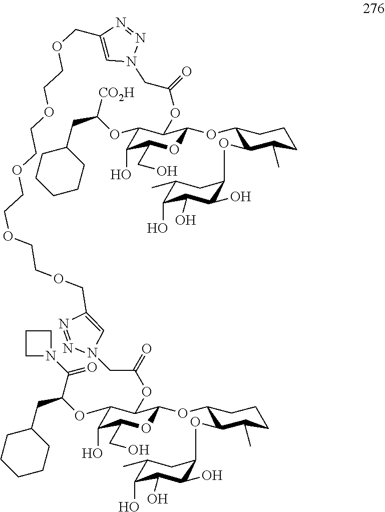

### Example 214

### Prophetic Synthesis of Multimeric Compound 277

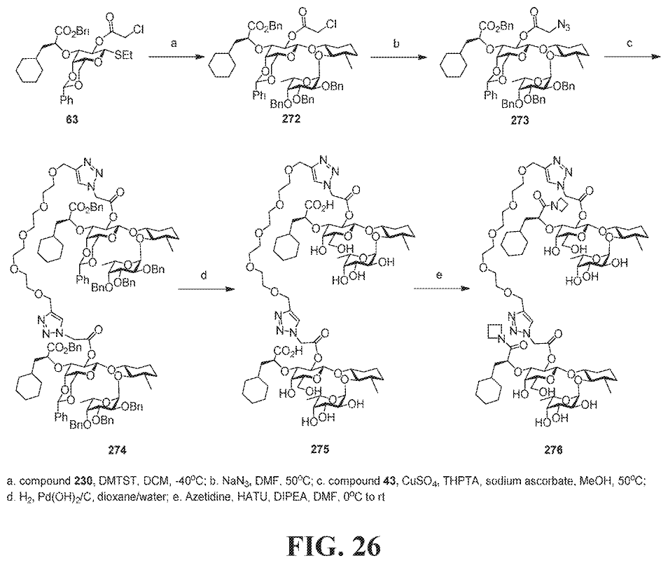

Compound 277: Compound 277 can be prepared in an analogous fashion to FIG. 26 by replacing compound 43 with PEG-8 bis propargyl ether in step c.

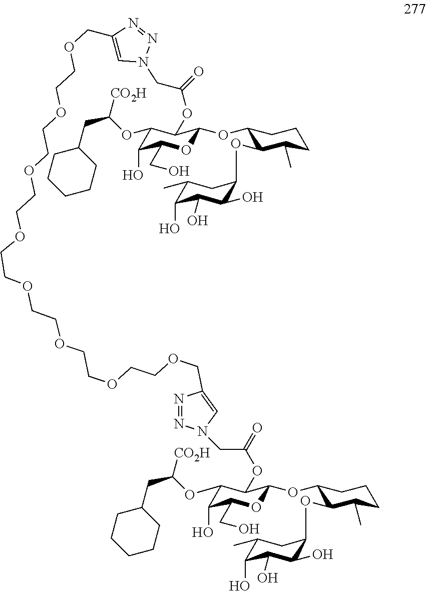

### Example 215

### Prophetic Synthesis of Multimeric Compound 278

Compound 278: Compound 278 can be prepared in an analogous fashion to FIG. 26 by replacing compound 43 with ethylene glycol bis propargyl ether in step c.

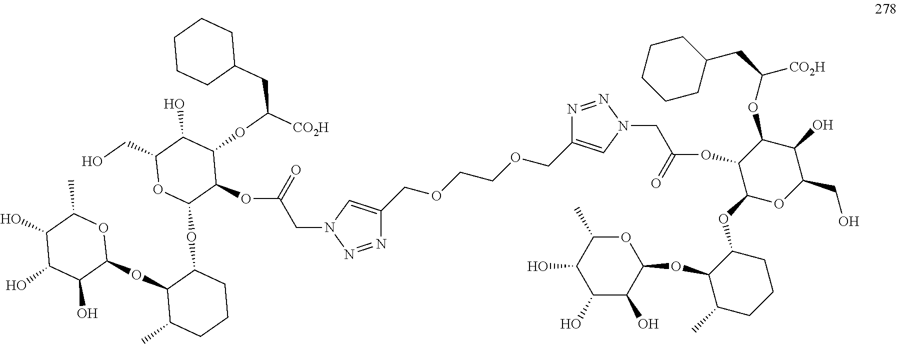 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)O)[C@H]2OC(=O)Cn2cc(COCCOCc3cn(CC(=O)O[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 216

### Prophetic Synthesis of Multimeric Compound 279

Compound 279: Compound 279 can be prepared in an analogous fashion to FIG. 26 using 3,3′-[[2,2-bis[(2-propyn-1-yloxy)methyl]-1,3-propanediyl]bis(oxy)]bis-1-propyne in place of compound 43 in step c.

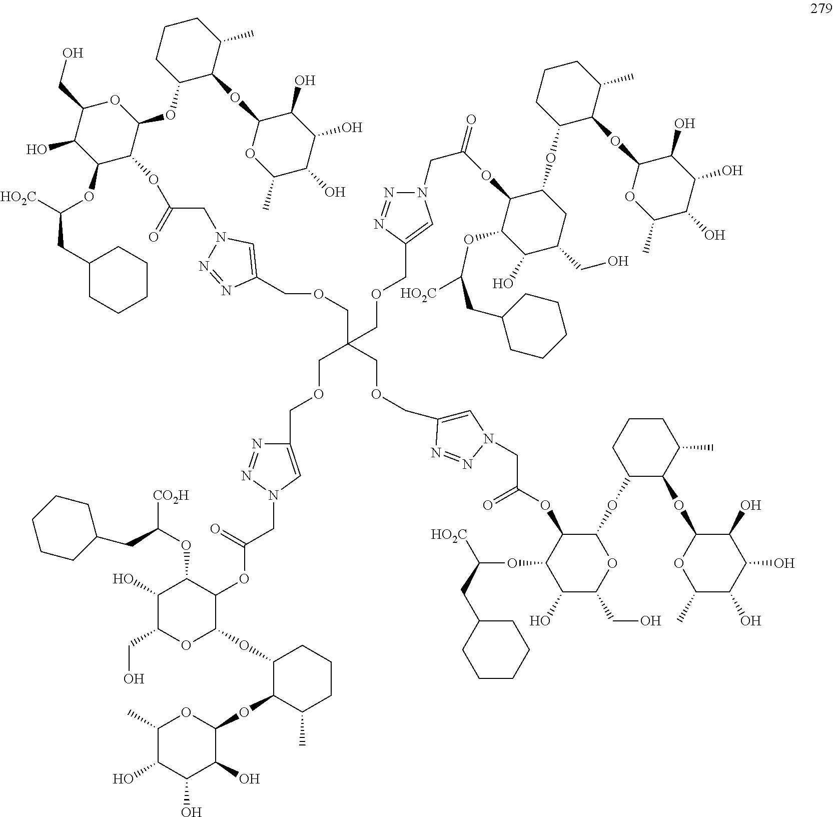

### Example 217

### Prophetic Synthesis of Multimeric Compound 280

Compound 280: Compound 280 can be prepared in an analogous fashion to FIG. 26 using propargyl ether in place of compound 43 in step c.

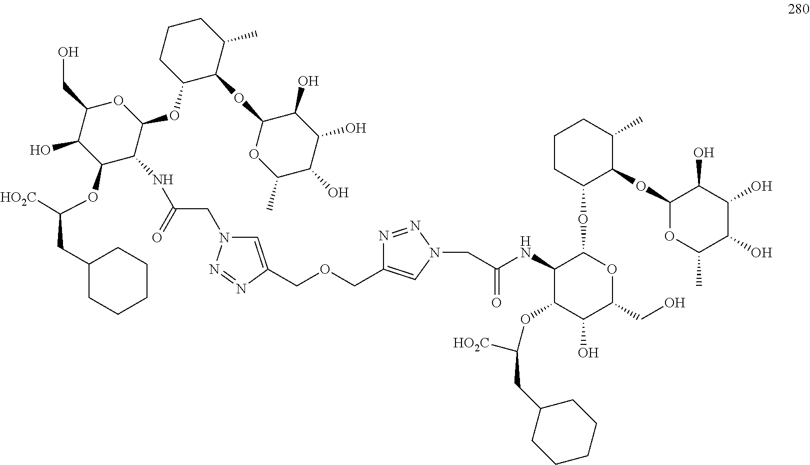 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)O)[C@H]2NC(=O)Cn2cc(COCc3cn(CC(=O)N[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 218

### Prophetic Synthesis of Multimeric Compound 281

Compound 281: Compound 281 can be prepared in an analogous fashion to FIG. 26 using propargyl ether in place of compound 36 in step c.

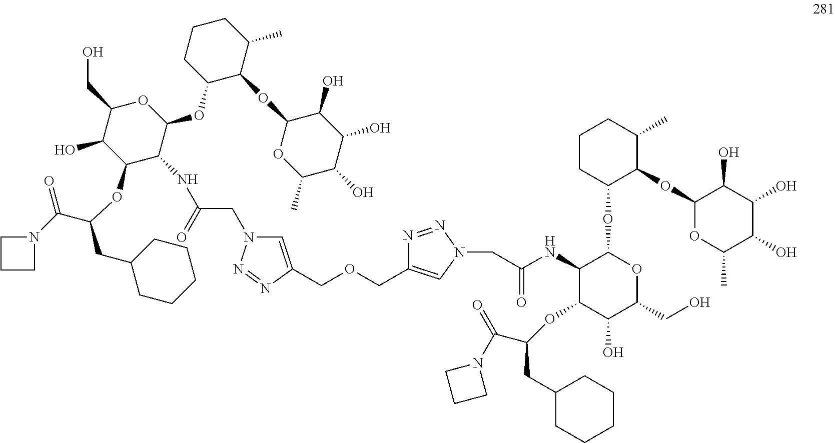 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)N3CCC3)[C@H]2NC(=O)Cn2cc(COCc3cn(CC(=O)N[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)N4CCC4)nn3)nn2)[C@@H](O)[C@H](O)[C@@H]1O]

### Example 219

### Prophetic Synthesis of Multimeric Compound 282

Compound 282: Compound 282 can be prepared in an analogous fashion to FIG. 26 by replacing compound 43 with ethylene glycol bis propargyl ether in step c.

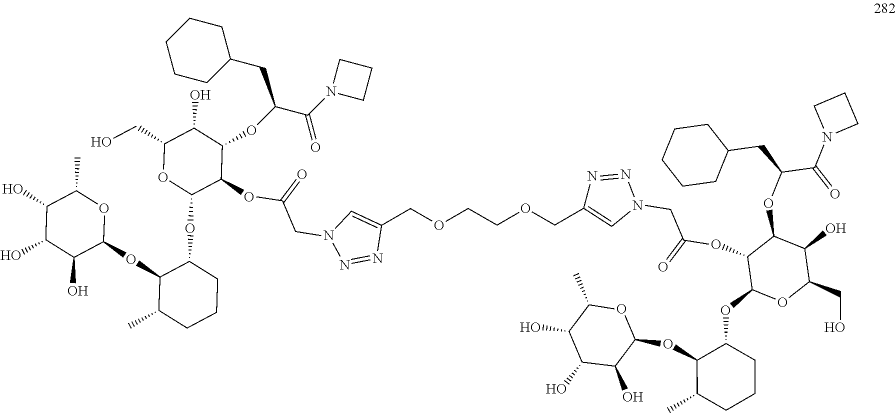 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)N3CCC3)[C@H]2OC(=O)Cn2cc(COCCOCc3cn(CC(=O)O[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)N4CCC4)nn3)nn2)[C@@H](O)[C@H](O)[C@@H]1O]

### Example 220

### Prophetic Synthesis of Multimeric Compound 294

Compound 284: A mixture of compound 283 (preparation described in WO 2007/028050) and compound 2 (preparation described in WO 2013/096926) (1.7 eq) is azeotroped 3 times from toluene. The mixture is dissolved in DCM under argon and cooled on an ice bath. To this solution is added boron trifluoride etherate (1.5 eq). The reaction mixture is stirred 12 hours at room temperature. The reaction is quenched by the addition of triethylamine (2 eq). The reaction mixture is transferred to a separatory funnel and washed 1 time with half saturated sodium bicarbonate solution and 1 time with water. The organic phase is dried over sodium sulfate, filtered, and concentrated. The residue is purified by flash chromatography to afford compound 284.

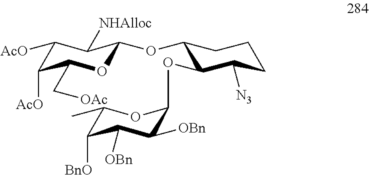

Compound 285: Compound 284 is dissolved in methanol at room temperature. A solution of sodium methoxide in methanol (0.1 eq) is added and the reaction mixture stirred overnight at room temperature. The reaction mixture is quenched by the addition of acetic acid. The reaction mixture is diluted with ethyl acetate, transferred to a separatory funnel and washed 2 times with water. The organic phase is dried over magnesium sulfate, filtered and concentrated. The residue is separated by flash chromatography to afford compound 285.

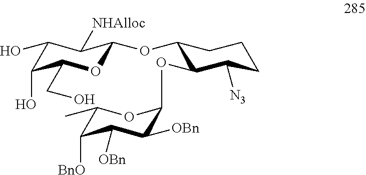

Compound 286: To a solution of compound 285 in dichloromethane cooled on an ice bath is added DABCO (1.5 eq) followed by monomethyoxytrityl chloride (1.2 eq). The reaction mixture is stirred overnight allowing to warm to room temperature. The reaction mixture is transferred to a separatory funnel and washed 2 times with water. The organic phase is concentrated and the residue is purified by flash chromatography to afford compound 286.

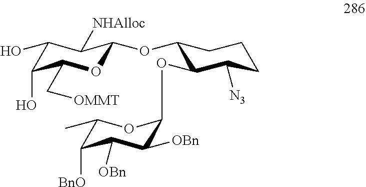

Compound 287: To a solution of compound 286 in methanol is added dibutyltin oxide (1.1 eq). The reaction mixture is refluxed for 3 hours then concentrated. The residue is suspended in DME. To this suspension is added compound 6 (preparation described in Thoma et. al. J. Med. Chem., 1999, 42, 4909) (1.5 eq) followed by cesium fluoride (1.2 eq). The reaction mixture is stirred at room temperature overnight. The reaction mixture is diluted with ethyl acetate, transferred to a separatory funnel, and washed with water. The organic phase is dried over sodium sulfate, filtered and concentrated. The residue is purified by flash chromatography to afford compound 287.

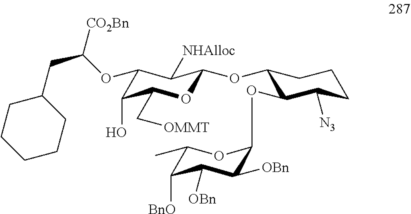

Compound 288: To a degassed solution of compound 287 in anhydrous DCM at 0° C. is added Pd(PPh₃)₄ (0.1 eq), Bu₃SnH (1.1 eq) and N-trifluoroacetyl glycine anhydride (2.0 eq) (preparation described in Chemische Berichte (1955), 88(1), 26). The resulting solution is stirred for 12 hrs allowing the temperature to increase to room temperature. The reaction mixture is diluted with DCM, transferred to a separatory funnel, and washed with water. The organic phase is dried over Na₂SO₄, then filtered and concentrated. The residue is purified by flash chromatography to afford compound 288.

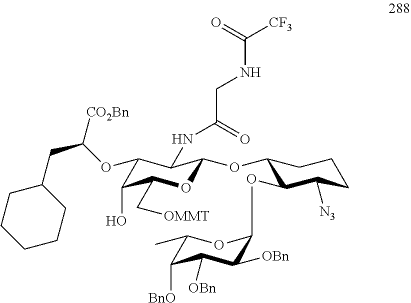

Compound 289: To a stirred solution of compound 288 in DCM/MeOH (25/1) at room temperature is added orotic acid chloride (5 eq) and triphenylphosphine (5 eq). The reaction mixture is stirred 24 hours. The solvent is removed and the residue is separated by column chromatography to afford compound 289.

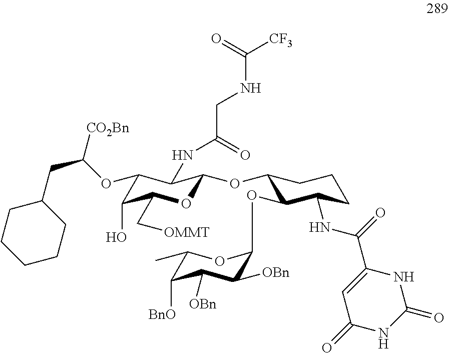

Compound 290: Compound 289 is dissolved in methanol and degassed. To this solution is added Pd(OH)₂/C. The reaction mixture is vigorously stirred under a hydrogen atmosphere for 12 hours. The reaction mixture is filtered through a Celite pad. The filtrate is concentrated under reduced pressure to give compound 290.

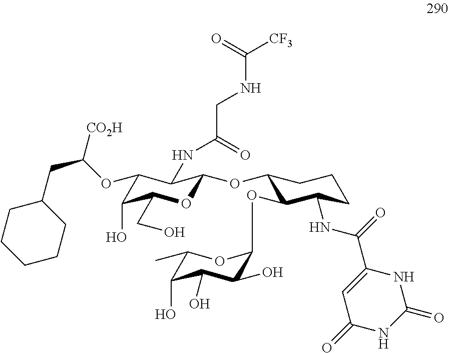

Compound 291: Compound 290 is dissolved in methanol at room temperature. A solution of sodium methoxide in methanol (1.1 eq) is added and the reaction mixture stirred overnight at room temperature. The reaction mixture is quenched by the addition of acetic acid. The reaction mixture is concentrated. The residue is separated by C-18 reverse phase chromatography to afford compound 291.

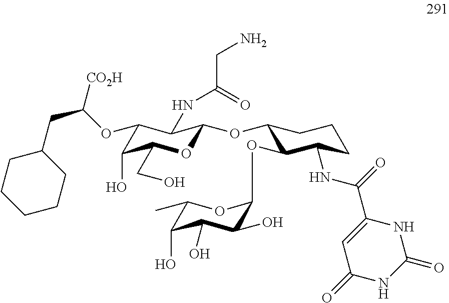

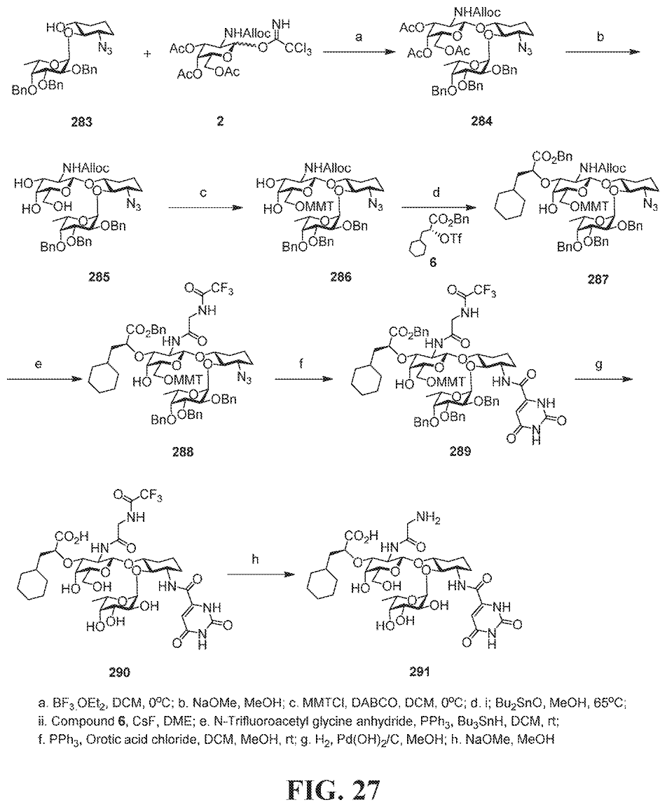

Compound 292: Compound 292 can be prepared in an analogous fashion to FIG. 27 by replacing orotic acid chloride with acetyl chloride in step f.

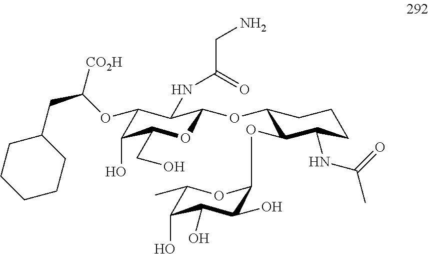

Compound 293: Compound 293 can be prepared in an analogous fashion to FIG. 27 by replacing orotic acid chloride with benzoyl chloride in step f.

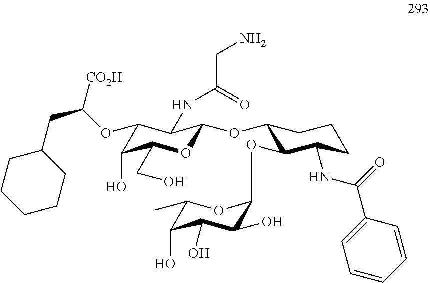

Compound 294: A solution of compound 291 (0.4 eq) in DMSO is added to a solution of compound 20 (1 eq) and DIPEA (10 eq) in anhydrous DMSO at room temperature. The resulting solution is stirred overnight. The reaction mixture is separated by reverse phase chromatography and the product lyophilized to give compound 294.

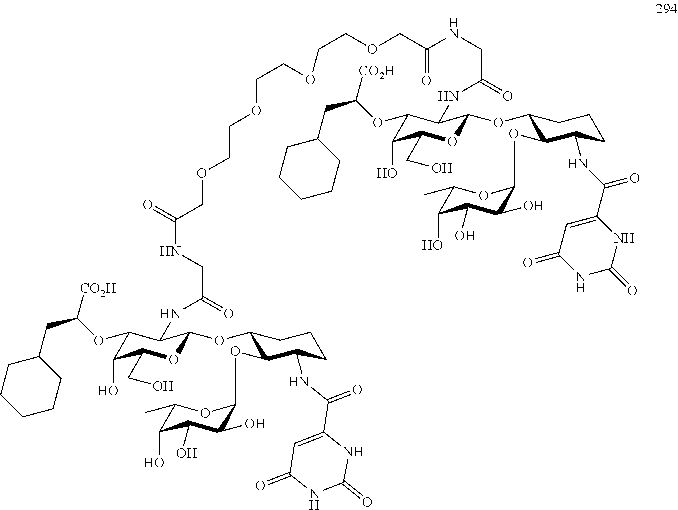

### Example 221

### Prophetic Synthesis of Multimeric Compound 295

Compound 295: Compound 294 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by reverse phase chromatography to afford compound 295.

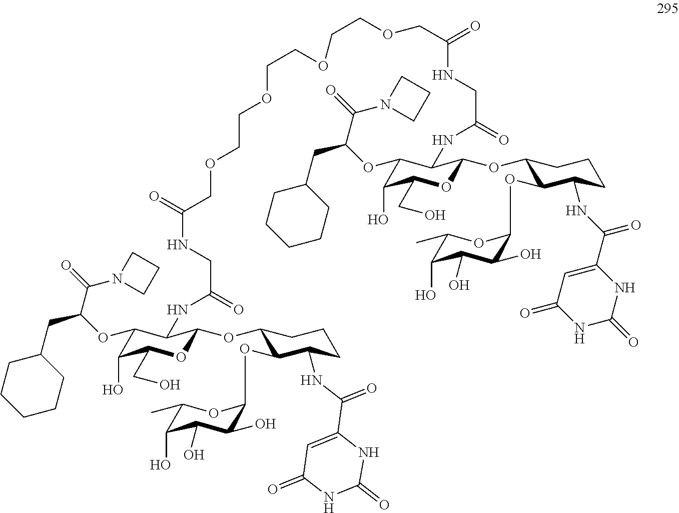

### Example 222

### Prophetic Synthesis of Multimeric Compound 2%

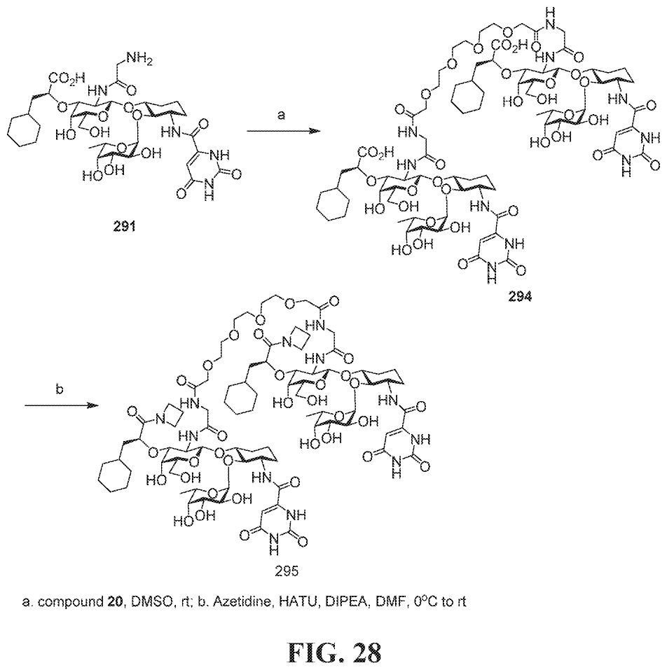

Compound 296: Compound 2% can be prepared in an analogous fashion to FIG. 28 by replacing compound 20 with ethylene glycol diacetic acid di-NHS ester in step a.

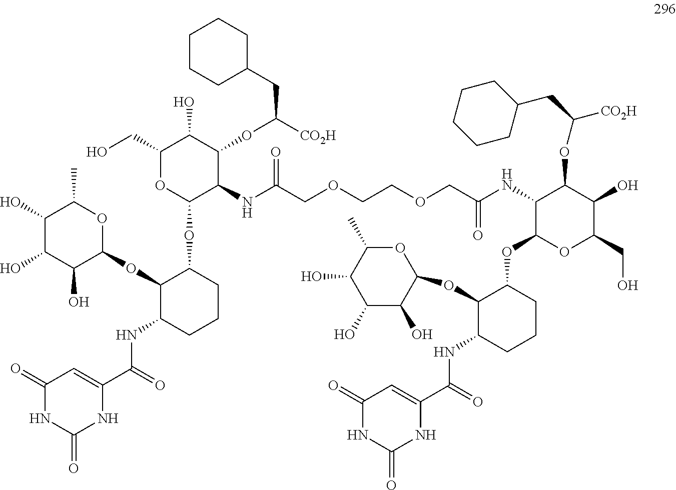 [SMILES: C[C@@H]1O[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3CCC[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H](O)[C@H](O)[C@@H]1O]

### Example 223

### Prophetic Synthesis of Multimeric Compound 297

Compound 297: Compound 297 can be prepared in an analogous fashion to FIG. 28 by replacing compound 20 with ethylene glycol diacetic acid di-NHS ester in step a.

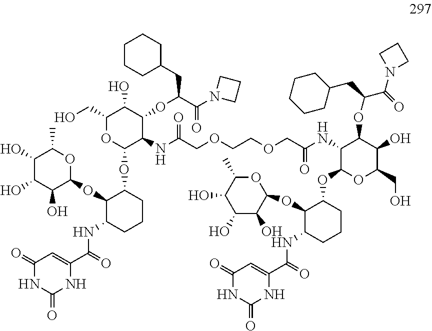 [SMILES: C[C@@H]1O[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)N3CCC3)[C@H]2NC(=O)COCCOCC(=O)N[C@H]2[C@H](O[C@@H]3CCC[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCC2)[C@@H](O)[C@H](O)[C@@H]1O]

### Example 224

### Prophetic Synthesis of Multimeric Compound 298

Compound 298: Compound 298 can be prepared in an analogous fashion to FIG. 28 by replacing compound 291 with compound 292 and compound 20 with ethylene glycol diacetic acid di-NHS ester in step a.

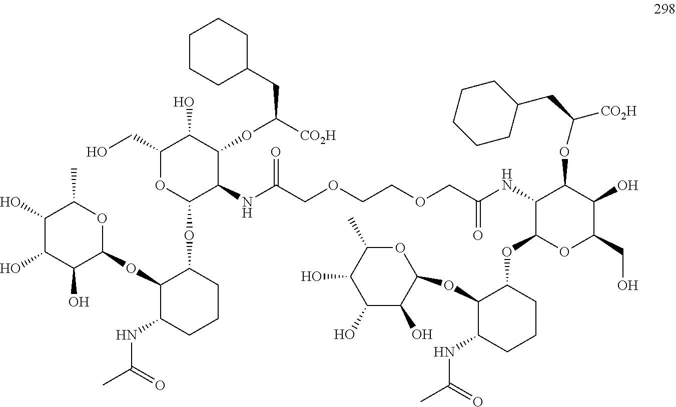 [SMILES: CC(=O)N[C@H]1CCC[C@@H](O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3CCC[C@H](NC(C)=O)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H]1O[C@@H]1O[C@@H](C)[C@@H](O)[C@@H](O)[C@@H]1O]

### Example 225

### Prophetic Synthesis of Multimeric Compound 299

Compound 299: Compound 299 can be prepared in an analogous fashion to FIG. 28 by replacing compound 291 with compound 292 and compound 20 with ethylene glycol diacetic acid di-NHS ester in step a.

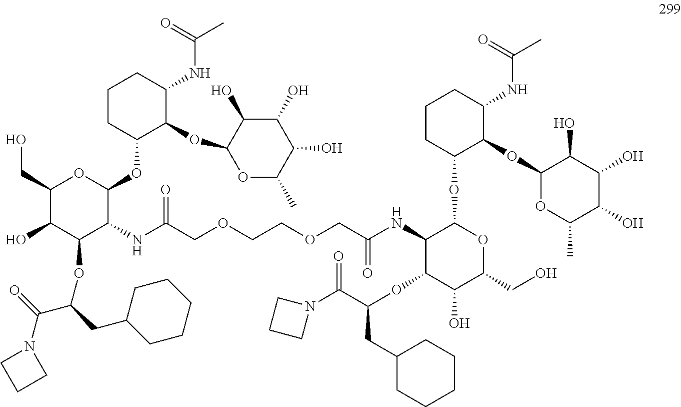 [SMILES: CC(=O)N[C@H]1CCC[C@@H](O[C@@H]2O[C@H](CO)[C@H](O)[C@H](O[C@@H](CC3CCCCC3)C(=O)N3CCC3)[C@H]2NC(=O)COCCOCC(=O)N[C@H]2[C@H](O[C@@H]3CCC[C@H](NC(C)=O)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCC2)[C@@H]1O[C@@H]1O[C@@H](C)[C@@H](O)[C@@H](O)[C@@H]1O]

### Example 226

### Prophetic Synthesis of Multimeric Compound 300

Compound 300: Compound 300 can be prepared in an analogous fashion to FIG. 28 by replacing compound 291 with compound 293 and compound 20 with ethylene glycol diacetic acid di-NHS ester in step a.

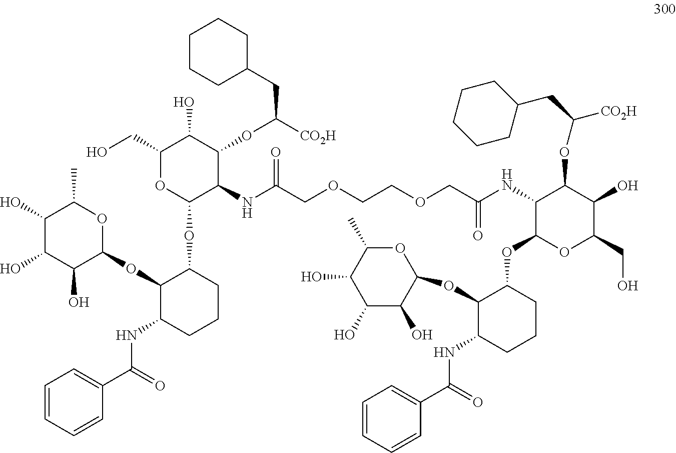 [SMILES: C[C@@H]1O[C@@H](O[C@@H]2[C@@H](NC(=O)c3ccccc3)CCC[C@H]2O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3CCC[C@H](NC(=O)c4ccccc4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H](O)[C@H](O)[C@@H]1O]

### Example 227

### Prophetic Synthesis of Multimeric Compound 301

Compound 301: Compound 301 can be prepared in an analogous fashion to FIG. 28 by replacing compound 291 with compound 293 and compound 20 with ethylene glycol diacetic acid di-NHS ester in step a.

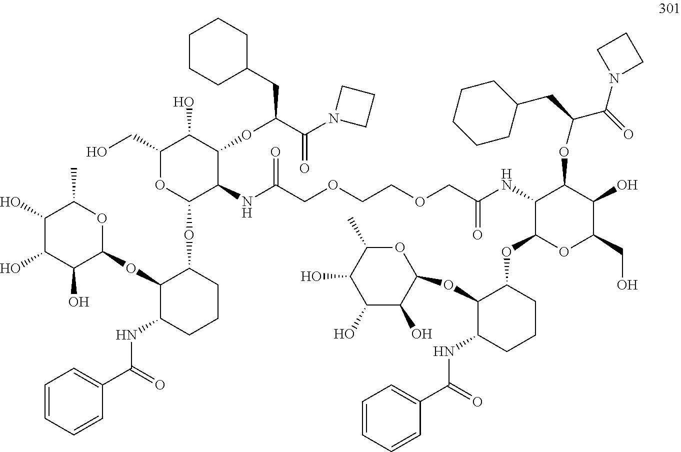 [SMILES: C[C@@H]1O[C@@H](O[C@@H]2[C@@H](NC(=O)c3ccccc3)CCC[C@H]2O[C@@H]2O[C@H](CO)[C@H](O)[C@H](O[C@@H](CC3CCCCC3)C(=O)N3CCC3)[C@H]2NC(=O)COCCOCC(=O)N[C@H]2[C@H](O[C@@H]3CCC[C@H](NC(=O)c4ccccc4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCC2)[C@@H](O)[C@H](O)[C@@H]1O]

### Example 228

### Prophetic Synthesis of Multimeric Compound 302

Compound 302: Compound 302 can be prepared in an analogous fashion to FIG. 28 by replacing compound 20 with 3,3′-[[2,2-bis[[3-[(2,5-dioxo-1-pyrrolidinyl)oxy]-3-oxopropoxy]methyl]-1,3-propanediyl]bis(oxy)]bis-, 1,1′-bis(2,5-dioxo-1-pyrrolidinyl)-propanoic acid ester in step a.

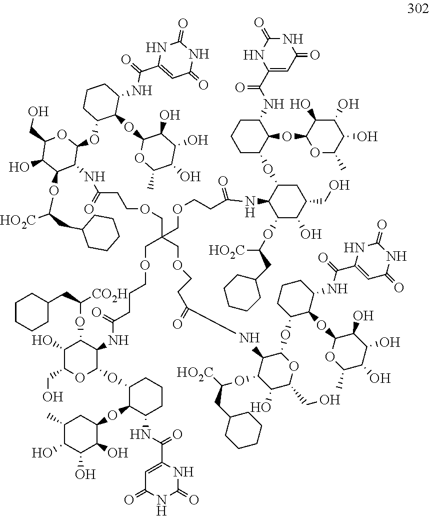

### Example 229

### Prophetic Synthesis of Multimeric Compound 305

Compound 303: To a stirred solution of compound 287 in DCM/MeOH (251) at room temperature is added orotic acid chloride (5 eq) and triphenylphosphine (5 eq). The reaction mixture is stirred 24 hours. The solvent is removed and the residue is separated by column chromatography to afford compound 303.

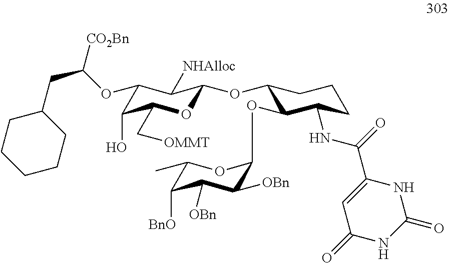

Compound 304: To a degassed solution of compound 303 in anhydrous DCM at 0° C. is added Pd(PPh₃)₄ (0.1 eq), Bu₃SnH (1.1 eq) and azidoacetic anhydride (2.0 eq). The ice bath is removed and the solution is stirred for 12 hrs under a N₂ atmosphere at room temperature. The reaction mixture is diluted with DCM, washed with water, dried over Na₂SO₄, then concentrated. The crude product is purified by column chromatography to give compound 304.

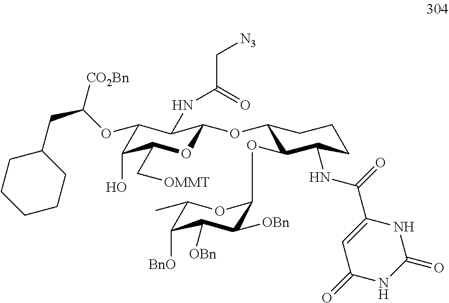

Compound 305: A solution of bispropagyl PEG-5 (compound 43) and compound 304 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at 50° C. The solution is cooled to room temperature and concentrated under reduced pressure. The crude product is purified by chromatography to give compound 305.

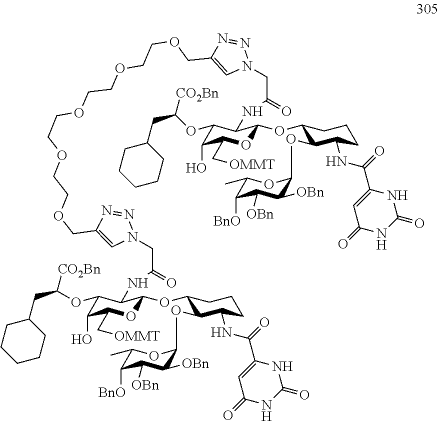

### Example 230

### Prophetic Synthesis of Multimeric Compound 306

Compound 306: Compound 305 is dissolved in MeOH and hydrogenated in the presence of Pd(OH)₂ (20 wt %) at 1 atm of H₂ gas pressure for 24 hrs at room temperature. The solution is filtered through a Celite pad. The filtrate is concentrated to give compound 306.

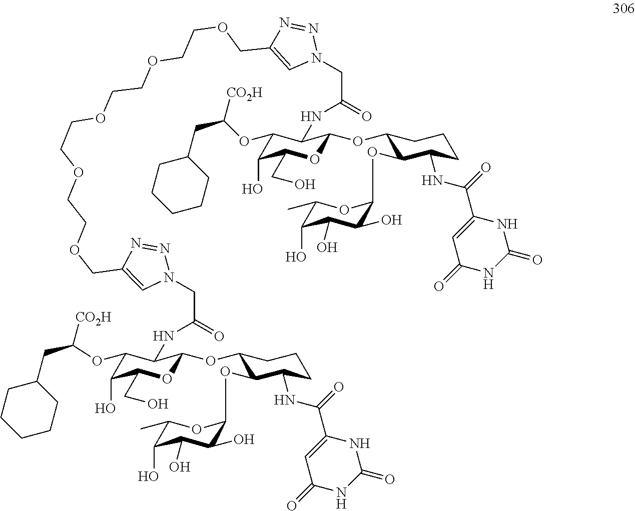

### Example 231

### Prophetic Synthesis of Multimeric Compound 307

Compound 307: Compound 306 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by reverse phase chromatography to afford compound 307.

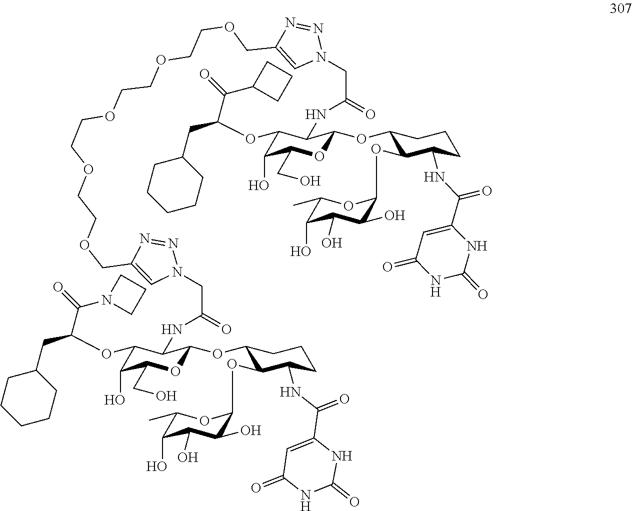

### Example 232

### Prophetic Synthesis of Multimeric Compound 308

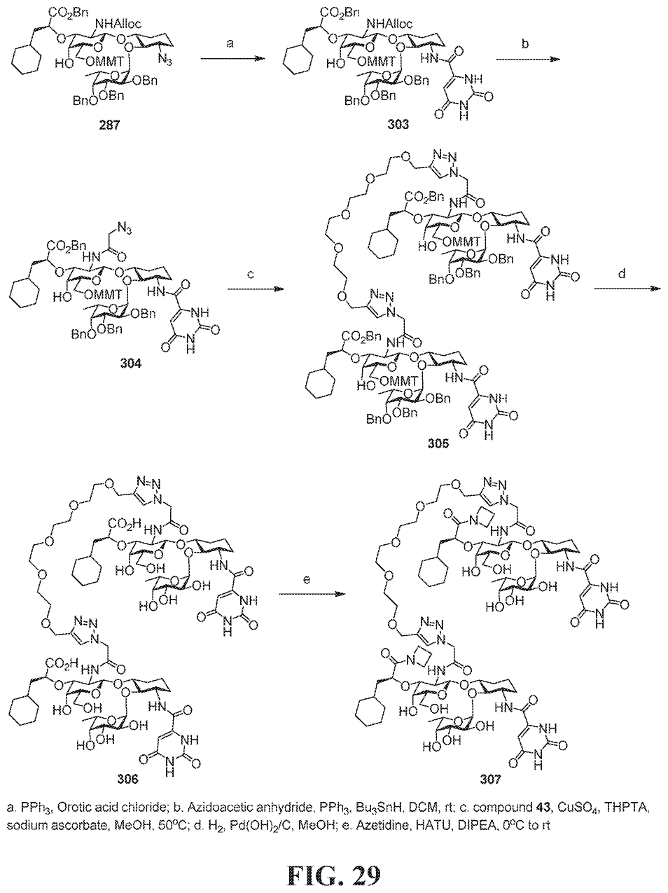

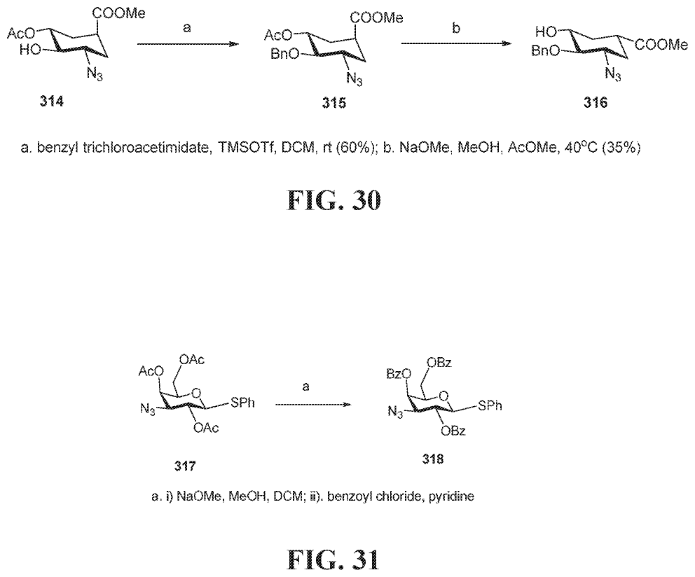

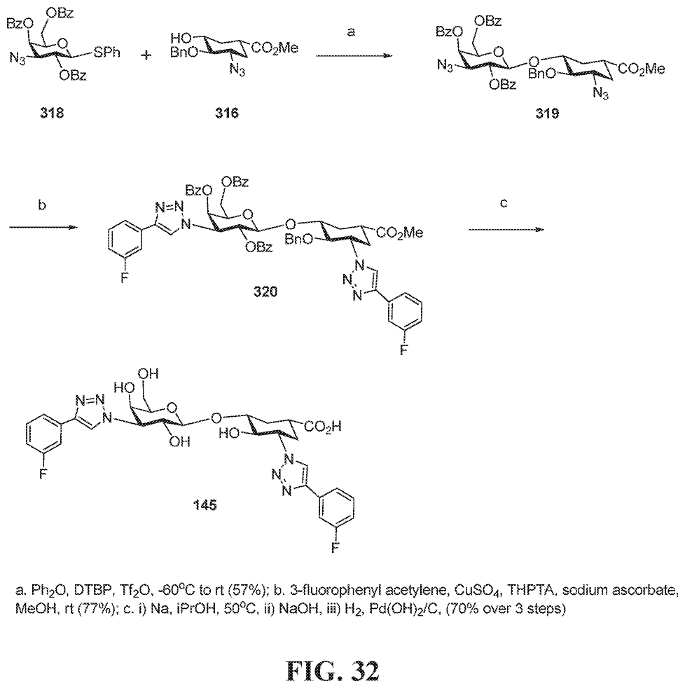

Compound 308: Compound 308 can be prepared in an analogous fashion to FIG. 29 using 3,3′-[[2,2-bis[(2-propyn-1-yloxy)methyl]-1,3-propanediyl]bis(oxy)]bis-1-propyne in place of compound 43 in step c.

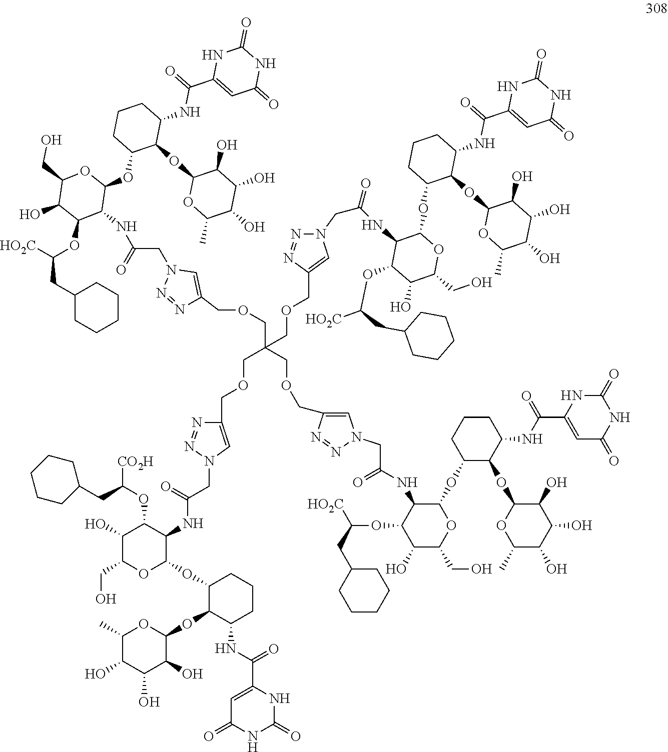

### Example 233

### Prophetic Synthesis of Multimeric Compound 309

Compound 309: Compound 309 can be prepared in an analogous fashion to FIG. 29 using 3,3′-[[2,2-bis[(2-propyn-1-yloxy)methyl]-1,3-propanediyl]bis(oxy)]bis-1-propyne in place of compound 43 in step c.

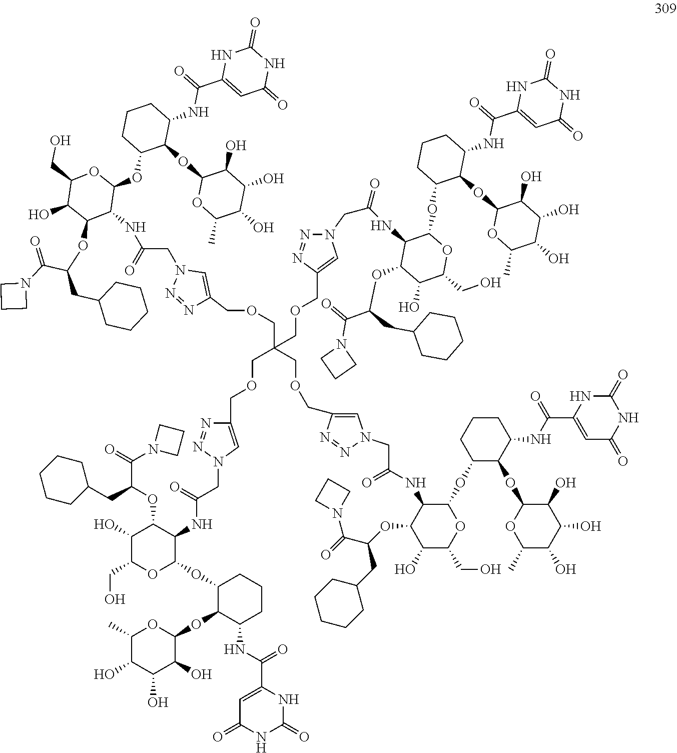

### Example 234

### PROPHETIC SYNTHESIS OF MULTIMERIC COMPOUND 310

Compound 310: Compound 310 can be prepared in an analogous fashion to FIG. 29 by replacing compound 43 with bis-propargyl ethylene glycol in step c.

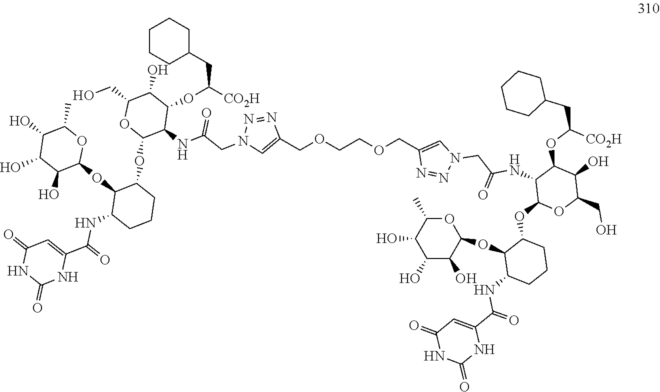 [SMILES: C[C@@H]1O[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)O)[C@H]2NC(=O)Cn2cc(COCCOCc3cn(CC(=O)N[C@H]4[C@H](O[C@@H]5CCC[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 235

### Prophetic Synthesis of Multimeric Compound 311

Compound 311: Compound 311 can be prepared in an analogous fashion to FIG. 29 by replacing compound 43 with bis-propargyl ethylene glycol in step c.

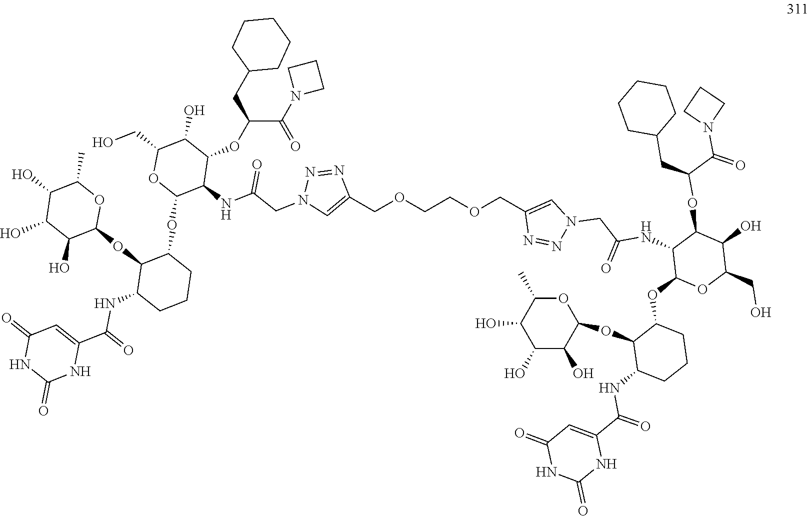 [SMILES: C[C@@H]1O[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)N3CCC3)[C@H]2NC(=O)Cn2cc(COCCOCc3cn(CC(=O)N[C@H]4[C@H](O[C@@H]5CCC[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)N4CCC4)nn3)nn2)[C@@H](O)[C@H](O)[C@@H]1O]

### Example 236

### Prophetic Synthesis of Multimeric Compound 312

Compound 312: Compound 312 can be prepared in an analogous fashion to FIG. 29 by replacing compound 43 with propargyl ether in step c.

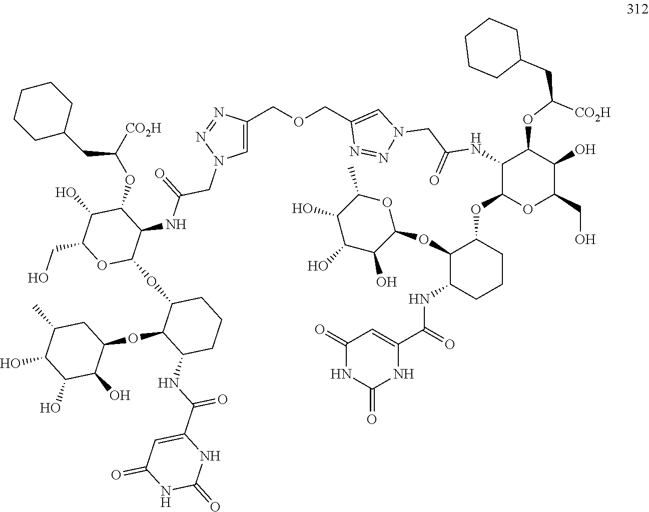 [SMILES: C[C@@H]1C[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)O)[C@H]2NC(=O)Cn2cc(COCc3cn(CC(=O)N[C@H]4[C@H](O[C@@H]5CCC[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 237

### Prophetic Synthesis of Multimeric Compound 313

Compound 313: Compound 313 can be prepared in an analogous fashion to FIG. 29 by replacing compound 43 with propargyl ether in step c.

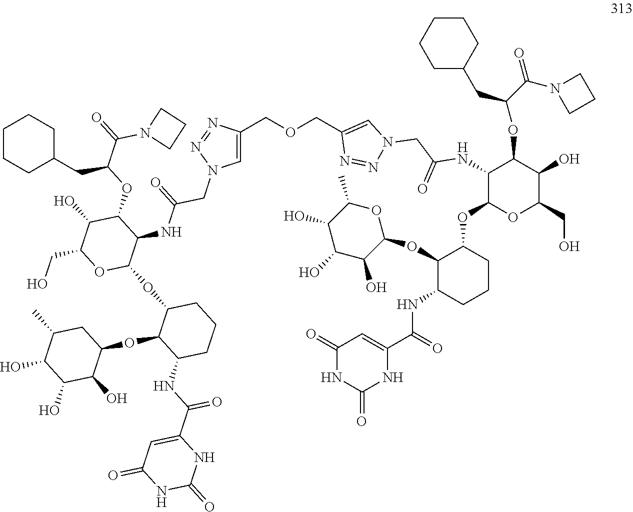 [SMILES: C[C@@H]1C[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)N3CCC3)[C@H]2NC(=O)Cn2cc(COCc3cn(CC(=O)N[C@H]4[C@H](O[C@@H]5CCC[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)N4CCC4)nn3)nn2)[C@@H](O)[C@H](O)[C@@H]1O]

### Example 238

### Synthesis of Building Block 332

Compound 321: Compound 317 (1.1 g, 2.60 mmoles) was dissolved in methanol (25 mL) at room temperature. Sodium methoxide (0.1 mL, 25% sol. in MeOH) was added and the reaction mixture was stirred at room temperature for 2 hours. The reaction mixture neutralized by the addition of Amberlyst acidic resin, filtered and concentrated to give crude 321, which was used for the next step without further purification. LCMS (ESI): m/z calculated for C₁₂H₁₅N₃O₄S: 297.3, found 298.1 (M+1); 320.1 (M+Na).

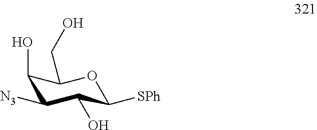

Compound 322: Crude compound 321 (2.60 mmoles), 3,4,5-trifluorophenyl-1-acetylene (2.5 equiv), THPTA (0.11 equiv), and copper (II) sulfate (0.1) were dissolved in methanol (15 mL) at room temperature. Sodium ascorbate (2.4 equiv) dissolved in water was added and the reaction mixture was stirred overnight at room temperature. The resultant precipitate was collected by filtration, washed with hexanes and water, and dried to give compound 322 as a pale yellow solid (1.2 g, 100% yield for 2 steps). LCMS (ESI): m/z calculated for C₂₀H₁₈F₃N₃O₄S: 453.1, found 454.2 (M+1); 476.2 (M+Na).

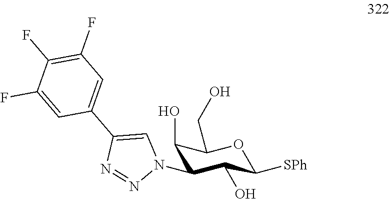

Compound 323: Compound 322 (1.2 g, 2.65 mmoles) was dissolved in DMF (15 mL) and cooled on an ice bath. Sodium hydride (60% oil dispersion, 477 mg, 11.93 mmoles) was added and the mixture stirred for 30 minutes. Benzyl bromide (1.42 mL, 11.93 mmoles) was added and the reaction was warmed to room temperature and stirred overnight. The reaction mixture was quenched by the addition of aqueous saturated ammonium chloride solution, transferred to a separatory funnel and extracted 3 times with ether. The combined organic phases were dried over magnesium sulfate, filtered, and concentrated. The residue was purified by flash chromatography to afford compound 323 (1.8 g, 94% yield). LCMS (ESI): m/z calculated for C₄₁H₃₆F₃N₃O₄S: 723.2, found 724.3 (M+1); 746.3 (M+Na).

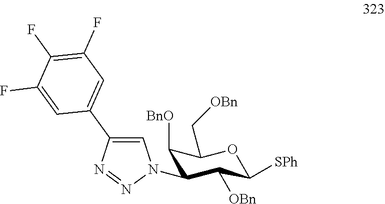

Compound 324: Compound 323 (1.8 g, 2.49 mmol) was dissolved in acetone (20 mL) and water (2 mL) and cooled on an ice bath. Trichloroisocyanuric acid (637 mg, 2.74 mmoles) was added and the reaction mixture stirred on the ice bath for 3 h. The acetone was removed in vacuo and the residue was diluted with DCM, transferred to a separatory funnel, and washed with saturated aqueous NaHCO₃. The organic phase was concentrated and the residue was purified by flash chromatography to afford compound 324 (1.5 g, 95%). LCMS (ESI): m/z calculated for C₃₅H₃₂F₃N₃O₅: 631.2, found 632.2 (M+1); 654.2 (M+Na).

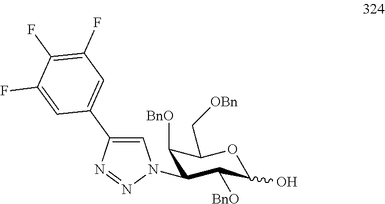

Compound 325: Compound 324 (1.0 g, 1.58 mmoles) was dissolved in DCM (20 mL) and cooled on an ice bath. Dess-Martin periodinane (1.0 g, 2.37 mmoles) was added and mixture was allowed to warm to room temperature and stirred overnight. The reaction mixture quenched by the addition of aqueous saturated NaHCO₃, transferred to a separatory funnel, and extracted 2 times with DCM. The combined organic phases were dried over sodium sulfate, filtered, and concentrated. The residue was purified by flash chromatography to afford compound 325 (520 mg, 52% yield). LCMS (ESI): m/z calculated for C₃₅H₃₀F₃N₃O₅: 629.2, found 652.2 (M+Na); 662.2 (M+MeOH+1); 684.2 (M+MeOH+Na).

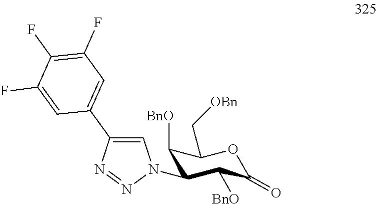

Compound 326: Methyl bromoacetate (253 mg, 1.65 mmoles) dissolved in 0.5 mL of THF was added dropwise to a solution of lithium bis(trimethylsilyl)amide (1.0 M in THF, 1.65 mL, 1.65 mmoles) cooled at −78° C. The reaction mixture was stirred for 30 minutes at −78° C. Compound 325 (260 mg, 0.41 mmoles) dissolved in THF (2.0 mL) was then added. The reaction mixture was stirred at −78° C. for 30 minutes. The reaction was quenched by the addition of aqueous saturated NH₄Cl and warmed to rt. The reaction mixture was transferred to a separatory funnel and extracted 3 times with ethyl acetate. The combined organic phases were dried over sodium sulfate, filtered and concentrated. The residue was separated by flash chromatography to afford compound 326 (183 mg, 64% yield).

¹H NMR (400 MHz, Chloroform-d) δ 7.38-7.22 (m, 9H), 7.15-7.11 (m, 3H), 7.09 (dd, J=8.4, 6.6 Hz, 1H), 7.06-7.00 (m, 2H), 6.98-6.93 (m, 2H), 5.11 (dd, J=11.3, 3.2 Hz, 1H), 4.60 (d, J=11.8 Hz, 1H), 4.57-4.49 (m, 2H), 4.49-4.42 (m, 2H), 4.35 (d, J=11.8 Hz, 1H), 4.14 (d, J=3.2 Hz, 1H), 4.05 (s, 1H), 4.02 (d, J=7.0 Hz, 1H), 3.84 (d, J=11.0 Hz, 1H), 3.81 (s, 3H), 3.70 (dd, J=9.5, 7.7 Hz, 1H), 3.62 (dd, J=9.4, 6.0 Hz, 1H). LCMS (ESI): m/z calculated for C₃₈H₃₄F₃N₃O₇: 701.2, found 702.3 (M+1); 724.3 (M+Na).

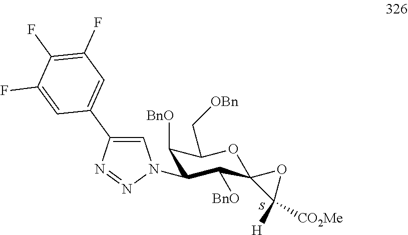

Compound 327: Compound 326 (5.0 g, 7.13 mmol) was azeotroped with toluene two times under reduced pressure, and then dried under high vacuum for 2 hours. It was then dissolved in anhydrous CH₂Cl₂ (125 mL) and cooled on an ice bath while stirring under an atmosphere of argon. Tributyltin hydride (15.1 mL, 56.1 mmol) was added dropwise and the solution was allowed to stir for 25 minutes on the ice bath. Trimethylsilyl triflate (2.1 mL, 11.6 mmol) dissolved in 20 mL of anhydrous CH₂C₁₂ was then added dropwise over the course of 5 minutes. The reaction was slowly warmed to ambient temperature and stirred for 16 hours. The reaction mixture was then diluted with CH₂Cl₂ (50 mL), transferred to a separatory funnel, and washed with saturated aqueous NaHCO₃ (50 mL). The aqueous phase was separated and extracted with CH₂C₁₂ (50 mL×2). The combined organic phases were washed with saturated aqueous NaHCO₃ (50 mL), dried over Na₂SO₄, filtered, and concentrated. The residue was purified by flash chromatography (hexanes to 40% EtOAc in hexanes, gradient) to afford compound 327 (2.65 g, 48%).

¹H-NMR (400 MHz, CDCl₃): δ 7.65 (s, 1H), 7.36-7.22 (m, 8H), 7.16-7.06 (m, 7H), 6.96-6.90 (m, 2H), 5.03 (dd, J=10.7, 3.2 Hz, 1H), 4.72 (d, J=2.3 Hz, 1H), 4.51 (dt, J=22.6, 11.4 Hz, 3H), 4.41 (d, J=10.9 Hz, 1H), 4.32 (dd, J=10.7, 9.2 Hz, 1H), 4.07 (d, J=3.1 Hz, 1H), 3.94 (d, J=10.9 Hz, 11H), 3.92-3.84 (m, 3H), 3.78-3.71 (m, 4H), 3.65 (dd, J=9.1, 5.5 Hz, 1H), 0.24 (s, 9H). LCMS (ESI): m/z (M+Na) calculated for C₄₁H₄₄F₃N₃O₇SiNa: 798.87, found 798.2.

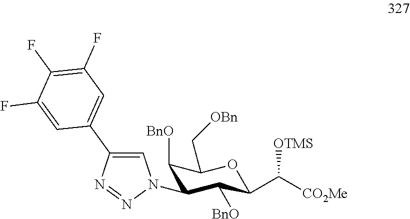

Compound 328: To a solution of compound 327 (2.65 g, 3.4 mmol) in anhydrous MeOH (40 mL) was added Pd(OH)₂ (0.27 g, 20% by wt). The mixture was cooled on an ice bath and stirred for 30 minutes. Triethylsilane (22 mL, 137 mmol) was added dropwise. The solution was allowed to slowly warm to ambient temperature and stirred for 16 hours. The reaction mixture was filtered through a bed of Celite and concentrated. The residue was purified by flash chromatography (hexanes to 100% EtOAc, gradient) to afford compound 328 (1.09 g, 73%).

¹H-NMR (400 MHz, CD₃OD): δ 8.57 (s, 1H), 7.77-7.53 (m, 2H), 4.91-4.82 (m, 1H), 4.66-4.59 (m, 1H), 4.55 (dd, J=10.8, 9.4 Hz, 1H), 4.13 (d, J=2.8 Hz, 1H), 3.86 (dd, J=9.4, 2.1 Hz, 1H), 3.81 (s, 3H), 3.77-3.74 (m, 1H), 3.71-3.68 (m, 2H). LCMS (ESI): m/z (M+Na) calculated for C₁₇H₁₈F₃N₃O₇Na: 456.33, found 456.0.

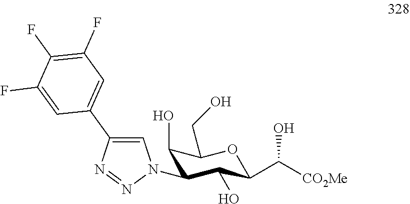

Compound 329: Compound 328 (1.09 g, 2.5 mmol) and CSA (0.115 g, 0.49 mmol) were suspended in anhydrous MeCN (80 mL) under an argon atmosphere. Benzaldehyde dimethyl acetal (0.45 mL, 2.99 mmol) was added dropwise. The reaction mixture was allowed to stir for 16 hours at ambient temperature, during which time it became a homogenous solution. The reaction mixture was then neutralized with a few drops of Et₃N, and concentrated. The residue was purified via flash chromatography (CH₂Cl₂ to 10% MeOH in CH₂Cl₂, gradient) to afford compound 329 (978 mg, 75%).

¹H NMR (400 MHz, DMSO-d₆): δ 8.84 (s, 1H), 7.95-7.73 (m, 2H), 7.33 (qdt, J=8.4, 5.6, 2.7 Hz, 5H), 5.51 (t, J=3.8 Hz, 2H), 5.47 (d, J=6.8 Hz, 1H), 5.14 (dd, J=10.8, 3.6 Hz, 1H), 4.54 (dd, J=6.7, 2.2 Hz, 1H), 4.47 (ddd, J=10.8, 9.3, 7.5 Hz, 1H), 4.40 (d, J=4.0 Hz, 1H), 4.09-3.99 (m, 2H), 3.85 (dd, J=9.3, 2.2 Hz, 1H), 3.81-3.76 (m, 1H), 3.71 (s, 3H). LCMS (ESI): m/z (M+Na) calculated for C₂₄H₂₂F₃N₃O₇Na: 544.43, found 544.1.

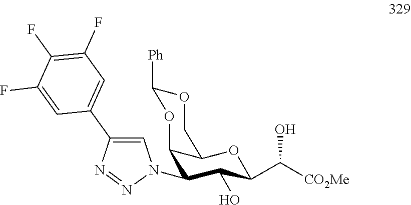

Compound 330: Compound 329 (25.2 mg, 0.048 mmol) was azeotroped with toluene 2 times under reduced pressure, dried under high vacuum for 2 hours, then dissolved in anhydrous DMF (2 mL) and cooled on an ice bath. Benzyl bromide (6 uL, 0.05 mmol) dissolved in 0.5 mL of anhydrous DMF was added and the reaction and was stirred under an atmosphere of argon for 30 minutes at 0° C. Sodium hydride (2 mg, 0.05 mmol, 60%) was added and the reaction was allowed to gradually warm to ambient temperature while stirring for 16 hours. The reaction mixture was diluted with EtOAc (20 mL), transferred to a separatory funnel, and washed with H₂O (10 mL). The aqueous phase was separated and extracted with EtOAc (10 mL×3). The combined organic phases were washed with H₂O (10 mL×3), dried over Na₂SO₄, filtered, and concentrated. The residue was purified via preparative TLC (5% MeOH in CH₂C₁₂) to afford compound 330 (6.3 mg, 21%). LCMS (EST): m/z (M+Na) calculated for C₃₁H₂₈F₃N₃O₇Na: 634.55, found 634.1.

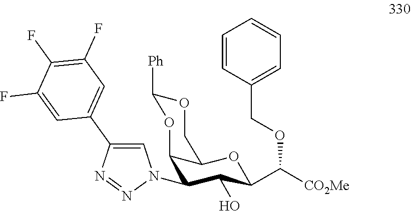

Compound 331: Compound 330 (6.3 mg, 0.01 mmol) was dissolved in anhydrous MeOH (1 mL) containing CSA (0.26 mg, 0.001 mmol). The reaction mixture was heated to 76° C. in a screw-cap scintillation vial while stirring. After 2 hours, an additional 0.13 mg of CSA in 0.5 mL of MeOH was added. The reaction mixture was stirred at 76° C. for 16 hours. The reaction mixture concentrated under reduced pressure. The residue was purified via preparative TLC (10% MeOH in CH₂Cl₂) to afford compound 331 (4.2 mg, 80%).

¹H NMR (400 MHz, DMSO-d₆) δ 8.80 (s, 1H), 7.94-7.86 (m, 2H), 7.48-7.42 (m, 2H), 7.38 (t, J=7.4 Hz, 2H), 7.36-7.28 (m, 1H), 5.46 (d, J=7.7 Hz, 1H), 5.28 (d, J=6.0 Hz, 1H), 4.85 (dd, J=10.7, 2.9 Hz, 1H), 4.67 (d, J=11.0 Hz, 1H), 4.62-4.58 (m, 1H), 4.54 (d, J=11.1 Hz, 1H), 4.44 (d, J=2.5 Hz, 1H), 4.36 (q, J=9.5 Hz, 1H), 3.95-3.90 (m, 1H), 3.78 (dd, J=9.3, 2.5 Hz, 1H), 3.71 (s, 3H), 3.61-3.54 (m, 1H), 3.52-3.43 (m, 1H), 3.43-3.38 (m, 1H). LCMS (ESI): m/z (M+Na) calculated for C₂₄H₂₄F₃N₃O₇Na: 546.45, found 546.0.

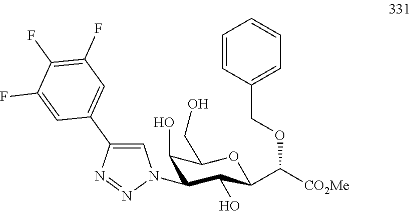

Compound 332: To a solution of compound 331 (3.5 mg, 0.007 mmoles) in methanol (0.5 mL) was added 1.0 M NaOH solution (0.1 mL). The reaction mixture was stirred overnight at room temperature then neutralized with acidic resin, filtered and concentrated. The residue was purified by reverse phase chromatography using a C-8 matrix to afford 3.0 mg compound 332 (90%).

¹H NMR (400 MHz, Deuterium Oxide) δ 8.39 (s, 1H), 8.37 (s, 2H), 7.54-7.45 (m, 1H), 7.43 (d, J=7.4 Hz, 2H), 7.35 (dt, J=14.3, 7.2 Hz, 3H), 4.86 (dd, J=11.0, 2.9 Hz, 1H), 4.76 (d, J=11.0 Hz, 1H), 4.40-4.30 (m, 2H), 4.16 (d, J=1.9 Hz, 1H), 4.04 (d, J=3.0 Hz, 1H), 3.81 (d, J=9.6 Hz, 11H), 3.73 (d, J=3.9 Hz, 0H), 3.67 (d, J=7.6 Hz, 1H), 3.56 (dd, J=11.7, 3.9 Hz, 1H). LCMS (ESI): m/z (M+Na) calculated for C₂₃H₂₂F₃N₃O₇: 509.1, found 508.2 (M−H).

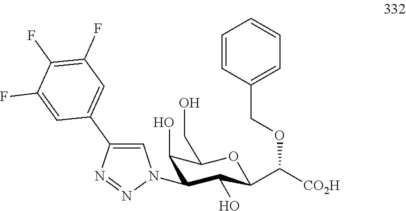

### Example 239

### Prophetic Synthesis of Building Block 333

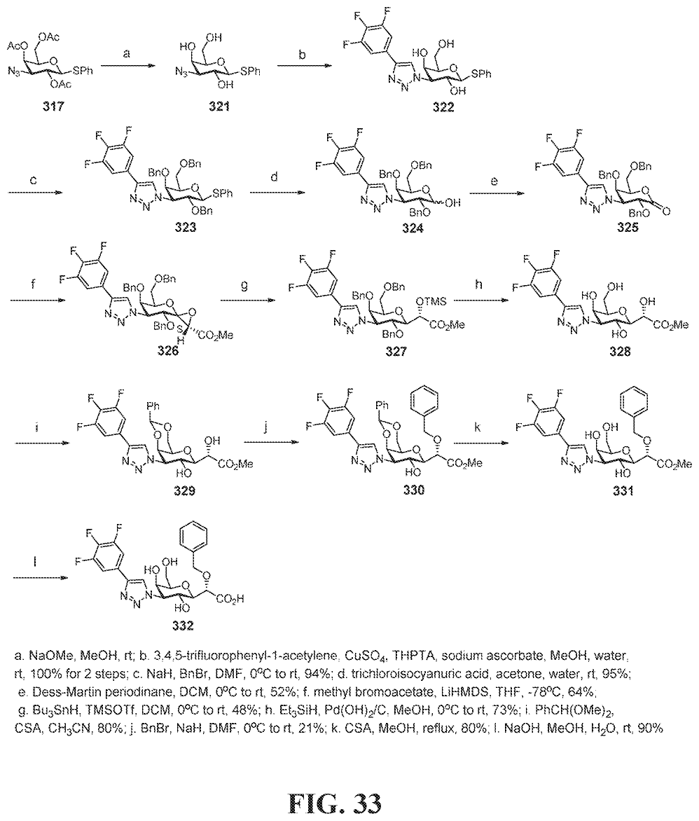

Compound 333: Compound 333 can be prepared in an analogous fashion to FIG. 33 by replacing benzyl bromide with 4-chlorobenzyl bromide in step j.

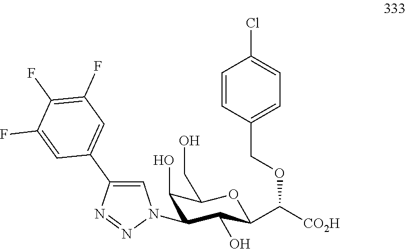

### Example 240

### Prophetic Synthesis of Building Block 334

Compound 334: Compound 334 can be prepared in an analogous fashion to FIG. 33 by replacing benzyl bromide with 4-methanesulfonylbenzyl bromide in step j.

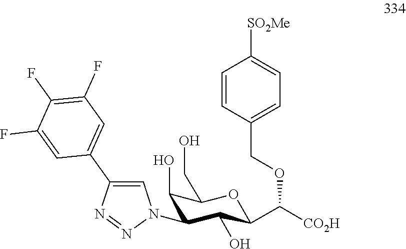

### Example 241

### Prophetic Synthesis of Building Block 335

Compound 335: Compound 335 can be prepared in an analogous fashion to FIG. 33 by replacing benzyl bromide with 3-picolyl bromide in step j.

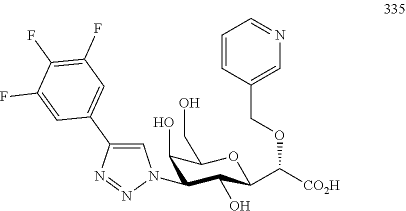

### Example 242

### Prophetic Synthesis of Multimeric Compound 336

Compound 336: Compound 336 can be prepared in an analogous fashion to FIG. 14 by replacing compound 145 with compound 332.

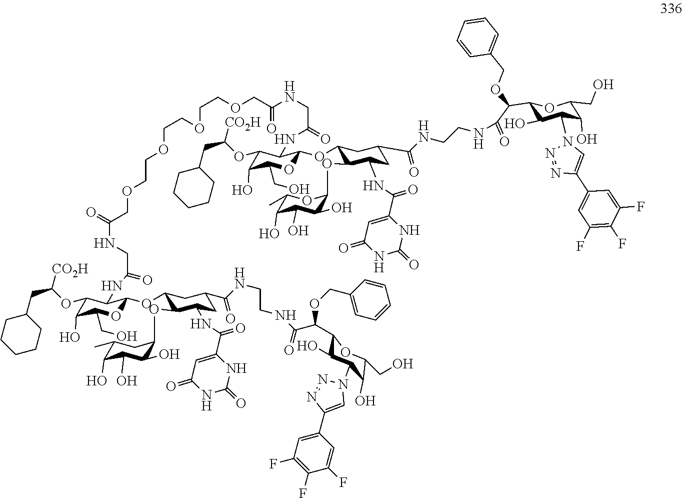

### Example 243

### Prophetic Synthesis of Multimeric Compound 337

Compound 337: Compound 337 can be prepared in an analogous fashion to FIG. 14 by replacing compound 145 with compound 333.

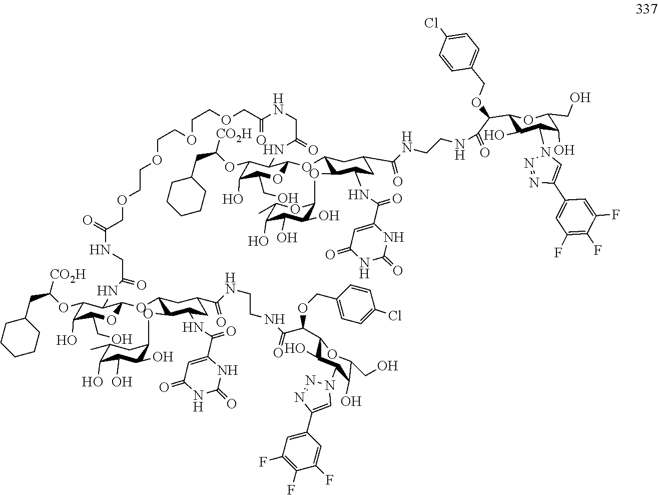

### Example 244

### Prophetic Synthesis of Multimeric Compound 338

Compound 338: Compound 338 can be prepared in an analogous fashion to FIG. 14 by replacing compound 145 with compound 334.

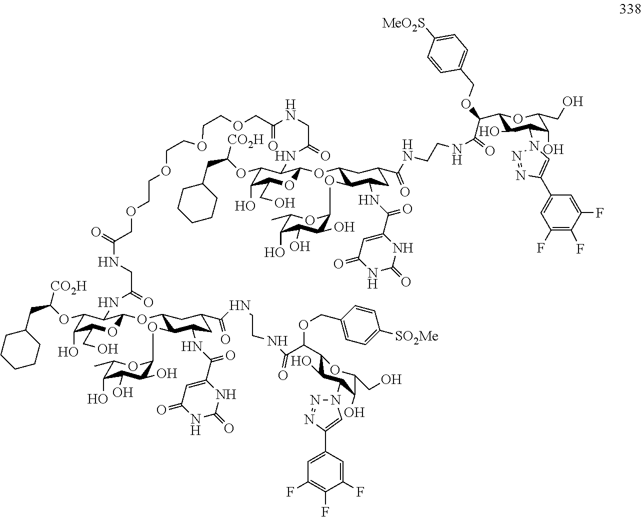

### Example 245

### Prophetic Synthesis of Multimeric Compound 339

Compound 339: Compound 339 can be prepared in an analogous fashion to FIG. 14 by replacing compound 145 with compound 335.

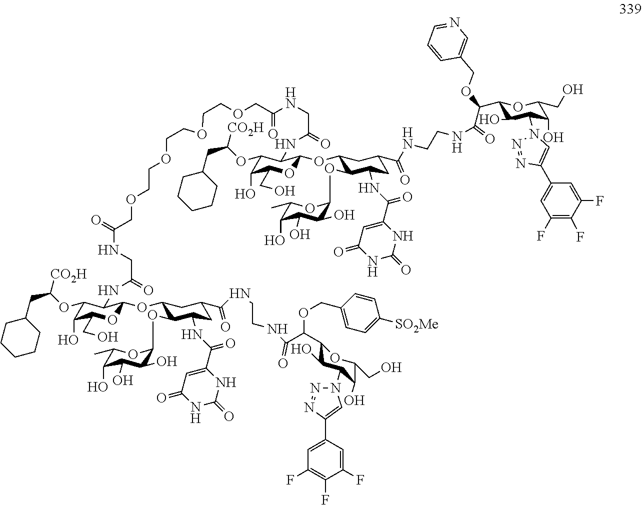

### Example 246

### Prophetic Synthesis of Multimeric Compound 340

Compound 340: Compound 340 can be prepared in an analogous fashion to FIG. 14 by replacing compound 22 with compound 40 and replacing compound 145 with compound 333.

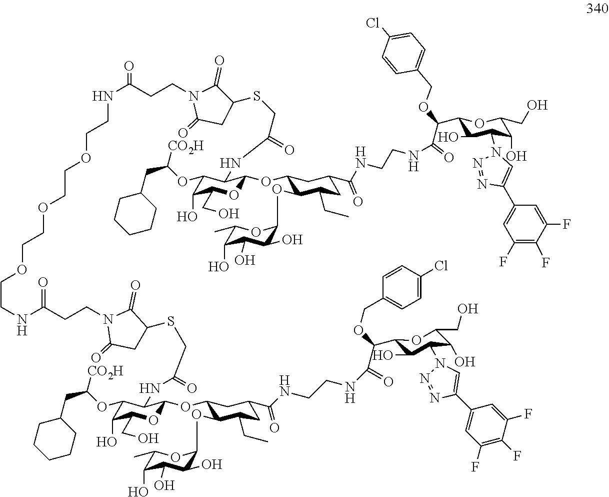

### Example 247

### Prophetic Synthesis of Multimeric Compound 341

Compound 341: Compound 341 can be prepared in an analogous fashion to FIG. 14 by replacing compound 22 with compound 78 and replacing compound 145 with compound 333.

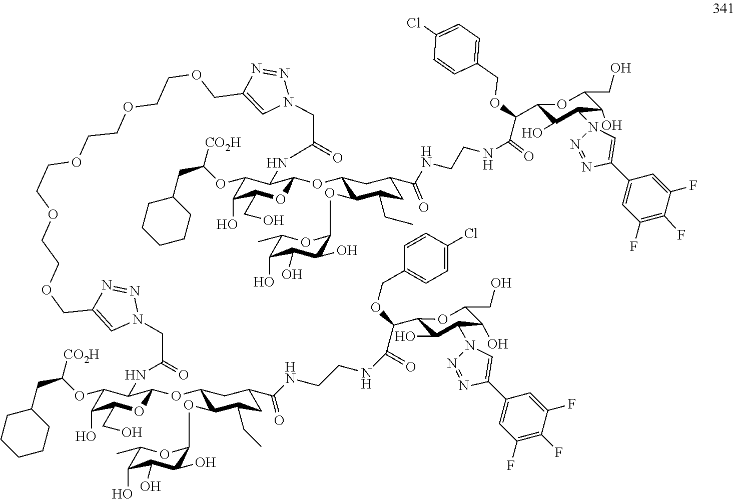

### Example 248

### Prophetic Synthesis of Multimeric Compound 342

Compound 342: Compound 342 can be prepared in an analogous fashion to FIG. 14 by replacing compound 22 with compound 87 and replacing compound 145 with compound 333.

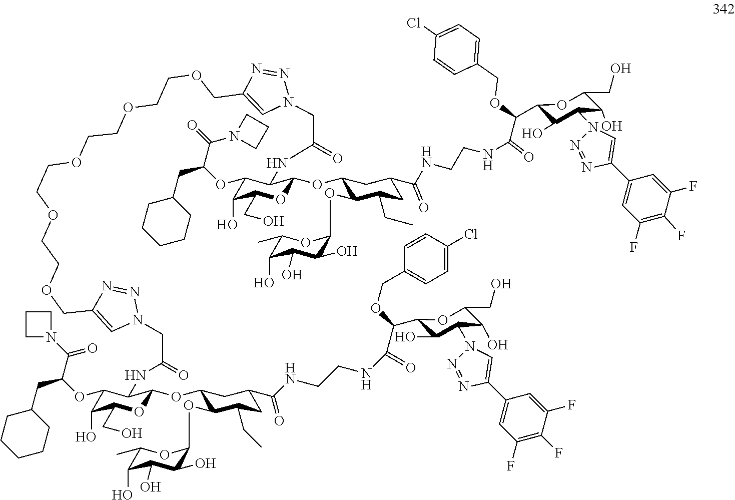

### Example 249

### Prophetic Synthesis of Multimeric Compound 343

Compound 343: Compound 342 can be prepared in an analogous fashion to FIG. 14 by replacing compound 22 with compound 88 and replacing compound 145 with compound 333.

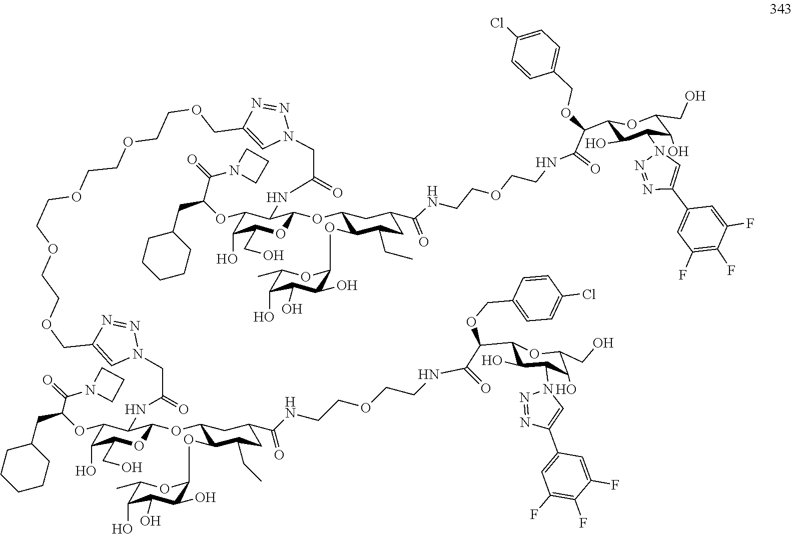

### Example 250

### E-Selectin Activity—Binding Assay

The inhibition assay to screen and characterize antagonists of E-selectin is a competitive binding assay, from which IC₅₀ values may be determined. E-selectin/Ig chimera are immobilized in 96 well microtiter plates by incubation at 37° C. for 2 hours. To reduce nonspecific binding, bovine serum albumin is added to each well and incubated at room temperature for 2 hours. The plate is washed and serial dilutions of the test compounds are added to the wells in the presence of conjugates of biotinylated, sLea polyacrylamide with streptavidin/horseradish peroxidase and incubated for 2 hours at room temperature.

To determine the amount of sLe^{a} bound to immobilized E-selectin after washing, the peroxidase substrate, 3,3′,5,5′ tetramiethylbenzidine (TMB) is added. After 3 minutes, the enzyme reaction is stopped by the addition of H₃PO₄, and the absorbance of light at a wavelength of 450 nm is determined. The concentration of test compound required to inhibit binding by 50% is determined.

### E-Selectin Antagonist Activity

| Compound | IC50 (nM) |
|---|---|
| Compound 206 | 1.6 |

### Example 251

### Galectin-3 Activity—ELISA Assay

Galectin-3 antagonists can be evaluated for their ability to inhibit binding of galectin-3 to a Galβ1-3GlcNAc carbohydrate structure. The detailed protocol is as follows. A 1 ug/mL suspension of a Galβ1-3GlcNAcβ1-3Galβ1-4GlcNAcβ-PAA-biotin polymer (Glycotech, catalog number 01-096) is prepared. A 100 uL aliquot of the polymer is added to the wells of a 96-well streptavidin-coated plate (R&D Systems, catalog number CP004). A 100 uL aliquot of 1× Tris Buffered Saline (TBS, Sigma, catalog number T5912-10X) is added to control wells. The polymer is allowed to bind to the streptavidin-coated wells for 1.5 hours at room temperature. The contents of the wells are discarded and 200 uL of 1×TBS containing 1% bovine serum albumin (BSA) is added to each well as a blocking reagent and the plate is kept at room temperature for 30 minutes. The wells are washed three times with 1×TBS containing 0.1% BSA. A serial dilution of test compounds is prepared in a separate V-bottom plate (Corning, catalog number 3897). A 75 uL aliquot of the highest concentration of the compound to be tested is added to the first well in a column of the V-bottom plate then 15 ul is serially transferred into 60 uL 1×TBS through the remaining wells in the column to generate a 1 to 5 serial dilution. A 60 uL aliquot of 2 ug/mL galectin-3 (IBL, catalog number IBATGP0414) is added to each well in the V-bottom plate. A 100 uL aliquot of the galectin-3/test compound mixture is transferred from the V-bottom plate into the assay plate containing the Galβ1-3GlcNAc polymer. Four sets of control wells in the assay plate are prepared in duplicate containing 1) both Galβ1-3GlcNAc polymer and galectin-3, 2) neither the polymer nor galectin-3, 3) galectin-3 only, no polymer, or 4) polymer only, no galectin-3. The plate is gently rocked for 1.5 hours at room temperature. The wells are washed four times with TBS/0.1% BSA. A 100 uL aliquot of anti-galectin-3 antibody conjugated to horse radish peroxidase (R&D Systems, from DGAL30 kit) is added to each well and the plate is kept at room temperature for 1 hour. The wells are washed four times with TBS/0.1% BSA. A 100 uL aliquot of TMB substrate solution is added to each well. The TMB substrate solution is prepared by making a 1:1 mixture of TMB Peroxidase Substrate (KPL, catalog number 5120-0048) and Peroxidase Substrate Solution B (KPL, catalog number 5120-0037). The plate is kept at room temperature for 10 to 20 minutes. The color development is stopped by adding 100 uL 10% phosphoric acid (RICCA Chemical Co., catalog number 5850-16). The absorbance at 450 nm (A₄₅₀) is measured using a FlexStation 3 plate reader (Molecular Devices). Plots of A₄₅₀ versus test compound concentration and IC₅₀ determinations are made using GraphPad Prism 6.

### Example 252

### CXCR4 Assay—Inhibition of Cyclic Amp

The CXCR4-cAMP assay measures the ability of a glycomimetic CXCR4 antagonist to inhibit the binding of CXCL12 (SDF-1α) to CHO cells that have been genetically engineered to express CXCR4 on the cell surface. Assay kits may be purchased from DiscoveRx (95-0081E2CP2M; cAMP Hunter eXpress CXCR4 CHO-K1). The Gᵢ-coupled receptor antagonist response protocol described in the kit instruction manual can be followed. GPCRs, such as CXCR4, are typically coupled to one of the 3 G-proteins: Gs, Gi, or Gq. In the CHO cells supplied with the kit, CXCR4 is coupled to Gi. After activation of CXCR4 by ligand binding (CXCL12), Gi dissociates from the CXCR4 complex, becomes activated, and binds to adenylyl cyclase, thus inactivating it, resulting in decreased levels of intracellular cAMP. Intracellular cAMP is usually low, so the decrease of the low level of cAMP by a Gi-coupled receptor will be difficult to detect. Forskolin is added to the CHO cells to directly activate adenylyl cyclase (bypassing all GPCRs), thus raising the level of cAMP in the cell, so that a Gi response can be more easily observed. CXCL12 interaction with CXCR4 decreases the intracellular level of cAMP and inhibition of CXCL12 interaction with CXCR4 by a CXCR4 antagonist increases the intracellular cAMP level, which is measured by luminescence.

### Example 253

Compound A, a specific antagonist of E-selectin, enhanced HSC quiescence by preventing differentiation. Studies further showed that therapeutic blockade of E-selectin in vivo with Compound A specifically augmented the mobilization of HSC with highest self-renewal potential following G-CSF administration, and markedly improved subsequent engraftment and reconstitution in mice. Noteworthy is the fact that the studies focused on the role of E-selectin and the use of Compound A during HSC mobilization in current harvesting procedures of donors to accelerate recovery in transplant recipients.

Antagonism of E-selectin in the recipient could lead to a beneficial effect on survival of HSC-reconstituted recipients. For example, hepatic veno-occlusive disease (VOD), also known as sinusoidal obstruction syndrome (SOS), is a major complication of HSC transplantation and it carries a high mortality. In a murine model of VOD, hepatic inflammation was characteristic of SOS, and mice deficient in P- and E-selectins on the surface of vascular endothelial cells showed markedly reduced SOS, demonstrating a major role for leukocytes recruited from blood. Inhibition of SOS with an E-selectin antagonist such as Compound A could have a positive impact on survival.

### Example 254

### Enhancing the Survival of Reconstituted, Bone Marrow Depleted Hosts

The survival outcome of lethally-irradiated, bone marrow depleted mice when reconstituted with HSC in combination with Compound A was investigated. C57BL/6 mice with bone marrow depleted by lethal-irradiation were reconstituted with bone-marrow derived from the congenic strain, B6.SJL-PtprcaPepcb/BoyJ (B6.SJL) mice. The use of a congenic strain in these studies allowed for the enumeration and differentiation between the donor strain (CD45.1+) and the recipient strain (CD45.2+).

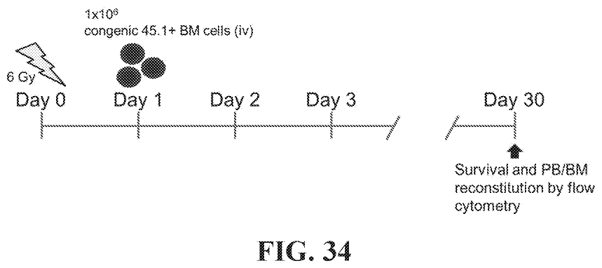

Twenty-four hours post irradiation (6Gy×2), cohorts of C57BL/6 mice (n=10/group) were injected i.v. with 1×10⁶ cells (study day 0) from B6.SJL donor mice with three IP dosing regimens with 40 mg/kg Compound A. These regimens were: (a) q12 h on study days 0 and 1; (b) q12h on study days 1 and 2; and (c) q12h on study day 1 only. Control groups in this study included irradiated mice alone (expected survival=0%), non-irradiated mice alone (expected survival=100%), and irradiated, reconstituted mice (no Compound A). The survival of mice was determined over the course of the study (study days 0 to 30) (see FIG. 34). Table 1, below, shows the study protocol to assess the effects of compound A on hematopoietic reconstitution of lethally irradiated C57BL/6 mice.

TABLE 1

| | | | | Compound A | |
|---|---|---|---|---|---|
| | | | | (40 mg/kg/ | Parameters |
| Group | N | Radiation | Transplant | IP lnjection) | Assessed |
| 1 | 10 | + | − | − | Survival, weight, |
| 2 | 10 | + | + | − | and reconsttuton |
| 3 | 10 | + | + | Day 0 and 1; q 12 h | of PB and BM |
| 4 | 10 | + | + | Day 1 and 2; q 12 h | |
| 5 | 10 | + | + | Day 1; q 12 h | |
| 6 | 3 | − | − | | |

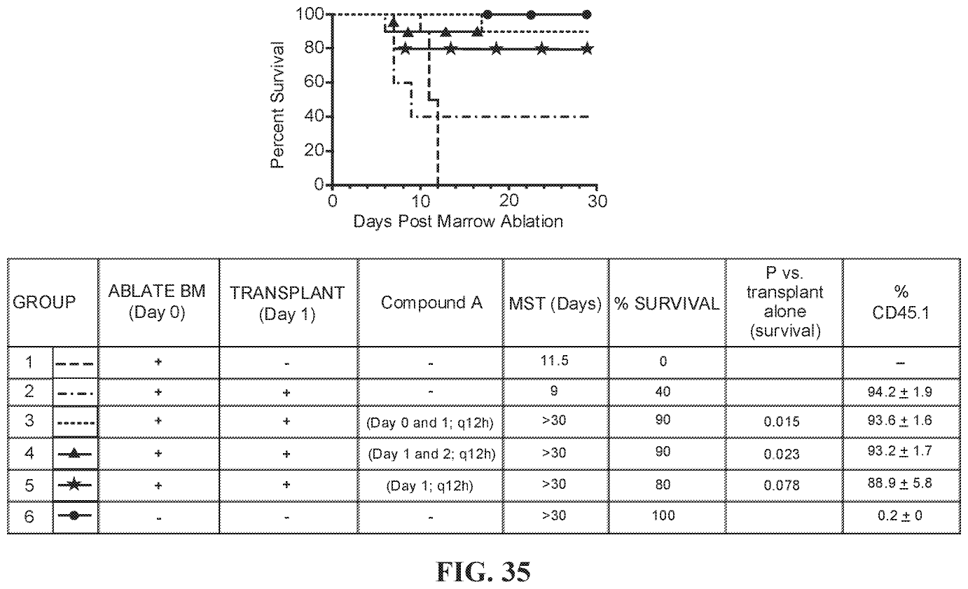

Treatment with Compound A as part of the transplant regimen significantly increased the median survival time (MST) of mice compared with the control group—the MST of mice treated with Compound A and HSC was >30 days with 80-90/o of mice alive at study completion. In contrast, the MST of irradiated mice (no transplant) was 11.5 days with no survivors at study completion. The MST of mice irradiated and transplanted with congenic HSC was 9 days with 40% survival at study completion. The impact of Compound A on survival represented a >233.3% increase in life span (See FIG. 35).

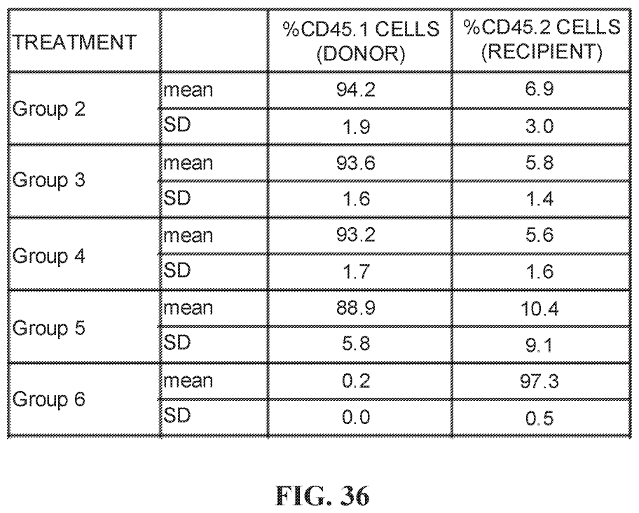

Flow cytometric analysis in all surviving mice on day 30 using PE-CD45.1 and APC-CD45.2 markers showed that the mean percentage of CD45.1+ cells from donor congenic mice was approximately 90% (blood and bone marrow), indicating that all surviving mice were successfully reconstituted (See FIG. 36). These data suggest that administration of Compound A did not inhibit engraftment or expansion of donor HSCs in the recipient mice. Although not specifically evaluated, incorporation of Compound A into the reconstitution regimen may be speculated to have attenuated a sinusoidal obstructive syndrome such as hepatic veno-occlusive disease known to be E-selectin dependent and a major complication of HSC transplantation.

Accordingly, this novel therapeutic use of inhibitors of E-selectin, such as Compound A, results in the increased survival of mice when combined with HSC transplantation for reconstitution of depleted and compromised bone marrow. The impact on increased host survival could extend to the use of peripheral blood and stem cell transplantations as a therapeutic option in various malignancies where curative intent is intended.

### REFERENCES

The following references are hereby incorporated by reference in their entirety.
I. G. Winkler, V. Barbier, B. Nowlan, R. N. Jacobsen, C. E. Forristal, J. T. Patton, J. L. Magnani, J. Lévesque. Vascular Niche E-selectin Regulates Hematopoietic Stem Cell Dormancy, Self-Renewal and Chemoresistance. Nature Medicine 18: 1651, 2012.
I. G. Winkler, V. Barbier, A. C. Perkins, J. L. Magnani, J. Levesque. Mobilisation of Reconstituting HSC Is Boosted by Synergy Between G-CSF and E-Selectin Antagonist GMI 1271. Blood 124: 317, 2014.
P. S. Frenette, S. Subbarao, I. B. Mazo, U. H. von Andrian, D. D. Wagner. Endothelial selectins and vascular cell adhesion molecule-1 promote hematopoietic progenitor homing to bone marrow. Proc Natl Acad Sci USA 95: 14423, 1998.
I. Oancea, C. W. Png, I. Das, R. Lourie. I. G Winkler, R. Eri, N Subramaniam, H. A. Jinnah, B. C. McWhinney, J. P. Levesque, M. A. McGuckin, J. A. Duley, T. H. J. Florin. A novel mouse model of veno-occlusive disease provides strategies to prevent thioguanine-induced hepatic toxicity Gut 62: 594, 2013.

1. A method of increasing survival of subjects that receive HSC transplantation, the method comprising administering to a subject in need thereof an effective amount of at least one E-selectin inhibitor.

2. (canceled)

3. The method according to claim 1, wherein HSC quiescence in the subject is increased.

4. The method according to claim 1, wherein HSC mobilization in the subject is increased.

5. The method according to claim 1, wherein the method further comprises inhibiting sinusoidal obstruction syndrome (SOS) in the subject.

6. The method according to claim 5, wherein the SOS is a hepatic veno-occlusive disease.

7. The method according to claim 1, wherein the at least one E-selectin inhibitor is chosen from: 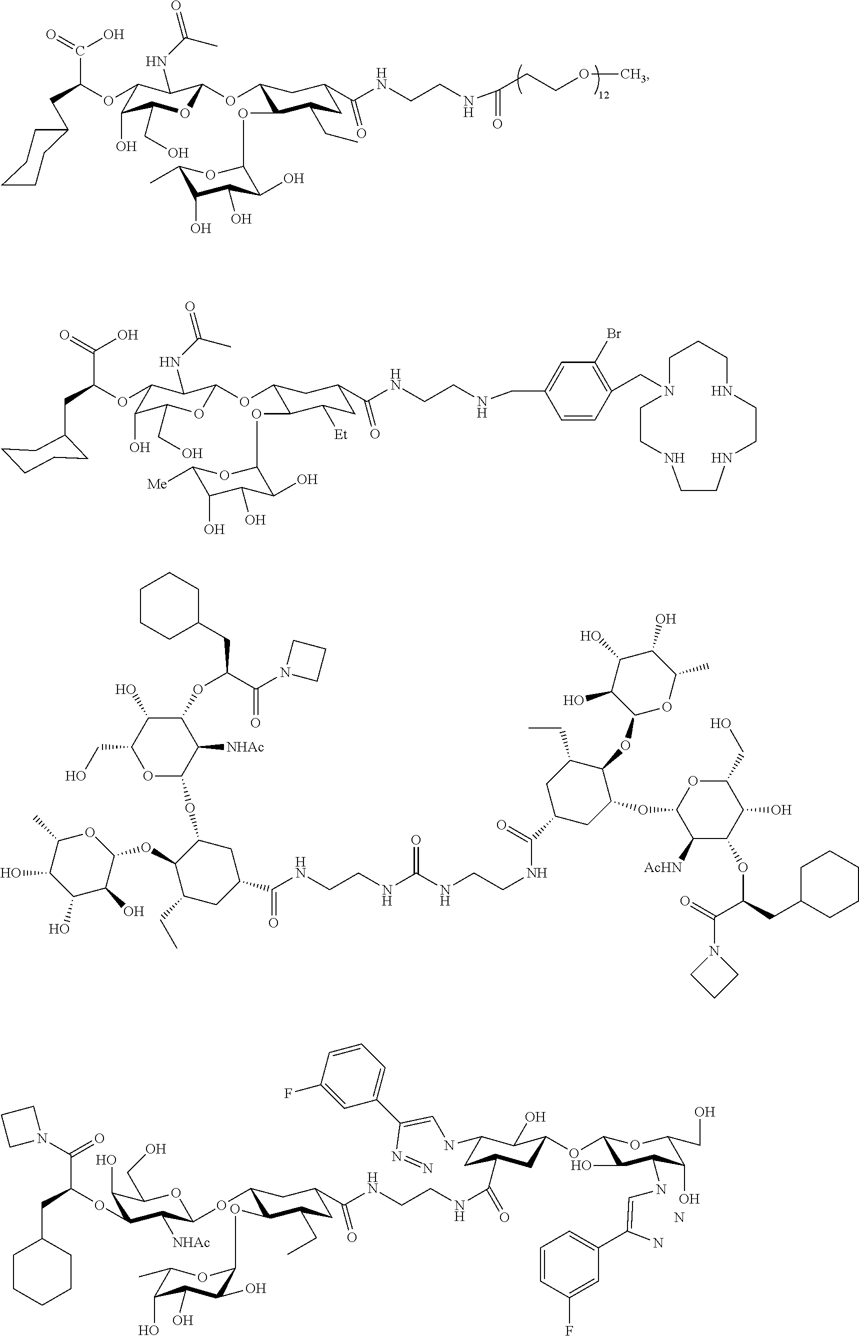
and pharmaceutically acceptable salts of any of the foregoing.

8. The method according to claim 7, wherein the at least one E-selectin inhibitor is chosen from: 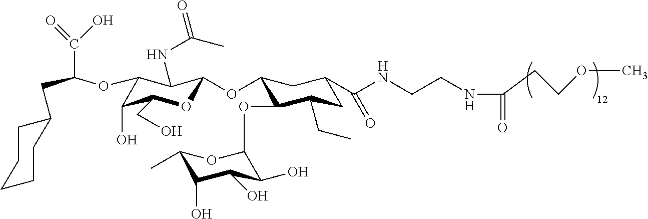
and pharmaceutically acceptable salts thereof.

9. The method according to claim 7, wherein the subject has depleted and/or compromised bone marrow.

10. The method according to claim 7, wherein the HSC transplantation is from peripheral blood.

11. The method according to claim 7, wherein the HSC transplantation is from bone marrow.

12. The method according to claim 7, wherein the subject is a transplant donor.

13. The method according to claim 7, wherein the subject is a transplant recipient.

14. The method according to claim 7, wherein the subject has received an effective amount of a granulocyte colony-stimulating factor (G-CSF).

15. The method according to claim 7, wherein the subject has a hematological disease chosen from malignant and non-malignant diseases.

16. The method according to claim 15, wherein the malignant diseases are chosen from multiple myeloma, Hodgkin and non-Hodgkin lymphoma, acute myeloid leukemia (AML), acute lymphoblastic leukemia (ALL), myelodysplastic syndrome, chronic myeloid leukemia (CML), chronic lymphocytic leukemia, myelofibrosis, essential thrombocytosis, polycythemia vera, and solid tumors.

17. The method according to claim 15, wherein the non-malignant diseases are chosen from immunodeficiency, autoimmune disorders, and genetic disorders.

18. The method according to claim 15, wherein the non-malignant diseases are chosen from aplastic anemia, severe combined immune deficiency syndrome (SCID), thalassemia, sickle cell anemia, chronic granulomatous disease, leukocyte adhesion deficiency, Chediak-Higashi syndrome, Kostman syndrome, Fanconi anemia, Blackfan-Diamond anemia, enzymatic disorders, systemic sclerosis, systemic lupus erythematosus, mucopolysaccharidosis, pyruvate kinase deficiency, and multiple sclerosis.

19. The method according to claim 1, wherein the at least one E-selectin inhibitor is chosen from: 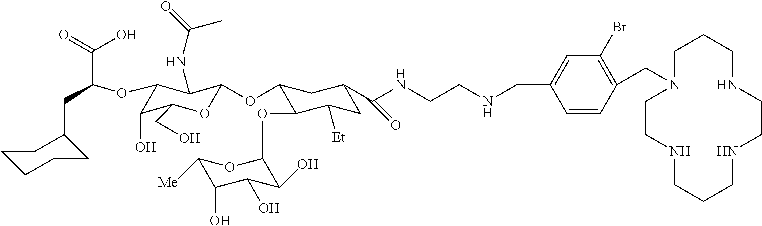 [SMILES: CCC1CC(C(=O)NCCNCc2ccc(CN3CCCNCCNCCCNCC3)c(Br)c2)CC(OC2OC(CO)C(O)C(O[C@@H](CC3CCCCC3)C(=O)O)C2NC(C)=O)C1OC1OC(C)C(O)C(O)C1O]
and pharmaceutically acceptable salts thereof.

20. The method according to claim 1, wherein the at least one E-selectin inhibitor is:

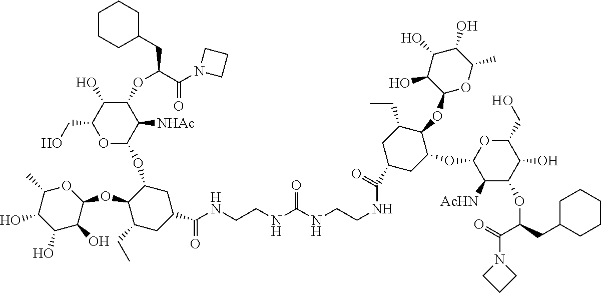

21. The method according to claim 1, wherein the at least one E-selectin inhibitor is:

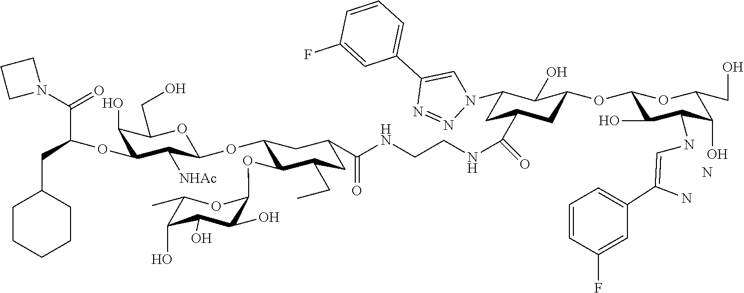

22. A method of increasing engraftment and reconstitution in a subject receiving HSC transplantation, the method comprising administering to a subject in need thereof an effective amount of at least one E-selectin inhibitor.

23. The method according to claim 22, wherein HSC quiescence in the subject is increased.

24. The method according to claim 22, wherein HSC mobilization in the subject is increased.

25. The method according to claim 22, wherein the method further comprises inhibiting sinusoidal obstruction syndrome (SOS) in the subject.

26. The method according to claim 25, wherein the SOS is a hepatic veno-occlusive disease.

27. The method according to claim 22, wherein the at least one E-selectin inhibitor is chosen from: 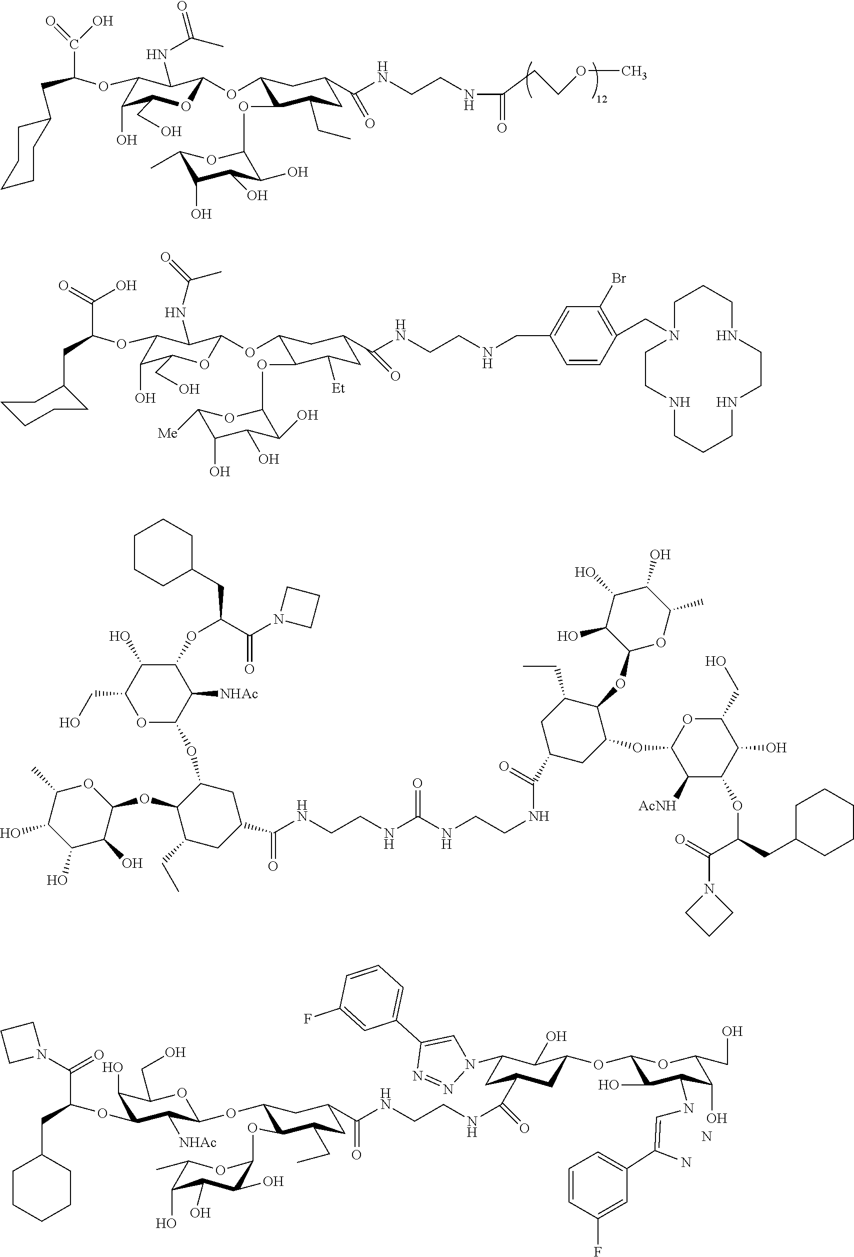
and pharmaceutically acceptable salts of any of the foregoing.

28. The method according to claim 27, wherein the at least one E-selectin inhibitor is chosen from: 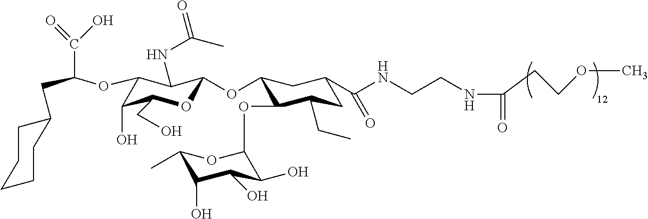
and pharmaceutically acceptable salts thereof.

29. The method according to claim 27, wherein the at least one E-selectin inhibitor is chosen from: 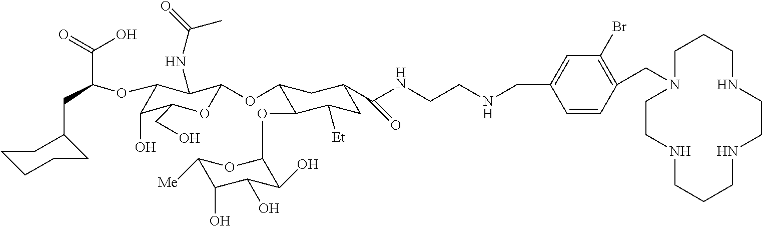 [SMILES: CCC1CC(C(=O)NCCNCc2ccc(CN3CCCNCCNCCCNCC3)c(Br)c2)CC(OC2OC(CO)C(O)C(O[C@@H](CC3CCCCC3)C(=O)O)C2NC(C)=O)C1OC1OC(C)C(O)C(O)C1O]
and pharmaceutically acceptable salts thereof.

30. The method according to claim 27, wherein the at least one E-selectin inhibitor is:

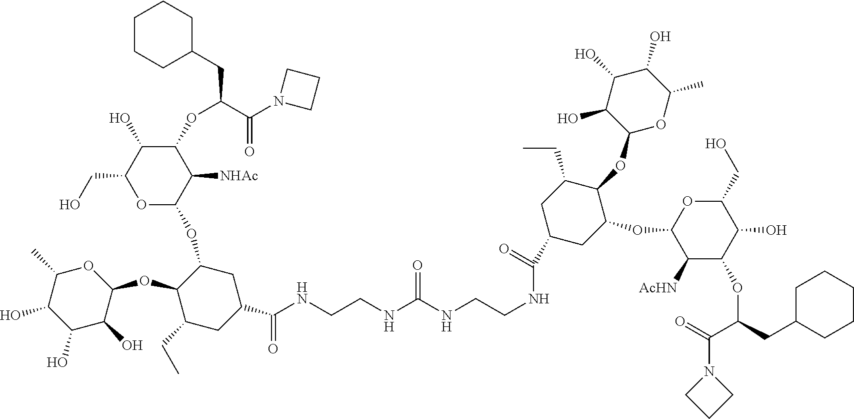

31. The method according to claim 27, wherein the at least one E-selectin inhibitor is:

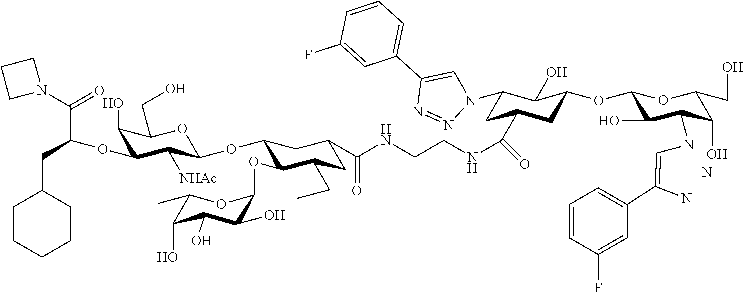

32. The method according to claim 27, wherein the subject has depleted and/or compromised bone marrow.

33. The method according to claim 27, wherein the HSC transplantation is from peripheral blood.

34. The method according to claim 27, wherein the HSC transplantation is from bone marrow.

35. The method according to claim 27, wherein the subject is a transplant donor.

36. The method according to claim 27, wherein the subject is a transplant recipient.

37. The method according to claim 27, wherein the subject has received an effective amount of a granulocyte colony-stimulating factor (G-CSF).

38. The method according to claim 27, wherein the subject has a hematological disease chosen from malignant and non-malignant diseases.

39. The method according to claim 38, wherein the malignant diseases are chosen from multiple myeloma, Hodgkin and non-Hodgkin lymphoma, acute myeloid leukemia (AML), acute lymphoblastic leukemia (ALL), myelodysplastic syndrome, chronic myeloid leukemia (CML), chronic lymphocytic leukemia, myelofibrosis, essential thrombocytosis, polycythemia vera, and solid tumors.

40. The method according to claim 38, wherein the non-malignant diseases are chosen from immunodeficiency, autoimmune disorders, and genetic disorders.

41. The method according to claim 38, wherein the non-malignant diseases are chosen from aplastic anemia, severe combined immune deficiency syndrome (SCID), thalassemia, sickle cell anemia, chronic granulomatous disease, leukocyte adhesion deficiency, Chediak-Higashi syndrome, Kostman syndrome, Fanconi anemia, Blackfan-Diamond anemia, enzymatic disorders, systemic sclerosis, systemic lupus erythematosus, mucopolysaccharidosis, pyruvate kinase deficiency, and multiple sclerosis.
